(19) United States
(12) Patent Application Publication
(10) Pub. No.: US 2019/0309271 A1
(43) Pub. Date: Oct. 10, 2019

(54) THERAPEUTIC CELL SYSTEMS AND METHODS FOR TREATING HOMOCYSTINURIA

(71) Applicant: Rubius Therapeutics, Inc., Cambridge, MA (US)

(72) Inventors: Lenka Hoffman, Watertown, MA (US); Thomas Joseph Wickham, Groton, MA (US)

(21) Appl. No.: 16/359,978

(22) Filed: Mar. 20, 2019

Related U.S. Application Data
(60) Provisional application No. 62/742,272, filed on Oct. 5, 2018.
(60) Provisional application No. 62/680,467, filed on Jun. 4, 2018.
(60) Provisional application No. 62/645,786, filed on Mar. 20, 2018.

(51) Int. Cl. C12N 9/10; C12N 9/88; A61P 3/00; A61K 35/18; A61K 35/19; C12N 5/078

(52) U.S. Cl. CPC C12N 9/1085; C12Y 205/01047; C12N 9/88; C12Y 402/01022; C12Y 404/01011; C12N 5/0644; A61P 3/00; A61K 35/18; A61K 35/19; C12N 5/0641; C12Y 404/01001

(57) ABSTRACT

The present disclosure relates to erythroid cells that have been engineered to express a homocysteine reducing polypeptide, or a variant thereof, or a homocysteine degrading polypeptide, or a variant thereof. The engineered erythroid cells may further comprise an amino acid transporter, for example a homocysteine transporter or a serine transporter, or a cystathionine degrading polypeptide. The engineered erythroid cells of the present disclosure are useful in reducing the level of homocysteine in a subject. The engineered erythroid cells of the present disclosure are further useful in methods of treating homocystinuria.

### RELATED APPLICATIONS

This application claims priority to U.S. Provisional Patent Application No. 62/645,786, filed on Mar. 20, 2018, U.S. Provisional Patent Application No. 62/680,467, filed on Jun. 4, 2018, and U.S. Provisional Patent Application No. 62/742,272, filed on Oct. 5, 2018, the entire contents of each of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Mar. 18, 2019, is named 129267-00804_SL.txt and is 391,780 bytes in size.

### BACKGROUND

Cystathionine beta-synthase (CBS), a central enzyme in the transsulfuration pathway, plays an essential role in homocysteine (Hcy) metabolism in eukaryotes (Mudd et al., 2001, in The Metabolic and Molecular Bases of Inherited Disease, 8 Ed., pp. 2007-2056, McGraw-Hill, New York). CBS catalyzes Hcy condensation with L-serine to form cystathionine. When CBS activity is dramatically reduced or absent, as a result of certain genetic mutations, Hcy builds up in tissues and blood. The CBS enzyme catalyzes a pyridoxal-5′-phosphate (PLP; Vitamin B6)-dependent condensation of serine and homocysteine to form cystathionine, which is then used to produce cysteine by another PLP-dependent enzyme, cystathionine γ-lyase. In mammalian cells that possess the transsulfuration pathway, CBS occupies a key regulatory position between the remethylation of Hcy to methionine or its alternative use in the biosynthesis of cysteine.

In healthy normal individuals, CBS-mediated conversion of Hcy to cystathionine is the rate-limiting intermediate step of methionine (Met) metabolism to cysteine (Cys). Vitamin B6 is an essential coenzyme for this process. In patients with certain genetic mutations in the CBS enzyme, the conversion of Hcy to cystathionine is slowed or absent, resulting in elevations in the serum concentrations of the enzymatic substrate (Hcy) and a corresponding decrease in the serum concentrations of the enzymatic product (cystathionine). Homocystinuria refers to a group of enzyme deficiency disorders that result in high levels of circulating homocysteine (Hcy) and its metabolite, and its concomitant excretion into the urine.

There is a wide range of reported disease prevalence, but a figure of 1:200,000 is often cited which translates into approximately 1,500-2,000 patients in the United States. Other studies based on genotyping, not clinical diagnosis, suggest a much larger but potentially asymptomatic population upwards of 100,000 patients in the United States. However, current screening, based on the level of methionine, is not particularly sensitive. Further, the symptoms of the disease, if not diagnosed in childhood, tend to mimic more common cerebrovascular and cardiovascular issues. Therefore, there is an opportunity to treat a larger population of patients.

A buildup of homocysteine results in a wide range of deforming and debilitating symptoms. Homocysteine is a highly reactive amino acid which can undergo auto-oxidation and generate reactive oxygen species which then cause lipid peroxidation and DNA damage, cellular metabolic disruption, cell death (apoptosis), and immune activation leading to atherogenesis. Untreated homocystinuria has a high rate of complications in the vasculature, connective tissue, and central nervous system. By age three, failure to thrive is generally apparent, and partial dislocation of the lens of the eyes and severe myopia are common. As with most of the inborn errors of metabolism, without treatment children may be affected by progressive and severe neurodegeneration. Many will develop psychiatric disturbances and seizures. A failure to effectively treat patients over time can also result in aberrant musculo skeletal development, including Marfanoid features (characterized by abnormally long limbs and digits) and scoliosis (spinal curvature). Perhaps most concerning, however, is that affected individuals suffer from extreme hypertension and are at an elevated risk for the development of thromboembolisms. If untreated, approximately 50% of patients will have a thromboembolic event and the overall mortality rate is approximately 20% by age 30, with death predominantly due to cerebrovascular or cardiovascular causes. It is not unusual for a previously undiagnosed individual to present in adult years with only a thromboembolic event.

The general therapeutic goal is to reduce serum and cellular Hcy accumulation and thus limit the development of existing symptoms, and prevent the onset of new symptoms. Early diagnosis, treatment and aggressive diet (methionine) restriction has been shown to slow the progression of disease as well as to reverse some of the symptoms.

Treatment practice varies widely, and compliance with diet drops with age. Precursor vitamin B6 (pyridoxine) can help to relieve some of the clinical symptoms of disease for approximately half of the patients, although a complementary moderately protein-restricted diet is necessary for these patients to achieve full metabolic control. Clinical consultants suggest that there is a continuum of response to B6 supplementation in practice and many of these patients would benefit from additional therapy. There is also the potential of overdosing which can result in severe respiratory problems in infants. B6 non-responders are subjected to a stringent protein restricted diet along with a methionine-free amino acid formulation supplement. However, their compliance with this diet is generally poor and treatment is often not successful.

The only approved medication for homocystinuria is betaine anhydrous (for oral solution) approved by the FDA in 1996 and marketed as Cystadane®. This drug provides an alternate metabolic pathway through which the body can convert homocysteine into methionine, and the product registration was based on a series of uncontrolled observational studies which suggested significant reductions in homocysteine from pre-treatment levels. Physician use of betaine varies widely in practice however, and there is preclinical data to suggest that patients tolerize to betaine supplementation and that efficacy wanes over time. Side effects of betaine supplementation are generally limited, but some patients may develop body odor and cerebral edema, which occurs as a result of hypermethioninemia. The therapy does not directly address the underlying driver of clinical disease.

Overall, treatment options for homocystinuria are limited, and there remains a need in the art for improved ways to treat homocystinuria.

### SUMMARY OF THE INVENTION

Homocysteine, which is an intermediate in the metabolism of methionine, an essential amino acid, can be metabolized by two distinct pathways: a re-methylation pathway to regenerate methionine, and a trans-sulfuration pathway, which degrades homocysteine into cysteine, and eventually into taurine. The present disclosure relates to erythroid cells, that are engineered to include a homocysteine reducing polypeptide, a homocysteine degrading polypeptide, a homocysteine transporter or a serine transporter, or any combination thereof. The engineered erythroid cells can be nucleated, e.g., erythroid precursor cells, or can be enucleated cells, e.g., reticulocytes or erythrocytes. In certain embodiments, the homocysteine reducing polypeptides reduce homocysteine levels (e.g. in the blood, plasma or serum of a subject) through the re-methylation pathway. In other embodiments, the homocysteine degrading polypeptides reduce homocysteine levels through the trans-sulfuration pathway. In some embodiments, the homocysteine degrading polypeptide functions as a replacement for a mutated or missing homocysteine degrading polypeptide in a subject, and thereby decrease homocysteine levels. Thus, the engineered erythroid cells of the present invention are useful in decreasing plasma total homocysteine levels, for example in homocystinuria. The erythroid cells that have been engineered to comprise a homocysteine degrading polypeptide are expected to overcome the limitations of existing therapies.

In some embodiments, pharmaceutical compositions comprising the engineered erythroid cells described herein are administered to a subject (e.g., a human subject) for treatment of a disease or disorder. For example, the pharmaceutical composition may be administered intravenously to the subject. In some embodiments, the engineered erythroid cells circulate for up to 120 days while shielding the homocysteine degrading polypeptide from the subject's immune system. These engineered erythroid cells may act as circulating metabolic factories to effectively replace the subject's mutated or missing enzymes, and reduce homocysteine levels in the subject.

In one aspect, the disclosure provides an erythroid cell engineered to reduce homocysteine levels, comprising a first exogenous polypeptide comprising a homocysteine reducing polypeptide, or a variant thereof. In some embodiments, the homocysteine reducing polypeptide is selected from the group consisting of methionine adenosyltransferase, alanine transaminase, L-alanine-L-anticapsin ligase, L-cysteine desulfidase, methylenetetrahydrofolate reductase, 5-methyltetrahydrofolate-homocysteine methyltransferase reductase, and methylmalonic aciduria and homocystinuria, cblD type, or variants thereof. In some embodiments, the erythroid cell when administered to a subject is capable of reducing homocysteine levels in the subject. In some embodiments, reducing homocysteine levels comprises reducing plasma total homocysteine in a subject to below about 50 μM (e.g., about 40 μM, about 30 μM, about 20 μM, about 15 μM, about 10 μM, about 5 μM, or less). In some embodiments, the erythroid cell comprises between about 100,000 to about 600,000 copies of the first exogenous polypeptide. In another embodiment, the engineered erythroid cell comprises between about 100,000-600,000, between about 100,000-500,000, between about 100,000-400,000, between about 100,000-300,000, or between 100,000-200,000 copies of the first exogenous polypeptide. In another embodiment, the engineered erythroid cell comprises at least about 100,000 copies of the first exogenous polypeptide. In another embodiment, the engineered erythroid cell comprises at least about 200,000 copies of the first exogenous polypeptide. In another embodiment, the engineered erythroid cell comprises at least about 300,000 copies of the first exogenous polypeptide. In another embodiment, the engineered erythroid cell comprises at least about 400,000 copies of the first exogenous polypeptide. In another embodiment, the engineered erythroid cell comprises at least about 500,000 copies of the first exogenous polypeptide. In some embodiments, the engineered erythroid cell is an enucleated cell.

In another aspect, the disclosure provides an erythroid cell engineered to reduce homocysteine levels, comprising a first exogenous polypeptide comprising a homocysteine degrading polypeptide, or variant thereof, wherein the homocysteine degrading polypeptide, or variant thereof, is not cystathionine beta-synthase. In some embodiments, the homocysteine degrading polypeptide, or variant thereof, is selected from the group consisting of sulfide:quinone reductase, or a variant thereof, methionine synthase, or a variant thereof, 5-methyltetrahydropteroyltriglutamate-homocysteine S-methyltransferase, or a variant thereof, adenosylhomocysteinase, or a variant thereof, cystathionine gamma-lyase, or a variant thereof, methionine gamma-lyase, or a variant thereof, L-amino-acid oxidase, or a variant thereof, thetin-homocysteine S-methyltransferase, or a variant thereof, betaine-homocysteine 5-methyltransferase, or a variant thereof, homocysteine S-methyltransferase, or a variant thereof, 5-methyltetrahydropteroyltriglutamate-homocysteine S-methyltransferase, or a variant thereof, selenocysteine Se-methyltransferase, or a variant thereof, cystathionine gamma-synthase, or a variant thereof, O-acetylhomoserine aminocarboxypropyltransferase, or a variant thereof, asparagine-oxo-acid transaminase, or a variant thereof, glutamine-phenylpyruvate transaminase, or a variant thereof, 3-mercaptopyruvate sulfurtransferase, or a variant thereof, homocysteine desulfhydrase, cystathionine beta-lyase, or a variant thereof, amino-acid racemase, or a variant thereof, methionine-tRNA ligase, or a variant thereof, glutamate-cysteine ligase, or a variant thereof, N-(5-amino-5-carboxypentanoyl)-L-cysteinyl-D-valine synthase, or a variant thereof, L-isoleucine 4-hydroxylase, or a variant thereof, L-lysine N6-monooxygenase (NADPH), or a variant thereof, methionine decarboxylase, or a variant thereof, 2,2-dialkylglycine decarboxylase (pyruvate), or a variant thereof, and cysteine synthase (CysO), or a variant thereof.

In some embodiments, the homocysteine degrading polypeptide, or variant thereof, comprises a methionine gamma-lyase, or a variant thereof. In some embodiments, the methionine gamma-lyase comprises or consists of an amino acid sequence having at least 70%, 75%, 80%, 85%, 90%, 91%, 92%, 93%, 94%, 95%, 96%, 97%, 98%, 99%, or 100% identity to an amino acid sequence selected from the group consisting of: SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46 and SEQ ID NO:47. In some embodiments, the methionine gamma-lyase is a mutated methionine gamma-lyase. In a further embodiment, the mutation comprises an amino acid substitution as compared to a wild-type amino acid sequence from which it was derived. In another further embodiment, the amino acid substitution is a C116H substitution in SEQ ID NO: 37. In some embodiments, the amino acid substitution is a C to H substitution at an amino acid corresponding to the amino acid at position 116 in SEQ ID NO: 37. In some embodiments, the methionine gamma-lyase comprises an amino acid sequence corresponding to SEQ ID NO: 47.

In some embodiments, the homocysteine degrading polypeptide comprises a cysteine synthase (CysO). In some embodiments, the CysO comprises an Aeropyrum pemix CysO, or a variant thereof. In some embodiments, the Aeropyrum pernix CysO comprises or consists of an amino acid sequence of SEQ ID NO:12.

In some embodiments, the erythroid cell when administered to a subject is capable of reducing homocysteine levels in the subject. In another embodiment, reducing homocysteine levels comprises reducing plasma total homocysteine in a subject to below about 50 μM (e.g., about 40 μM, about 30 μM, about 20 μM, about 15 μM, about 10 μM, about 5 μM, or less). In some embodiments, the engineered erythroid cell comprises between about 100,000 to about 600,000 copies of the first exogenous polypeptide, between about 100,000-600,000, between about 100,000-500,00, between about 100,000-400,000 or between about 100,000-300,000 copies of the first exogenous polypeptide. In another embodiment, the engineered erythroid cell comprises at least about 100,000 copies of the first exogenous polypeptide. In another embodiment, the engineered erythroid cell comprises at least about 200,000 copies of the first exogenous polypeptide. In another embodiment, the engineered erythroid cell comprises at least about 300,000 copies of the first exogenous polypeptide. In another embodiment, the engineered erythroid cell comprises at least about 400,000 copies of the first exogenous polypeptide. In another embodiment, the engineered erythroid cell comprises at least about 500,000 copies of the first exogenous polypeptide. In some embodiments, the engineered erythroid cell is an enucleated cell.

In another aspect, the disclosure provides an erythroid cell engineered to reduce homocysteine levels, comprising a first exogenous polypeptide comprising a cystathionine beta-synthase (CBS) polypeptide, or variant thereof. In some embodiments, the erythroid cell when administered to a subject is capable of reducing homocysteine levels in the subject. In some embodiments, reducing homocysteine levels comprises reducing total plasma homocysteine to below about 50 μM (e.g., about 40 μM, about 30 μM, about 20 μM, about 15 μM, about 10 μM, about 5 μM, or less). In some embodiments, the erythroid cell comprises between about 150,000 to about 600,000 copies of the first exogenous polypeptide. In some embodiments, the engineered erythroid cell comprises between about 150,000-600,000, between about 150,000-500,000, between about 150,000-400,000, between about 150,000-300,000, or between about 150,000-200,000 copies of the first exogenous polypeptide. In some embodiments, the engineered erythroid cell comprises at least about 150,000 copies of the first exogenous polypeptide. In some embodiments, the engineered erythroid cell comprises at least about 190,000 copies of the first exogenous polypeptide. In some embodiments, the engineered erythroid cell comprises at least about 200,000 copies of the first exogenous polypeptide. In some embodiments, the engineered erythroid cell comprises at least about 250,000 copies of the first exogenous polypeptide. In some embodiments, the engineered erythroid cell comprises at least about 300,000 copies of the first exogenous polypeptide. In some embodiments, the engineered erythroid cell comprises at least about 400,000 copies of the first exogenous polypeptide. In some embodiments, the engineered erythroid cell comprises at least about 500,000 copies of the first exogenous polypeptide. In some embodiments, the engineered erythroid cell has a homocysteine degrading activity of between about 1e-12 units/cell and about 1e-10 units/cell. In another embodiment, the engineered erythroid cell has a homocysteine degrading activity of between about 5e-12 units/cell and about 5e-11 units/cell. In some embodiments, the engineered erythroid cell has a homocysteine degrading activity of at least about 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units/cell. In some embodiments, the CBS polypeptide comprises a truncation as compared to the wild-type polypeptide from which it was derived (i.e., is a truncated cystathionine beta-synthase. In some embodiments, the CBS polypeptide lacks a C-terminal regulatory domain. In some embodiments, the CBS polypeptide lacks an N-terminal heme-binding region. In some embodiments, the truncated cystathionine beta-synthase comprises at least the proteolytically resistant core. In some embodiments, the CBS polypeptide contains at least one mutated amino acid residue (e.g., an amino acid substitution) as compared to a wild-type polypeptide from which it was derived. In some embodiments, the CBS polypeptide comprises a mutation (e.g., a substitution) of one or more cysteine residues. In some embodiments, the cystathionine beta-synthase polypeptide is selected from the group consisting of Homo sapiens cystathionine beta-synthase, Saccharomyces cerevisiae cystathionine beta synthase, Mus musculus cystathionine beta-synthase, Oryctolagus cuniculus cystathionine beta-synthase, Mycobacterium tuberculosis cystathionine beta-synthase, Rattus norvegicus cystathionine beta-synthase, Dictyostellium discoideum cystathionine beta-synthase, Drosophila melanogaster cystathionine beta-synthase, Emericella nidulan cystathionine beta-synthase, Monodelphis domestica cystathionine beta-synthase, Ornithorhynchus anatinus cystathionine beta-synthase. In some embodiments, an engineered erythroid cell described herein comprises an exogenous polypeptide, wherein the exogenous polypeptide comprises a CBS polypeptide that comprises or consists of either: a Homo sapiens cystathionine beta-synthase comprising an amino acid sequence that is at least 95% (e.g., 96%, 97%, 98%, 99%, or 100% identical) identical to the amino acid sequence set forth in SEQ ID NO:1; a Saccharomyces cerevisiae cystathionine beta-synthase comprising an amino acid sequence that is at least 95% identical (e.g., 96%, 97%, 98%, 99%, or 100% identical) to the amino acid sequence set forth in SEQ ID NO:2; a Mus musculus cystathionine beta-synthase comprising an amino acid sequence that is at least 95% identical (e.g., 96%, 97%, 98%, 99%, or 100% identical) to the amino acid sequence set forth in SEQ ID NO:3; a Oryctolagus cuniculus cystathionine beta-synthase comprising an amino acid sequence that is at least 95% identical (e.g., 96%, 97%, 98%, 99%, or 100% identical) to the amino acid sequence set forth in SEQ ID NO:4; a Mycobacterium tuberculosis cystathionine beta-synthase comprising an amino acid sequence that is at least 95% identical (e.g., 96%, 97%, 98%, 99%, or 100% identical) to the amino acid sequence set forth in SEQ ID NO:5; a Rattus norvegicus cystathionine beta-synthase comprising an amino acid sequence that is at least 95% identical (e.g., 96%, 97%, 98%, 99%, or 100% identical) to the amino acid sequence set forth in SEQ ID NO:6; a Dictyostellium discoideum cystathionine beta-synthase comprising an amino acid sequence that is at least 95% identical (e.g., 96%, 97%, 98%, 99%, or 100% identical) to the amino acid sequence set forth in SEQ ID NO:7; a Drosophila melanogaster cystathionine beta-synthase comprising an amino acid sequence that is at least 95% identical ((e.g., 96%, 97%, 98%, 99%, or 100% identical) to the amino acid sequence set forth in SEQ ID NO:8; an Emericella nidulan cystathionine beta-synthase comprising an amino acid sequence that is at least 95% identical (e.g., 96%, 97%, 98%, 99%, or 100% identical) to the amino acid sequence set forth in SEQ ID NO:9; a Monodelphis domestica cystathionine beta-synthase comprising an amino acid sequence that is at least 95% identical (e.g., 96%, 97%, 98%, 99%, or 100% identical) to the amino acid sequence set forth in SEQ ID NO:10; or an Ornithorhynchus anatinus cystathionine beta-synthase comprising an amino acid sequence that is at least 95% identical (e.g., 96%, 97%, 98%, 99%, or 100% identical) to the amino acid sequence set forth in SEQ ID NO:11.

In some embodiments, the cystathionine beta-synthase comprises an amino acid sequence that is at least 95% identical to the amino acid sequence set forth in SEQ ID NO:1. In some embodiments, the cystathionine beta-synthase is a mutated cystathionine beta-synthase as compared to the wild-type protein from which it was derived. In one embodiment, the cystathionine beta-synthase comprises or consists of the amino acid sequence set forth in SEQ ID NO: 1 with a C15S amino acid substitution. In one embodiment, the CBS polypeptide is a truncated cystathionine beta-synthase. In one embodiment, the truncated cystathionine beta-synthase comprises or consists of amino acid residues 1-413 of SEQ ID NO:1. In one embodiment, the truncated cystathionine beta-synthase comprises or consists of amino acid residues 1-413 of SEQ ID NO: 1, and a C15S amino acid substitution. In one embodiment, the truncated cystathionine beta-synthase comprises or consists of amino acid residues 40-413 of SEQ ID NO:1. In one embodiment, the truncated cystathionine beta-synthase comprises or consists of amino acid residues 1-550, 1-543, 1-533, 1-523, 1-496, 1-488, 1-441, 40-551, 71-413, 71-551, 70-413, or 70-551 of SEQ ID NO:1.

In one embodiment, the cystathionine beta-synthase is not a human cystathionine beta-synthase. In one embodiment, the cystathionine beta-synthase is not a human cystathionine beta-synthase comprising an amino acid sequence at least 80% identical (e.g., at least 80%, 85%, 90%, 95%, 96%, 97%, 98%, or 99%) to SEQ ID NO: 1. In one embodiment, the cystathionine beta-synthase is not a human cystathionine beta-synthase consisting of SEQ ID NO: 1.

In another aspect, the disclosure provides an enucleated cell engineered to reduce homocysteine levels, comprising a first exogenous polypeptide comprising a methionine gamma-lyase polypeptide, or variant thereof. In one embodiment, the enucleated cell when administered to a subject is capable of reducing homocysteine levels in the subject. In one embodiment, reducing homocysteine levels comprises reducing total plasma homocysteine to below about 50 μM (e.g., about 40 μM, about 30 μM, about 20 μM, about 15 μM, about 10 μM, about 5 μM, or less). In one embodiment, the engineered enucleated cell has a homocysteine degrading activity of between about 1e-12 units/cell and about 1e-10 units/cell as compared to a wild-type methionine gamma-lyase from which it was derived. In one embodiment, the engineered enucleated cell has a homocysteine degrading activity of between about 1e-11 units/cell and about 3e-11 units/cell. In one embodiment, the engineered enucleated cell has a homocysteine degrading activity of between about 5e-12 units/cell and about 5e-11 units/cell. In one embodiment of any of the above aspects or embodiments, the engineered enucleated cell has a homocysteine degrading activity of at least about 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units/cell. In one embodiment, the methionine gamma-lyase is a mutated methionine gamma-lyase. In one embodiment, the mutation comprises an amino acid substitution as compared to a wild-type amino acid sequence from which it was derived. In one embodiment, the amino acid substitution is a C116H substitution in SEQ ID NO: 37. In one embodiment, the amino acid substitution is a C to H substitution at an amino acid residue corresponding to the amino acid at position 116 in SEQ ID NO: 37. In one embodiment, the methionine gamma-lyase polypeptide is selected from the group consisting of: a Pseudomonas putida methionine gamma-lyase, a Saccharomyces cerevisiae methionine gamma-lyase, a Fusobacterium nucleatum methionine gamma-lyase, a Streptomyces ambofaciens methionine gamma-lyase, a Clostridium saccharobutylicum methionine gamma-lyase, a Bacillus mycoides methionine gamma-lyase, a Bordetella trematum methionine gamma-lyase, a Citrobacter freundii methionine gamma-lyase, a Entamoeba histolytica methionine gamma-lyase, a Yersinia frederiksenii methionine gamma-lyase, and a Bacillus subtilis methionine gamma-lyase. In one embodiment, the methionine gamma-lyase comprises or consists of either: a Pseudomonas putida methionine gamma-lyase comprises an amino acid sequence that is at least 95% identical (e.g., 96%, 97%, 98%, 99%, or 100% identical) to the amino acid sequence set forth in SEQ ID NO: 37; a Fusobacterium nucleatum methionine gamma-lyase comprising an amino acid sequence that is at least 95% identical (e.g., 96%, 97%, 98%, 99%, or 100% identical) to the amino acid sequence set forth in SEQ ID NO: 38; a Streptomyces ambofaciens methionine gamma-lyase comprising an amino acid sequence that is at least 95% identical (e.g., 96%, 97%, 98%, 99%, or 100% identical) to the amino acid sequence set forth in SEQ ID NO: 39; a Clostridium saccharobutylicum methionine gamma-lyase comprising an amino acid sequence that is at least 95% identical to the amino acid sequence set forth in SEQ ID NO: 40; a Bacillus mycoides methionine gamma-lyase comprising an amino acid sequence that is at least 95% identical (e.g., 96%, 97%, 98%, 99%, or 100% identical) to the amino acid sequence set forth in SEQ ID NO: 41; a Bordetella trematum methionine gamma-lyase comprising an amino acid sequence that is at least 95% identical (e.g., 96%, 97%, 98%, 99%, or 100% identical) to the amino acid sequence set forth in SEQ ID NO: 42; a Citrobacter freundii methionine gamma-lyase comprising an amino acid sequence that is at least 95% identical (e.g., 96%, 97%, 98%, 99%, or 100% identical) to the amino acid sequence set forth in SEQ ID NO: 43; an Entamoeba histolytica methionine gamma-lyase comprising an amino acid sequence that is at least 95% identical (e.g., 96%, 97%, 98%, 99%, or 100% identical) to the amino acid sequence set forth in SEQ ID NO: 44; a Yersinia frederiksenii methionine gamma-lyase comprising an amino acid sequence that is at least 95% identical to the amino acid sequence set forth in SEQ ID NO: 45; or a Bacillus subtilis methionine gamma-lyase comprising an amino acid sequence that is at least 95% identical (e.g., 96%, 97%, 98%, 99%, or 100% identical) to the amino acid sequence set forth in SEQ ID NO: 46. In one embodiment, the methionine gamma-lyase comprises an amino acid sequence that is at least 95% identical to the amino acid sequence set forth in SEQ ID NO: 47. In one embodiment, the methionine gamma-lyase is a mutated methionine gamma-lyase as compared to the wild-type methionine gamma-lyase from which it was derived. In one embodiment, the methionine gamma-lyase comprises the amino acid sequence set forth in SEQ ID NO: 37, with a C116H amino acid substitution. In some embodiments of any of the foregoing aspects, the first exogenous polypeptide is located inside of the erythroid cell.

In some embodiments of any of the foregoing aspects, the engineered erythroid cell further comprises a second exogenous polypeptide. In one embodiment, the second exogenous polypeptide is an amino acid transporter. In one embodiment, the second exogenous polypeptide is a homocysteine transporter. In one embodiment, the second exogenous polypeptide is a serine transporter. In some embodiments of any of the foregoing aspects, the engineered erythroid cell further comprises a second exogenous polypeptide comprising a homocysteine transporter and a third exogenous polypeptide comprising a serine transporter. In some embodiments, the second exogenous polypeptide is present at the surface of the erythroid cell. In some embodiments, an engineered erythroid cell described herein comprises a third exogenous polypeptide. In one embodiment, the third exogenous polypeptide is present at the surface of the erythroid cell. In one embodiment, the homocysteine transporter has a rate of transport of homocysteine into the cell of between about 1e-12 and about 1e-10 μmole/min/cell. In one embodiment, the homocysteine transporter has a rate of transport of homocysteine into the cell of between about 5e-12 and about 5e-11 μmole/min/cell. In one embodiment, the serine transporter has a rate of transport of serine into the cell of between about 1e-12 and about 1e-10 μmole/min/cell. In one embodiment, the serine transporter has a rate of transport of serine into the cell of between about 5e-12 and about 5e-11 μmole/min/cell. In some embodiments, the first exogenous polypeptide is present at a copy number of no more than 10%, 20%, 30%, 40%, 50%, 60%, 70%, 80%, or 90% greater, or no more than 2, 3, 4, 5, 6, 7, 8, 9, 10, 20, 50, 100, 200, 500, or 1000 times greater than the copy number of the second exogenous polypeptide or the third exogenous polypeptide. In some embodiments, the second exogenous polypeptide or the third polypeptide is present at a copy number of no more than 10%, 20%, 30%, 40%, 50%, 60%, 70%, 80%, or 90% greater, or no more than 2, 3, 4, 5, 6, 7, 8, 9, 10, 20, 50, 100, 200, 500, or 1000 times greater than the copy number of the first exogenous polypeptide. In some embodiments, the homocysteine or serine transporter is selected from the group consisting of: sodium-coupled neutral amino acid transporter 1 (SLC38A1) (SAT1), sodium-coupled neutral amino acid transporter 2 (SLC38A2) (SAT2), sodium-coupled neutral amino acid transporter 4 (SLC38A4) (SAT3), neutral amino acid transporter A (SLC1A4) (ASCT1), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1), excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), proton-coupled amino acid transporter 1 (SLC36A1), proton-coupled amino acid transporter 2 (SLC36A2), sodium- and chloride-dependent neutral and basic amino acid transporter B(0+) (SLC6A14), Y+L amino acid transporter 1 (SLC7A7) Y+L amino acid transporter 2 (SLC7A6), Organic anion transporter 1 (SLC22A6) (OAT1), T-type amino acid transporter (SLC16A10) (TAT1), AGT1 (SLC7A13), xCT cystine/glutamate transporter (SLC7A11), solute carrier family 13 member 3 (SLC13A3). In some embodiments, the homocysteine or serine transporter is large neutral amino acids transporter small subunit 1 (SLC7A5) (LAT1). In a further embodiment, the Homo sapiens sodium-coupled neutral amino acid transporter 1 (SLC38A1) (SAT1) comprises an amino acid sequence that is at least 95% identical (e.g., 96%, 97%, 98%, 99% or 100% identical) to the amino acid sequence set forth in SEQ ID NO:13; wherein the Homo sapiens sodium-coupled neutral amino acid transporter 2 (SLC38A2) (SAT2) comprises an amino acid sequence that is at least 95% identical (e.g., 96%, 97%, 98%, 99% or 100% identical) to the amino acid sequence set forth in SEQ ID NO:14; wherein the Homo sapiens sodium-coupled neutral amino acid transporter 4 (SLC38A4) (SAT4) comprises an amino acid sequence that is at least 95% identical (e.g., 96%, 97%, 98%, 99% or 100% identical) to the amino acid sequence set forth in SEQ ID NO:15; wherein the Homo sapiens neutral amino acid transporter A (SLC1A4) (ASCT1) comprises an amino acid sequence that is at least 95% identical (e.g., 96%, 97%, 98%, 99% or 100% identical) to the amino acid sequence set forth in SEQ ID NO:16; wherein the Homo sapiens neutral amino acid transporter B(0) (SLC1A5) (ASCT2) comprises an amino acid sequence that is at least 95% identical (e.g., 96%, 97%, 98%, 99% or 100% identical) to the amino acid sequence set forth in SEQ ID NO:17; wherein the Homo sapiens large neutral amino acids transporter small subunit 1 (SLC7A5) (LAT1) comprises an amino acid sequence that is at least 95% identical (e.g., 96%, 97%, 98%, 99% or 100% identical) to the amino acid sequence set forth in SEQ ID NO:18; wherein the Homo sapiens large neutral amino acids transporter small subunit 2 (SLC7A8) (LAT2) comprises an amino acid sequence that is at least 95% identical (e.g., 96%, 97%, 98%, 99% or 100% identical) to the amino acid sequence set forth in SEQ ID NO:19; wherein the Homo sapiens excitatory amino acid transporter 1 (SLC1A3) (EAAT1) comprises an amino acid sequence that is at least 95% identical (e.g., 96%, 97%, 98%, 99% or 100% identical) to the amino acid sequence set forth in SEQ ID NO:20; wherein the Homo sapiens excitatory amino acid transporter 2 (SLC1A2) (EAAT2) comprises an amino acid sequence that is at least 95% identical (e.g., 96%, 97%, 98%, 99% or 100% identical) to the amino acid sequence set forth in SEQ ID NO:21; wherein the Homo sapiens excitatory amino acid transporter 3 (SLC1A1) (EAAT3) comprises an amino acid sequence that is at least 95% identical (e.g., 96%, 97%, 98%, 99% or 100% identical) to the amino acid sequence set forth in SEQ ID NO:22; wherein the Homo sapiens excitatory amino acid transporter 4 (SLC1A6) (EAAT4) comprises an amino acid sequence that is at least 95% identical (e.g., 96%, 97%, 98%, 99% or 100% identical) to the amino acid sequence set forth in SEQ ID NO:23; wherein the Homo sapiens excitatory amino acid transporter 5 (SLC1A7) (EAAT5) comprises an amino acid sequence that is at least 95% identical (e.g., 96%, 97%, 98%, 99% or 100% identical) to the amino acid sequence set forth in SEQ ID NO:24; wherein the Homo sapiens 4F2 cell-surface antigen heavy chain (SLC3A2) CD98 comprises an amino acid sequence that is at least 95% identical (e.g., 96%, 97%, 98%, 99% or 100% identical) to the amino acid sequence set forth in SEQ ID NO:25; wherein the Homo sapiens sodium-coupled neutral amino acid transporter 3 (SLC38A3) (SN1) comprises an amino acid sequence that is at least 95% identical (e.g., 96%, 97%, 98%, 99% or 100% identical) to the amino acid sequence set forth in SEQ ID NO:26; wherein the Homo sapiens sodium-coupled neutral amino acid transporter 5 (SLC38A5) (SN2) comprises an amino acid sequence that is at least 95% identical (e.g., 96%, 97%, 98%, 99% or 100% identical) to the amino acid sequence set forth in SEQ ID NO:27; wherein the Homo sapiens Asc-type amino acid transporter 1 (SLC7A10) (Asc1) comprises an amino acid sequence that is at least 95% identical (e.g., 96%, 97%, 98%, 99% or 100% identical) to the amino acid sequence set forth in SEQ ID NO:28; wherein the Homo sapiens b(0,+)-type amino acid transporter 1 (SLC7A9) comprises an amino acid sequence that is at least 95% identical (e.g., 96%, 97%, 98%, 99% or 100% identical) to the amino acid sequence set forth in SEQ ID NO:29; wherein the Homo sapiens neutral and basic amino acid transport protein rBAT (SLC3A1) comprises an amino acid sequence that is at least 95% identical (e.g., 96%, 97%, 98%, 99% or 100% identical) to the amino acid sequence set forth in SEQ ID NO:30; wherein the Homo sapiens proton-coupled amino acid transporter 1 (SLC36A1) comprises an amino acid sequence that is at least 95% identical (e.g., 96%, 97%, 98%, 99% or 100% identical) to the amino acid sequence set forth in SEQ ID NO:31; wherein the Homo sapiens proton-coupled amino acid transporter 2 (SLC36A2) comprises an amino acid sequence that is at least 95% identical (e.g., 96%, 97%, 98%, 99% or 100% identical) to the amino acid sequence set forth in SEQ ID NO:32; wherein the Homo sapiens sodium- and chloride-dependent neutral and basic amino acid transporter B(0+) (SLC6A14) comprises an amino acid sequence that is at least 95% identical (e.g., 96%, 97%, 98%, 99% or 100% identical) to the amino acid sequence set forth in SEQ ID NO:33; wherein the Homo sapiens Y+L amino acid transporter 1 (SLC7A7) comprises an amino acid sequence that is at least 95% identical (e.g., 96%, 97%, 98%, 99% or 100% identical) to the amino acid sequence set forth in SEQ ID NO:34; wherein the Homo sapiens Y+L amino acid transporter 2 (SLC7A6) comprises an amino acid sequence that is at least 95% identical (e.g., 96%, 97%, 98%, 99% or 100% identical) to the amino acid sequence set forth in SEQ ID NO:35, wherein the Homo sapiens organic anion transporter 1 (SLC22A6) comprises an amino acid sequence that is at least 95% identical (e.g., 96%, 97%, 98%, 99% or 100% identical) to the amino acid sequence set forth in SEQ ID NO:48, wherein the Homo sapiens T-type amino acid transporter (SLC16A10) comprises an amino acid sequence that is at least 95% identical (e.g., 96%, 97%, 98%, 99% or 100% identical) to the amino acid sequence set forth in SEQ ID

NO:49, wherein the Homo sapiens AGT1 (SLC7A13) comprises an amino acid sequence that is at least 95% identical (e.g., 96%, 97%, 98%, 99% or 100% identical) to the amino acid sequence set forth in SEQ ID NO:51, wherein the Homo sapiens xCT cystine/glutamate transporter (SLC7A11) comprises an amino acid sequence that is at least 95% identical (e.g., 96%, 97%, 98%, 99% or 100% identical) to the amino acid sequence set forth in SEQ ID NO:52 or wherein the Homo sapiens solute carrier family 13 member 3 (SLC13A3) comprises an amino acid sequence that is at least 95% identical (e.g., 96%, 97%, 98%, 99% or 100% identical) to the amino acid sequence set forth in SEQ ID NO:61. In some embodiments, of the above aspects and embodiments, the engineered enucleated cell further comprises an exogenous polypeptide comprising a cystathionine degrading polypeptide, or a variant thereof. In some embodiments, the cystathionine degrading polypeptide is cystathionine gamma-lyase, or a variant thereof. In some embodiments, of the above aspects and embodiments, the engineered erythroid cell is an enucleated cell. In some embodiments of the above aspects and embodiments, the engineered erythroid cell is a reticulocyte.

In one embodiment, the homocysteine or serine transporter is not a human homocysteine or serine transporter. In one embodiment, the homocysteine or serine transporter is not sodium-coupled neutral amino acid transporter 2 (SLC38A2) (SAT2). In one embodiment, the homocysteine or serine transporter is not a human homocysteine or serine transporter comprising an amino acid sequence at least 80% identical (e.g., at least 80%, 85%, 90%, 95%, 96%, 97%, 98%, or 99%) to SEQ ID NO: 14. In one embodiment, the homocysteine or serine transporter is not a human homocysteine or serine transporter consisting of SEQ ID NO: 14.

In one embodiment, the homocysteine or serine transporter is not a human neutral amino acid transporter. In one embodiment, the homocysteine or serine transporter is not neutral amino acid transporter A (SLC1A4) (ASCT1). In one embodiment, the homocysteine or serine transporter is not a human neutral amino acid transporter A (SLC1A4) (ASCT1) comprising an amino acid sequence at least 80% identical (e.g., at least 80%, 85%, 90%, 95%, 96%, 97%, 98%, or 99%) to SEQ ID NO: 16. In one embodiment, the homocysteine or serine transporter is not a human homocysteine or serine transporter consisting of SEQ ID NO: 16.

In another aspect, the disclosure provides an engineered erythroid cell comprising a first exogenous polypeptide comprising a homocysteine or serine transporter, or a variant thereof. In one embodiment, the first exogenous polypeptide is presented at the surface of the engineered erythroid cell. In one embodiment, the erythroid cell comprises at least about 10,000 copies of the first exogenous polypeptide. In one embodiment, the erythroid cell comprises at least about 20,000 copies of the first exogenous polypeptide. In one embodiment, the erythroid cell comprises at least about 30,000 copies of the first exogenous polypeptide. In one embodiment, the erythroid cell comprises about 10,000-100,000 copies of the first exogenous polypeptide. In one embodiment, the homocysteine transporter transports homocysteine from outside the erythroid cell to the inside of the erythroid cell at a rate of between about 1×10e-12 to about 1×10e-10 μmole/min/cell. In one embodiment, the homocysteine transporter transports homocysteine from outside the erythroid cell to the inside of the erythroid cell at a rate of between about 5×10e-12 to about 5×10e-11 μmole/min/cell. In one embodiment, the homocysteine transporter transports homocysteine from outside the erythroid cell to the inside of the erythroid cell at a rate of at least about 1.0×10e-11 μmole/min/cell. In one embodiment, the serine transporter transports serine from outside the erythroid cell to the inside of the erythroid cell at a rate of between about 1×10e-12 to about 1×10e-10 μmole/min/cell. In one embodiment, the serine transporter transports serine from outside the erythroid cell to the inside of the erythroid cell at a rate of between about 5×10e-12 to about 5×10e-11 μmole/min/cell. In one embodiment, the serine transporter transports serine from outside the erythroid cell to the inside of the erythroid cell at a rate of at least about 1.0×10e-11 μmole/min/cell. In some embodiments, the engineered erythroid cell is a reticulocyte. In one embodiment, the homocysteine or serine transporter is selected from the group consisting of: sodium-coupled neutral amino acid transporter 1 (SLC38A1) (SAT1), sodium-coupled neutral amino acid transporter 2 (SLC38A2) (SAT2), sodium-coupled neutral amino acid transporter 4 (SLC38A4) (SAT3), neutral amino acid transporter A (SLC1A4) (ASCT1), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1), excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), proton-coupled amino acid transporter 1 (SLC36A1), Proton-coupled amino acid transporter 2 (SLC36A2), sodium- and chloride-dependent neutral and basic amino acid transporter B(0+) (SLC6A14), Y+L amino acid transporter 1 (SLC7A7), Y+L amino acid transporter 1 (SLC7A7) Y+L amino acid transporter 2 (SLC7A6), organic anion transporter 1 (SLC22A6) (OAT1), T-type amino acid transporter (SLC16A10) (TAT1), AGT1 (SLC7A13), xCT cystine/glutamate transporter (SLC7A11), solute carrier family 13 member 3 (SLC13A3). In one embodiment, the homocysteine or serine transporter is Large neutral amino acids transporter small subunit 1 (SLC7A5) (LAT1).

In some embodiments, the engineered erythroid cell is an enucleated cell, e.g. an erythrocyte or a reticulocyte

In another aspect, the disclosure features a pharmaceutical composition comprising a plurality of the engineered erythroid cells of any one of the aspects and embodiments herein, and a pharmaceutically acceptable carrier. In one embodiment, the pharmaceutical composition comprises a therapeutically effective dose of the engineered erythroid cells. In one embodiment, the pharmaceutical composition comprises between 1e10 and 1e12 engineered erythroid cells. In one embodiment, the pharmaceutical composition comprises at least 1e10, 2e10, 3e10, 4e10, 5e10, 6e10, 7e10, 8e10, 9e10, or lell cells. In one embodiment, the plurality of engineered erythroid cells have an average homocysteine degrading activity of between 1e-12 and 1e-10 units per cell. In one embodiment, the plurality of engineered erythroid cells have an average homocysteine degrading activity of between 5e-12 and 5e-11 units per cell. In one embodiment, the engineered erythroid cell is an enucleated cell.

In another aspect, the disclosure provides a method of treating or preventing homocystinuria in a subject, comprising administering to the subject the engineered erythroid cell of any one of the aspects and embodiments herein, in an amount effective to treat or prevent homocystinuria in the subject. In one embodiment, the homocystinuria is symptomatic homocystinuria. In one embodiment, the homocystinuria is asymptomatic homocystinuria.

In another aspect, the disclosure provides a method of reducing the level of homocysteine in a subject, comprising administering to the subject the engineered erythroid cell of any one of the aspects and embodiments herein, in an amount effective to reduce the level of homocysteine in the subject. In one embodiment, the level of homocysteine is total plasma homocysteine.

In another aspect, the disclosure provides a method of reducing the level of methionine in a subject, comprising administering to the subject the engineered erythroid cell of any one of the aspects and embodiments herein, in an amount effective to reduce the level of methionine in the subject. In one embodiment, the level of methionine is total plasma methionine. In one embodiment, the subject is a pediatric subject. In some embodiments, the subject has a mutation in the cystathionine beta-synthase gene or a gene that regulates the production of CBS. In one embodiment, the mutation in the CBS gene is I278T. In some embodiments, the subject has a plasma total homocysteine level greater than 50 μM prior to administering the engineered erythroid cell. In some embodiments, the subject has a plasma total homocysteine level greater than 100 μM prior to administering the engineered erythroid cell. In some embodiments, the subject has a plasma total homocysteine level greater than 500 μM prior to administering the engineered erythroid cell. In some embodiments, the plasma total homocysteine level is reduced to about 50 μM or less after administering the engineered erythroid cell to the subject. In some embodiments, the subject has not responded to precursor vitamin B6 (pyridoxine) therapy. In some embodiments, the subject has become tolerized to betaine therapy. In some embodiments, the engineered erythroid cell has a homocysteine degrading activity of between about 1e-12 units/cell and about 1e-10 units/cell, or between about 5e-12 units/cell and about 5e-11 units/cell. In one embodiment, the homocysteine degrading activity is at least about 1e-12, 2e-12, 3e-12, 4e-12, 5e-12, 6e-12, 7e-12, 8e-12, 9e-12, 1.0e-11, 1.1e-12, 1.2e-11, 1.3e-11, 1.4e-11, or 1.5e-11 units/cell. In some embodiments, the effective amount of engineered erythroid cells comprises between about 0.1 and 10 units, between about 0.5 and 5 units, or between about 1.0 and 2.0 units of homocysteine degrading activity per dose. In some embodiments, the effective amount of engineered erythroid cells comprises at least about 0.1 units, at least about 0.5 units, at least about 1 units, at least about 1.5 units, at least about 2 units, at least about 5 units, or at least about 10 units of homocysteine degrading activity per dose. In some embodiments, the subject is administered about 1×10¹⁰-1×10¹² engineered erythroid cells. In some embodiments, the subject is administered about 1×10¹¹ engineered erythroid cells. In some embodiments, the engineered erythroid cell is administered intravenously. In some embodiments, the engineered erythroid cell is administered to the subject about once every four weeks. In some embodiments, the engineered erythroid cell remains in the circulatory system of the subject for at least 60 days, 70 days, 80 days, 90 days, 100 days, 110 days or 120 days. In some embodiments, the method further comprises administration of a second agent. In one embodiment, the second agent is precursor vitamin B6 (pyridoxine). In some embodiments, the second agent is betaine. In some embodiments of the above aspects, the engineered erythroid cell is an enucleated cell, e.g., an erythrocyte or a reticulocyte.

In one aspect, the disclosure provides an engineered enucleated cell, comprising a first exogenous polypeptide comprising a homocysteine reducing polypeptide, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell, comprising a first exogenous polypeptide comprising a homocysteine degrading polypeptide, or variant thereof, wherein the homocysteine degrading polypeptide, or variant thereof, is not a cystathionine beta-synthase, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell, comprising a first exogenous polypeptide comprising a cystathionine beta-synthase (CBS) polypeptide, or variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell, comprising a first exogenous polypeptide comprising a homocysteine or serine transporter, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell, comprising a first exogenous polypeptide comprising a homocysteine reducing polypeptide, or a variant thereof, and a second exogenous polypeptide comprising an amino acid transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide and the second exogenous polypeptide.

In another aspect, the disclosure provides an engineered enucleated cell, comprising a first exogenous polypeptide comprising a homocysteine degrading polypeptide, or variant thereof, and a second exogenous polypeptide comprising an amino acid transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide and the second exogenous polypeptide.

In another aspect, the disclosure provides an engineered enucleated cell, comprising a first exogenous polypeptide comprising a cystathionine beta-synthase (CBS) polypeptide, or variant thereof, and a second exogenous polypeptide comprising an amino acid transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide and the second exogenous polypeptide. In one embodiment of the above aspects and embodiments, the second exogenous polypeptide is a homocysteine transporter or a serine transporter.

In another aspect, the disclosure provides an engineered enucleated cell, comprising at a first exogenous polypeptide comprising a homocysteine reducing polypeptide, or a variant thereof, and a second exogenous polypeptide comprising a homocysteine transporter, or a variant thereof, and a third exogenous transporter comprising a serine transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); introducing an exogenous nucleic acid encoding the third exogenous polypeptide into a nucleated erythroid cell (e.g., erythroid precursor cell); culturing the nucleated erythroid cell under conditions suitable for enucleation of the nucleated erythroid cell and for production of the first exogenous polypeptide, the second exogenous polypeptide and the third exogenous polypeptide.

In another aspect, the disclosure provides an engineered enucleated cell, comprising at a first exogenous polypeptide comprising a homocysteine degrading polypeptide, or variant thereof, and a second exogenous polypeptide comprising a homocysteine transporter, or a variant thereof, and a third exogenous transporter comprising a serine transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); introducing an exogenous nucleic acid encoding the third exogenous polypeptide into a nucleated erythroid cell (e.g., erythroid precursor cell); culturing the nucleated erythroid cell under conditions suitable for enucleation of the nucleated erythroid cell and for production of the first exogenous polypeptide, the second exogenous polypeptide and the third exogenous polypeptide.

In another aspect, the disclosure provides an engineered enucleated cell, comprising at a first exogenous polypeptide comprising a cystathionine beta-synthase (CBS) polypeptide, or variant thereof, and a second exogenous polypeptide comprising a homocysteine transporter, or a variant thereof, and a third exogenous transporter comprising a serine transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); introducing an exogenous nucleic acid encoding the third exogenous polypeptide into a nucleated erythroid cell (e.g., erythroid precursor cell); culturing the nucleated erythroid cell under conditions suitable for enucleation of the nucleated erythroid cell and for production of the first exogenous polypeptide, the second exogenous polypeptide and the third exogenous polypeptide.

In one embodiment, the exogenous nucleic acid comprises DNA or RNA. In one embodiment, the introducing step comprises viral transduction. In one embodiment, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector. In some embodiments, the introducing step comprises introducing the first exogenous nucleic acid encoding the first exogenous polypeptide by transfection of a lentiviral vector. In some embodiments, the introducing step comprises introducing the first exogenous nucleic acid encoding the first exogenous polypeptide and the second exogenous nucleic acid encoding the second exogenous polypeptide by transfection of a lentiviral vector, wherein the first exogenous nucleic acid and the second exogenous nucleic acid are contained in the same lentiviral vector. In some embodiments, the introducing step comprises introducing the first exogenous nucleic acid encoding the first exogenous polypeptide by transfection of a first lentiviral vector, and introducing the second exogenous nucleic acid encoding the second exogenous polypeptide by transfection of a second lentiviral vector. In some embodiments, the introducing step comprises introducing the first exogenous nucleic acid encoding the first exogenous polypeptide, the second exogenous nucleic acid encoding the second exogenous polypeptide, and the third exogenous nucleic acid encoding the third exogenous polypeptide, by transfection of a lentiviral vector, wherein the first exogenous nucleic acid and the second exogenous nucleic acid are contained in the same lentiviral vector. In some embodiments, the introducing step comprises introducing the first exogenous nucleic acid encoding the first exogenous polypeptide by transfection of a first lentiviral vector, introducing the second exogenous nucleic acid encoding the second exogenous polypeptide by transfection of a second lentiviral vector, and introducing the third exogenous nucleic acid encoding the third exogenous polypeptide by transfection of a third lentiviral vector. In some embodiments, the lentiviral vector comprises a promoter selected from the group consisting of: beta-globin promoter, murine stem cell virus (MSCV) promoter, Gibbon ape leukemia virus (GALV) promoter, human elongation factor 1alpha (EF1alpha) promoter, CAG CMV immediate early enhancer and the chicken beta-actin (CAG), and human phosphoglycerate kinase 1 (PGK) promoter. In some embodiments,the engineered erythroid cell comprises between about 100,000 to about 600,000 copies of the first exogenous polypeptide. In some embodiments, the engineered erythroid cell comprises at least about 200,000 copies of the first exogenous polypeptide. In some embodiments, the engineered erythroid cell comprises at least about 300,000 copies of the first exogenous polypeptide. In some embodiments, the engineered erythroid cell comprises at least about 500,000 copies of the first exogenous polypeptide. In one embodiment, the erythroid cell comprises at least about 10,000 copies of the first exogenous polypeptide. In one embodiment, the erythroid cell comprises at least about 20,000 copies of the first exogenous polypeptide. In one embodiment, the erythroid cell comprises at least about 30,000 copies of the first exogenous polypeptide. In one embodiment, the erythroid cell comprises about 10,000-100,000 copies of the first exogenous polypeptide. In some embodiments, the engineered erythroid cell comprises at least about 10,000 copies of the second exogenous polypeptide. In some embodiments, the engineered erythroid cell comprises at least about 20,000 copies of the second exogenous polypeptide. In some embodiments, the engineered erythroid cell comprises at least about 30,000 copies of the second exogenous polypeptide. In some embodiments, the engineered erythroid cell comprises at least about 10,000-100,000 copies of the second exogenous polypeptide. In some embodiments, the engineered erythroid cell comprises at least about 10,000 copies of the second exogenous polypeptide. In some embodiments, the engineered erythroid cell comprises at least about 20,000 copies of the second exogenous polypeptide. In some embodiments, the engineered erythroid cell comprises at least about 30,000 copies of the second exogenous polypeptide. In some embodiments, the engineered erythroid cell comprises at least about 10,000-100,000 copies of the second exogenous polypeptide.

In one embodiment, the invention provides an engineered erythroid cell, comprising at a first exogenous polypeptide comprising a homocysteine degrading polypeptide, or variant thereof, a second exogenous polypeptide comprising a homocysteine transporter, or a variant thereof, and/or a third exogenous transporter comprising a serine transporter, or a variant thereof. In one embodiment, the first exogenous polypeptide, the second exogenous polypeptide, and/or the third exogenous polypeptide, comprised in an engineered erythroid cell have a prolonged in vivo half-life. In one embodiment, the first exogenous polypeptide, the second exogenous polypeptide, and/or the third exogenous polypeptide have an in vivo half-life that is longer than the half-life of the first exogenous polypeptide, the second exogenous polypeptide, and/or the third exogenous polypeptide, or a pegylated version of the first exogenous polypeptide, the second exogenous polypeptide, and/or the third exogenous polypeptide, which are not comprised in an engineered erythroid cell.

In one embodiment, the first exogenous polypeptide, the second exogenous polypeptide, and/or the third exogenous polypeptide have an in vivo half-life of between about 24 hours and 60 days. In another embodiment, the first exogenous polypeptide, the second exogenous polypeptide, and/or the third exogenous polypeptide have an in vivo half-life of at least 24 hours. In another embodiment, the first exogenous polypeptide, the second exogenous polypeptide, and/or the third exogenous polypeptide have an in vivo half-life of greater than 36 hours. In another embodiment, the first exogenous polypeptide, the second exogenous polypeptide, and/or the third exogenous polypeptide have an in vivo half-life of greater than 48 hours. In another embodiment, the first exogenous polypeptide, the second exogenous polypeptide, and/or the third exogenous polypeptide have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide, the second exogenous polypeptide, and/or the third exogenous polypeptide have an in vivo half-life of 1 week, 2 weeks, 3 weeks, 1 month, 2 months, 3 months, 4 months or longer.

In some embodiments, the engineered erythroid cell comprises a first exogenous polypeptide, wherein the first exogenous polypeptide has an in vivo half-life of at least 24 hours. In another embodiment, the first exogenous polypeptide has an in vivo half-life of greater than 36 hours. In another embodiment, the first exogenous polypeptide has an in vivo half-life of greater than 48 hours. In another embodiment, the first exogenous polypeptide has an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide has an in vivo half-life of 1 week, 2 weeks, 3 weeks, 1 month, 2 months, 3 months, 4 months or longer. In one embodiment, the first exogenous polypeptide comprises a homocysteine reducing polypeptide, or a variant thereof. In another embodiment, the first exogenous polypeptide comprises a homocysteine degrading polypeptide, or a variant thereof. In another embodiment, the first exogenous polypeptide comprises a cystathionine beta-synthase (CBS) polypeptide, or variant thereof. In another embodiment, the first exogenous polypeptide comprises a homocysteine or serine transporter, or variant thereof.

In some embodiments, the engineered erythroid cell comprises a first exogenous polypeptide and further comprises a second exogenous polypeptide, wherein the first and second exogenous polypeptides have an in vivo half-life of at least 24 hours. In another embodiment, the first and second exogenous polypeptides have an in vivo half-life of greater than 36 hours. In another embodiment, the first and second exogenous polypeptides have an in vivo half-life of greater than 48 hours. In another embodiment, the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first and second exogenous polypeptides have an in vivo half-life of 1 week, 2 weeks, 3 weeks, 1 month, 2 months, 3 months, 4 months or longer. In one embodiment, the first exogenous polypeptide comprises a homocysteine reducing polypeptide, or a variant thereof. In another embodiment, the first exogenous polypeptide comprises a homocysteine degrading polypeptide, or a variant thereof. In another embodiment, the first exogenous polypeptide comprises a cystathionine beta-synthase (CBS) polypeptide, or variant thereof. In one embodiment, the second exogenous polypeptide comprises an amino acid transporter, or a variant thereof.

In some embodiments, the engineered erythroid cell comprises a first exogenous polypeptide, a second exogenous polypeptide and a third exogenous polypeptide, wherein the first exogenous polypeptide, second exogenous polypeptide and third exogenous polypeptide have an in vivo half-life of at least 24 hours. In another embodiment, the first exogenous polypeptide, second exogenous polypeptide and third exogenous polypeptide have an in vivo half-life of greater than 36 hours. In another embodiment, the first exogenous polypeptide, second exogenous polypeptide and third exogenous polypeptide have an in vivo half-life of greater than 48 hours. In another embodiment, the first exogenous polypeptide, second exogenous polypeptide and third exogenous polypeptide have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide, second exogenous polypeptide and third exogenous polypeptide have an in vivo half-life of 1 week, 2 weeks, 3 weeks, 1 month, 2 months, 3 months, 4 months or longer. In one embodiment, the first exogenous polypeptide comprises a homocysteine degrading polypeptide, or variant thereof, the second exogenous polypeptide comprises a homocysteine transporter, or a variant thereof, and/or the third exogenous transporter comprises a serine transporter, or a variant thereof.

In another embodiment, the engineered erythroid cells of the invention do not cause an immune reaction when administered to a subject. In another embodiment, the engineered erythroid cells of the invention produce a reduced immune reaction when administered to a subject as compared to the exogenous polypeptides administered without the cell.

### BRIEF DESCRIPTION OF THE DRAWINGS

FIG. 1A is a schematic that shows one method of producing cystathionine beta-synthase and homocysteine and/or serine transporter in engineered erythroid cells. In this method, the cystathionine beta-synthase and homocysteine and/or serine transporter are driven by two different promoters located on the same vector.

FIG. 1B is a schematic that shows one method of producing cystathionine beta-synthase and homocysteine and/or serine transporter in engineered erythroid cells. In this method, the T2A cleavage sequence is inserted between the cystathionine beta-synthase and homocysteine and/or serine transporter proteins.

FIG. 1C is a schematic that shows one method of producing cystathionine beta-synthase and homocysteine and/or serine transporter in engineered erythroid cells. In this method, an internal ribosome entry site (IRES) is inserted between the cystathionine beta-synthase and homocysteine and/or serine transporter genes.

FIG. 2 is a schematic that shows one method of producing cystathionine beta-synthase and homocysteine and/or serine transporter in engineered erythroid cells. In this method, cystathionine beta-synthase and homocysteine and/or serine transporter are expressed as direct peptide fusions separated by a linker.

FIG. 3A is a schematic that shows one method of producing cystathionine beta-synthase, homocysteine transporter, and serine transporter in engineered erythroid cells. In this method, the cystathionine beta-synthase, homocysteine transporter and serine transporter are driven by three different promoters located on the same vector.

FIG. 3B is a schematic that shows one method of producing cystathionine beta-synthase, homocysteine transporter, and serine transporter in engineered erythroid cells. In this method, T2A cleavage sequences are inserted between the cystathionine beta-synthase, homocysteine transporter, and serine transporter proteins.

FIG. 3C is a schematic that shows one method of producing cystathionine beta-synthase, homocysteine transporter, and serine transporter in engineered erythroid cells. In this method, internal ribosome entry sites (IRES) are inserted between the cystathionine beta-synthase, homocysteine transporter, and serine transporter genes.

FIG. 4 is a schematic that shows one method of producing cystathionine beta-synthase, homocysteine transporter, and serine transporter in engineered erythroid cells. In this method, cystathionine beta-synthase, homocysteine transporter, and serine transporter are expressed as direct peptide fusions separated by linkers.

FIG. 5 shows the presence of truncated human CBS-eGFP in a mixture of nucleated and enucleated engineered erythroid cells on differentiation day 17. The amount of CBS-eGFP in the cells was quantified using eGFP-conjugated bead standards. It was calculated that engineered erythroid cells comprising CBS-eGFP contained about 300,000 CBS-eGFP molecules per cell.

FIG. 6 is a graph that shows dose estimation (in units (U)) of erythroid cells engineered to comprise cystathionine beta-synthase and optionally homocysteine and/or serine transporter(s).

FIG. 7 is a graph that shows the measured homocystine transport into erythroid cells that were not engineered to comprise homocysteine transporter.

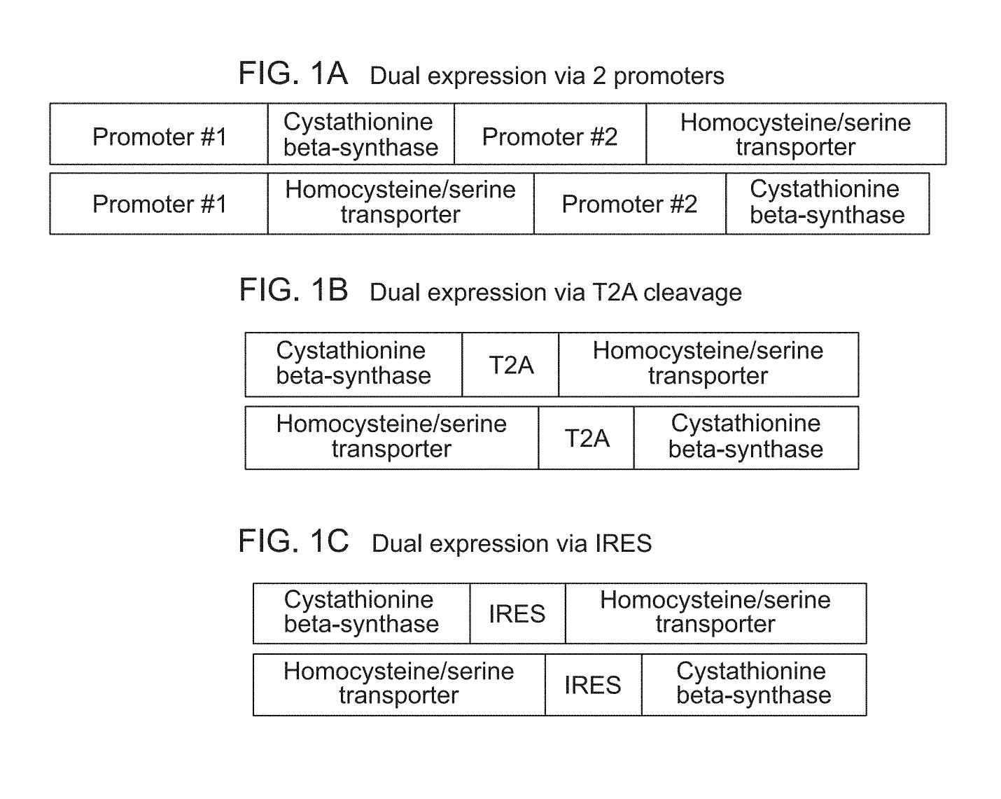

### DETAILED DESCRIPTION

The present disclosure is based on the development of cells, e.g., erythroid cells or enucleated cells, that are engineered to include a homocysteine reducing polypeptide, a homocysteine degrading polypeptide (e.g., a cystathionine beta-synthase polypeptide or a methionine gamma-lyase polypeptide), a homocysteine transporter or a serine transporter, a cystathionine degrading polypeptide, or any combination thereof. According to embodiments of the present disclosure, the homocysteine reducing polypeptide or the homocysteine degrading polypeptide is comprised inside the cell (e.g., an erythroid precursor cell). According to embodiments of the present disclosure, the cystathionine beta-synthase polypeptide or methionine gamma-lyase polypeptide is comprised inside the cell. In certain embodiments, homocysteine is effectively transported into the engineered erythroid cell or enucleated cell without the inclusion of a second exogenous polypeptide comprising a homocysteine transporter or serine transporter, e.g., the measured homocysteine transport into the enucleated cells or enucleated cells that are not engineered to comprise a homocysteine or a serine transporter is sufficiently close to a target rate (e.g., a rate of between about 1×10e-10 to about 1×10e-12 mole/min/cell, between about 1×10e-10 to about 1×10e-11 μmole/min/cell, between about 1×10e-11 to about 1×10e-12 μmole/min/cell; preferably, a rate of between about 1×10e-11 to about 3×10e-11). In other embodiments, the engineered erythroid cell or enucleated cell may comprise a second or a third exogenous polypeptide comprising a homocysteine transporter or a serine transporter, respectively, present at the surface of the engineered erythroid cell or enucleated cell. In other embodiments, the engineered erythroid cell or enucleated cell may comprise a second or a third exogenous polypeptide comprising a homocysteine transporter or a serine transporter, respectively, present at the surface of the engineered erythroid cell or enucleated cell, and a third or fourth polypeptide comprising a cystathionine degrading polypeptide. According to embodiments of the present disclosure, the engineered erythroid cells are nucleated erythroid cells, or are enucleated erythroid cells (e.g., reticuloyctes or erythrocytes)

The engineered erythroid cells of the present invention provide advantages to, for example, hypotonically loaded cells. In contrast to the erythroid cells of the present invention, which are engineered to include a homocysteine reducing polypeptide or homocysteine degrading polypeptide inside the cell and at a high copy number, a hypotonically loaded erythroid cell is limited with respect to the levels of polypeptide that may be loaded into the cell, and in addition sometimes displays aberrant physical characteristics such as increased osmotic fragility, altered cell size, reduced hemoglobin concentration, or increased phosphatidylserine levels on the outer leaflet of the cell membrane.

Moreover, the engineered erythroid cells of the invention confer a prolonged in vivo half-life to the homocysteine reducing polypeptide, the homocysteine degrading polypeptide (e.g., the cystathionine beta-synthase (CBS) polypeptide or the methionine gamma-lyase (MGL) polypeptide), the amino acid transporter, and/or the cystathionine degrading polypeptide included in the cells, as compared to the in vivo half-life of the homocysteine reducing polypeptide, the homocysteine degrading polypeptide (e.g., the cystathionine beta-synthase (CBS) polypeptide or the methionine gamma-lyase (MGL) polypeptide), the amino acid transporter, and/or the cystathionine degrading polypeptide, when either of these polypeptides are administered to a subject alone (i.e., not present in or on an erythroid cell).

In addition, the engineered erythroid cells of the invention may not cause an immune reaction when administered to a subject, or may produce a reduced immune reaction when administered to a subject as compared to the immune reaction caused by the same exogenous polypeptides when administered to a subject without the cell. Without wishing to be bound by any particular theory, it is believed that a reduced immune reaction may result from the shielding or protection that the erythroid cells confer to the exogenous polypeptide against antibodies within a subject, thereby allowing the activity (e.g., enzymatic activity) of the one or more exogenous polypeptides to be preserved in vivo.

The disclosure provides, in some embodiments, engineered cells (e.g., enucleated erythroid cells) comprising exogenous polypeptides comprising a cystathionine beta-synthase and/or a cystathionine gamma-lyase. Both cystathionine beta-synthase and a cystathionine gamma-lyase are responsible for the production of hydrogen sulfide (H₂S) in mammalian cells (see, e.g., Yang et al. (2004) J. Biol. Chem. 279:49199-205, incorporated herein by reference). Hydrogen sulfide inhibits cell proliferation and may induce cell death, and overexpression of cystathionine gamma lyase in mammalian cells has been shown to inhibit cell proliferation (Yang et al. (2004)). Notwithstanding the foregoing, surprisingly, erythroid precursor cells are capable of proliferating and differentiating into enucleated erythroid cells (e.g., reticulocytes or erythrocytes) despite being genetically modified to express exogenous polypeptides comprising a cystathionine beta-synthase and/or a cystathionine gamma-lyase.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, encompasses variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, an “additional therapeutic” refers to any therapeutic that is used in addition to another treatment. For example, when the method is one directed to treatment with the engineered erythroid cells described herein, and the method comprises the use of an additional therapeutic, the additional therapeutic is in addition to the engineered erythroid cells described herein. Generally, the additional therapeutic will be a different therapeutic. The additional therapeutic may be administered at the same time or at a different time and/or via the same mode of administration or via a different mode of administration, as that of the other therapeutic. In preferred embodiments, the additional therapeutic will be given at a time and in a way that will provide a benefit to the subject during the effective treatment window of the other therapeutic. When two compositions are administered with a specific time period, generally the time period is measured from the start of the first composition to the start of the second composition. As used herein, when two compositions are given within an hour, for example, the time before the start of the administration of the first composition is about an hour before the start of the administration of the second composition. In some embodiments, the additional therapeutic is another therapeutic for the treatment of homocystinuria, or a condition associated with elevated levels of homocysteine.

As used herein, “dose” refers to a specific quantity of a pharmacologically active material for administration to a subject for a given time. Unless otherwise specified, the doses recited refer to an engineered erythroid cell comprising a homocysteine reducing polypeptide or homocysteine degrading polypeptide as described herein, or an engineered erythroid cell comprising a homocysteine reducing polypeptide or homocysteine degrading polypeptide and a homocysteine transporter and/or a serine transporter, as described herein. In one embodiment, a dose of engineered erythroid cells refers to an effective amount of engineered erythroid cells. In one embodiment, a dose or effective amount of engineered erythroid cells refers to about 1×10¹⁰-1×10¹² engineered erythroid cells, or about 1×10¹¹engineered erythroid cells per dose. In one embodiment, a dose or effective amount of engineered erythroid cells comprises between about 0.1 and 10 units, or between 0.5 and 5 units, or between about 1.0 and 2.0 units of homocysteine degrading activity per dose. In one embodiment, a dose or effective amount of engineered erythroid cells comprises at least about 0.1, 0.5, 1, 1.5, 2, 5 or 10 units of homocysteine degrading activity per dose. In one embodiment, the engineered erythroid cells have a homocysteine degrading activity of between about 1e-12 units/cell and about 1e-10 units/cell, or between about 5e-12 units/cell and about 1e-11 units/cell, for example at least 1e-12 units/cell, at least 5e-12 units/cell, at least 1e-11 units/cell, at least 1.5e-11 units/cell. When referring to a dose for administration, in an embodiment of any one of the methods, compositions or kits provided herein, any one of the doses provided herein is the dose as it appears on a label/label dose.

As used herein, the term “endogenous” is meant to refer to a native form of compound (e.g., a small molecule) or process. For example, in some embodiments, the term “endogenous” refers to the native form of a nucleic acid or polypeptide in its natural location in the organism or in the genome of an organism.

As used herein, the term “an engineered cell” is meant to refer to a genetically-modified cell or progeny thereof. In some embodiments, an engineered cell (e.g. an engineered enucleated cell) can be produced using coupling reagents to link an exogenous polypeptide to the surface of the cell (e.g., using click chemistry).

As used herein, an “homocysteine level” refers to a concentration of homocysteine in the blood or a blood fraction (e.g., serum or plasma of a subject (e.g. a mammal (human or animal)).

As used herein, an “increased homocysteine level” refers to a concentration of homocysteine that is increased relative to the normal or average concentration of homocysteine for that subject. For example, and depending on the context, an “elevated homocysteine level” refers to a concentration of homocysteine in the blood or blood fraction, e.g., serum or plasma of a subject that is (1) higher than the concentration of homocysteine in the blood or blood fraction of an average subject (i.e., a hypothetical subject having the average concentration of homocysteine for individuals of the same species, gender and age); (2) higher than the blood homocysteine level in the upper tertile for control subjects of the same species, gender and age;

and/or (3) higher than the average homocysteine blood levels of normal or control subjects of the same species, gender and age. An “increased homocysteine level” may be at least at least 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70% or more above the level of homocysteine in the blood or blood fraction, e.g., serum or plasma of an average or control subject. Whether or not a subject has elevated homocysteine levels can be determined by a clinician, and in some embodiments, the subject is one in which a clinician has identified or would identify as having elevated homocysteine levels.

As used herein, a “decreased homocysteine level” refers to a concentration of homocysteine that is decreased relative to a level of homocysteine that is indicative of a metabolic disorder of homocysteine metabolism (e.g. hyprocystinuria). For example, and depending on the context, a “decreased homocysteine level” refers to a concentration of homocysteine in the serum or plasma of a subject that is (1) lower than the concentration of homocysteine in the blood or blood fraction of a subject with a metabolic disorder of homocysteine metabolism. A “decreased homocysteine level” may be at least at least 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70% or more below the level of homocysteine in the serum or plasma of a subject with a metabolic disorder of homocysteine metabolism.

As used herein, the term “effective amount” refers to that amount of an engineered erythroid cell effective to produce the intended pharmacological, therapeutic or preventive result. For example, if a given clinical treatment is considered effective when there is at least a 25% reduction in a disease symptom, a therapeutically effective amount is the amount necessary to effect at least a 25% reduction in that parameter. The exact amount required will vary from subject to subject, depending on the species, age, general condition of the subject, the particular delivery form, bioavailability, and the like.

As used herein, the term “enucleated” refers to a cell, e.g., a reticulocyte or mature red blood cell (erythrocyte) that lacks a nucleus. In some embodiments an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In some embodiments an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell. In some embodiments an enucleated cell lacks DNA. In some embodiments an enucleated cell is incapable of expressing a polypeptide, e.g., incapable of transcribing and/or translating DNA into protein, e.g., lacks the cellular machinery necessary to transcribe and/or translate DNA into protein. In some embodiments, an enucleated cell is an erythrocyte, a reticulocyte, or a platelet.

In some embodiments, the enucleated cells are not platelets, and therefore are “platelet free enucleated” cells (“PFE” cells). It should be understood that platelets do not have nuclei, and in this particular embodiment, platelets are not intended to be encompassed.

As used herein, “erythroid cell” includes a nucleated red blood cell, a red blood cell precursor, an enucleated mature red blood cell, and a reticulocyte. As used herein, an erythroid cell includes an erythroid precursor cell, a cell capable of differentiating into a reticulocyte or erythrocyte. For example, erythroid precursor cells include any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid precursor cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al., (2014) Mol. Ther. 22(2): 451-63, the entire contents of which are incorporated by reference herein). In addition, the cells may be intended for autologous use or provide a source for allogeneic transfusion. In some embodiments, erythroid cells are cultured. In some embodiments an erythroid cell is an enucleated red blood cell.

As used herein, the term “exogenous,” when used in the context of nucleic acid, includes a transgene and recombinant nucleic acids.

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell, or a progenitor of the cell, which nucleic acid is optionally not retained by the cell. In some embodiments, an exogenous polypeptide is a polypeptide conjugated to the surface of the cell by chemical or enzymatic means.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, the terms “first” and “second”, and “third” with respect to exogenous polypeptides are used for convenience of distinguishing when there is more than one type of exogenous polypeptide. Use of these terms is not intended to confer a specific order or orientation of the exogenous polypeptides unless explicitly so stated.

As used herein, the term “fragment” refers to sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence. Fragments may be derived from any contiguous portion of a nucleic acid or amino acid sequence of choice.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein, the term “homocysteine” or “Hcy” refers to a compound with the empirical formula: C₄H₉NO₂S and molecular weight of 135.18, and CAS Number 454-28-4. Biologically, homocysteine is produced by demethylation of methionine and is an intermediate in the biosynthesis of cysteine from methionine. The term “homocysteine” encompasses free homocysteine (in the reduced form) and conjugated homocysteine (in the oxidized form). Homocysteine can conjugate with proteins, peptides, itself or other thiols through a disulfide bond. Homocysteine can conjugate through a disulfide bond to form a dimer, i.e. disulfide homocystine (Hcy-S—S-Hcy)), which is rapidly reduced to homocysteine under reducing conditions (e.g. inside of a cell). In human serum, both homocysteine and homocystine are generally present, and a large fraction of the homocysteine in serum is bound to proteins.

As used herein, a “homocysteine transporter” refers to a membrane transport protein that transports free homocysteine (in the reduced monomeric form) and/or homocystine (homocysteine in the dimeric oxidized form) across the cell membrane. Preferably the homocysteine transporter effectively increase the amount or concentration of homocysteine in the cell. In some embodiments the transporter is specific for homocysteine (the reduced monomeric form). In some embodiments, the transporter is specific for homocystine (the dimeric oxidized form).

As used herein, a “homocysteine reducing polypeptide” refers to any polypeptide, or variant thereof, that, when administered to a subject (e.g., comprised within or on an erythroid cell) has the effect of reducing the level of homocysteine, or any one or more of its metabolites in the subject, e.g., in the plasma or serum of the subject. As used herein, a homocysteine reducing polypeptide does not utilize homocysteine as a substrate, i.e., does not include a “homocysteine degrading polypeptide” as used herein. In some embodiments, homocysteine metabolites include, e.g., disulfide homocystine (Hcy-S—S-Hcy), mixed disulfide of Hcy and Cys (Hcy-S—S-Cys), mixed disulfide of Hcy with plasma protein (S-Hcy-protein), Hcy-thiolactone, N-Hcy-protein, Nε-Hcy-Lys, AdoHcy, cystathionine, homocysteine sulfinic acid, homocysteic acid, and methionine.

As used herein, a “homocysteine degrading polypeptide” refers to any polypeptide, or variant thereof, that utilizes homocysteine as a substrate and converts homocysteine to a metabolite or degradation product of homocysteine.

As used herein, “homocysteine degrading activity” refers to the activity of degradation of homocysteine. Homocysteine degrading activity is measured in units, where one unit of activity is defined as degradation of 1 umol of homocysteine per minute.

As used herein, the term “cystathionine” refers to an intermediate in the synthesis of cysteine. Cystathionine is produced by the transsulfuration pathway which converts homocysteine into cystathionine. Cystathionine can then be utilized as a substrate by the enzyme cystathionine gamma-lyase (CTH).

As used herein, a “cystathionine degrading polypeptide” refers to any polypeptide, or variant thereof, that utilizes cystathionine as a substrate and converts cystathionine into one or more metabolites or degradation products of cystathionine. In some embodiments, a cystathionine degrading polypeptide converts cystathionine into cysteine, α-ketobutyrate, and ammonia. In some embodiments, the cystathionine degrading polypeptide is cystathionine gamma-lyase.

As used herein the term “nucleic acid molecule” refers to a single or double-stranded polymer of deoxyribonucleotide or ribonucleotide bases. It includes chromosomal DNA and self-replicating plasmids, vectors, mRNA, tRNA, siRNA, etc. which may be recombinant and from which exogenous polypeptides may be expressed when the nucleic acid is introduced into a cell.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.” (a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence. (b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995). Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination. (c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA). (d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. (e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. Mutations may also be made to the nucleotide sequences of the present proteins by reference to the genetic code, including taking into account codon degeneracy.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered agent.

As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. According to some embodiments, the peptide is of any length or size.

As used herein, polypeptides referred to herein as “recombinant” refers to polypeptides which have been produced by recombinant DNA methodology, including those that are generated by procedures which rely upon a method of artificial recombination, such as the polymerase chain reaction (PCR) and/or cloning into a vector using restriction enzymes.

“Recombinant” polypeptides are also polypeptides having altered expression, such as a naturally occurring polypeptide with recombinantly modified expression in a cell, such as a host cell.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in particular embodiments the subject is a human.

As used herein, the phrase “subject in need” refers to a subject that (i) will be administered an engineered erythroid cell (or pharmaceutical composition comprising an engineered erythroid cell) according to the described invention, (ii) is receiving an engineered erythroid cell (or pharmaceutical composition comprising an engineered erythroid cell) according to the described invention; or (iii) has received an engineered erythroid cell (or pharmaceutical composition comprising an engineered erythroid cell) according to the described invention; or (iv) is in need of and/or would benefit from administration of an engineered erythroid cell (or pharmaceutical composition comprising an engineered erythroid cell) according to the described invention, unless the context and usage of the phrase indicates otherwise

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “therapeutic amount”, “therapeutically effective amount”, an “amount effective”, or “pharmaceutically effective amount” of an active agent (e.g. an engineered erythroid cell as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “variant” (e.g., a mutant) refers to a polypeptide which differs from the original protein from which it was derived (e.g., a wild-type protein) by one or more amino acid substitutions, deletions, insertions, or other modifications. In some embodiments, these modifications do not significantly change the biological activity of the original protein. Such changes include, but are not limited to: changes in one, few, or even several amino acid side chains; changes in one, few or several amino acids; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A variant can have enhanced, decreased, changed, or essentially similar properties as compared to the naturally occurring protein or peptide. In many cases, a variant retains at least 10%, 20%, 30%, 40%, 50%, 60%, 70%, 80%, 90%, 95%, or 100% of the biological activity of original protein. The biological activity of a variant can also be higher than that of the original protein. A variant can be naturally-occurring, such as by allelic variation or polymorphism, or be deliberately engineered. For example, a variant may comprise a substitution at one or more amino acid residue positions to replace a naturally-ocurring amino acid residue for a structurally similar amino acid residue. Structurally similar amino acids include: (I, L and V); (F and Y); (K and R); (Q and N); (D and E); and (G and A). In some embodiments, variants include (i) polymorphic variants and natural or artificial mutants, (ii) modified polypeptides in which one or more residues is modified, and (iii) mutants comprising one or more modified residues.

The amino acid sequence of a variant is substantially identical to that of the original protein. In many embodiments, a variant shares at least 50%, 60%, 70%, 75%, 80%, 85%, 90%, 91%, 92%, 93%, 94%, 95%, 96%, 97%, 98%, or 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm) The amino acid sequences of a variant and the original protein can be substantially identical in one or more regions, but divergent in other regions. A variant may include a fragment (e.g., a biologically active fragment of a polypeptide). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide.

### I. Engineered Cells

The present disclosure relates to cells (e.g., erythroid cells and enucleated cells) that are engineered to include an exogenous polypeptide comprising at least one of: a homocysteine reducing polypeptide, a homocysteine degrading polypeptide, a homocysteine transporter or a serine transporter, or any combination thereof. In some embodiments an enucleated cell is a erythroid cell, for example, that has lost its nucleus through differentiation from an erythroid precursor cell. It will be understood, however, that not all enucleated cells are erythroid cells and, accordingly, enucleated cells encompassed herein can also include, e.g., platelets. In some embodiments, populations of enucleated cells that do not include platelets are provided, and are therefore a population of platelet-free enucleated cells. In certain aspects of the disclosure, the erythroid cell is a reticulocyte or an erythrocyte (e.g., fully mature red blood cell (RBC)). Erythrocytes offer a number of advantages over other cells, including being non-autologous due to lack of major histocompatibility complex (MHC), having long circulation time, and being amenable to production in large numbers. In certain aspects of the disclosure, the engineered erythroid cells are nucleated. In certain embodiments of the disclosure, the engineered erythroid cells are nucleated.

### Engineered Erythroid Cells and Enucleated Cells

The present disclosure features erythroid cells and enucleated cells that are engineered to reduce homocysteine levels. In some embodiments an enucleated cell is a erythroid cell, for example, that has lost its nucleus through differentiation from an erythroid precursor cell. It will be understood, however, that not all enucleated cells are erythroid cells and, accordingly, enucleated cells encompassed herein can also include, e.g., platelets. In some embodiments, enucleated cells are not platelets and are therefore platelet free enucleated cells. In certain aspects of the disclosure, the erythroid cell is a reticulocyte or an erythrocyte (red blood cell (RBC)). Erythrocytes offer a number of advantages over other cells, including being non-autologous due to lack of major histocompatibility complex (MHC), having longer circulation time, and being amenable to production in large numbers. In certain aspects of the disclosure, the engineered erythroid cells are nucleated.

The engineered cells may be advantageously used to reduce homocysteine in the milieu surrounding the cell (e.g., in vitro or in vivo). For example, the engineered cells provided herein may be administered to a subject (e.g., a human subject) to reduce homocysteine levels in the subject (e.g., in the blood, plasma, or serum of the subject). Any condition, disease or disorder in which a reduction of homocysteine levels is desired may be treated by administering the engineered cells provided herein. In one aspect of the disclosure, an erythroid cell engineered to reduce homocysteine levels comprises a first exogenous polypeptide comprising a homocysteine reducing polypeptide, or a variant thereof. In one aspect of the disclosure, an erythroid cell engineered to reduce homocysteine levels comprises a homocysteine degrading polypeptide, or variant thereof, wherein the homocysteine degrading polypeptide, or variant thereof, is not a cystathionine beta-synthase. In one aspect of the disclosure, an erythroid cell engineered to reduce homocysteine levels comprises a first exogenous polypeptide comprising a cystathionine beta-synthase (CBS) polypeptide, or variant thereof. In one aspect of the disclosure, an erythroid cell engineered to reduce homocysteine levels comprises a first exogenous polypeptide comprising a homocysteine or serine transporter, or a variant thereof.

In some embodiments of any of the aspects herein, the engineered erythroid cell is a reticulocyte. In some embodiments of any of the aspects herein, the engineered erythroid cell is an erythrocyte.

### Homocysteine and Homocysteine Metabolites

Homocysteine (Hcy) is a thiol group-containing amino acid metabolite formed in the methionine (Met) cycle (Selhub, Annu Rev Nutr. 1999; 19:217-246). As a branch point metabolite, Hcy can be remethylated back to methionine or converted to cysteine through the transsulphuration pathway (Selhub 1999). Perturbation of these metabolic pathways leads to hyperhomocysteinaemia (HHcy), a condition in which the plasma concentration of total Hcy (tHcy), comprising reduced and oxidized forms of Hcy, is elevated. Plasma tHcy is regulated by several factors including nutritional deficiencies in the vitamins that act as co-factors or co-substrates in Hcy metabolism (folate, vitamins B₁₂ and B₆), or genetic defects in the enzymes responsible for Hcy metabolism (Refsum et al. Annu Rev Med. 1998; 49:31-62; and Selhub 1999).

In some embodiments, homocysteine metabolites include, e.g., disulfide homocystine (Hcy-S—S-Hcy), mixed disulfide of Hcy and Cys (Hcy-S—S-Cys), mixed disulfide of Hcy with plasma protein (S-Hcy-protein), Hcy-thiolactone, N-Hcy-protein, Nε-Hcy-Lys, AdoHcy, cystathionine, homocysteine sulfinic acid, homocysteic acid, and methionine.

### Homocysteine Reducing Polypeptides

In one aspect, the present disclosure provides an erythroid cell engineered to reduce homocysteine levels, comprising a first exogenous polypeptide comprising a homocysteine reducing polypeptide, or a variant thereof.

In one embodiment, the erythroid cell comprises more than one (e.g., two, three, four, five, or more) exogenous polypeptides, each comprising a homocysteine reducing polypeptide, or variants thereof. Exogenous polypeptides comprising any one or more of the enzymes involved in homocysteine conversion or catalysis can be included in the erythroid cells described herein. In some embodiments, the engineered cells described herein comprising more than one type of exogenous polypeptide, wherein each exogenous polypeptide comprises a homocysteine reducing polypeptide, and the homocysteine reducing polypeptides are not the same (e.g., the homocysteine reducing polypeptides may be different types of polypeptides or variants of the same type of polypeptide).

In another embodiment, the erythroid cell may comprise an exogenous polypeptide comprising a homocysteine degrading polypeptide, such as cystathionine beta-synthase or a variant thereof, and an exogenous polypeptide comprising a homocysteine reducing polypeptide, or a variant thereof.

In another embodiments, the erythroid cell can comprise a homocysteine degrading polypeptide, such as the methionine gamma-lyase polypeptide, or a variant thereof, and a homocysteine reducing polypeptide, or a variant thereof.

In one embodiment, the engineered cells described herein may comprise at least one exogenous polypeptide, wherein the exogenous polypeptide comprises at least one (e.g., two, three, four, or more) homocysteine reducing polypeptides, or variants thereof. Any homocysteine reducing polypeptide, or variant thereof, that, when administered to a subject (e.g., comprised within an erythroid cell) has the effect of reducing the level of homocysteine, or any one or more of its metabolitess, may be used as described herein. The homocysteine reducing polypeptides, or variants thereof, can be derived from any source or species, e.g., mammalian, fungal (including yeast), plant or bacterial sources, or can be recombinantly engineered. For example, the homocysteine reducing polypeptide may be a chimeric homocysteine reducing polypeptide, e.g., derived from two different species. In some embodiments, the engineered cells provided herein comprise at least one exogenous polypeptide comprising a enzymatically-active fragment or truncation of homocysteine reducing polypeptide.

The exogenous polypeptides included in the engineered cells provided herein may comprise any homocysteine reducing polypeptide. Multiple homocysteine reducing polypeptides are known in the art and may be used. For example, in one embodiment, the homocysteine reducing polypeptide is a methionine adenosyltransferase (EC 2.5.1.6), or a variant thereof. In another embodiment, the homocysteine reducing polypeptide is an alanine transaminase (EC 2.6.1.2), or a variant thereof. In another embodiment, the homocysteine reducing polypeptide is a L-alanine-L-anticapsin ligase (EC 6.3.2.49), or a variant thereof. In another embodiment, the homocysteine reducing polypeptide is a L-cysteine desulfidase (E.C. 4.4.1.28), or a variant thereof. In another embodiment, the homocysteine reducing polypeptide is a methylenetetrahydrofolate reductase (MTHFR; EC 1.5.1.20), or a variant thereof. In another embodiment, the homocysteine reducing polypeptide is a 5-methyltetrahydrofolate-homocysteine methyltransferase reductase (EC 1.16.1.8; MTRR), or a variant thereof. In another embodiment, the homocysteine reducing polypeptide is a methylmalonic aciduria and homocystinuria, cblD type (MMADHC), or a variant thereof.

In some embodiments, the exogenous polypeptide included in the engineered cells described herein comprise a homocysteine reducing polypeptide that is a variant of a wild-type homocysteine reducing polypeptide, wherein the variant comprises an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type homocysteine reducing polypeptide (e.g., a wild-type methionine adenosyltransferase, alanine transaminase, L-alanine-L-anticapsin ligase, or L-cysteine desulfidase enzyme).

### Homocysteine Degrading Polypeptides

In some embodiments, the engineered cells provided herein comprise at least two exogenous polypeptides each comprising a homocysteine degrading polypeptide, wherein the homocysteine degrading polypeptides are not the same (e.g. not of the same type or are variants of the same type of homocysteine degrading polypeptide). For example, an erythroid cell provided herein may comprise, in one embodiment, a first exogenous polypeptide comprising a homocysteine degrading polypeptide comprising a cystathionine beta-synthase, or a variant thereof, and a second exogenous polypeptide comprising a homocysteine degrading polypeptide that is not a cystathionine beta-synthase.

In some embodiments, the erythroid cell can comprise an exogenous polypeptide comprising a homocysteine degrading polypeptide, such as cystathionine beta-synthase or a variant thereof, and an exogenous polypeptide comprising a homocysteine reducing polypeptide, or a variant thereof. In some embodiments, an erythroid cell may comprise an exogenous polypeptide, wherein the exogenous polypeptide comprises both a homocysteine degrading polypeptide and a homocysteine reducing polypeptide. In some embodiments, an erythroid cell may comprise an exogenous polypeptide comprising two or more copies of the same homocysteine reducing polypeptide. In some embodiments, an erythroid cell may comprise an exogenous polypeptide comprising two or more homocysteine degrading polypeptides (e.g., of the same type (e.g., identical or variants of each other) or of different types).

In another embodiment, the erythroid cell may comprise an exogenous polypeptide comprising a homocysteine degrading polypeptide, such as the methionine gamma-lyase polypeptide, or a variant thereof, and an exogenous polypeptide comprising a homocysteine reducing polypeptide, or a variant thereof.

The homocysteine degrading polypeptides, or variants thereof, can be derived from any source or species, e.g., mammalian, fungal (including yeast), plant or bacterial sources, or can be recombinantly engineered. In some embodiments, the homocysteine degrading polypeptide may be a chimeric homocysteine degrading polypeptide, e.g., derived from two different species. In some embodiments, the engineered cells provided herein comprise at least one exogenous polypeptide comprising a enzymatically-active fragment or truncation of homocysteine degrading polypeptide. The exogenous polypeptides included in the engineered cells provided herein may comprise any homocysteine degrading polypeptide. Multiple homocysteine degrading polypeptides are known in the art and may be used. In one embodiment, the homocysteine degrading polypeptide is a cystathionine beta-synthase, or a variant thereof. In another embodiment, the homocysteine degrading polypeptide is a methionine gamma-lyase (E.C. 4.4.1.11), or a variant thereof. In another embodiment, the homocysteine degrading polypeptide is a sulfide:quinone reductase (E.C. 1.8.5.4), or a variant thereof. In another embodiment, the homocysteine degrading polypeptide is a methionine synthase (E.C. 2.1.1.13), or a variant thereof. In another embodiment, the homocysteine degrading polypeptide is a 5-methyl-tetrahydropteroyltriglutamate-homocysteine S-methyltransferase (E.C. 2.1.1.14), or a variant thereof. In another embodiment, the homocysteine degrading polypeptide is an adenosylhomocysteinase (E.C. 3.3.1.1), or a variant thereof. In another embodiment, the homocysteine degrading polypeptide is a cystathionine gamma-lyase (CGL; E.C. 4.4.1.1), or a variant thereof. In another embodiment, the homocysteine degrading polypeptide is an L-amino-acid oxidase (E.C. 1.4.3.2), or a variant thereof. In another embodiment, the homocysteine degrading polypeptide is a thetin-homocysteine S-methyltransferase (E.C. 2.1.1.3), or a variant thereof. In another embodiment, the homocysteine degrading polypeptide is a betaine-homocysteine S-methyltransferase (E.C. 2.1.1.5), or a variant thereof. In another embodiment, the homocysteine degrading polypeptide is a homocysteine S-methyltransferase (E.C. 2.1.1.10), or a variant thereof. In another embodiment, the homocysteine degrading polypeptide is a selenocysteine Se-methyltransferase (E.C. 2.1.1.280), or a variant thereof. In another embodiment, the homocysteine degrading polypeptide is a cystathionine gamma-synthase (E.C. 2.5.1.48), or a variant thereof. In another embodiment, the homocysteine degrading polypeptide is an O-acetylhomoserine aminocarboxypropyltransferase (E.C. 2.5.1.49), or a variant thereof. In another embodiment, the homocysteine degrading polypeptide is an asparagine-oxo-acid transaminase (E.C. 2.6.1.14), or a variant thereof. In another embodiment, the homocysteine degrading polypeptide is a glutamine-phenylpyruvate transaminase (E.C. 2.6.1.64), or a variant thereof. In another embodiment, the homocysteine degrading polypeptide is a 3-mercaptopyruvate sulfurtransferase (E.C. 2.8.1.2), or a variant thereof. In another embodiment, the homocysteine degrading polypeptide is a homocysteine desulfhydrase (E.C. 4.4.1.2), or a variant thereof. In another embodiment, the homocysteine degrading polypeptide is a cystathionine beta-lyase (E.C. 4.4.1.8), or a variant thereof. In another embodiment, the homocysteine degrading polypeptide is an amino-acid racemase (E.C. 5.1.1.10), or a variant thereof. In another embodiment, the homocysteine degrading polypeptide is a methionine-tRNA ligase (E.C. 6.1.1.10), or a variant thereof. In another embodiment, the homocysteine degrading polypeptide is a glutamate-cysteine ligase (E.C. 6.3.2.2), or a variant thereof. In another embodiment, the homocysteine degrading polypeptide is a N-(5-amino-5-carboxypentanoyl)-L-cysteinyl-D-valine synthase (E.C. 6.3.2.26), or a variant thereof. In another embodiment, the homocysteine degrading polypeptide is a L-isoleucine 4-hydroxylase (E.C. 1.14.11.45), or a variant thereof. In another embodiment, the homocysteine degrading polypeptide is a L-lysine N6-monooxygenase (NADPH) (E.C. 1.14.13.59), or a variant thereof. In another embodiment, the homocysteine degrading polypeptide is a methionine decarboxylase (E.0 4.1.1.57), or a variant thereof. In another embodiment, the homocysteine degrading polypeptide is a 2,2-dialkylglycine decarboxylase (pyruvate) (E.C. 4.1.1.64), or a variant thereof. The aforementioned enzymes can be derived from any species or source, and can be recombinantly produced.

In some embodiments, the exogenous polypeptide comprises a homocysteine degrading polypeptide, wherein the homocysteine degrading polypeptide comprises or consists of a cysteine synthase (CysO) (E.C. 2.5.1.47). In some embodiments, the CysO is derived from a prokaryote, e.g., Aeropyrum pemix. In some embodiments, the Aeropyrum pemix CysO comprises an amino acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or 100% identical) to the amino acid sequence set forth in SEQ ID NO: 12 (MALADISGYLDVLDSVRGFSYLENARE VLRSGEARCLGNPRSEPEYVKALYVIGASRIPVDGCSHTLEEGVFDISVPGEMVFPSPLDF FERGKPTPLVRSRLQLPNGVRVWLKLEWYNPFSLSVKDRPAVEIISRLSRRVEKGSLVAD ATSSNFGVALSAVARLYGYRARVYLPGAAEEFGKLLPRLLGAQVIVDPEAPSTVHLLPR VMKDSKNEGFVHVNQFYNDANFEAHMRGTAREIFVQSRRGGLALRGVAGSLGTSGHM SAAAFYLQSVDPSIRAVLVQPAQGDSIPGIRRVETGMLWINMLDISYTLAEVTLEEAMEA VVEVARSDGLVIGPSGGAAVKALAKKAAEGDLEPGDYVVVVPDTGFKYLSLVQNALE GAGDSV). In a particular embodiment, the CysO consists of the amino acid sequence of SEQ ID NO:12.

In some embodiments, the homocysteine degrading polypeptide comprises a variant of a wild-type homocysteine degrading polypeptide having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type homocysteine degrading polypeptide (e.g., a wild-type sulfide:quinone reductase, methionine synthase, 5-methyltetrahydropteroyltriglutamate-homocysteine S-methyltransferase, adeno sylhomocysteinase, cystathionine gamma-lyase, methionine gamma-lyase, L-amino-acid oxidase, thetin-homocysteine S-methyltransferase, betaine-homocysteine S-methyltransferase, homocysteine S-methyltransferase, selenocysteine Se-methyltransferase, cysteine synthase, cystathionine gamma-synthase, O-acetylhomoserine aminocarboxypropyltransferase, asparagine-oxo-acid transaminase, glutamine-phenylpyruvate transaminase, 3-mercaptopyruvate sulfurtransferase, homocysteine desulfhydrase, cystathionine beta-lyase, amino-acid racemase, methionine-tRNA ligase, glutamate-cysteine ligase, N-(5-amino-5-carboxypentanoyl)-L-cysteinyl-D-valine synthase, L-isoleucine 4-hydroxylase, L-lysine N6-monooxygenase (NADPH), methionine decarboxylase, or 2,2-dialkylglycine decarboxylase (pyruvate) enzyme).

In some embodiments, an engineered erythroid cell or an enucleated cell comprises an exogenous polypeptide comprising a homocysteine degrading polypeptide that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the homocysteine degrading polypeptide).

In some embodiments, the exogenous polypeptide may include a linker disposed between the homocysteine degrading polypeptide and the at least one polypeptide of interest. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 95), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGG (SEQ ID NO: 96), GGGGSGGGGS (SEQ ID NO: 97), GSGSGSGSGS (SEQ ID NO: 98), PSTSTST (SEQ ID NO: 99), and EIDKPSQ (SEQ ID NO: 100), and multimers thereof.

In some embodiments, the exogenous polypeptide comprises a transmembrane domain or a transmembrane polypeptide (e.g., SMIM1, GPA, or Kell). In some embodiments, the transmembrane domain is derived from GPA. For example, in some embodiments, the transmembrane domain is derived from GPA and comprises or consists of the amino acid sequence:

(SEQ ID NO: 102)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

In some embodiments, the transmembrane domain is derived from SMIM1. For example, in some embodiments, the transmembrane domain comprises or consists of the amino acid sequence:

(SEQ ID NO: 103)
MQPQESHVHYSRWEDGSRDGVSLGAVSSTEEASRCRRISQRLCTGKLGIA
MKVLGGVALFWIIFILGYLTGYYVHKCK

In some embodiments, the transmembrane domain or transmembrane polypeptide is disposed in the exogenous polypeptide such that the homocysteine degrading polypeptide present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the homocysteine degrading polypeptide present in the exogenous polypeptide locates in the outer surface of the cell (e.g., facing the extracellular milieu of the cell). In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the homocysteine degrading polypeptide.

In some embodiments the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA. For example, in some embodiments, the leader or signal sequence is derived from GPA and comprises or consists of the amino acid sequence MYGKIIFVLLLSEIVSISA (SEQ ID NO: 101).

Cystathionine Beta-Synthase

The disclosure provides, in one aspect, an erythroid cell engineered to degrade homocysteine and its metabolites, comprising a first exogenous polypeptide comprising a homocysteine degrading polypeptide, or variant thereof, wherein the homocysteine degrading polypeptide, or variant thereof, is a cystathionine beta-synthase, or a variant thereof. In some embodiments, the erythroid cell comprises more than one exogenous polypeptide, wherein each exogenous polypeptide comprises a cystathionine beta-synthase, e.g., cystathionine beta-synthase from the same (e.g., variants) or different sources or species.

Cystathionine beta-synthase (also referred to as CBS; beta-thionase; serine sulfhydrase; (E.C. 4.2.1.22)) plays an essential role in homocysteine metabolism in eukaryotes (Mudd et al., 2001, in The Metabolic and Molecular, Bases of Inherited Disease, 8 Ed., pp. 2007-2056, McGraw-Hill, New York). CBS governs the unidirectional flow of sulfur from methionine to cysteine by operating at the intersection of the transmethylation, transsulfuration, and remethylation pathways. It catalyzes a β-replacement reaction in which serine condenses with homocysteine in a pyridoxal-5′-phosphate-dependent (PLP-dependent) manner to form cystathionine. Cystathionine can then be converted to cysteine by cystathionine γ-lyase (CGL). (Bublil et al. 2016 J Clin Invest. June 1; 126(6): 2372-2384). CBS can be allosterically regulated by effectors such as the ubiquitous cofactor S-adenosyl-L-methionine (adoMet). This enzyme belongs to the hydro-lyase family, which cleave carbon-oxygen bonds.

An engineered erythroid cell of the disclosure may comprise an exogenous polypeptide comprising a cystathionine beta-synthase, or variant thereof, wherein the cystathionine beta-synthase is derived from any source(s) known in the art, including mammalian (e.g., human, mouse, rat, Oryctolagus cuniculus, Monodelphis domestica, or Ornithorhynchus anatinus), insect (e.g., Drosophila melanogaster), bacterial (e.g., Mycobacterium tuberculosis), fungal (including yeast (e.g., Saccharomyces cerevisiae or, Emericella nidulan), or protozoa (e.g., Dictyostellium discoideum) sources, as well as cystathionine beta-synthases generated by recombinant technologies.

In some preferred embodiments of the disclosure, the cystathionine beta-synthase (or variant thereof) is a cystathionine beta-synthase selected from those set forth in Table 1, below, including a cystathionine beta-synthase, or variant thereof, derived from a human, Mus musculus, Rattus norvegicus, Oryctolagus cuniculus, Monodelphis domestica, Ornithorhynchus anatinus, Drosophila melanogaster, Mycobacterium tuberculosis, Saccharomyces cerevisiae, Emericella nidulan, or Dictyostellium discoideum.

TABLE 1
Exemplary Cystathionine Beta-Synthases (Full-length sequences)

| SEQ. ID | Cystathionine Beta- | |
|---|---|---|
| NO | Synthase source | Amino acid sequence |
| 1 | Homo sapiens | MPSETPQAEV GPTGCPHRSG PHSAKGSLEK GSPEDKEAKE |
| | | PLWIRPDAPS RCTWQLGRPA SESPHHHTAP AKSPKILPDI |
| | | LKKIGDTPMV RINKIGKKFG LKCELLAKCE FFNAGGSVKD |
| | | RISLRMIEDA ERDGTLKPGD TIIEPTSGNT GIGLALAAAV |
| | | RGYRCIIVMP EKMSSEKVDV LRALGAEIVR TPTNARFDSP |
| | | ESHVGVAWRL KNEIPNSHIL DQYRNASNPL AHYDTTADEI |
| | | LQQCDGKLDM LVASVGTGGT ITGIARKLKE KCPGCRIIGV |
| | | DPEGSILAEP EELNQTEQTT YEVEGIGYDF IPTVLDRTVV |
| | | DKWFKSNDEE AFTFARMLIA QEGLLCGGSA GSTVAVAVKA |
| | | AQELQEGQRC VVILPDSVRN YMTKFLSDRW MLQKGFLKEE |
| | | DLTEKKPWWW HLRVQELGLS APLTVLPTIT CGHTIEILRE |
| | | KGFDQAPVVD EAGVILGMVT LGNMLSSLLA GKVQPSDQVG |
| | | KVIYKQFKQI RLTDTLGRLS HILEMDHFAL VVHEQIQYHS |
| | | TGKSSQRQMV FGVVTAIDLL NFVAAQERDQ K |
| 2 | Saccharomyces | MTKSEQQADS RHNVIDLVGN TPLIALKKLP KALGIKPQIY |
| | cerevisiae | AKLELYNPGG SIKDRIAKSM VEEAEASGRI HPSRSTLIEP |
| | | TSGNTGIGLA LIGAIKGYRT IITLPEKMSN EKVSVLKALG |
| | | AEIIRTPTAA AWDSPESHIG VAKKLEKEIP GAVILDQYNN |
| | | MMNPEAHYFG TGREIQRQLE DLNLFDNLRA VVAGAGTGGT |
| | | ISGISKYLKE QNDKIQIVGA DPFGSILAQP ENLNKTDITD |
| | | YKVEGIGYDF VPQVLDRKLI DVWYKTDDKP SFKYARQLIS |
| | | NEGVLVGGSS GSAFTAVVKY CEDHPELTED DVIVAIFPDS |
| | | IRSYLTKFVD DEWLKKNNLW DDDVLARFDS SKLEASTTKY |
| | | ADVFGNATVK DLHLKPVVSV KETAKVTDVI KILKDNGFDQ |
| | | LPVLTEDGKL SGLVTLSELL RKLSINNSNN DNTIKGKYLD |
| | | FKKLNNFNDV SSYNENKSGK KKFIKFDENS KLSDLNRFFE |
| | | KNSSAVITDG LKPIHIVTKM DLLSYLA |
| 3 | Mus musculus | MPSGTSQCED GSAGGFQHLD MHSEKRQLEK GPSGDKDRVW |
| | | IRPDTPSRCT WQLGRAMADS PHYHTVLTKS PKILPDILRK |
| | | IGNTPMVRIN KISKNAGLKC ELLAKCEFFN AGGSVKDRIS |
| | | LRMIEDAERA GNLKPGDTII EPTSGNTGIG LALAAAVKGY |
| | | RCIIVMPEKM SMEKVDVLRA LGAEIVRTPT NARFDSPESH |
| | | VGVAWRLKNE IPNSHILDQY RNASNPLAHY DDTAEEILQQ |
| | | CDGKLDMLVA SAGTGGTITG IARKLKEKCP GCKIIGVDPE |
| | | GSILAEPEEL NQTEQTAYEV EGIGYDFIPT VLDRAVVDKW |
| | | FKSNDEDSFA FARMLIAQEG LLCGGSSGSA MAVAVKAARE |
| | | LQEGQRCVVI LPDSVRNYMS KFLSDKWMLQ KGFMKEELSV |
| | | KRPWWWRLRV QELSLSAPLT VLPTVTCEDT IAILREKGFD |
| | | QAPVVNESGA ILGMVTLGNM LSSLLAGKVR PSDEVCKVLY |
| | | KQFKPIHLTD TLGTLSHILE MDHFALVVHE QIQSRDQAWS |
| | | GVVGGPTDCS NGMSSKQQMV FGVVTAIDLL NFVAAREQTQ T |
| 4 | Oryctolagus | MPSETAQAGE GPAGCPHLSG AQGSDRSLDQ RPPGNKDAPE |
| | cuniculus | RVWIRPDVPS RCTWELGRPV ADSPHQHAAL AKSPKILPDI |
| | | LQKIGDTPMV RINKIGKNFG LKCELLAKCE FFNAGGSVKD |
| | | RISLRMIEDA ERAGTLRPGD TIIEPTSGNT GIGLALAAAV |
| | | KGYRCIIVMP EKMSLEKVDV LRALGAEIVR TPTNARFDSP |
| | | ESHVGVAWRL KQEIPNSHIL DQYRNASNPL AHYDTTAEEI |
| | | LQQCDGKLDM LVASAGTGGT ITGIARKLKE KCPGCQIIGV |
| | | DPEGSILAEP EELNQTEVTA YEVEGIGYDF IPTVLDRTVV |
| | | DRWFKSTDKE AFAFARMLIA QEGLLCGGSA GSAVAVAVKA |
| | | AQELQEGQRC VVILPDSVRN YMSKFLSDRW MLQKGFLEEE |
| | | ELSVKRPWWW HLRVQELSLS VPLTVLPGVT CSDTIDILRG |
| | | KGFDQAPVVD ETGEILGMVT LGNMLSSLLA GKVQPSDQVC |
| | | KVLYKQFKQI RLTDTLGALS HILEMDHFAL VVHEQIQYGG |
| | | DEQPSKRQTV FGVVTAMDLL HFVASRGQDQ Q |
| 5 | Mycobacterium | MRIAQHISEL IGGTPLVRLN SVVPDGAGTV AAKVEYLNPG |
| | tuberculosis | GSSKDRIAVK MIEAAEASGQ LKPGGTIVEP TSGNTGVGLA |
| | | LVAQRRGYKC VFVCPDKVSE DKRNVLIAYG AEVVVCPTAV |
| | | PPHDPASYYS VSDRLVRDID GAWKPDQYAN PEGPASHYVT |
| | | TGPEIWADTE GKVTHFVAGI GTGGTITGAG RYLKEVSGGR |
| | | VRIVGADPEG SVYSGGAGRP YLVEGVGEDF WPAAYDPSVP |
| | | DEIIAVSDSD SFDMTRRLAR EEAMLVGGSC GMAVVAALKV |
| | | AEEAGPDALI VVLLPDGGRG YMSKIFNDAW MSSYGFLRSR |
| | | LDGSTEQSTV GDVLRRKSGA LPALVHTHPS ETVRDAIGIL |
| | | REYGVSQMPV VGAEPPVMAG EVAGSVSERE LLSAVFEGRA |
| | | KLADAVSAHM SPPLRMIGAG ELVSAAGKAL RDWDALMVVE |
| | | EGKPVGVITR YDLLGFLSEG AGRR |
| 6 | Rattus norvegicus | MPSGTSQCED GSAGCPQDLE VQPEKGQLEK GASGDKERVW |
| | | ISPDTPSRCT WQLGRPMADS PHYHTVPTKS PKILPDILRK |
| | | IGNTPMVRIN RISKNAGLKC ELLAKCEFFN AGGSVKDRIS |
| | | LRMIEDAERA GTLKPGDTII EPTSGNTGIG LALAAAVKGY |
| | | RCIIVMPEKM SMEKVDVLRA LGAEIVRTPT NARFDSPESH |
| | | VGVAWRLKNE IPNSHILDQY RNASNPLAHY DDTAEEILQQ |
| | | CDGKVDMLVA SAGTGGTITG IARKLKEKCP GCKIIGVDPE |
| | | GSILAEPEEL NQTEQTAYEV EGIGYDFIPT VLDRAVVDRW |
| | | FKSNDDDSFA FARMLISQEG LLCGGSSGSA MAVAVKAAQE |
| | | LKEGQRCVVI LPDSVRNYMS KFLSDKWMLQ KGFMKEELSV |
| | | KRPWWWHLRV QELSLSAPLT VLPTVTCEHT IAILREKGFD |
| | | QAPVVNESGA ILGMVTLGNM LSSLLAGKVR PSDEVCKVLY |
| | | KQFKPIHLTD TLGMLSHILE MDHFALVVHE QIQSRDQAWS |
| | | GVVGGPTDRN NGVSSKQLMV FGVVTAIDLL NFVAAREQTR K |
| 7 | Dictyostellium | MSAPEGPSKC TWTPNTTENT PHTTRRTPKK LIMDNILDNI |
| | discoideum | GGTPLVRVNK VSSDLECELV AKCEFFNAGG SVKDRIGHRM |
| | | IVDAEESGRI KKGDTLIEPT SGNTGIGLAL TAAIKGYKMI |
| | | ITLPEKMSQE KVDVLKALGA EIIRTPTEAA FDAPESHIGV |
| | | AKKLNSEIPN SHILDQYGNP SNPLAHYDGT AEELLEQCEG |
| | | KIDMIVCTAG TGGTITGIAR KIKERLPNCI VVGVDPHGSI |
| | | LAQPESLNNT NKSYKIEGIG YDFIPNVLER KLVDCMIKTD |
| | | DKESFIMARR LIKEEGLLCG GSSGSAMVGA LLAAKQLKKG |
| | | QRCVVLLADS IRNYMTKHLN DDWLVDNGFV DPEYKTKDQQ |
| | | EEEKYHGATV KDLTLPKPIT ISATTTCAAA VQLLQQYGFD |
| | | QLPVVSESKK VLGQLTLGNL LSHIASKKAV PTDAVSKVMF |
| | | RFTKNEKYIP ITQSTSLATL SKFFENHSSA IVTENDEIIS IVTKIDLLTY |
| | | LMKSQQKN |
| 8 | Drosophila | MPQPKPYERP ADFIDPGKPS KCKWHLGTAE KSPHIHRGIA |
| | melanogaster | HRQQITPNIL EVIGCTPLVK LNNIPASDGI ECEMYAKCEF |
| | | LNPGGSVKDR IGYRMVQDAE EQGLLKPGYT IIEPTSGNTG |
| | | IGLAMACAVK GYKCIIVMPE KMSNEKVSAL RTLGAKIIRT |
| | | PTEAAYDSPE GLIYVAQQLQ RETPNSIVLD QYRNAGNPLA |
| | | HYDGTAAEIL WQLDNKVDMI VVSAGTAGTI SGIGRKIKEQ |
| | | VPSCQIVGVD PYGSILARPA ELNKTDVQFY EVEGIGYDFP |
| | | PTVFDDTVVD VWTKIGDSDC FPMSRRLNAE EGLLCGGSSG |
| | | GAMHAALEHA RKLKKGQRCV VILPDGIRNY MTKFVSDNWM |
| | | EARNFKEPVN EHGHWWWSLA IAELELPAPP VILKSDATVG |
| | | EAIALMKKHR VDQLPVVDQD DGSVLGVVGQ ETLITQIVSM |
| | | NRQQSDPAIK ALNKRVIRLN ESEILGKLAR VLEVDPSVLI |
| | | LGKNPAGKVE LKALATKLDV TTFIAAGKQK PKANGTTNGG SH |
| 9 | Emericella nidulan | MSSKTTPTVP PVALDSITQH IGNTPLVRLN KLPQNLGINA |
| | | TVYAKLEYFN AGGSVKDRIA LRMIEEAERS GRIKPGDTLI |
| | | EPTSGNTGIG LALVAAVKGY KTIITLPEKM SAEKVSVLRA |
| | | LNATIIRTPN EAAYDSPESH IGVAKRLEKE LPNAHILDQY |
| | | GNENNPLAHE LGTAQEIWSQ TKGQIKAIVA GAGTGGTITG |
| | | LSRGLKKHNS NVQVIAADPQ GSILALPAAL NEEHANEPYK |
| | | VEGIGYDFIP QVLDQHAVDK WYKTDDKESF QYARRLIAEE |
| | | GLLVGGSSGS AIAALVKAAR DNMFKEGDVV VVILPDSIRS |
| | | YLTKFADDDW LAANDLLPSL PTEVILQSSN KQTQGQDDAF |
| | | AGSKVSSLRL KPVTTVHSNV PCETAIEVMR DKGFDQLPVL |
| | | APSGRKLVGL VTLGNVLSRL THNRATGRSP VADVMFDFRT |
| | | ISEVVTDPRD MGLASLEPNQ NGRDAPKPQT RSRKFVEITM |
| | | DTSLSVLNRF FEWNSAAIVT ERDEGGTLRP VAVVTKVDLL |
| | | TWMLHHKKNG ASE |
| 10 | Monodelphis | MPHEFPQIER ENGVCPHLSS NSMENVAKET SSNCGNKVEK |
| | domestica | ERKWIRPDNP SKCTWQLGKS LSESPHHHVS WTKTPKIIPD |
| | | ILKKIGDTPM VRINKIGKNF GLKCELLAKC EFFNAGGSVK |
| | | DRISLRMVED AERAGILKPG DTIIEPTSGN TGIGLALAAA |
| | | VKGYRCIIVM PEKMSMEKVD VLRALGAEIV RTPTNARFDS |
| | | PESHVGVAWR LKNEIPNSHI LDQYRNASNP LTHYDSTAEE |
| | | ILQQCDGKLD MLVASAGTGG TITGIARKLK EKCPGCKIIG |
| | | VDPEGSILAE PEELNQTDKT AYEVEGIGYD FIPTVLDRKV |
| | | VDKWYKSNDE ESFAFARMLI AQEGLLCGGS AGSSMSVAVK |
| | | AAKELKEGQR CVVILPDSVR NYMSKFLSDK WMLQKGFVKE |
| | | EDITITKPWW WHLKIQELSL SAPLTVLPTV TCERTIEILR |
| | | EKGFDQVPVV DESGVILGMV TLGNMLSSLL AGKVQPSDQV |
| | | RNVIYKQFRQ IHLTDTLGML SHILETDHFA LVVHEQIQSQ |
| | | DQSLSEMIGG ISDHSNGNSI LRQMVFGVVT AIDLLNFVAA |
| | | REREQK |
| 11 | Ornithorhynchus | MPSELSKAEE KPGVCPHRPG NPAENGKSDT ARCTGLSEKE |
| | anatinus | RKWIRPDMPS KCTWQLGKPA SESPHYHVSL AQTPQIMPDI |
| | | LKKIGNTPLV RINKIGKAYG LKCELLAKCE FFNAGGSVKD |
| | | RIGLRMLEDA ERAGILKPGD TIIEPTSGNT GIGLALAAAV |
| | | KGYRCIIVMP EKMSMEKVDV LRALGAEIVR TPTTASFDSP |
| | | ESHVGVAWRL KHEIPNSHIL DQYRNASNPL AHYDTTAEEI |
| | | LQQCDGKLDM LVASAGTGGT ITGIGRKLKE KCPGCKIIGV |
| | | DPEGSLLAEP EDLNKTDKTI YEVEGIGYDF IPTVLDRAMV |
| | | DKWYKCNDEE SFAFARMLIS QEGLLCGWGS SGSTMSLAVK |
| | | AAQELKDGQR CVFILPDFVQ KYMSKFLSDK WMLQKGFLKE |
| | | EDIMINKPWW WHVKVQELSL SAPLTVLPAV TCERTIEILR |
| | | EKGFDQVPVV DESGVILGMV TLANMLSSLL AGKVRPSDQV |
| | | RKVIYKQFQK IRLTDTLGKL SHILEIDHFA LVVHEQIQSQ |
| | | DQALPAMVGG TTAGIQHGLE NSSSSIDKPE HRGIDLKSSY |
| | | RFIRHLSPES KHKFNAISA |

In one embodiment, the cystathionine beta-synthase comprises or consists of the amino acid sequence of SEQ ID NO:1, or a variant thereof. In one embodiment, the cystathionine beta-synthase comprises or consists of the amino acid sequence of SEQ ID NO:2, or a variant thereof. In one embodiment, the cystathionine beta-synthase comprises or consists of the amino acid sequence of SEQ ID NO:3, or a variant thereof. In one embodiment, the cystathionine beta-synthase comprises or consists of the amino acid sequence of SEQ ID NO:4, or a variant thereof. In one embodiment, the cystathionine beta-synthase comprises or consists of the amino acid sequence of SEQ ID NO:5, or a variant thereof. In one embodiment, the cystathionine beta-synthase comprises or consists of the amino acid sequence of SEQ ID NO:6, or a variant thereof. In one embodiment, the cystathionine beta-synthase comprises or consists of the amino acid sequence of SEQ ID NO:7, or a variant thereof. In one embodiment, the cystathionine beta-synthase comprises or consists of the amino acid sequence of SEQ ID NO:8, or a variant thereof. In one embodiment, the cystathionine beta-synthase comprises or consists of the amino acid sequence of SEQ ID NO:9, or a variant thereof. In one embodiment, the cystathionine beta-synthase comprises or consists of the amino acid sequence of SEQ ID NO:10, or a variant thereof. In one embodiment, the cystathionine beta-synthase comprises or consists of the amino acid sequence of SEQ ID NO:11, or a variant thereof.

In one embodiment, the cystathionine beta-synthase comprises an amino acid sequence that is at least 95% identical (e.g., 96%, 97%, 98%, 99% or 100% identical) to the amino acid sequence set forth in SEQ ID NO:1. In one embodiment, the cystathionine beta-synthase comprises an amino acid sequence that is at least 95% identical (e.g., 96%, 97%, 98%, 99% or 100% identical) to the amino acid sequence set forth in SEQ ID NO:2. In one embodiment, the cystathionine beta-synthase comprises an amino acid sequence that is at least 95% identical (e.g., 96%, 97%, 98%, 99% or 100% identical) to the amino acid sequence set forth in SEQ ID NO:3. In one embodiment, the cystathionine beta-synthase comprises an amino acid sequence that is at least 95% identical (e.g., 96%, 97%, 98%, 99% or 100% identical) to the amino acid sequence set forth in SEQ ID NO:4. In one embodiment, the cystathionine beta-synthase comprises an amino acid sequence that is at least 95% identical (e.g., 96%, 97%, 98%, 99% or 100% identical) to the amino acid sequence set forth in SEQ ID NO:5. In one embodiment, the cystathionine beta-synthase comprises an amino acid sequence that is at least 95% identical (e.g., 96%, 97%, 98%, 99% or 100% identical) to the amino acid sequence set forth in SEQ ID NO:6. In one embodiment, the cystathionine beta-synthase comprises an amino acid sequence that is at least 95% identical (e.g., 96%, 97%, 98%, 99% or 100% identical) to the amino acid sequence set forth in SEQ ID NO:7. In one embodiment, the cystathionine beta-synthase comprises an amino acid sequence that is at least 95% identical (e.g., 96%, 97%, 98%, 99% or 100% identical) to the amino acid sequence set forth in SEQ ID NO:8. In one embodiment, the cystathionine beta-synthase comprises an amino acid sequence that is at least 95% identical (e.g., 96%, 97%, 98%, 99% or 100% identical) to the amino acid sequence set forth inSEQ ID NO:9. In one embodiment, the cystathionine beta-synthase comprises an amino acid sequence that is at least 95% identical (e.g., 96%, 97%, 98%, 99% or 100% identical) to the amino acid sequence set forth in SEQ ID NO:10. In one embodiment, the cystathionine beta-synthase comprises an amino acid sequence that is at least 95% identical (e.g., 96%, 97%, 98%, 99% or 100% identical) to the amino acid sequence set forth in SEQ ID NO:11.

In one embodiment, the cystathionine beta-synthase comprises a Homo sapiens cystathionine beta-synthase. In one embodiment, the Homo sapiens cystathionine beta-synthase comprises an amino acid sequence that is at least 95% identical (e.g., 96%, 97%, 98%, 99% or 100% identical) to the amino acid sequence set forth in SEQ ID NO:1. U.S. Pat. No. 5,523,225, incorporated herein by reference in its entirety, describes the nucleic acid and amino acid sequences of human cystathionine beta-synthase.

In one embodiment, the cystathionine beta-synthase comprises a Saccharomyces cerevisiae cystathionine beta-synthase. In one embodiment, the Saccharomyces cerevisiae cystathionine beta-synthase comprises an amino acid sequence that is at least 95% identical (e.g., 96%, 97%, 98%, 99% or 100% identical) to the amino acid sequence set forth in SEQ ID NO:2.

In one embodiment, the cystathionine beta-synthase comprises a Mus musculus cystathionine beta-synthase. In one embodiment, the Mus musculus cystathionine beta-synthase comprises an amino acid sequence that is at least 95% identical (e.g., 96%, 97%, 98%, 99% or 100% identical) to the amino acid sequence set forth in SEQ ID NO:3.

In one embodiment, the cystathionine beta-synthase comprises a Oryctolagus cuniculus (European rabbit) cystathionine beta-synthase. In one embodiment, the Oryctolagus cuniculus cystathionine beta-synthase comprises an amino acid sequence that is at least 95% identical (e.g., 96%, 97%, 98%, 99% or 100% identical) to the amino acid sequence set forth in SEQ ID NO:4.

In one embodiment, the cystathionine beta-synthase comprises a Mycobacterium tuberculosis cystathionine beta-synthase. In one embodiment, the Mycobacterium tuberculosis cystathionine beta-synthase comprises an amino acid sequence that is at least 95% identical (e.g., 96%, 97%, 98%, 99% or 100% identical) to the amino acid sequence set forth in SEQ ID NO:5.

In one embodiment, the cystathionine beta-synthase comprises a Rattus norvegicus cystathionine beta-synthase. In one embodiment, the Rattus norvegicus cystathionine beta-synthase comprises an amino acid sequence that is at least 95% identical (e.g., 96%, 97%, 98%, 99% or 100% identical) to the amino acid sequence set forth in SEQ ID NO:6.

In one embodiment, the cystathionine beta-synthase comprises as Dictyostellium discoideum cystathionine beta-synthase. In one embodiment, the Dictyostellium discoideum cystathionine beta-synthase comprises an amino acid sequence that is at least 95% identical (e.g., 96%, 97%, 98%, 99% or 100% identical) to the amino acid sequence set forth in SEQ ID NO:7.

In one embodiment, the cystathionine beta-synthase comprises a Drosophila melanogaster cystathionine beta-synthase. In one embodiment, the Drosophila melanogaster cystathionine beta-synthase comprises an amino acid sequence that is at least 95% identical (e.g., 96%, 97%, 98%, 99% or 100% identical) to the amino acid sequence set forth in SEQ ID NO:8.

In one embodiment, the cystathionine beta-synthase comprises a Emericella nidulan cystathionine beta-synthase. In one embodiment, the Emericella nidulan cystathionine beta-synthase comprises an amino acid sequence that is at least 95% identical (e.g., 96%, 97%, 98%, 99% or 100% identical) to the amino acid sequence set forth in SEQ ID NO:9.

In one embodiment, the cystathionine beta-synthase comprises a Monodelphis domestica (short-tailed opossum) cystathionine beta-synthase. In one embodiment, the Monodelphis domestica cystathionine beta-synthase comprises an amino acid sequence that is at least 95% identical (e.g., 96%, 97%, 98%, 99% or 100% identical) to the amino acid sequence set forth in SEQ ID NO:10.

In one embodiment, the cystathionine beta-synthase comprises aOrnithorhynchus anatinus (platypus) cystathionine beta-synthase. In one embodiment, the Ornithorhynchus anatinus cystathionine beta-synthase comprises an amino acid sequence that is at least 95% identical (e.g., 96%, 97%, 98%, 99% or 100% identical) to the amino acid sequence set forth in SEQ ID NO:11.

In some embodiments, the cystathionine beta-synthase comprises a variant of a wild-type cystathionine beta-synthase having at least 40%, at least 50%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11. In some embodiments, the variant of a wild-type cystathionine beta-synthase possesses a function of the wild-type cystathionine beta-synthase from which it was derived, e.g., the variant can catalyze the pyridoxal 5′-phosphate (PLP)-dependent condensation of serine and homocysteine to form cystathionine (i.e., the enzymatic activity or catalytic activity), bind heme, bind PLP, bind to AdoMet, and/or respond to AdoMet.

In a particular embodiment, the cystathionine beta-synthase consists of the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

A cystathionine beta-synthase can also include proteins having an amino acid sequence comprising at least 10 contiguous amino acid residues of any one of SEQ ID NOs:1-11 (i.e., 10 contiguous amino acid residues having 100% identity with 10 contiguous amino acids of any one of SEQ ID NOs:1-11). In other embodiments, a cystathionine beta-synthase amino acid sequence includes amino acid sequences comprising at least 20, or at least 30, or at least 40, or at least 50, or at least 75, or at least 100, or at least 125, or at least 150, or at least 175, or at least 150, or at least 200, or at least 250, or at least 300, or at least 350, or at least 400, or at least 450, or at least 500, or at least 550, contiguous amino acid residues of the amino acid sequence represented by SEQ ID NO:1, and any whole integer in between 10 and 550 contiguous amino acid residues. In a preferred embodiment, a cystathionine beta-synthase has measurable or detectable cystathionine beta-synthase biological activity. In some embodiments, fragments or variants of the cystathionine beta-synthase enzyme retain at least 50%, at least 55%, at least 60%, at least 65%, at least 70%, at least 75%, at least 80%, at least 85%, at least 90%, at least 95%, at least 98% or at least 99% of the activity as compared to the wild-type cystathionine beta-synthase from which they were derived.

In general, a variant cystathionine beta-synthase, from any origin, may be produced, for example, to enhance production of the protein in an engineered cell, to improve turnover/half-life of the protein or mRNA encoding the protein, and/or to modulate (enhance or reduce) the enzymatic activity of the cystathionine beta-synthase. The cystathionine beta-synthase, whatever the source, may also be in a form that is truncated, either at the amino terminal, or at the carboxyl terminal, or at both terminals. In one embodiment, the truncation is a deletion of the N-terminal heme-binding region of the cystathionine beta-synthase. In another embodiment, the truncated cystathionine beta-synthase comprises at least the proteolytically resistant core. In another embodiment, the truncated cystathionine beta-synthase polypeptide contains at least one mutated amino acid residue (e.g., a deletion, addition or substitution). In one embodiment, the mutation is a mutation of one or more cysteine residues.

In some embodiments, the invention provides an engineered erythroid cell (e.g. an engineered erythroid precursor cell) comprising a nucleic acid sequence encoding a cystathionine beta-synthase as described herein. In some embodiments, the invention provides an engineered erythroid cell prepared by using a nucleic acid sequence encoding a cystathionine beta-synthase (e.g. a cystathionine beta-synthase, or variant thereof, derived from a human, Mus musculus, Rattus norvegicus, Oryctolagus cuniculus, Monodelphis domestica, Ornithorhynchus anatinus, Drosophila melanogaster, Mycobacterium tuberculosis, Saccharomyces cerevisiae, Emericella nidulan, or Dictyostellium discoideum) as described herein. In some embodiments, the nucleic acid sequence encodes a cystathionine beta-synthase as described herein.

In some embodiments, the cystathionine beta-synthase is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the corresponding wild-type cystathionine beta-synthase nucleic acid (from any source) that encodes a protein that possesses a function of a cystathionine beta-synthase described herein, e.g., the encoded protein can catalyze the pyridoxal 5′-phosphate (PLP)-dependent condensation of serine and homocysteine to form cystathionine (i.e., the enzymatic activity or catalytic activity), bind heme, bind PLP, bind to AdoMet, and/or respond to AdoMet.

Truncated versions of cystathionine beta-synthase are known in the art and are described in detail in, for example, U.S. Patent No. 8,007,787, the contents of which are hereby incorporated by reference herein. In some embodiments, the erythroid cells of the present disclosure include an exogenous polypeptide comprising a truncated version of a cystathionine beta-synthase.

In one embodiment, the truncated cystathionine beta-synthase has an amino acid sequence that comprises, consists essentially of, or consists of, a truncated version of SEQ ID NO:1. SEQ ID NO:1 represents a full-length human cystathionine beta-synthase of 551 amino acids. In some embodiments, the truncated cystathione beta-synthase has an amino acid sequence that comprises, consists essentially of, or consists of a truncated vedrsion of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

In one embodiment, the truncated or full-length cystathionine beta-synthase contains a mutation at amino acid residue 15 of SEQ ID NO:1, wherein the mutation is a substitution of the cysteine residue at amino acid residue 15 with serine (C15S) (see, e.g., Frank et al. Biochemistry 2006, 45, 11021-11029). Preferably, the variant cystathionine beta-synthase has at least one cystathionine beta-synthase biological activity as described previously herein, and most preferably, has at least detectable cystathionine beta-synthase catalytic activity as described herein.

In one embodiment, the truncated version of cystathionine beta-synthase is a human truncated version of cystathionine beta-synthase (htCBS). In one embodiment, the truncated version of cystathionine beta-synthase is a human truncated version of cystathionine beta-synthase, wherein the human cystathionine beta-synthase amino acid sequence is set forth as SEQ ID NO:1. In one embodiment, an engineered erythroid cell comprises an exogenous polypeptide comprising a truncated version of cystathionine beta-synthase that comprises or consists of amino acid residues 1-413 of SEQ ID NO:1.

In another embodiment, the truncated version of cystathionine beta-synthase also comprises one or more mutated amino acid residues as compared to the wild-type cystathionine beta-synthase from which it was derived. In one embodiment, the truncated version of cystathionine beta-synthase comprising or consisting of amino acid residues 1-413 of SEQ ID NO:1, and contains a mutation at amino acid residue 15, wherein the mutation is a substitution of the cysteine residue at amino acid residue 15 with serine (C15S) (see, for example, Bubil et al. J. Clin. Investigation 2016 126(6).

In one embodiment, the truncated cystathionine beta-synthase consists or comprises amino acid residues at positions 1-550, 1-543, 1-533, 1-523, 1-496, 1-488, 1-441, 1-413, 40-413, 40-551, 71-413, 71-551, 70-413, or 70-551 of SEQ ID NO:1.

In one embodiment, truncated versions of cystathionine beta-synthase derived from SEQ ID NO:1 include N-terminal deletion variants, C-terminal deletion variants, and variants having both N-terminal and C-terminal deletions. With regard to the N-terminal deletion variants, such variants include proteins that have an amino acid sequence that differs from SEQ ID NO:1 by at least one, and up to about 83 deleted amino acid residues from the N-terminal 83 amino acid residues of SEQ ID NO:1. Such variants can include any number of deletions from between position 1 and about 83 of SEQ ID NO:1, inclusive, in whole integers (e.g., a deletion of the amino acid residue at position 1, a deletion of the amino acid residue at positions 1 and 2, a deletion of the amino acid residue at positions 1-6, a deletion of the amino acid residue at positions 1-14, a deletion of the amino acid residue at positions 1-28, a deletion of the amino acid residue at positions 1-68, etc., and any number in between, up to a deletion of all of the amino acid residue at positions 1-83).

Truncated variants of cysteine beta-synthase include, but are not limited to, variants having a deletion of the amino acid residues (relative to SEQ ID NO:1) at position 1, at positions 1-39, at positions 1-52, at positions 1-65, at positions 1-69, at positions 1-70, or at positions 1-83. The first amino acid residue of such variants, relative to SEQ ID NO:1, are 2, 40, 53, 66, 70, 71, or 84, respectively. Notwithstanding, the variant may be engineered such that the first amino acid residue of the truncated cystathione beta-synthase is any one of the amino acid residuesfrom position 2 to position 84. Any of the N-terminal truncation variants, C-terminal truncation variants, or both N-terminal and C-terminal truncations variants provided herein may comprise the remainder of the full-length wild-typecystathionine beta-synthase amino acid sequence, or or one or more modifications (e.g., mutations) as compared to the wild-type cystathionine beta-synthase amino acid. Preferably, these cystathionine beta-synthase variants catalyze the formation of cystathionine and may have one, more or all of the other biological activities of a wild-type cystathionine beta-synthase protein.

In one embodiment, a cystathionine beta-synthase variant has one or more mutations or deletions that result in decreased heme binding by the variant or substantially no heme binding by the variant. In one aspect, a non-heme binding cystathionine beta-synthase lacks the amino acid residues present from between about the amino acid residue at position 65 to about the amino acid residue at position 83 of the N-terminal amino acid residues of SEQ ID NO:1, including any number of amino acid residues in whole integers between the amino acid residue at position 65 and to about position 83. Therefore, such a truncated variant (deletion mutant) of SEQ ID NO:1 would have a starting, or first, amino acid residue corresponding to the amino acid residue at, relative to SEQ ID NO:1, about position 66 through about position 84. In one embodiment, the cystathionine beta-synthase variant lacks the amino acid residues (relative to SEQ ID NO:1) from about positions 1-65, about 1-69, about 1-70, or about 1-83. Such variants would have a starting amino acid position, relative to SEQ ID NO:1, of about 66, 70, 71, or 84, respectively. The cystathionine beta-synthase variants may comprise an amino acid sequence beginning at an amino acid residue from SEQ ID NO: 1 from about the amino acid residue present at position 66 to about the amino acid residue present at position 84. In one embodiment, any one of the cystathionine beta-synthase variants catalyze the formation of cystathionine and do not bind heme.

In one embodiment, the cystathionine beta-synthase does not bind heme and comprises an amino acid sequence of SEQ ID NO:1, having either a deletion or a mutation at Cys52 and His 65.

In some embodiments, the cystathionine beta-synthase variant comprises an amino acid sequence that differs from SEQ ID NO:1 from by at least one to about 169 C-terminal amino acid residues. Such variants can lack any number of amino acid residues corresponding to from about the amino acid residue at position 551 to about the amino acid residue at position 383 of SEQ ID NO:1, inclusive, in whole integers (e.g., a deletion of position 551, a deletion of positions 550-551, a deletion of positions 544-551, a deletion of positions 482-551, a deletion of positions 437-551, a deletion of positions 391-551, etc., and any number in between, up to a deletion of all of positions 383-551). In one embodiment, the cystathionine beta-synthase variant is derived from the amino acid sequence of SEQ ID NO:1 and lacks the amino acid residues at positions (relative to SEQ ID NO:1) of from about 544-551, about 524-551, about 497-551, about 489-551, about 442-551, about 414-551, about 401-551, or 383-551. In some embodiments, the cystathionine beta-synthase variant is derived from the amino acid sequence of SEQ ID NO:1, and lacks the amino acid residues at positions 414-551 (relative to SEQ ID NO: 1). In some embodiments, the cystathionine beta-synthase variant comprises the amino acid residues at positions 1-413 of SEQ ID NO: 1. In some embodiments, the cystathionine beta-synthase variant is derived from the amino acid sequence of SEQ ID NO:1, and lacks the amino acid residues at positions 399-551 (relative to SEQ ID NO: 1). In some embodiments, the cystathionine beta-synthase variant comprises the amino acid residues at positions 1-413 of SEQ ID NO: 1. In some embodiments, the cystathionine beta-synthase variant is derived from the amino acid sequence of SEQ ID NO:2, and lacks the amino acid residues at positions 354-507 (relative to SEQ ID NO: 2). In some embodiments, the cystathionine beta-synthase variant comprises the amino acid residues at positions 1-353 of SEQ ID NO: 2.

In some embodiments, the cystathionine beta-synthase is selected from: (a) a protein having an amino acid sequence comprising the amino acid residues beginning at position 1, 40, or any of the amino between positions 66-84 to position 413, any of the amino acid residues between positions 382-532, or any of the amino acid residues between positions 543-551 of SEQ ID NO:1; and (b) a protein comprising an amino acid sequence that is at least about 70%, 80%, 85%, 90%, or 95% identical to the amino acid sequence of (a). In one embodiment, the human cystathionine beta-synthase can catalyze the pyridoxal 5′-phosphate (PLP)-dependent condensation of serine and homocysteine to form cystathionine (i.e., the enzymatic activity or catalytic activity), bind heme, bind PLP, bind to AdoMet, and/or respond to AdoMet.

In another embodiment, the cystathionine beta-synthase variant comprises the amino acid residues from any one of positions 66, 67, 68, 69, 70, 71, 72, 73, 74, 75, 76, 77, 78, 80, 81, 82, 83, or 84, through any one of the amino acid residues present at a position from about 385-551 (e.g., 400-523 or 543-551) of SEQ ID NO:1. In some embodiments, the cystathionine beta-synthase variant comprises the amino acid residues from any one of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, through any one of the amino acid residues present at positions 385-551 (e.g., positions 400, 413, 488, 496, 523, 543, 551) of SEQ ID NO:1.

In one embodiment, the cystathionine beta-synthase variant differs from SEQ ID NO:1 by a deletion of at least the amino acid residues at positions 1-39 of SEQ ID NO:1. In another embodiment, the cystathionine beta-synthase variant differs from SEQ ID NO:1 by a deletion of at least the amino acid residues at positions 1-50 of SEQ ID NO:1. In another embodiment, the cystathionine beta-synthase variant differs from SEQ ID NO:1 by a deletion of at least the amino acid residues at positions 1-60 of SEQ ID NO:1. In yet another embodiment, the cystathionine beta-synthase variant differs from SEQ ID NO:1 by a deletion of at least the amino acid residues at positions 1-70 of SEQ ID NO:1. In yet another embodiment, the cystathionine beta-synthase variants differs from SEQ ID NO:1 by a deletion of between about 1 and about 8 amino acid residues from the C-terminus of SEQ ID NO:1. In another embodiment, the cystathionine beta-synthase variant differs from SEQ ID NO:1 by a deletion of between about 19 and about 169 amino acid residues from the C-terminus of SEQ ID NO:1. I another embodiment, the cystathionine beta-synthase variant differs from SEQ ID NO:1 by a deletion of between about 28 and about 169 amino acid residues from the C-terminus of SEQ ID NO:1. In yet another embodiment, the cystathionine beta-synthase variant differs from SEQ ID NO:1 by a deletion of between about 28 and about 151 amino acid residues from the C-terminus of SEQ ID NO:1.

In some embodiments the human cystathionine beta-synthase variant comprises a deletion of the first or both the first and second amino acid residues at the N-terminus of a naturally occurring human cystathionine β-synthase amino acid sequence. For example, the human cystathionine beta-synthase can comprise an amino acid sequence selected from of: (a) the amino acid residues at positions 2-551 of SEQ ID NO:1; (b) an amino acid sequence that is at least about 70%, 80%, 85%, 90%, or 95% identical to the amino acid residues at positions 2-551 of SEQ ID NO:1; or (c) an enzymatically active fragment of SEQ ID NO:1, wherein the fragment catalyzes the formation of cystathionine. In some embodiments, the cystathionine beta-synthase comprises an amino acid sequence comprising the amino acid residues present at positions 2-551 of SEQ ID NO:1, with at least one deletion or mutation of: Cys52 and/or His65, wherein the variant is capable of catalyzing the formation of cystathionine and/or has a reduced ability to bind heme as compared to wild-type cystathionine beta-synthase. In some embodiments, the enzymatically active fragment of cystathionine beta-synthase comprises or consists of the amino acid residues from positions 40-551 of SEQ ID NO:1. In some embodiments, the enzymatically active fragment of cystathionine beta-synthase comprises or consists of the amino acid residues from positions 66-551 of SEQ ID NO:1. In yet another embodiment, the enzymatically active fragment of cystathionine beta-synthase comprises or consists of the amino acid residues from positions 70-551 of SEQ ID NO:1. In some embodiments, the enzymatically active fragment of cystathionine beta-synthase comprises or consists of the amino acid residues from positions 71-551 of SEQ ID NO:1. In some embodiments, the enzymatically active fragment of cystathionine beta-synthase comprises or consists of the amino acid residues from positions 84-551 of SEQ ID NO:1. In some embodiments, the enzymatically active fragment of cystathionine beta-synthase comprises the amino acid residues from about position 1, 2, 3, 4, 5, 6, 7, or 8 to position 551 of SEQ ID NO:1. In another embodiment, the enzymatically active fragment of cystathionine beta-synthase comprises the amino acid residues from position 2 to about any one of positions 382 through 532 (e.g., 382, 400, 523, or 532) of SEQ ID NO: 1.

In one embodiment, any of the above-described proteins comprise no more than one or two amino acid residues at the N-terminus that is not a residue of the naturally occurring human cystathionine beta-synthase amino acid sequence.

In some embodiments the exogenous polypeptide provided herein is a fusion protein comprising any of the cystathionine beta-synthases or variants described herein linked to a heterologous protein sequence (e.g., via a linker).

Methionine Gamma-Lyase

The disclosure provides, in one aspect, an erythroid cell engineered to degrade homocysteine and its metabolites, comprising a first exogenous polypeptide comprising a homocysteine degrading polypeptide, or variant thereof, wherein the homocysteine degrading polypeptide, or variant thereof is a methionine gamma-lyase, or a variant thereof. In one embodiment, the erythroid cell comprises more than one exogenous polypeptide, wherein each exogenous polypeptide comprises a methionine gamma-lyase, e.g., methionine gamma-lyase from the same (e.g., variants) or from different sources or species.

Methionine gamma-lyase (E.C. 4.4.1.11) is an enzyme which requires pyridoxal 5′-phosphate (PLP) as a coenzyme and can catalyze one or more of: α, γ-dissociation and γ-substitution of L-methionine or its derivatives, and α, β-dissociation and β-substitution of S-substituted L-Cysteine or its derivatives, such as homocysteine (Tanaka, H. et al., Biochemistry, 16, 100-106 (1977)). This enzyme has been isolated and purified mainly from Pseudomonas putida and its physicochemical and enzymatic properties have been investigated (Nakayama, T. et al., Anal. Biochem., 138, 421-424 (1984)]). In one embodiment, the enzymatic activity of the methionine gamma-lyase has specificity for homocysteine as compared to methionine and/or cysteine. In one embodiment, the methionine gamma-lyase has reduced or minimal enzymatic activity for methionine (e.g., methionine catalytic activity) as compared to a wild-type methionine gamma-lyase, while retaining activity for homocysteine. In one embodiment, the methionine gamma-lyase has reduced or minimal activity for cysteine (e.g., cysteine catalytic activity), while retaining activity for homocysteine. In one embodiment, the methionine gamma-lyase has a kcat (i.e., the number of substrate molecule each enzyme site converts to product per unit time) for homocysteine that is at least about 10-fold greater (e.g., at least 10-fold, 15-fold, 20-fold, 25-fold, 30-fold, 35-fold, 40-fold, 45-fold, 50-fold, 100-fold, 150-fold, 200-fold, 250-fold, 300-fold, 350-fold, 400-fold, 450-fold, 500-fold or greater) than the kcat for cysteine. In some embodiments, the methionine gamma-lyase has a kcat for homocysteine that is at least about 10-fold greater (e.g., at least 10-fold, 15-fold, 20-fold, 25-fold, 30-fold, 35-fold, 40-fold, 45-fold, 50-fold, 100-fold, 150-fold, 200-fold, 250-fold, 300-fold, 350-fold, 400-fold, 450-fold, 500-fold or greater) than the kcat for methionine.

An engineered erythroid cell of the disclosure can comprise an exogenous polypeptide comprising a methionine gamma-lyase, or variant thereof, wherein the methionine gamma-lyase is derived from any source(s) known in the art, including mammalian, bacterial, fungal (including yeast), or protozoan sources, as well as methionine gamma-lyases generated by recombinant technologies.

In some preferred embodiments, the methionine gamma-lyase (or variant thereof) comprises an amino acid sequence set forth in Table 2, below (e.g., SEQ ID NOs: 37-46), including a methionine gamma-lyase, or variant thereof, derived from a Pseudomonas Putida.

TABLE 2
Exemplary Methionine Gamma-Lyases (Full-length sequences)

| SEQ. ID | Methionine Gamma | |
|---|---|---|
| NO | Lyase source | Amino acid sequence |
| 37 | Pseudomonas | MHGSNKLPGFATRAIHHGYDPQDHGGALVPPVYQTATFTFPTVEYG |
| | putida | AACFAGEQAGHFYSRISNPTLNLLEARMASLEGGEAGLALASGMGAI |
| | | TSTLWTLLRPGDEVLLGNTLYGCTFAFLHHGIGEFGVKLRHVDMADL |
| | | QALEAAMTPATRVIYFESPANPNMHMADIAGVAKIARKHG |
| | | ATVVVDNTYCTPYLQRPLELGADLVVHSATKYLSGHGDITAGIVVGS |
| | | QALVDRIRLQGLKDMTGAVLSPHDAALLMRGIKTLNLRMDRHCAN |
| | | AQVLAEFLARQPQVELIHYPGLASFPQYTLARQQMSQPGGMIAFEL |
| | | KGGIGAGRRFMNALQLFSRAVSLGDAESLAQHPASMTHSSYT |
| | | PEERAHYGISEGLVRLSVGLEDIDDLLADVQQALKAS |
| 38 | Fusobacterium | MEMKKSGLGTTAIHAGTLKNLYGTLAMPIYQTSTFIFDSAEQGGRRF |
| | nucleatum | ALEEAGYIYTRLGNPTTTVLENKIAALEEGEAGIAMSSGMGAISSTLW |
| | | TVLKAGDHVVTDKTLYGCTFALMNHGLTRFGVEVTFVDTSNLEEVK |
| | | NAMKKNTRVVYLETPANPNLKIVDLEALSKIAHTNPNTL |
| | | VIVDNTFATPYMQKPLKLGVDIVVHSATKYLNGHGDVIAGLVVTRQE |
| | | LADQIRFVGLKDMTGAVLGPQEAYYIIRGLKTFEIRMERHCKNARTIV |
| | | DFLNKHPKVEKVYYPGLETHPGYEIAKKQMKDFGAMISFELKGGFEA |
| | | GKTLLNNLKLCSLAVSLGDTETLIQHPASMTHSPYTKE |
| | | EREVAGITDGLVRLSVGLENVEDIIADLEQGLEK |
| 39 | Streptomyces | MSQPIDSVTAGHTPEGAPAEPDAPGWSFETRQLHAGAAPDTATGA |
| | ambofaciens | RATPIYQTTSFVFQDTQHAADLFSLAEPGNIYTRIHNPTQDVFEQRVA |
| | | SLEGGVAAVALASGQAAETLAVLTLAGAGDHIVSSASLYGGTYNLFR |
| | | HTLPRFGIEVSFVDDPDDIEAWRAAIRPGTKALFAETLGN |
| | | PRGNVLDVRAVADVAHEAGVPLIVDNTVPTPYLLRPIEHGADIVVHS |
| | | ATKFLGGHGTTIGGVVVDGGTFDFGAHAERFPGFTEPDPSYHGLRY |
| | | WPALGPGAFAVKLRVQLLRDLGPALSPHSAFLLLQGVETLSLRIERHS |
| | | ANAQALAEWLEQRDEVAAVHYPGLPSNRWYDAGQRYLPR |
| | | GGGAVLAFELRDGVKAGKRFVDAVELFSHLANIGDVRSLIIHPASTTH |
| | | SQLDEEQLAATGASPGLVRLSVGIENLADLKADLEAGFRAAKGA |
| 40 | Clostridium | MKIETKCLHEGYKPQNGEPVALPIYQSITYRYDNADEIGKLFDLTSSG |
| | saccharobutylicum | HMYSRISNPTVAAVEEKIAALEGGVAALCTTSGQAAALISILNILSAGD |
| | | HIVSVSTIYGGTINLFAVTLKKFGIQCTFVDAEASEEEIQKAFKANTKVV |
| | | FGETIANPAIAIFDIEKFANIAHKNNVPLIIDNTFATPILCRPIEFGADIVI |
| | | HSTSKYMDGHAVQIGGVIVDSGKFDWTNGKFPEFTEPDASY |
| | | HGIVYTEKFGSAAYITKARVQLMRDLGAYPSANAAFLLNLGLESLPVR |
| | | IEKHCRNAEAVAEFLNSNDKVEFVNYPTIKCNKYNDLAKKYLPNGCS |
| | | GVISFSIKGDRDDAIRFMNSLKLIENVVHVADIRTSVLHPASSTHRQLT |
| | | DEQLVAAGITPGLIRISVGLENIDDIIEDIKQALEQ |
| 41 | Bacillus mycoides | MKKKHMETALIHHGYTSEEHKGSLTPPLFQTSTFTFETAQQGEASFA |
| | | GVDPSYIYSRLGNPTVKLFEERMAVLEGGEEALAFGSGMAAISATLIG |
| | | FLKAGDHIICSNGLYGCTYGFLEVLEEKFMITHSFCDMETETDIENKIR |
| | | PNTKLIFVETPINPTMKLIDLEKVIGVAKRNDLLVI |
| | | VDNTFCSPYLQRPLELGCDAVVHSATKYIGGHGDVVAGVTICKTKAL |
| | | AEKIRPMRKDIGGIMAPFDAWLLLRGLKTLAVRMDRHCDNAEKIVSF |
| | | LKNHDAVEGVWYPEGELASRQMKRGG |
| | | GVISFSIKGGKEETQAFINDLYFITIAVSLGDTETLIQHPATMTHAAIPA |
| | | DLRKEMGIYDNLIRLSVGLESWEDIVSDLEQALKKISTVN |
| 42 | Bordetella | MSDSNKNWRLETIAVHGGYRPDPTTRAVAVPIYQTVAYAFDDTQH |
| | trematum | GADLFDLKVPGNIYTRIMNPTNDVLEQRVAALEGGIAALALASGQAA |
| | | VTYSILTLAEAGDNIVSSSTLYGGTYNLLAHTLPQYGISTRFADPSDLAS |
| | | FERQIDARTKAIFAESVGNPLGNVTDIQALADLAHRHG |
| | | VPLIVDNTVPSPYLLRPIEHGADIVVQSLTKYLGGHGTSLGGAIIDSGK |
| | | FPWAQHKERFKRLNEPDVSYHGVVYTEAFGAAAYIGRARVVPLRNT |
| | | GAALSPFNAFQILOGIETLALRVDRIVENAVRIAQYLKQHPKVEWVN |
| | | YAGLPEHPDHALVQKYLGGKAPGLFTFGVKGGREAGAR |
| | | FQDALNLFTRLVNIGDAKSLATHPASTTHRQLNPEELAKAGVREETV |
| | | RLSIGIEHIDDLIADLDQALAQ |
| 43 | Citrobacter | MSDCRTYGFNTQIVHAGQQPDPSTGALSTPIFQTSTFVFDSAEQGA |
| | freundii | ARFALEESGYIYTR |
| | | LGNPTTDALEKKLAVLERGEAGLATASGISAITTTLLTLCQQGDHIVSA |
| | | SAIYGCTHAFL |
| | | SHSMPKFGINVRLVDAGKPEEIRAAMRPETKVVYIETPANPTLSLVDI |
| | | ETVAGIAHQQGA |
| | | LLVVDNTFMSPYCQQPLQLGADIVVHSVTKYINGHGDVIGGIIVGKQ |
| | | EFIDQARFVGLKD |
| | | ITGGCMSPFNAWLTLRGVKTLGIRMERHCENALKIARFLEGHPSITRV |
| | | YYPGLSSHPQYE |
| | | LGQRQMSLPGGIISFEIAGGLEAGRRMINSVELCLLAVSLGDTETLIQ |
| | | HPASMTHSPVAP |
| | | EERLKAGITDGLIRLSVGLEDPEDIINDLEHAIRKATF |
| 44 | Entamoeba | MTAQDITTTLLHPKGDHVLHSHAYPIFQTSTFCFDSTQQG |
| | histolytica | ADLFMGKGEGHIYSRLGNPTVEQFEEMVCSIEGAAGSAAF |
| | | GSGMGAISSSTLAFLQKGDHLIAGDTLYGCTVSLFTHWLP |
| | | RFGIEVDLIDTSDVEKVKAAWKPNTKMVYLESPANPTCKV |
| | | SDIKGIAVVCHERGARLVVDATFTSPCFLKPLELGADIAL |
| | | HSVSKYINGHGDVIGGVSSAKTAEDIATIKFYRKDAGSLM |
| | | APMDAFLCARGMKTLPIRMQIHMENGLKVAKFLEQHEKIV |
| | | KVNHPGLESFPGHDIAKKQMTGYGSTFSFEMKSFEAAKKL |
| | | MEHLKVCTLAVSLGCVDTLIEHPASMTHAAVPENIMRKQG |
| | | ITPELVRISVGIENVDDIIADLKQALELW |
| 45 | Yersinia | MKLETLSIHAGYSPDPTTKAVAVPIYQTSSFAFDDTQHGADLFDLKVA |
| | frederiksenii | GNIYSRIMNPTNDVLEKRVAALEGGIAALAVASGMAAITYAIQTIAEV |
| | | GDNIVSVAKLYGGTYNLLAHTLPRYGIETRFADHDDIAALEALIDDRTK |
| | | AVFCESIGNPAGNIVDLKKLADAAHRHGVPLIVDNTVATPILCRPFEH |
| | | GADIVVHSLTKYIGGHGSSIGGIVVDSGKFPWAQYPERFAQLNTPDP |
| | | SYHGVTYTEQFGAAAYIGRCRVVPLRNTGAALSPFNAFLILQGLETLA |
| | | LRMERHTENALKVAHYLQNHPQVSWVKYAGLPDHPEHELAQRYFG |
| | | GKPASILSFGIHGGQEAGGRFIDALNLIVRLVNIGDAKSLACHPASTTH |
| | | RQLNDEELIKAGVPRDMVRLSIGIEHIDDILNDLAQAL |
| | | DAAK |
| 46 | Bacillus subtilis | MGESWGKGTICVQGGYTPKNGEPRVLPLYQSTTYKYDTSDDLAALF |
| | | NLEAEGYIYTRIGNPTLAAFEQKLAELEGGVGAVATASGQAAIMLAVL |
| | | NICSSGDHLLCSSTVYGGTFNLFGVSLRKLGIDVTFFNPNLTADEIVAL |
| | | ANDKTKLVYAESLGNPAMNVLNFKEFSDAAKELEVPF |
| | | IVDNTLATPYLCQAFEHGANIIVHSTTKYIDGHASSLGGIVIDGGNFD |
| | | WTNGKYPELVEPDPSYHGVSYVQNFGAAAYIVKARVQLLRDYGNC |
| | | MSPFNAYISNIGLETLHLRMERHSENA |
| | | LAVAKWLADHERIEWVNYPGLDSNENYSLAQKYLKKGASGVLTFGIK |
| | | GGLEAAKEFIANVKLATLVTHVADARTCVIHPASTTHRQLSAEDQRL |
| | | AGVTSDLIRLSVGIEDVSDIIADLEAALVGGKQHADH |

In some embodiments, the methionine gamma-lyase, or variant thereof, comprises or consists of an amino acid sequence comprising SEQ ID NO: 37, with a cysteine to histidine substitution at position 116 (cysteine position determined based on SEQ ID NO: 37; C116H). The C116H mutant MGL is described, for example,in Kudou et al. (Bioscience, Biotechnology and Biochemistry, 72:7, 1722-1730 (2008), the entire content of which is incorporated by reference herein). In some embodiments, the methionine gamma-lyase, or variant thereof, comprises an amino acid substitution C to H at an amino acid residue corresponding to the amino acid at position 116 in SEQ ID NO: 37. In some preferred embodiments of the disclosure, the methionine gamma-lyase, or variant thereof, comprises or consists of the amino acid sequence set forth in SEQ ID NO: 47, below.

SEQ ID NO: 47
MHGSNKLPGFATRAIHHGYDPQDHGGALVPPVYQTATFTFPTVEYGAACF
AGEQAGHFYSRISNPTLNLLEARMASLEGGEAGLALASGMGAITSTLWTL
LRPGDEVLLGNTLYGHTFAFLHHGIGEFGVKLRHVDMADLQALEAAMTPA
TRVIYFESPANPNMHMADIAGVAKIARKHGATVVVDNTYCTPYLQRPLEL
GADLVVHSATKYLSGHGDITAGIVVGSQALVDRIRLQGLKDMTGAVLSPH
DAALLMRGIKTLNLRMDRHCANAQVLAEFLARQPQVELIHYPGLASFPQY
TLARQQMSQPGGMIAFELKGGIGAGRRFMNALQLFSRAVSLGDAESLAQH
PASMTHSSYTPEERAHYGISEGLVRLSVGLEDIDDLLADVQQALKAS

In one embodiment, the methionine gamma-lyase comprises or consists of an amino acid sequence of SEQ ID NO:37, or a variant thereof. In one embodiment, the methionine gamma-lyase comprises or consists of an amino acid sequence of SEQ ID NO:38, or a variant thereof. In one embodiment, the methionine gamma-lyase comprises or consists of an amino acid sequence of SEQ ID NO:39, or a variant thereof. In one embodiment, the methionine gamma-lyase comprises or consists of an amino acid sequence of SEQ ID NO:40, or a variant thereof. In one embodiment, the methionine gamma-lyase comprises or consists of an amino acid sequence of SEQ ID NO:42, or a variant thereof. In one embodiment, the methionine gamma-lyase comprises or consists of an amino acid sequence of SEQ ID NO:42, or a variant thereof. In one embodiment, the methionine gamma-lyase comprises or consists of an amino acid sequence of SEQ ID NO:43, or a variant thereof. In one embodiment, the methionine gamma-lyase comprises or consists of an amino acid sequence of SEQ ID NO:44, or a variant thereof. In one embodiment, the methionine gamma-lyase comprises or consists of an amino acid sequence of SEQ ID NO:45, or a variant thereof. In one embodiment, the methionine gamma-lyase comprises or consists of an amino acid sequence of SEQ ID NO:46, or a variant thereof. In one embodiment, the methionine gamma-lyase comprises or consists of an amino acid sequence of SEQ ID NO:47, or a variant thereof.

In one embodiment, the methionine gamma-lyase comprises an amino acid sequence that is at least 95% identical (e.g., 96%, 97%, 98%, 99% or 100% identical) to the amino acid sequence set forth in SEQ ID NO:37. In one embodiment, the methionine gamma-lyase comprises an amino acid sequence that is at least 95% identical to the amino acid sequence set forth in SEQ ID NO:38. In one embodiment, the methionine gamma-lyase comprises an amino acid sequence that is at least 95% identical (e.g., 96%, 97%, 98%, 99% or 100% identical) to the amino acid sequence set forth in SEQ ID NO:39. In one embodiment, the methionine gamma-lyase comprises an amino acid sequence that is at least 95% identical (e.g., 96%, 97%, 98%, 99% or 100% identical) to the amino acid sequence set forth in SEQ ID NO:40. In one embodiment, the methionine gamma-lyase comprises an amino acid sequence that is at least 95% identical (e.g., 96%, 97%, 98%, 99% or 100% identical) to the amino acid sequence set forth in SEQ ID NO:41. In one embodiment, the methionine gamma-lyase comprises an amino acid sequence that is at least 95% identical (e.g., 96%, 97%, 98%, 99% or 100% identical) to the amino acid sequence set forth in SEQ ID NO:42. In one embodiment, the methionine gamma-lyase comprises an amino acid sequence that is at least 95% identical (e.g., 96%, 97%, 98%, 99% or 100% identical) to the amino acid sequence set forth in SEQ ID NO:43. In one embodiment, the methionine gamma-lyase comprises an amino acid sequence that is at least 95% identical (e.g., 96%, 97%, 98%, 99% or 100% identical) to the amino acid sequence set forth in SEQ ID NO:44. In one embodiment, the methionine gamma-lyase comprises an amino acid sequence that is at least 95% identical (e.g., 96%, 97%, 98%, 99% or 100% identical) to the amino acid sequence set forth in SEQ ID NO:45. In one embodiment, the methionine gamma-lyase comprises an amino acid sequence that is at least 95% identical (e.g., 96%, 97%, 98%, 99% or 100% identical) to the amino acid sequence set forth in SEQ ID NO:46. In one embodiment, the methionine gamma-lyase comprises an amino acid sequence that is at least 95% identical (e.g., 96%, 97%, 98%, 99% or 100% identical) to the amino acid sequence set forth in SEQ ID NO:47.

In one embodiment, the methionine gamma-lyase comprises a Pseudomonas putida methionine gamma-lyase. In one embodiment, the Pseudomonas putida methionine gamma-lyase comprises an amino acid sequence that is at least 95% identical (e.g., 96%, 97%, 98%, 99% or 100% identical) to the amino acid sequences set forth in SEQ ID NO: 37.

In one embodiment, the methionine gamma-lyase comprises a Fusobacterium nucleatum methionine gamma-lyase. In one embodiment, the Fusobacterium nucleatum methionine gamma-lyase comprises an amino acid sequence that is at least 95% identical (e.g., 96%, 97%, 98%, 99% or 100% identical) to the amino acid sequences set forth in SEQ ID NO: 38.

In one embodiment, the methionine gamma-lyase comprises a Streptomyces ambofaciens methionine gamma-lyase. In one embodiment, the Streptomyces ambofaciens methionine gamma-lyase comprises an amino acid sequence that is at least 95% identical (e.g., 96%, 97%, 98%, 99% or 100% identical) to the amino acid sequences set forth in SEQ ID NO: 39.

In one embodiment, the methionine gamma-lyase comprises a Clostridium saccharobutylicum methionine gamma-lyase. In one embodiment, the Clostridium saccharobutylicum methionine gamma-lyase comprises an amino acid sequence that is at least 95% identical (e.g., 96%, 97%, 98%, 99% or 100% identical) to the amino acid sequences set forth in SEQ ID NO: 40.

In one embodiment, the methionine gamma-lyase comprises a Bacillus mycoides methionine gamma-lyase. In one embodiment, the Bacillus mycoides methionine gamma-lyase comprises an amino acid sequence that is at least 95% identical (e.g., 96%, 97%, 98%, 99% or 100% identical) to the amino acid sequences set forth in SEQ ID NO: 41.

In one embodiment, the methionine gamma-lyase comprises a Bordetella trematum methionine gamma-lyase. In one embodiment, the Bordetella trematum methionine gamma-lyase comprises an amino acid sequence that is at least 95% identical (e.g., 96%, 97%, 98%, 99% or 100% identical) to the amino acid sequences set forth in SEQ ID NO: 42.

In one embodiment, the methionine gamma-lyase comprises a Citrobacter freundii methionine gamma-lyase. In one embodiment, the Citrobacter freundii methionine gamma-lyase comprises an amino acid sequence that is at least 95% identical (e.g., 96%, 97%, 98%, 99% or 100% identical) to the amino acid sequences set forth in SEQ ID NO: 43.

In one embodiment, the methionine gamma-lyase comprises a Entamoeba histolytica methionine gamma-lyase. In one embodiment, the Entamoeba histolytica methionine gamma-lyase comprises an amino acid sequence that is at least 95% identical (e.g., 96%, 97%, 98%, 99% or 100% identical) to the amino acid sequences set forth in SEQ ID NO: 44.

In one embodiment, the methionine gamma-lyase comprises aYersinia frederiksenii methionine gamma-lyase. In one embodiment, the Yersinia frederiksenii methionine gamma-lyase comprises an amino acid sequence that is at least 95% identical (e.g., 96%, 97%, 98%, 99% or 100% identical) to the amino acid sequences set forth in SEQ ID NO: 45.

In one embodiment, the methionine gamma-lyase comprises a Bacillus subtilis methionine gamma-lyase. In one embodiment, the Bacillus subtilis methionine gamma-lyase comprises an amino acid sequence that is at least 95% identical (e.g., 96%, 97%, 98%, 99% or 100% identical) to the amino acid sequences set forth in SEQ ID NO: 46.

In some embodiments, the methionine gamma-lyase comprises a variant of a wild-type methionine gamma-lyase having at least 40%, at least 50%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46 or SEQ ID NO:47. In some embodiments, the methionine gamma-lyase variant possesses a function of a methionine gamma-lyase as described herein, e.g., the variant can catalyze one or more of α, γ-dissociation and γ-substitution of L-methionine or its derivatives and α, β-dissociation and β-substitution of S-substituted L-Cysteine or its derivatives (such as homocysteine).

In a particular embodiment, the methionine gamma-lyase consists of the amino acid sequence of any one of SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46 or SEQ ID NO:47.

A methionine gamma-lyase can also include proteins having an amino acid sequence comprising at least 10 contiguous amino acid residues of any one of SEQ ID NOs:37-47 (i.e.,10 contiguous amino acid residues having 100% identity with 10 contiguous amino acid residues of any one of SEQ ID NOs:37-47). In other embodiments, a methionine gamma-lyase amino acid sequence includes amino acid sequences comprising at least 20, or at least 30, or at least 40, or at least 50, or at least 75, or at least 100, or at least 125, or at least 150, or at least 175, or at least 150, or at least 200, or at least 250, or at least 300, or at least 350, or at least 400, or at least 450, or at least 500, or at least 550, contiguous amino acid residues of the amino acid sequence represented by any one of SEQ ID NOs:37-47, and any whole integer in between 10 and 550 contiguous amino acid residues. In a preferred embodiment, a methionine gamma-lyase has measurable or detectable methionine gamma-lyase biological activity. In some embodiments, fragments or variants of the methionine gamma-lyase enzyme retain at least 50%, at least 55%, at least 60%, at least 65%, at least 70%, at least 75%, at least 80%, at least 85%, at least 90%, at least 95%, at least 98% or at least 99% of the activity as compared to the wild-type methionine gamma-lyase from which they were derived.

In general, a variant methionine gamma-lyase, from any origin, may be produced, for example, to enhance production of the protein in an engineered cell, to improve turnover/half-life of the protein or mRNA encoding the protein, and/or to modulate (enhance or reduce) the enzymatic activity of the methionine gamma-lyase. The methionine gamma-lyase, whatever the source, may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding a methionine gamma-lyase as described herein. In some embodiments, the invention provides an engineered erythroid cell prepared by using a nucleic acid sequence encoding a methionine gamma-lyase as described herein. In some embodiments, the nucleic acid sequence encodes a methionine gamma-lyase (e.g. Pseudomonas putida methionine gamma-lyase, Saccharomyces cerevisiae methionine gamma-lyase, Fusobacterium nucleatum methionine gamma-lyase, Streptomyces ambofaciens methionine gamma-lyase, Clostridium saccharobutylicum methionine gamma-lyase, Bacillus mycoides methionine gamma-lyase, Bordetella trematum methionine gamma-lyase, Citrobacter freundii methionine gamma-lyase, Entamoeba histolytica methionine gamma-lyase, Yersinia frederiksenii methionine gamma-lyase, and Bacillus subtilis methionine gamma-lyase) as described herein.

In some embodiments, the methionine gamma-lyase is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the corresponding wild-type methionine gamma-lyase nucleic acid (from any source) that encodes a protein that possesses a function of a methionine gamma-lyase described herein, e.g., the encoded protein is an enzyme which requires pyridoxal 5′-phosphate (PLP) as a coenzyme and can catalyze one or more of α, γ-dissociation and γ-substitution of L-methionine or its derivatives and α, β-dissociation and β-substitution of S-substituted L-Cysteine or its derivatives (such as homocysteine).

In some embodiments, the exogenous polypeptide is a fusion protein comprising any of the methionine gamma-lyases, or variants, described herein linked to a heterologous protein sequence (e.g., via a linker).

### Cystathionine Degrading Polypeptide

As described herein, the present disclosure provides an erythroid cell engineered to reduce homocysteine levels, comprising an exogenous polypeptide comprising at least one cystathionine degrading polypeptide, or a variant thereof (e.g. cystathioinine beta-synthase, or a variant thereof).

In some embodiments, the present disclosure provides erythroid cells comprising both an exogenous polypeptide comprising at least one homocysteine degrading polypeptide and an exogenous polypeptide comprising a cystathioinine degrading polypeptide. Without being bound by theory, it is possible that if, as a result of homocysteine degradation (e.g., as catalyzed by an exogenous polypeptide comprising a homocysteine degrading polypeptide such as cystathionine beta-synthase), cystathionine accumulates in the engineered erythroid cell and the concentration of cystathionine becomes too high, thereby promoting a reversal of the forward reaction catalyzed by cystathionine beta-synthase (i.e., the reversal of the reaction Homocystine+Serine→Cystathionine+H2O). Accordingly, accumulation of too great a concentration of cystathionine inside the engineered erythroid cell may inhibit the forward reaction of cystathioinine beta-synthase.

Accordingly, in order to prevent or overcome cystathionine accumulation, in certain aspects the engineered erythroid cells described herein may be engineered to further comprise an exogenous polypeptide comprising at least one cystathionine degrading polypeptide, or a variant thereof. In some embodiments, the erythroid cell comprises more than one exogenous polypeptude comprising a cystathionine degrading polypeptide, or a variant thereof. In some embodiments, the exogenous polypeptide comprising a cystathionine degrading polypeptide catalyzes the conversion of cystathionine into cysteine, α-ketobutyrate, and ammonia. In some embodiments, the cystathionine degrading polypeptide is cystathionine gamma-lyase.

In one aspect, the present disclosure provides an erythroid cell engineered to reduce homocysteine levels, comprising a first exogenous polypeptide comprising at least one homocysteine degrading polypeptide, or a variant thereof, and further comprising an exogenous polypeptide comprising at least one cystathionine degrading polypeptide (e.g. cystathionine gamma-lyase), or a variant thereof.

In one aspect, the present disclosure provides an erythroid cell engineered to reduce homocysteine levels, comprising a first exogenous polypeptide comprising at least one homocysteine degrading polypeptide, or a variant thereof, a second exogenous polypeptide comprising a homocysteine and/or serine transporter.

In another aspect, the present disclosure provides an erythroid cell engineered to reduce homocysteine levels, comprising a first exogenous polypeptide comprising at least one homocysteine degrading polypeptide, or a variant thereof, a second exogenous polypeptide comprising a homocysteine and/or serine transporter and a third exogenous polypeptide comprising at least one cystathionine degrading polypeptide.

Without wishing to be bound by theory, the cystathionine degrading polypeptide (e.g. cystathionine gamma-lyase) degrades cystathionine (e.g., generated by the catalytic activity of an exogenous polypeptide comprising a homocysteine degrading polypeptide) into metabolites or degradation products of cystathioinine (e.g. cysteine, α-ketobutyrate, and ammonia). In some embodiments, the cystathionine degrading polypeptide is cystathionine gamma-lyase ((EC 4.4.1.1) or a variant thereof.

An engineered erythroid cell of the disclosure may comprise an exogenous polypeptide comprising at least one cystathionine degrading polypeptide, or variant thereof, wherein the at least one cystathionine degrading polypeptide is derived from any source or species, e.g., mammalian, fungal (including yeast), protozoal, plant or bacterial sources, or can be recombinantly engineered. Insome embodiments, the cystathionine degrading polypeptide is a chimeric cystathionine degrading polypeptide, e.g., derived from two different species.

The engineered erythroid cells provided herein may comprising more than one exogenous polypeptide, wherein each exogenous polypeptide comprises a cystathionine degradation polypeptide, or variant thereof, from the same (e.g., variants) or different sources or species.

### Cystathionine Gamma-Lyase

In one aspect, the disclosure provides engineered erythroid cells comprising at least one exogenous polypeptide, wherein the exogenous polypeptide comprises a cystathionine degrading polypeptide such as a cystathionine gamma-lyase. Cystathionine gamma-lyase (also referred to as CGL; L-cystathionine cysteine-lyase (deaminating; 2-oxobutanoate-forming); (E.C.4.4.1.1)) is a multifunctional pyridoxal-phosphate protein. CGL cleaves a carbon-sulfur bond in cystathionine, releasing L-cysteine and an unstable enamine product that tautomerizes to an imine form, which undergoes a hydrolytic deamination to form 2-oxobutanoate and ammonia. Cystathionine gamma-lyase uses L-cysteine as a substrate to produce hydrogen sulfide (H₂S). The cystathionine gamma-lyase/hydrogen sulfide system has been shown to play an important role in regulating cellular functions in different systems. Hydrogen sulfide inhibits cell proliferation and induces cell death predominantly by an apoptotic mechanism in polymorphonuclear cells (Valitutti, S., et al. (1990) Ann. Allergy 65, 463-468; Mariggio, M. A., et al. (1998) Immunopharmacol. Immunotoxicol. 20, 399-408). Yang et al. (J Biol Chem. 2004 Nov. 19; 279(47):49199-205) have shown that cystathionine gamma-lyase overexpression resulted in an increase in intracellular H2S production rates, and an inhibition of cellular proliferation and DNA synthesis in HEK-293 cells.

It is a surprising finding of the present invention that the engineered erythroid cells comprising a cystathionine gamma-lyase, or variant thereof, are not detrimental to cell proliferation.

An engineered erythroid cell of the disclosure can comprise an exogenous polypeptide comprising cystathionine gamma-lyase, or variant thereof, derived from any source(s) known in the art, including mammalian, e.g., human, mouse, rat, Oryctolagus cuniculus, Monodelphis domestica, or Ornithorhynchus anatinus, insect, e.g., Drosophila melanogaster, bacterial, e.g., Mycobacterium tuberculosis, fungal (including yeast), e.g., Saccharomyces cerevisiae or, Emericella nidulan, or protozoa, e.g., Dictyostellium discoideum, as well as CGLs generated by recombinant technologies.

In some preferred embodiments of the disclosure, the cystathionine gamma-lyase (or variant thereof) comprises an amino acid sequence set forth in Table 3, below, including a cystathionine gamma-lyase, or variant thereof, derived from a human.

TABLE 3
Exemplary Cystathionine Gamma-Lyases (Full-length sequences)

| SEQ. ID | Cystathionine Gamma- | |
|---|---|---|
| NO | Lyase source | Amino acid sequence |
| 50 | Homo sapiens | MQEKDASSQG FLPHFQHFAT QAIHVGQDPE QWTSRAVVPP |
| | | ISLSTTFKQG APGQHSGFEYSRSGNPTRNC LEKAVAALDG |
| | | AKYCLAFASG LAATVTITHL LKAGDQIICM DDVYGGTNRY |
| | | FRQVASEFGL KISFVDCSKI KLLEAAITPE TKLVWIETPT |
| | | NPTQKVIDIE GCAHIVHKHGDIILVVDNTF MSPYFQRPLA |
| | | LGADISMYSA TKYMNGHSDV VMGLVSVNCE SLHNRLRFLQ |
| | | NSLGAVPSPI DCYLCNRGLK TLHVRMEKHF KNGMAVAQFL |
| | | ESNPWVEKVI YPGLPSHPQHELVKRQCTGC TGMVTFYIKG |
| | | TLQHAEIFLK NLKLFTLAES LGGFESLAEL |
| | | PAIMTHASVLKNDRDVLGIS DTLIRLSVGL EDEEDLLEDL |
| | | DQALKAAHPP SGSHS |
| 51 | Mus musculus | MQKDASLSGF LPSFQHFATQ AIHVGQEPEQ WNSRAVVLPI |
| | | SLATTFKQDF PGQSSGFEYSRSGNPTRNCL EKAVAALDGA |
| | | KHSLAFASGL AATITITHLL KAGDEIICMD EVYGGTNRYF |
| | | RRVASEFGLK ISFVDCSKTK LLEAAITPQT KLVWIETPTN |
| | | PTLKLADIGA CAQIVHKRGDIILVVDNTFM SAYFQRPLAL |
| | | GADICMCSAT KYMNGHSDVV MGLVSVNSDD LNSRLRFLQN |
| | | SLGAVPSPFD CYLCCRGLKT LQVRMEKHFK NGMAVARFLE |
| | | TNPRVEKVVY PGLPSHPQHELAKRQCSGCP GMVSFYIKGA |
| | | LQHAKAFLKN LKLFTLAESL GGYESLAELP AIMTHASVPE |
| | | KDRATLGIND TLIRLSVGLE DEQDLLEDLD RALKAAHP |
| 52 | Rattus norvegicus | MQKDASSSGF LPSFQHFATQ AIHVGQEPEQ WSSRAVVLPI |
| | | SLATTFKODS PGQSSGFVYSRSGNPTRNCL EKAVAALDGA |
| | | KHCLTFASGL AATTTITHLL KAGDEVICMD EVYGGTNRYF |
| | | RRVASEFGLK ISFVDCSKTK LLEAAITPQT KLVWIETPTN |
| | | PTLKLADIKA CAQIVHKHKDIILVVDNTFM SAYFQRPLAL |
| | | GADICMCSAT KYMNGHSDVV MGLVSVNSDD LNERLRFLQN |
| | | SLGAVPSPFD CYLCCRGLKT LQIRMEKHFR NGMAVARFLE |
| | | SNPRVEKVIY PGLPSHPQHELAKRQCTGCP GMVSFYIKGT |
| | | LQHAQVFLKN IKLFALAESL GGYESLAELP AIMTHASVPE |
| | | KDRATLGISD TLIRLSVGLE DEKDLLEDLG QALKAAHP |
| 53 | Saccharomyces | MTLQESDKFA TKAIHAGEHV DVHGSVIEPI SLSTTFKQSS |
| | cerevisiae | PANPIGTYEY SRSQNPNRENLERAVAALEN AQYGLAFSSG |
| | | SATTATILQS LPQGSHAVSI GDVYGGTHRY FTKVANAHGV |
| | | ETSFTNDLLN DLPQLIKENT KLVWIETPTN PTLKVTDIQK |
| | | VADLIKKHAA GQDVILVVDNTFLSPYISNP LNFGADIVVH |
| | | SATKYINGHS DVVLGVLATN NKPLYERLQF |
| | | LQNAIGAIPPFDAWLTHRG LKTLHLRVRQ AALSANKIAE |
| | | FLAADKENVV AVNYPGLKTH PNYDVVLKQHRDALGGGMIS |
| | | FRIKGGAEAA SKFASSTRLF TLAESLGGIE SLLEVPAVMT |
| | | HGGIPKEARASGVFDDLVR ISVGIEDTDD LLEDIKQALK QATN |
| 54 | Neurospora crassa | MTASSDHPIT TPPRAPSPVH GFGTLAVHAG SPHDPATGAV |
| | | IEAISLSTTF AQTAVGKPVGEFEYSRSSNP NRANFEKMVA |
| | | ALEHAKYALA YSSGSATTAN ILQSLAAGSH VISVSDVYGG |
| | | THRYFTQVAK AHGVKVTFTP EIEVDIRDHI TDATKLVWIE |
| | | TPSNPTLRLV DIRAVATAAHERGILVVVDN TFLSPYVQNP |
| | | LDHGADIVVH SVTKYINGHS DVVMGVAAFN SDELYARLSF |
| | | LQNAIGAVPS AFDSWLAHRG AKTLHLRARE ATTNATAIAH |
| | | ALEASPLVIS VNYPGLESHPHRAIALKQHR NGMGGGMLSF |
| | | RIHGGHAAAE KFCQYTKIFT LAESLGGVES LCEIPSSMTH |
| | | AGIPKAQREA VGIFDDLVRI SCGVEDAEDL KADVLQALER |
| | | AVADAANGVS NGVNGTH |
| 55 | Leishmania major | MSSQQHLVSD FTAGSGSWLP QSQGFDTLQV HAGVRPDPVT |
| | | GAILTPIYQS TTFVQESINSYQAKGYSYTR SANPTVAVLE |
| | | QKLCALENGS YCTVYNTGMA ATTTAISSFM NAGDHAILTN |
| | | CCYGGTNRAC RVFFSRLGME FTFVDMRDPQ NVIDSIKPNT |
| | | KLVISETPAN PTLILIDVAAVSKICKERGI VHMCDNTFAT |
| | | AYIMRPLDHG ADVTLISTTK YVDGHDMTVG GALVTNSKEL |
| | | DAKVRLTQNI LGNVMSPQVA FLQLQTVKTM SLRVTKQSHN |
| | | AQKIAEFLET HRAVDRVVYPGGMLWFEVKG GTAAGRRLMD |
| | | TVPRPWSLCE NLGASESIITCPSVMTHANM TSEDRMKVGI |
| | | TDGFVRVSCG IEDVDDLIAA LKVAMDALV |
| 56 | Corynebacterium | MKFPTQDELR ARFTRKWTQF EPDVLPLFIA ESDFPTAPVI |
| | ammonia genes | KEVLLDYTER ECFGYSPAPGTVDLGGAVSE FHAKRYGWRP |
| | | DPKKVFWIGD VVRGLLLGIQ YFTRPDSPVV VPLPAYPPFL |
| | | ELPETAGRKR IDVGVSPANG DLKEGEKPRL DLESIERAFA |
| | | DGAGSILLAN PYNPLGYVFDEEHLHELVEL ADKYDARILS |
| | | DEIHAPLVLD GQHISVAGLS DAAARVTMTI TATSKAWNFA |
| | | GLKCAQIIFS NDEDVKTWNS LPHVAQDGVG TLGIIAAEAA |
| | | YRDGIPHLEE EIEYLRSTRDWLVEELPKRI PGLVTSKPDS |
| | | TYLLWLDFRN TAIGDKEHPA AWLIQHARVA FNEGVTFGEG |
| | | GAHQARMNFA TSPEILEEAIDRIATAIAKV SDN |
| 57 | Emericella nidulans | MTMMAGHPDL PSNGQNGDSN THQQRQFATL |
| | | AVHAGAPHDP TTGAVIAPIS LSTTFAQESVGKPVGLYEYT |
| | | RSSNPNRDNF EEAVASLEHA KYALAFSSGS ATTATILHSL |
| | | APGSHVVSVSDVYGGTHRYF TKVAAAHGVN VSFSSCLELD |
| | | VEKLIRPNET KLVWIETPSN PTLALVDIRKVAAVAHRHGV |
| | | LVVVDNTFMS PYVQNPLDHG ADVVIHSVTK YINGHSDVLM |
| | | GVAAFNSDELKERFTFLQNA IGAVPSPFDC WLAHRGLKTL |
| | | HLRAREATAN ATAVALALES SPHVISVNYPGLNSHPNREI |
| | | AVKQHRKGMG GGMLSFRIKG GHKAAHLFCE YTKIFTLAES |
| | | LGGVESLCEVPSSMTHAGIP KEEREAAGVY DDLVRMSCGI |
| | | EDVEDLTADT MQALERAVAA SQALENGSA |
| 58 | Arabidopsis thaliana | MEAGERRNGD SMSHNHRAPK KPRLAGLLTE SDIDSEFAHH |
| | | QTGVARINNG SFGCCPGSVLEAQREWQLRY LRQPDEFYFN |
| | | GLRRGLLASR TVISDLINAD DVDEVSLVDN ATTAAAIVLQ |
| | | KVGRCFSEGK YKKEDTVVMF HCAFQSVKKS IQAYVSRVGG |
| | | STVEVRLPFP VNSNEEIISKFREGLEKGRA NGRTVRLAII |
| | | DHITSMPCVL MPVRELVKIC REEGVEQVFV DAAHAIGSVK |
| | | VDVKEIGADY YVSNLHKWFF CPPSIAFFYC KKRGSESDVH |
| | | HPVVSHEFGN GLPIESAWIGTRDYSSQLVV PSVMEFVNRF |
| | | EGGMEGIMMK NHDEAVRMGL MLADAWGTNL |
| | | GSPPEMCVGM VMIGLPSKLC VGSDEDAIKL RSYLRVHYSV |
| | | EVPVFYLGLR DGEEGVKDKD SGLITAYVRI |
| | | SHQVYNKTED YERLRDAITE LVKDQMTCQN LPAL |
| 70 | Pongo abelii (Sumatran | MQEKEASSQGFLPHFQHFATQAIHVGQEPEQWTSRAVVPPISP |
| | orangutan) | SVTFKQGAPGQHSGFVYSRSGNPTRNCLEKAVAALDGAKYCLAF |
| | cystathionine gamma | ASGLAATVTITHLLKAGDQIICMDDVYAGTNRYFRQVASEFGLKI |
| | lyase | SFVDCSKIKLLEAAITPETKLVWIETPTNPTQKMTDIEACAHIVHK |
| | (Genbank Accession | HGDIILVVDNTFMSPYFQRPLALGADICMCSATKYMNGHSDVV |
| | No. NP_001124635) | MGLVSVNCESLYNRLRFLQNSLGAVPSPIDCYLCNRGLKTLQVR |
| | | MEKHFKNGMAVAQFLESNPWVEKVIYPGLPSHPQHELVKRQC |
| | | TGCTGMVTFYIKGTLQHAEIFLKNLKLFTLAESLGGFESLVELPAV |
| | | MTHASVLKKDRDVLGISDTLIRLSVGLEDEEDLLEDLDQALKAAH |
| | | PPSGSHS |
| 71 | Macaca fascicularis | MQEKDASSQGFLPHFQHFATQAIHVGQEPEQWTSRAVVPLISL |
| | (long-tailed macaque) | STTFKQAAPGQHSGFEYSRSGNPTRNCLEKAVAALDGAKYCLAF |
| | cystathionine gamma | ASGLAATVTITHLLKAGDQIICMDDVYGGTNRYFRQVASEFGLKI |
| | lyase | SFVDCSKIKLLEAAITPETKLVWIETPTNPVLKMIDIEACAHIVHKR |
| | (Genbank Accession | GDIILVVDNTFMSPYFQRPLALGADICMCSATKYMNGHSDVVM |
| | No. AAW71993) | GLVSVNCERLHNRLRFLQNSLGAVPSPLDCYLCNRGLKTLHVRM |
| | | EKHFKNGMAVAQFLESNPGVEKVIYPGLPSHPQHELAKRQCTG |
| | | CTGMITFYIKGTLQHAEIFLKNLKLFTLAESLGGFESLVELPAIMTH |
| | | ASVPKNDRDVLGISDTLIRLSVGLEDEKDLLEDLDQALKAAHPPS |
| | | GSHN |
| 72 | Pan troglodytes | MQEKDASSQGFLPHFQHFATQAIHVGQDPEQWTSRALVPPISL |
| | (chimpanzee) | STTFKQGAPGQHSGFEYSRSGNPTRNCLEKAVAALDGAKYCLAF |
| | cystathionine gamma | ASGLAATVTITHLLKAGDQIICMDDVYGGTNRYFRQVASEFGLKI |
| | lyase | SFVDCSKIKLLEAAITPETKLVWIETPTNPTQKVIDIEACAHIVHKH |
| | (NCBI Reference | GDIILVVDNTFMSPYFQRPLALGADICMYSATKYMNGHSDVVM |
| | Sequence No. | GLVSVNCESLHNRLRFLQNSLGAVPSPIDCYLCNRGLKTLHVRME |
| | XP_513486.2) | KHFKNGMAVAQFLESNPWVEKVIYPGLPSHPQHELVKRQCTGC |
| | | TGMVTFYIKGTLQHAEIFLKNLKLFTLAESLGGFESLAELPAIMTH |
| | | ASVLKNDRDVLGISDTLIRLSVGLEDEEDLLEDLDQALKAAHPPSG |
| | | SHS |
| 73 | Pan paniscus (bonobo) | MQEKDASSQGFLPHFQHFATQAIHVGQDPEQWTSKALVPPISL |
| | cystathionine gamma | STTFKQGAPGQHSGFEYSRSGNPTRNCLEKAVAALDGAKYCLAF |
| | lyase | ASGLAATVTITHLLKAGDQIICMDDVYGGTNRYFRQVASEFGLKI |
| | (NCBI Reference | SFVDCSKIKLLEAAITPETKLVWIETPTNPTQKVIDIEACAHIVHKH |
| | Sequence No.: | GDIILVVDNTFMSPYFQRPLALGADICMYSATKYMNGHSDVVIG |
| | XP_003830652.1) | LVSVNCESLHNRLRFLQNSLGAVPSPIDCYLCNRGLKTLHVRMEK |
| | | HFKNGMAVAQFLESNPWVEKVIYPGLPSHPQHELVKRQCTGCT |
| | | GMVTFYIKGTLQHAEIFLKNLKLFTLAESLGGFESLAELPAIMTHA |
| | | SVLKNDRDVLGISDTLIRLSVGLEDEEDLLEDLDQALKAAHPPSGS |
| | | HS |

In one embodiment, the cystathionine gamma-lyase comprises or consists of an amino acid sequence of SEQ ID NO:50, or a variant thereof. In one embodiment, the cystathionine gamma-lyase comprises or consists of an amino acid sequence of SEQ ID NO:51, or a variant thereof. In one embodiment, the cystathionine gamma-lyase comprises an amino acid sequence of SEQ ID NO:52, or a variant thereof. In one embodiment, the cystathionine gamma-lyase comprises or consists of an amino acid sequence of SEQ ID NO:53, or a variant thereof. In one embodiment, the cystathionine gamma-lyase comprises or consists of an amino acid sequence of SEQ ID NO:54, or a variant thereof. In one embodiment, the cystathionine gamma-lyase comprises or consists of an amino acid sequence of SEQ ID NO:55, or a variant thereof. In one embodiment, the cystathionine gamma-lyase comprises or consists of an amino acid sequence of SEQ ID NO:56, or a variant thereof. In one embodiment, the cystathionine gamma-lyase comprises or consists of an amino acid sequence of SEQ ID NO:57, or a variant thereof. In one embodiment, the cystathionine gamma-lyase comprises an amino acid sequence of SEQ ID NO:58, or a variant thereof. In one embodiment, the cystathionine gamma-lyase comprises or consists of an amino acid sequence of SEQ ID NO:70, or a variant thereof. In one embodiment, the cystathionine gamma-lyase comprises or consists of an amino acid sequence of SEQ ID NO:71, or a variant thereof. In one embodiment, the cystathionine gamma-lyase comprises or consists of an amino acid sequence of SEQ ID NO:72, or a variant thereof. In one embodiment, the cystathionine gamma-lyase comprises or consists of an amino acid sequence of SEQ ID NO:73, or a variant thereof.

In one embodiment, the cystathionine gamma-lyase comprises an amino acid sequence that is at least 95% identical (e.g., 96%, 97%, 98%, 99% or 100% identical) to the amino acid sequence set forth in SEQ ID NO:50. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:51. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:52. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:53. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:54. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:55. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:56. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:57. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:58. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:70. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:71. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:72. In one embodiment, the cystathionine gamma-lyase comprises an amino acid sequence that is at least 95% identical (e.g., 96%, 97%, 98%, 99% or 100% identical) to the amino acid sequence set forth in SEQ ID NO:73.

In one embodiment, the cystathionine gamma-lyase comprises a Homo sapiens cystathionine gamma-lyase. In one embodiment, the Homo sapiens cystathionine gamma-lyase comprises an amino acid sequence that is at least 95% identical (e.g., 96%, 97%, 98%, 99% or 100% identical) to the amino acid sequence set forth in SEQ ID NO: 50.

In one embodiment, the cystathionine gamma-lyase comprises a Mus musculus cystathionine gamma-lyase. In one embodiment, the Mus musculus cystathionine gamma-lyase comprises an amino acid sequence that is at least 95% identical (e.g., 96%, 97%, 98%, 99% or 100% identical) to the amino acid sequence set forth in SEQ ID NO:51.

In one embodiment, the cystathionine gamma-lyase comprises a Rattus norvegicus cystathionine gamma-lyase. In one embodiment, the Rattus norvegicus cystathionine gamma-lyase comprises an amino acid sequence that is at least 95% identical (e.g., 96%, 97%, 98%, 99% or 100% identical) to the amino acid sequence set forth in SEQ ID NO:52.

In one embodiment, the cystathionine gamma-lyase comprises a Saccharomyces cerevisiae cystathionine gamma-lyase. In one embodiment, the Saccharomyces cerevisiae cystathionine gamma-lyase comprises an amino acid sequence that is at least 95% identical (e.g., 96%, 97%, 98%, 99% or 100% identical) to the amino acid sequence set forth in SEQ ID NO:53.

In one embodiment, the cystathionine gamma-lyase comprises a Neurospora crassa cystathionine gamma-lyase. In one embodiment, the Neurospora crassa cystathionine gamma-lyase comprises an amino acid sequence that is at least 95% identical (e.g., 96%, 97%, 98%, 99% or 100% identical) to the amino acid sequence set forth in SEQ ID NO:54.

In one embodiment, the cystathionine gamma-lyase comprises a Leishmania major cystathionine gamma-lyase. In one embodiment, the Leishmania major cystathionine gamma-lyase comprises an amino acid sequence that is at least 95% identical (e.g., 96%, 97%, 98%, 99% or 100% identical) to the amino acid sequence set forth in SEQ ID NO:55.

In one embodiment, the cystathionine gamma-lyase comprises a Corynebacterium ammoniagenes cystathionine gamma-lyase. In one embodiment, the Corynebacterium ammoniagenes cystathionine gamma-lyase comprises an amino acid sequence that is at least 95% identical (e.g., 96%, 97%, 98%, 99% or 100% identical) to the amino acid sequence set forth in SEQ ID NO:56.

In one embodiment, the cystathionine gamma-lyase comprises a Emericella nidulans cystathionine gamma-lyase. In one embodiment, the Emericella nidulans cystathionine gamma-lyase comprises an amino acid sequence that is at least 95% identical (e.g., 96%, 97%, 98%, 99% or 100% identical) to the amino acid sequence set forth in SEQ ID NO:57.

In one embodiment, the cystathionine gamma-lyase comprises a Arabidopsis thaliana cystathionine gamma-lyase. In one embodiment, the Arabidopsis thaliana cystathionine gamma-lyase comprises an amino acid sequence that is at least 95% identical (e.g., 96%, 97%, 98%, 99% or 100% identical) to the amino acid sequence set forth in SEQ ID NO:58.

In one embodiment, the cystathionine gamma-lyase comprises a Pongo abelii cystathionine gamma-lyase. In one embodiment, the Pongo abelii cystathionine gamma-lyase comprises an amino acid sequence that is at least 95% identical (e.g., 96%, 97%, 98%, 99% or 100% identical) to the amino acid sequence set forth in SEQ ID NO:70.

In one embodiment, the cystathionine gamma-lyase comprises a Macaca fascicularis cystathionine gamma-lyase. In one embodiment, the Macaca fascicularis cystathionine gamma-lyase comprises an amino acid sequence that is at least 95% identical (e.g., 96%, 97%, 98%, 99% or 100% identical) to the amino acid sequence set forth in SEQ ID NO:71.

In one embodiment, the cystathionine gamma-lyase comprises a Pan troglodytes cystathionine gamma-lyase. In one embodiment, the Pan troglodytes cystathionine gamma-lyase comprises an amino acid sequence that is at least 95% identical (e.g., 96%, 97%, 98%, 99% or 100% identical) to the amino acid sequence set forth in SEQ ID NO:72.

In one embodiment, the cystathionine gamma-lyase comprises a Pan paniscus cystathionine gamma-lyase. In one embodiment, the Pan paniscus cystathionine gamma-lyase comprises an amino acid sequence that is at least 95% identical (e.g., 96%, 97%, 98%, 99% or 100% identical) to the amino acid sequence set forth in SEQ ID NO:73.

In some embodiments, the cystathionine gamma-lyase comprises a variant of a wild-type cystathionine gamma-lyase having at least at least 40%, at least 50%, 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:70, SEQ ID NO:71, SEQ ID NO:72, or SEQ ID NO:73.

In a particular embodiment, the cystathionine gamma-lyase consists of the amino acid sequence of any one of SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:70, SEQ ID NO:71, SEQ ID NO:72, or SEQ ID NO:73.

A cystathionine gamma-lyase can also include proteins having an amino acid sequence comprising at least 10 contiguous amino acid residues of any one of SEQ ID NOs:50-58 and 70-73 (i.e., 10 contiguous amino acid residues having 100% identity with 10 contiguous amino acids of any one of SEQ ID NOs:50-58 and 70-73). In other embodiments, a cystathionine gamma-lyase amino acid sequence includes amino acid sequences comprising at least 20, or at least 30, or at least 40, or at least 50, or at least 75, or at least 100, or at least 125, or at least 150, or at least 175, or at least 150, or at least 200, or at least 250, or at least 300, or at least 350, or at least 400, or at least 450, or at least 500, or at least 550, contiguous amino acid residues of the amino acid sequence represented by any one of SEQ ID NOs:50-58 and 70-73, and any whole integer in between 10 and 550 contiguous amino acid residues. In a preferred embodiment, a cystathionine gamma-lyase has measurable or detectable cystathionine gamma-lyase biological activity. In some embodiments, fragments or variants of the cystathionine gamma-lyase enzyme retain at least 50%, at least 55%, at least 60%, at least 65%, at least 70%, at least 75%, at least 80%, at least 85%, at least 90%, at least 95%, at least 98% or at least 99% of the activity of cystathionine gamma-lyase enzyme.

In general, a variant cystathionine gamma-lyase, from any origin, may be produced, for example, to enhance production of the protein in an engineered cell, to improve turnover/half-life of the protein or mRNA encoding the protein, and/or to modulate (enhance or reduce) the enzymatic activity of the cystathionine gamma-lyase. The cystathionine gamma-lyase, whatever the source, may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding a cystathionine gamma-lyase as described herein. In some embodiments, the invention provides an engineered erythroid cell prepared by using a nucleic acid sequence encoding a cystathionine gamma-lyase as described herein. In some embodiments, the nucleic acid sequence encodes a cystathionine gamma-lyase (e.g. Homo sapiens cystathionine gamma-lyase, Mus musculus cystathionine gamma-lyase, Rattus norvegicus cystathionine gamma-lyase, Saccharomyces cerevisiae cystathionine gamma-lyase, Neurospora crassa cystathionine gamma-lyase, Leishmania major cystathionine gamma-lyase, Corynebacterium ammoniagenes cystathionine gamma-lyase, Emericella nidulans cystathionine gamma-lyase, and Arabidopsis thaliana cystathionine gamma-lyase) as described herein.

In some embodiments, the cystathionine gamma-lyase is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the corresponding wild-type cystathionine gamma-lyase nucleic acid (from any source) that encodes a protein that possesses a function of a cystathionine gamma-lyase described herein, e.g., the encoded protein cleaves a carbon-sulfur bond, releasing L-cysteine and an unstable enamine product that tautomerizes to an imine form, which undergoes a hydrolytic deamination to form 2-oxobutanoate and ammonia.

In some embodiments, an engineered erythroid cell or an enucleated cell comprises an exogenous polypeptide comprising a cystathionine degrading polypeptide (e.g., a CGL or a variant thereof) that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the cystathionine degrading polypeptide).

In some embodiments, the exogenous polypeptide may include a linker (e.g., a linker described herein) disposed between the cystathionine degrading polypeptide and the at least one polypeptide of interest. In some embodiments, the linker comprises or consists of a poly-glycine poly-serine linker with one or more amino acid substitutions, deletions, and/or additions and which lacks the amino acid sequence GSG.

In some embodiments, the exogenous polypeptide comprises a transmembrane domain or a transmembrane polypeptide (e.g., SMIM1, GPA, or Kell) and a cystathionine degrading polypeptide. In some embodiments, the transmembrane domain is derived from GPA. In some embodiments, the transmembrane domain is derived from SMIM1.

In some embodiments, the transmembrane domain or transmembrane polypeptide is disposed in the exogenous polypeptide such that the cystathionine degrading polypeptide present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the cystathionine degrading polypeptide present in the exogenous polypeptide locates in the outer surface of the cell (e.g., facing the extracellular milieu of the cell). In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the cystathionine degrading polypeptide.

In some embodiments the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA.

In some embodiments, the exogenous polypeptide is a fusion protein comprising any of the cystathionine gamma-lyases or variants described herein linked to a heterologous protein sequence (e.g., via a linker). In some embodiments, the exogenous polypeptide is a fusion protein comprising a cystathionine gamma-lyase, or variant thereof, described herein fused to a homocysteine degrading polypeptide (e.g., cystathionine beta synthase). In some embodiments, the exogenous polypeptide comprises a linker (e.g., a flexible linker) disposed between the cystathionine gamma-lyase and the homocysteine degrading polypeptide.

In some embodiments, an engineered cell provided herein comprises an exogenous polypeptide comprising a cystathionine gamma-lyase variant. Multiple cystathionine gamma-lyase variants are known in the art and may be used as described herein (see, e.g., Zhu et al. (2008) Biochemistry 47(23): 6226-32, and U.S. Patent Application Publication No. 2018/0326025 A1; the entire contents of each of which are incorporated herein by reference). In some embodiments, the cystathionine gamma lyase variant is derived from a primate wild-type cystathionine gamma-lyase (e.g., a human or a non-human primate cystathionine gamma-lyase).

In some embodiments, the cystathionine gamma-lyase variant comprises a decreased Vₘₐₓ as compared to the Vₘₐₓ of the wild-type cystathionine gamma-lyase from which it was derived. For example, in some embodiments, the cystathionine gamma-lyase variant comprises at least about a 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more decrease in Vₘₐₓ as compared to the wild-type cystathionine gamma-lyase from which it was derived.

In some embodiments, the cystathionine gamma lyase variant comprises the amino acid sequence of SEQ ID NO: 50 with a threonine to isoleucine substitution at position 67 (i.e., T67I). In some embodiments, the cystathionine gamma lyase variant comprises the amino acid sequence set forth in SEQ ID NO: 92, below.

Homo sapiens (human) cystathionine gamma lyase with amino acid substitution T67I:

(SEQ ID NO: 92)
MQEKDASSQGFLPHFQHFATQAIHVGQDPEQWTSRAVVPPISLSTTFKQG
APGQHSGFEYSRSGNPIRNCLEKAVAALDGAKYCLAFASGLAATVTITHL
LKAGDQIICMDDVYGGTNRYFRQVASEFGLKISFVDCSKIKLLEAAITPE
TKLVWIETPTNPTQKVIDIEGCAHIVHKHGDIILVVDNTFMSPYFQRPLA
LGADISMYSATKYMNGHSDVVMGLVSVNCESLHNRLRFLQNSLGAVPSPI
DCYLCNRGLKTLHVRMEKHFKNGMAVAQFLESNPWVEKVIYPGLPSHPQH
ELVKRQCTGCTGMVTFYIKGTLQHAEIFLKNLKLFTLAESLGGFESLAEL
PAIMTHASVLKNDRDVLGISDTLIRLSVGLEDEEDLLEDLDQALKAAHPP
SGSHS

In some embodiments, the cystathionine gamma lyase variant comprises the amino acid sequence of SEQ ID NO: 50 with a glutamine to glutamic acid substitution at position 240 (i.e., Q240E). In some embodiments, the cystathionine gamma lyase variant comprises the amino acid sequence set forth in SEQ ID NO: 93, below.

Homo sapiens (human) cystathionine gamma lyase with amino acid substitution Q240E

(SEQ ID NO: 93)
MQEKDASSQGFLPHFQHFATQAIHVGQDPEQWTSRAVVPPISLSTTFKQG
APGQHSGFEYSRSGNPTRNCLEKAVAALDGAKYCLAFASGLAATVTITHL
LKAGDQIICMDDVYGGTNRYFRQVASEFGLKISFVDCSKIKLLEAAITPE
TKLVWIETPTNPTQKVIDIEGCAHIVHKHGDIILVVDNTFMSPYFQRPLA
LGADISMYSATKYMNGHSDVVMGLVSVNCESLHNRLRFLENSLGAVPSPI
DCYLCNRGLKTLHVRMEKHFKNGMAVAQFLESNPWVEKVIYPGLPSHPQH
ELVKRQCTGCTGMVTFYIKGTLQHAEIFLKNLKLFTLAESLGGFESLAEL
PAIMTHASVLKNDRDVLGISDTLIRLSVGLEDEEDLLEDLDQALKAAHPP
SGSHS

In some embodiments, the cystathionine gamma lyase variant comprises the amino acid sequence of SEQ ID NO: 50 with a threonine to isoleucine substitution at position 67 and a glutamine to glutamic acid substitution at position 240 (i.e., both T67I and Q240E). In some embodiments, the cystathionine gamma lyase variant comprises the amino acid sequence set forth in SEQ ID NO: 94, below. Homo sapiens (human) cystathionine gamma lyase with amino acid substitution T67I and Q240E

(SEQ ID NO: 94)
MQEKDASSQGFLPHFQHFATQAIHVGQDPEQWTSRAVVPPISLSTTFKQG
APGQHSGFEYSRSGNPIRNCLEKAVAALDGAKYCLAFASGLAATVTITHL
LKAGDQIICMDDVYGGTNRYFRQVASEFGLKISFVDCSKIKLLEAAITPE
TKLVWIETPTNPTQKVIDIEGCAHIVHKHGDIILVVDNTFMSPYFQRPLA
LGADISMYSATKYMNGHSDVVMGLVSVNCESLHNRLRFLENSLGAVPSPI
DCYLCNRGLKTLHVRMEKHFKNGMAVAQFLESNPWVEKVIYPGLPSHPQH
ELVKRQCTGCTGMVTFYIKGTLQHAEIFLKNLKLFTLAESLGGFESLAEL
PAIMTHASVLKNDRDVLGISDTLIRLSVGLEDEEDLLEDLDQALKAAHPP
SGSHS

In some embodiments, the cystathionine gamma lyase variant comprises at least one (e.g., one, two, three, four five, six, seven, eight or more) amino acid substitution. Said amino acid substitutions may alter the substrate recognition site or the substrate specificity of the polypeptide. For example, in some embodiments, the cystathionine gamma lyase variant may have at least one amino acid substitution at amino acid positions corresponding to E59, S63, L91, R119, K268, T311, T336, E339, and/or 1353 of SEQ ID NO: 50 or amino acid positions of 59, 63, 91, 119, 268, 311, 336, 339, and/or 353 of any one of SEQ ID NOs: 74-91. In some embodiments, the substitution at any one of the amino acid residues at positions 59, 63, 91, 119, 268, 311, 339, and/or 353, and can be to an aspartic acid (N), a valine (V), a leucine (L), a methionine (M), an arginine (R), a glycine (G), an alanine (A), or a serine (S). In some embodiments, the cystathionine gamma lyase variant comprises one or more substitutions selected from the group consisting of E/V59N, E59I, S63L, L91M, R119L, R119A, R119D, R119H, R119G, K268R, T311G, T336D, T336E, E339V, and I/V353S (e.g., in SEQ ID NO: 50). In some embodiments, the cystathionine gamma lyase variant comprises the following amino acid substitutions: S63L, L91M, K268R, T311G, E339V, and I/V353S. In some embodiments, the cystathionine gamma lyase variant comprises S63L, L91M, K268R, T311G, E339V, I/V353S, and either E/V59N or E/V59I (e.g., in SEQ ID NO: 50). In some embodiments, the cystathionine gamma lyase variant comprises S63L, L91M, K268R, T311G, E339V, I/V353S, and any one of R119L, R119A, R119D, R119H, and R119G (e.g., in SEQ ID NO: 50). In some embodiments, the cystathionine gamma lyase variant comprises S63L, L91M, K268R, T311G, E339V, I/V353S, and either T336D or T336E (e.g., in SEQ ID NO: 50).

In some embodiments, the cystathionine gamma lyase variant comprises the amino acid sequence of any one of SEQ ID NOs: 74-91, with one or more substitutions selected from the group consisting of 59N, 591, 63L, 91M, 119L, 119A, 119D, 119H, 119G, 268R, 311G, 336D, 336E, 339V, and 353S (the number of the amino acid residue position and the amino acid residue with which the native amino acid residue at that position is replaced is indicated). In some embodiments, the cystathionine gamma lyase variant comprises the amino acid sequence of any one of SEQ ID NOs: 75-78, 80-83, 85-91, with the following amino acid substitutions: 63L, 91M, 268R, 311G, 339V, and 353S. In some embodiments, the cystathionine gamma lyase variant comprises the amino acid sequence of any one of SEQ ID NOs: 75-78, 80-83, 85-91, with the following amino acid substitutions: 63L, 91M, 268R, 311G, 339V, 353S, and either 59N or 591. In some embodiments, the cystathionine gamma lyase variant comprises the amino acid sequence of any one of SEQ ID NOs: 75-78, 80-83, 85-91, with the following amino acid substitutions: 63L, 91M, 268R, 311G, 339V, 353S, and any one of 119L, 119A, 119D, 119H, and 119G. In some embodiments, the cystathionine gamma lyase variant comprises the amino acid sequence of any one of SEQ ID NOs: 80-83, 85-88, with the following amino acid substitutions: 63L, 91M, 268R, 311G, 339V, 353S, and either 336D or 336E.

In some embodiments, the cystathionine gamma lyase variant comprises the amino acid sequence of SEQ ID NO: 50 with the following amino acid substitutions: E59N, S63L, L91M, R119L, K268R, T311G, I353S, and E339V. In some embodiments, the cystathionine gamma lyase variant comprises the amino acid sequence of SEQ ID NO: 50 with the following amino acid substitutions: E59N, S63L, L91M, R119L, K268R, T311G, I353S, E339V, and either T336D or T336E.

In some embodiments, the cystathionine gamma lyase variant comprises the amino acid sequence of SEQ ID NO: 50 with the following amino acid substitutions: E59I, S63L, L91M, R119L, K268R, T311G, E339V, and I353S. In some embodiments, the cystathionine gamma lyase variant comprises the amino acid sequence of SEQ ID NO: 50 with the following amino acid substitutions: E59I, S63L, L91M, R119L, K268R, T311G, E339V, I353S, and either T336D or T336E.

In some embodiments, the cystathionine gamma lyase variant comprises the amino acid sequence of SEQ ID NO: 50 with the following amino acid substitutions: E59N, S63L, L91M, R119A, K268R, T311G, E339V, and I353S. In some embodiments, the cystathionine gamma lyase variant comprises the amino acid sequence of SEQ ID NO: 50 with the following amino acid substitutions: E59N, S63L, L91M, R119A, K268R, T311G, E339V, I353S, and either T336D or T336E.

In some embodiments, the cystathionine gamma lyase variant comprises the amino acid sequence of SEQ ID NO: 50 with the following amino acid substitutions: E59I, S63L, L91M, R119A, K268R, T311G, E339V, and I353S. In some embodiments, the cystathionine gamma lyase variant comprises the amino acid sequence of SEQ ID NO: 50 with the following amino acid substitutions: E59I, S63L, L91M, R119A, K268R, T311G, E339V, I353S, and either T336D or T336E.

In some embodiments, the cystathionine gamma lyase variant comprises the amino acid sequence of SEQ ID NO: 50 with the following amino acid substitutions: E59I, S63L, L91M, R119D, K268R, T311G, E339V, and I353S. In some embodiments, the cystathionine gamma lyase variant comprises the amino acid sequence of SEQ ID NO: 50 with the following amino acid substitutions: E59I, S63L, L91M, R119D, K268R, T311G, E339V, I353S, and either T336D or T336E.

In some embodiments, the cystathionine gamma lyase variant comprises the amino acid sequence of SEQ ID NO: 50 with the following amino acid substitutions: E59I, S63L, L91M, R119H, K268R, T311G, E339V, and I353S. In some embodiments, the cystathionine gamma lyase variant comprises the amino acid sequence of SEQ ID NO: 50 with the following amino acid substitutions: E59I, S63L, L91M, R119H, K268R, T311G, E339V, I353S, and either T336D or T336E.

In some embodiments, the cystathionine gamma lyase variant comprises the amino acid sequence of SEQ ID NO: 50 with the following amino acid substitutions: E59I, S63L, L91M, R119G, K268R, T311G, E339V, and I353S. In some embodiments, the cystathionine gamma lyase variant comprises the amino acid sequence of SEQ ID NO: 50 with the following amino acid substitutions: E59I, S63L, L91M, R119G, K268R, T311G, E339V, I353S, and either T336D or T336E.

In some preferred embodiments, the cystathionine gamma-lyase variant comprises an amino acid sequence set forth in Table 4, below. In some embodiments, the cystathionine gamma-lyase comprises or consists of the amino acid sequence set forth in any one of SEQ ID NOs: 74-94.

TABLE 4
Exemplary Cystathionine gamma-lyase variants

| | |
|---|---|
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRSGNPTRNCLEKAVAA |
| R119L and E339V | LDGAKYCLAFASGLAATVTITHLLKAGDQIICMDDVYGGT |
| (SEQ ID NO: 74) | NLYFRQVASEFGLKISFVDCSKIKLLEAAITPETKLVWIETPT |
| | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMEKHFKNGMAVA |
| | QFLESNPWVEKVIYPGLPSHPQHELVKRQCTGCTGMVTF |
| | YIKGTLQHAEIFLKNLKLFTLAVSLGGFESLAELPAIMTHAS |
| | VLKNDRDVLGISDTLIRLSVGLEDEEDLLEDLDQALKAAHP |
| | PSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119L, K268R, T311G, E339V, and | LDGAKYCLAFASGMAATVTITHLLKAGDQIICMDDVYGG |
| I353S | TNLYFRQVASEFGLKISFVDCSKIKLLEAAITPETKLVWIETP |
| (SEQ ID NO: 75) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119L, K268R, T311G, E339V, and | DGAKYCLAFASGMAATVTITHLLKAGDQIICMDDVYGGT |
| I353S | NLYFRQVASEFGLKISFVDCSKIKLLEAAITPETKLVWIETPT |
| (SEQ ID NO: 76) | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMERHFKNGMAVA |
| | QFLESNPWVEKVIYPGLPSHPQHELVKRQCTGCGGMVT |
| | FYIKGTLQHAEIFLKNLKLFTLAVSLGGFESLAELPASMTHA |
| | SVLKNDRDVLGISDTLIRLSVGLEDEEDLLEDLDQALKAAH |
| | PPSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119A, K268R, T311G, E339V, | LDGAKYCLAFASGMAATVTITHLLKAGDQIICMDDVYGG |
| and I353S | TNAYFRQVASEFGLKISFVDCSKIKLLEAAITPETKLVWIET |
| (SEQ ID NO: 77) | PTNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| VPPISLSTTFKQGAPGQHSGFIYSRLGNPTRNCLEKAVAAL | lyasewithaminoacidsubstitutionsE59I,563L, |
| L91M, R119A, K268R, T311G, E339V, and | DGAKYCLAFASGMAATVTITHLLKAGDQIICMDDVYGGT |
| I353S | NAYFRQVASEFGLKISFVDCSKIKLLEAAITPETKLVWIETP |
| (SEQ ID NO: 78) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRSGNPTRNCLEKAVAA |
| R119L, T336D, and E339V | LDGAKYCLAFASGLAATVTITHLLKAGDQIICMDDVYGGT |
| (SEQ ID NO: 79) | NLYFRQVASEFGLKISFVDCSKIKLLEAAITPETKLVWIETPT |
| | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMEKHFKNGMAVA |
| | QFLESNPWVEKVIYPGLPSHPQHELVKRQCTGCTGMVTF |
| | YIKGTLQHAEIFLKNLKLFDLAVSLGGFESLAELPAIMTHAS |
| | VLKNDRDVLGISDTLIRLSVGLEDEEDLLEDLDQALKAAHP |
| | PSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119L, K268R, T311G, T336D, | LDGAKYCLAFASGMAATVTITHLLKAGDQIICMDDVYGG |
| E339V, and I353S | TNLYFRQVASEFGLKISFVDCSKIKLLEAAITPETKLVWIETP |
| (SEQ ID NO: 80) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFDLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119L, K268R, T311G, T336D, | DGAKYCLAFASGMAATVTITHLLKAGDQIICMDDVYGGT |
| E339V, and I353S | NLYFRQVASEFGLKISFVDCSKIKLLEAAITPETKLVWIETPT |
| (SEQ ID NO: 81) | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMERHFKNGMAVA |
| | QFLESNPWVEKVIYPGLPSHPQHELVKRQCTGCGGMVT |
| | FYIKGTLQHAEIFLKNLKLFDLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119A, K268R, T311G, T336D, | LDGAKYCLAFASGMAATVTITHLLKAGDQIICMDDVYGG |
| E339V, and I353S | TNAYFRQVASEFGLKISFVDCSKIKLLEAAITPETKLVWIET |
| (SEQ ID NO: 82) | PTNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFDLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119A, K268R, T311G, T336D, | DGAKYCLAFASGMAATVTITHLLKAGDQIICMDDVYGGT |
| E339V, and I353S | NAYFRQVASEFGLKISFVDCSKIKLLEAAITPETKLVWIETP |
| (SEQ ID NO: 83) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFDLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRSGNPTRNCLEKAVAA |
| R119L, T336E, and E339V | LDGAKYCLAFASGLAATVTITHLLKAGDQIICMDDVYGGT |
| (SEQ ID NO: 84) | NLYFRQVASEFGLKISFVDCSKIKLLEAAITPETKLVWIETPT |
| | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMEKHFKNGMAVA |
| | QFLESNPWVEKVIYPGLPSHPQHELVKRQCTGCTGMVTF |
| | YIKGTLQHAEIFLKNLKLFELAVSLGGFESLAELPAIMTHAS |
| | VLKNDRDVLGISDTLIRLSVGLEDEEDLLEDLDQALKAAHP |
| | PSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119L, K268R, T311G, T336E, | LDGAKYCLAFASGMAATVTITHLLKAGDQIICMDDVYGG |
| E339V, and I353S | TNLYFRQVASEFGLKISFVDCSKIKLLEAAITPETKLVWIETP |
| (SEQ ID NO: 85) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFELAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119L, K268R, T311G, T336E, | DGAKYCLAFASGMAATVTITHLLKAGDQIICMDDVYGGT |
| E339V, and I353S | NLYFRQVASEFGLKISFVDCSKIKLLEAAITPETKLVWIETPT |
| SEQ ID NO: 86 | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMERHFKNGMAVA |
| | QFLESNPWVEKVIYPGLPSHPQHELVKRQCTGCGGMVT |
| | FYIKGTLQHAEIFLKNLKLFELAVSLGGFESLAELPASMTHA |
| | SVLKNDRDVLGISDTLIRLSVGLEDEEDLLEDLDQALKAAH |
| | PPSGSHS |
| Homo sapiens (human) cystathionine gamma | MQEKDASSQGFLPHFQHFATQAIHVGQDPEQWTSRAV |
| lyase with amino acid substitutions E59N, | VPPISLSTTFKQGAPGQHSGFNYSRLGNPTRNCLEKAVAA |
| S63L, L91M, R119A, K268R, T311G, T336E, | LDGAKYCLAFASGMAATVTITHLLKAGDQIICMDDVYGG |
| E339V, and I353S | TNAYFRQVASEFGLKISFVDCSKIKLLEAAITPETKLVWIET |
| SEQ ID NO: 87 | PTNPTQKVIDIEGCAHIVHKHGDI1LVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFELAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119A, K268R, T311G, T336E, | DGAKYCLAFASGMAATVTITHLLKAGDQIICMDDVYGGT |
| E339V, and I353S | NAYFRQVASEFGLKISFVDCSKIKLLEAAITPETKLVWIETP |
| SEQ ID NO: 88 | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFELAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119D, K268R, T311G, E339V, and | DGAKYCLAFASGMAATVTITHLLKAGDQIICMDDVYGGT |
| I353S | NDYFRQVASEFGLKISFVDCSKIKLLEAAITPETKLVWIETP |
| (SEQ ID NO: 89) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119H, K268R, T311G, E339V, I353S | DGAKYCLAFASGMAATVTITHLLKAGDQIICMDDVYGGT |
| (SEQ ID NO: 90) | NHYFRQVASEFGLKISFVDCSKIKLLEAAITPETKLVWIETP |
| | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitutions E59I, | VPPISLSTTFKQGAPGQHSGFIYSRLGNPTRNCLEKAVAAL |
| S63L, L91M, R119G, K268R, T311G, E339V, and | DGAKYCLAFASGMAATVTITHLLKAGDQIICMDDVYGGT |
| I353S | NGYFRQVASEFGLKISFVDCSKIKLLEAAITPETKLVWIETP |
| (SEQ ID NO: 91) | TNPTQKVIDIEGCAHIVHKHGDIILVVDNTFMSPYFQRPL |
| | ALGADISMYSATKYMNGHSDVVMGLVSVNCESLHNRLR |
| | FLQNSLGAVPSPIDCYLCNRGLKTLHVRMERHFKNGMAV |
| | AQFLESNPWVEKVIYPGLPSHPQHELVKRQCTGCGGMV |
| | TFYIKGTLQHAEIFLKNLKLFTLAVSLGGFESLAELPASMTH |
| | ASVLKNDRDVLGISDTLIRLSVGLEDEEDLLEDLDQALKAA |
| | HPPSGSHS |
| Homo sapiens (human) cystathionine gamma | MQEKDASSQGFLPHFQHFATQAIHVGQDPEQWTSRAV |
| lyase with amino acid substitution T67I | VPPISLSTTFKQGAPGQHSGFEYSRSGNPIRNCLEKAVAAL |
| (SEQ ID NO: 92) | DGAKYCLAFASGLAATVTITHLLKAGDQIICMDDVYGGTN |
| | RYFRQVASEFGLKISFVDCSKIKLLEAAITPETKLVWIETPT |
| | NPTQKVIDIEGCAHIVHKHGDIILVVDNTFMSPYFQRPLAL |
| | GADISMYSATKYMNGHSDVVMGLVSVNCESLHNRLRFL |
| | QNSLGAVPSPIDCYLCNRGLKTLHVRMEKHFKNGMAVA |
| | QFLESNPWVEKVIYPGLPSHPQHELVKRQCTGCTGMVTF |
| | YIKGTLQHAEIFLKNLKLFTLAESLGGFESLAELPAIMTHAS |
| | VLKNDRDVLGISDTLIRLSVGLEDEEDLLEDLDQALKAAHP |
| | PSGSHS |
| Homo sapiens (human) cystathionine gamma | MQEKDASSQGFLPHFQHFATQAIHVGQDPEQWTSRAV |
| lyase with amino acid substitution Q240E | VPPISLSTTFKQGAPGQHSGFEYSRSGNPTRNCLEKAVAA |
| (SEQ ID NO: 93) | LDGAKYCLAFASGLAATVTITHLLKAGDQIICMDDVYGGT |
| | NRYFRQVASEFGLKISFVDCSKIKLLEAAITPETKLVWIETP |
| | TNPTQKVIDIEGCAHIVHKHGDIILVVDNTFMSPYFQRPL |
| | ALGADISMYSATKYMNGHSDVVMGLVSVNCESLHNRLR |
| | FLENSLGAVPSPIDCYLCNRGLKTLHVRMEKHFKNGMAV |
| | AQFLESNPWVEKVIYPGLPSHPQHELVKRQCTGCTGMVT |
| | FYIKGTLQHAEIFLKNLKLFTLAESLGGFESLAELPAIMTHA |
| | SVLKNDRDVLGISDTLIRLSVGLEDEEDLLEDLDQALKAAH |
| | PPSGSHS |
| Homo sapiens (human) cystathionine gamma | MQEKDASSQGFLPHFQHFATQAIHVGQDPEQWTSRAV |
| lyase with amino acid substitution T67I and | VPPISLSTTFKQGAPGQHSGFEYSRSGNPIRNCLEKAVAAL |
| Q240E | DGAKYCLAFASGLAATVTITHLLKAGDQIICMDDVYGGTN |
| (SEQ ID NO: 94) | RYFRQVASEFGLKISFVDCSKIKLLEAAITPETKLVWIETPT |
| | NPTQKVIDIEGCAHIVHKHGDIILVVDNTFMSPYFQRPLAL |
| | GADISMYSATKYMNGHSDVVMGLVSVNCESLHNRLRFL |
| | ENSLGAVPSPIDCYLCNRGLKTLHVRMEKHFKNGMAVA |
| | QFLESNPWVEKVIYPGLPSHPQHELVKRQCTGCTGMVTF |
| | YIKGTLQHAEIFLKNLKLFTLAESLGGFESLAELPAIMTHAS |
| | VLKNDRDVLGISDTLIRLSVGLEDEEDLLEDLDQALKAAHP |
| | PSGSHS |

In some embodiments, the cystathionine gamma lyase variant comprises the amino acid sequence of SEQ ID NO: 50 with the following amino acid substitutions: isoleucine at position 59, leucine at position 63, methionine at position 91, aspartic acid at position 119, arginine at position 268, glycine at position 311, valine at position 339, and serine at position 353. In some embodiments, the cystathionine gamma lyase comprises the amino acid sequence of SEQ ID NO: 50 with the following amino acid substitutions: asparagine at position 59, leucine at position 119, aspartic acid at position 336, and valine at position 339.

In some embodiments, the cystathionine gamma lyase comprises the amino acid sequence of SEQ ID NO: 50 with the following amino acid substitutions: asparagine at position 59, leucine at position 63, methionine at position 91, leucine at position 119, arginine at position 268, glycine at position 311, aspartic acid at position 336, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, leucine at position 119, arginine at position 268, glycine at position 311, aspartic acid at position 336, valine at position 339, and serine at position 353.

In some embodiments, the cystathionine gamma lyase comprises the amino acid sequence of SEQ ID NO: 50 with the following amino acid substitutions: asparagine at position 59, leucine at position 63, methionine at position 91, alanine at position 119, arginine at position 268, glycine at position 311, aspartic acid at position 336, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, alanine at position 119, arginine at position 268, glycine at position 311, aspartic acid at position 336, valine at position 339, and serine at position 353.

In some embodiments, the cystathionine gamma lyase comprises the amino acid sequence of SEQ ID NO: 50 with the following amino acid substitutions: asparagine at position 59, leucine at position 119, glutamic acid at position 336, and valine at position 339.

In some embodiments, the cystathionine gamma lyase comprises the amino acid sequence of SEQ ID NO: 50 with the following amino acid substitutions: asparagine at position 59, leucine at position 63, methionine at position 91, leucine at position 119, arginine at position 268, glycine at position 311, glutamic acid at position 336, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, leucine at position 119, arginine at position 268, glycine at position 311, glutamic acid at position 336, valine at position 339, and serine at position 353.

In some embodiments, the cystathionine gamma lyase comprises the amino acid sequence of SEQ ID NO: 50 with the following amino acid substitutions: asparagine at position 59, leucine at position 63, methionine at position 91, leucine at position 119, arginine at position 268, glycine at position 311, glutamic acid at position 336, valine at position 339, and serine at position 353.

In some embodiments, the cystathionine gamma lyase comprises the amino acid sequence of SEQ ID NO: 50 with the following amino acid substitutions: asparagine at position 59, leucine at position 63, methionine at position 91, alanine at position 119, arginine at position 268, glycine at position 311, glutamic acid at position 336, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, alanine at position 119, arginine at position 268, glycine at position 311, glutamic acid at position 336, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, histidine at position 119, arginine at position 268, glycine at position 311, valine at position 339, and serine at position 353.

In some embodiments, the cystathionine gamma lyase comprises the amino acid sequence of SEQ ID NO: 50 with the following amino acid substitutions: isoleucine at position 59, leucine at position 63, methionine at position 91, glycine at position 119, arginine at position 268, glycine at position 311, valine at position 339, and serine at position 353.

In some embodiments, the cystathionine gamma lyase comprises the amino acid sequence of SEQ ID NO: 70 with any one of the following combinations of amino acid substitutions:

(a) V59I, S63L, L91M, R119D, K268R, T311G, E339V, and V353S;

(b) V59N, R119L, T336D, and E339V;

(c) V59N, S63L, L91M, R119L, K268R, T311G, T336D, E339V, and V353S;

(d) V59I, S63L, L91M, R119L, K268R, T311G, T336D, E339V, and V353S;

(e) V59N, S63L, L91M, R119A, K268R, T311G, T336D, E339V, and V353S;

(f) V59I, S63L, L91M, R119A, K268R, T311G, T336D, E339V, and V353S;

(g) V59N, R119L, T336E, and E339V;

(h) V59N, S63L, L91M, R119L, K268R, T311G, T336E, E339V, and V353S;

(i) V59I, S63L, L91M, R119L, K268R, T311G, T336E, E339V, and V353S;

(j) V59N, S63L, L91M, R119A, K268R, T311G, T336E, E339V, and V353S;

(k) V59I, S63L, L91M, R119A, K268R, T311G, T336E, E339V, and V353S;

(l) V59I, S63L, L91M, R119H, K268R, T311G, E339V, and V353S; and

(m) V59I, S63L, L91M, R119G, K268R, T311G, E339V, and V353S.

In some embodiments, the cystathionine gamma lyase comprises the amino acid sequence of any one of SEQ ID NOs: 71-73 with any one of the following combinations of amino acid substitutions:

(a) E59I, S63L, L91M, R119D, K268R, T311G, E339V, and I353S;

(b) E59N, R119L, T336D, and E339V;

(c) E59N, S63L, L91M, R119L, K268R, T311G, T336D, E339V, and I353S;

(d) E59I, S63L, L91M, R119L, K268R, T311G, T336D, E339V, and I353S;

(e) E59N, S63L, L91M, R119A, K268R, T311G, T336D, E339V, and I353S;

(f) E59I, S63L, L91M, R119A, K268R, T311G, T336D, E339V, and I353S;

(g) E59N, R119L, T336E, and E339V;

(h) E59N, S63L, L91M, R119L, K268R, T311G, T336E, E339V, and I353S;

(i) E59I, S63L, L91M, R119L, K268R, T311G, T336E, E339V, and I353S;

(j) E59N, S63L, L91M, R119A, K268R, T311G, T336E, E339V, and I353S;

(k) E59I, S63L, L91M, R119A, K268R, T311G, T336E, E339V, and I353S;

(l) E59I, S63L, L91M, R119H, K268R, T311G, E339V, and I353S; and

(m) E59I, S63L, L91M, R119G, K268R, T311G, E339V, and I353S.

In some embodiments, an engineered erythroid cell or an enucleated cell comprises an exogenous polypeptide comprising an cystathionine gamma-lyase, or a variant thereof, that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the cystathionine degrading polypeptide).

In some embodiments, the exogenous polypeptide may include a linker (e.g., a linker described herein) disposed between the cystathionine gamma-lyase, or a variant thereof, and the at least one polypeptide of interest. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 95), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGG (SEQ ID NO: 96), GGGGSGGGGS (SEQ ID NO: 97), GSGSGSGSGS (SEQ ID NO: 98), PSTSTST (SEQ ID NO: 99), and EIDKPSQ (SEQ ID NO: 100), and multimers thereof.

In some embodiments, the exogenous polypeptide comprises a transmembrane domain or a transmembrane polypeptide (e.g., SMIM1, GPA, or Kell) and an cystathionine gamma-lyase, or a variant thereof. In some embodiments, the transmembrane domain is derived from GPA. For example, in some embodiments, the transmembrane domain is derived from GPA and comprises or consists of the amino acid sequence:

(SEQ ID NO: 102)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

In some embodiments, the transmembrane domain is derived from SMIM1. For example, in some embodiments, the transmembrane domain comprises or consists of the amino acid sequence:

(SEQ ID NO: 103)
MQPQESHVHYSRWEDGSRDGVSLGAVSSTEEASRCRRISQRLCTGKLGIA
MKVLGGVALFWIIFILGYLTGYYVHKCK

In some embodiments, the transmembrane domain or transmembrane polypeptide is disposed in the exogenous polypeptide such that the cystathionine gamma-lyase, or a variant thereof, present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the cystathionine gamma-lyase, or a variant thereof, in the exogenous polypeptide locates in the outer surface of the cell (e.g., facing the extracellular milieu of the cell). In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the cystathionine gamma-lyase, or a variant thereof.

In some embodiments, the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA. For example, in some embodiments, the leader or signal sequence is derived from GPA and comprises or consists of the amino acid sequence MYGKIIFVLLLSEIVSISA (SEQ ID NO: 101).

In some embodiments, the exogenous polypeptide comprises an HA tag and a cystathionine gamma-lyase, or a variant thereof. In some embodiments, the exogenous polypeptide comprises a leader sequence, a cystathionine gamma-lyase, or a variant thereof, and a trasmembrane domain (e.g., a GPA transmembrane domain). For example, in some embodiments, the exogenous polypeptide comprises or consists of an amino acid sequence provided in Table 5 provided below. In some embodiments, the exogenous polypeptide comprises or consists of an amino acid sequence provided in Table 5 without a leader sequence (underlined).

TABLE 5
Exemplary Exogenous Polypeptides Comprising a Human CGL Variant

| | |
|---|---|
| Leader sequence-human CGL variant (with | MYGKIIFVLLLSEIVSISAMQEKDASSQGFLPHFQHFAT |
| amino acid substitutions E59I, S63L, L91M, | QAIHVGQDPEQWTSRAVVPPISLSTTFKQGAPGQHS |
| R119A, K268R, T311G, E339V, and I353S)-linker- | GFIYSRLGNPTRNCLEKAVAALDGAKYCLAFASGMAA |
| HA-linker-GPA transmembrane domain | TVTITHLLKAGDQIICMDDVYGGTNAYFRQVASEFGLK |
| (Leader sequence underlined) | ISFVDCSKIKLLEAAITPETKLVWIETPTNPTQKVIDIEGC |
| (SEQ ID NO: 104) | AHIVHKHGDIILVVDNTFMSPYFQRPLALGADISMYSA |
| | TKYMNGHSDVVMGLVSVNCESLHNRLRFLQNSLGAV |
| | PSPIDCYLCNRGLKTLHVRMERHFKNGMAVAQFLESN |
| | PWVEKVIYPGLPSHPQHELVKRQCTGCGGMVTFYIKG |
| | TLQHAEIFLKNLKLFTLAVSLGGFESLAELPASMTHASV |
| | LKNDRDVLGISDTLIRLSVGLEDEEDLLEDLDQALKAAH |
| | PPSGSHSGGGGSGGGGYPYDVPDYAGGGGSGGGGL |
| | STTEVAMHTSTSSSVTKSYISSQTNDTHKRDTYAATPR |
| | AHEVSEISVRTVYPPEEETGERVQLAHHFSEPEITLIIFG |
| | VMAGVIGTILLISYGIRRLIKKSPSDVKPLPSPDTDVPLSS |
| | VEIENPETSDQ |
| Human CGL variant (with amino acid | MQEKDASSQGFLPHFQHFATQAIHVGQDPEQWTSR |
| substitutions E59I, S63L, L91M, R119A, K268R, | AVVPPISLSTTFKQGAPGQHSGFIYSRLGNPTRNCLEK |
| T311G, E339V, and I353S)-linker-HA-linker-GPA | AVAALDGAKYCLAFASGMAATVTITHLLKAGDQIICM |
| transmembrane region | DDVYGGTNAYFRQVASEFGLKISFVDCSKIKLLEAAITP |
| (excludes leader sequence) | ETKLVWIETPTNPTQKVIDIEGCAHIVHKHGDIILVVDN |
| (SEQ ID NO: 105) | TFMSPYFQRPLALGADISMYSATKYMNGHSDVVMGL |
| | VSVNCESLHNRLRFLQNSLGAVPSPIDCYLCNRGLKTL |
| | HVRMERHFKNGMAVAQFLESNPWVEKVIYPGLPSHP |
| | QHELVKRQCTGCGGMVTFYIKGTLQHAEIFLKNLKLFT |
| | LAVSLGGFESLAELPASMTHASVLKNDRDVLGISDTLIR |
| | LSVGLEDEEDLLEDLDQALKAAHPPSGSHSGGGGSGG |
| | GGYPYDVPDYAGGGGSGGGGLSTTEVAMHTSTSSSV |
| | TKSYISSQTNDTHKRDTYAATPRAHEVSEISVRTVYPPE |
| | EETGERVQLAHHFSEPEITLIIFGVMAGVIGTILLISYGIR |
| | RLIKKSPSDVKPLPSPDTDVPLSSVEIENPETSDQ |
| Leader sequence- human CGL variant (with | MYGKIIFVLLLSEIVSISAMQEKDASSQGFLPHFQHFAT |
| amino acid substitutions E59I, S63L, L91M, | QAIHVGQDPEQWTSRAVVPPISLSTTFKQGAPGQHS |
| R119D, K268R, T311G, E339V, and I353S)-linker- | GFIYSRLGNPTRNCLEKAVAALDGAKYCLAFASGMAA |
| HA-linker-GPA transmembrane region | TVTITHLLKAGDQIICMDDVYGGTNDYFRQVASEFGLK |
| (leader sequence underlined) | ISFVDCSKIKLLEAAITPETKLVWIETPTNPTQKVIDIEGC |
| (SEQ ID NO: 106) | AHIVHKHGDIILVVDNTFMSPYFQRPLALGADISMYSA |
| | TKYMNGHSDVVMGLVSVNCESLHNRLRFLQNSLGAV |
| | PSPIDCYLCNRGLKTLHVRMERHFKNGMAVAQFLESN |
| | PWVEKVIYPGLPSHPQHELVKRQCTGCGGMVTFYIKG |
| | TLQHAEIFLKNLKLFTLAVSLGGFESLAELPASMTHASV |
| | LKNDRDVLGISDTLIRLSVGLEDEEDLLEDLDQALKAAH |
| | PPSGSHSGGGGSGGGGYPYDVPDYAGGGGSGGGGL |
| | STTEVAMHTSTSSSVTKSYISSQTNDTHKRDTYAATPR |
| | AHEVSEISVRTVYPPEEETGERVQLAHHFSEPEITLIIFG |
| | VMAGVIGTILLISYGIRRLIKKSPSDVKPLPSPDTDVPLSS |
| | VEIENPETSDQ |
| Human CGL variant (with amino acid | MQEKDASSQGFLPHFQHFATQAIHVGQDPEQWTSR |
| substitutions E59I, S63L, L91M, R119D, K268R, | AVVPPISLSTTFKQGAPGQHSGFIYSRLGNPTRNCLEK |
| T311G, E339V, and I353S)-linker-HA-linker-GPA | AVAALDGAKYCLAFASGMAATVTITHLLKAGDQIICM |
| (SEQ ID NO: 107) | DDVYGGTNDYFRQVASEFGLKISFVDCSKIKLLEAAITP |
| | ETKLVWIETPTNPTQKVIDIEGCAHIVHKHGDIILVVDN |
| | TFMSPYFQRPLALGADISMYSATKYMNGHSDVVMGL |
| | VSVNCESLHNRLRFLQNSLGAVPSPIDCYLCNRGLKTL |
| | HVRMERHFKNGMAVAQFLESNPWVEKVIYPGLPSHP |
| | QHELVKRQCTGCGGMVTFYIKGTLQHAEIFLKNLKLFT |
| | LAVSLGGFESLAELPASMTHASVLKNDRDVLGISDTLIR |
| | LSVGLEDEEDLLEDLDQALKAAHPPSGSHSGGGGSGG |
| | GGYPYDVPDYAGGGGSGGGGLSTTEVAMHTSTSSSV |
| | TKSYISSQTNDTHKRDTYAATPRAHEVSEISVRTVYPPE |
| | EETGERVQLAHHFSEPEITLIIFGVMAGVIGTILLISYGIR |
| | RLIKKSPSDVKPLPSPDTDVPLSSVEIENPETSDQ |
| SMIM1 transmembrane region-linker-HA-linker- | MQPQESHVHYSRWEDGSRDGVSLGAVSSTEEASRCR |
| human CGL variant (with amino acid substitutions | RISQRLCTGKLGIAMKVLGGVALFWIIFILGYLTGYYVH |
| E59I, S63L, L91M, R119A, K268R, T311G, E339V, | KCKGGGGSGGGGYPYDVPDYAGGGGSGGGGMQEK |
| and I353S) | DASSQGFLPHFQHFATQAIHVGQDPEQWTSRAVVPP |
| (SEQ ID NO: 108) | ISLSTTFKQGAPGQHSGFIYSRLGNPTRNCLEKAVAAL |
| | DGAKYCLAFASGMAATVTITHLLKAGDQIICMDDVYG |
| | GTNAYFRQVASEFGLKISFVDCSKIKLLEAAITPETKLV |
| | WIETPTNPTQKVIDIEGCAHIVHKHGDIILVVDNTFMS |
| | PYFQRPLALGADISMYSATKYMNGHSDVVMGLVSVN |
| | CESLHNRLRFLQNSLGAVPSPIDCYLCNRGLKTLHVRM |
| | ERHFKNGMAVAQFLESNPWVEKVIYPGLPSHPQHEL |
| | VKRQCTGCGGMVTFYIKGTLQHAEIFLKNLKLFTLAVS |
| | LGGFESLAELPASMTHASVLKNDRDVLGISDTLIRLSVG |
| | LEDEEDLLEDLDQALKAAHPPSGSHS |
| SMIM1 transmembrane region-linker-HA-linker- | MQPQESHVHYSRWEDGSRDGVSLGAVSSTEEASRCR |
| human CGL variant (with amino acid substitutions | RISQRLCTGKLGIAMKVLGGVALFWIIFILGYLTGYYVH |
| E59I, S63L, L91M, R119D, K268R, T311G, E339V, | KCKGGGGSGGGGYPYDVPDYAGGGGSGGGGMQEK |
| and I353S) | DASSQGFLPHFQHFATQAIHVGQDPEQWTSRAVVPP |
| (SEQ ID NO: 109) | ISLSTTFKQGAPGQHSGFIYSRLGNPTRNCLEKAVAAL |
| | DGAKYCLAFASGMAATVTITHLLKAGDQIICMDDVYG |
| | GTNDYFRQVASEFGLKISFVDCSKIKLLEAAITPETKLV |
| | WIETPTNPTQKVIDIEGCAHIVHKHGDI1LVVDNTFMS |
| | PYFQRPLALGADISMYSATKYMNGHSDVVMGLVSVN |
| | CESLHNRLRFLQNSLGAVPSPIDCYLCNRGLKTLHVRM |
| | ERHFKNGMAVAQFLESNPWVEKVIYPGLPSHPQHEL |
| | VKRQCTGCGGMVTFYIKGTLQHAEIFLKNLKLFTLAVS |
| | LGGFESLAELPASMTHASVLKNDRDVLGISDTLIRLSVG |
| | LEDEEDLLEDLDQALKAAHPPSGSHS |
| Wild-type human CGL-linker-HA | MQEKDASSQGFLPHFQHFATQAIHVGQDPEQWTSR |
| (SEQ ID NO: 110) | AVVPPISLSTTFKQGAPGQHSGFEYSRSGNPTRNCLEK |
| | AVAALDGAKYCLAFASGLAATVTITHLLKAGDQIICMD |
| | DVYGGTNRYFRQVASEFGLKISFVDCSKIKLLEAAITPET |
| | KLVWIETPTNPTQKVIDIEGCAHIVHKHGDIILVVDNTF |
| | MSPYFQRPLALGADISMYSATKYMNGHSDVVMGLVS |
| | VNCESLHNRLRFLQNSLGAVPSPIDCYLCNRGLKTLHV |
| | RMEKHFKNGMAVAQFLESNPWVEKVIYPGLPSHPQ |
| | HELVKRQCTGCTGMVTFYIKGTLQHAEIFLKNLKLFTL |
| | AESLGGFESLAELPAIMTHASVLKNDRDVLGISDTLIRL |
| | SVGLEDEEDLLEDLDQALKAAHPPSGSHSGGGSGGGY |
| | PYDVPDYA |
| mutant hCGL (with amino acid substitutions E59I, | MQEKDASSQGFLPHFQHFATQAIHVGQDPEQWTSR |
| S63L, L91M, R119A, K268R, T311G, E339V, and | AVVPPISLSTTFKQGAPGQHSGFIYSRLGNPTRNCLEK |
| I353S)-linker-HA | AVAALDGAKYCLAFASGMAATVTITHLLKAGDQIICM |
| (SEQ ID NO: 111) | DDVYGGTNAYFRQVASEFGLKISFVDCSKIKLLEAAITP |
| | ETKLVWIETPTNPTQKVIDIEGCAHIVHKHGDI1LVVDN |
| | TFMSPYFQRPLALGADISMYSATKYMNGHSDVVMGL |
| | VSVNCESLHNRLRFLQNSLGAVPSPIDCYLCNRGLKTL |
| | HVRMERHFKNGMAVAQFLESNPWVEKVIYPGLPSHP |
| | QHELVKRQCTGCGGMVTFYIKGTLQHAEIFLKNLKLFT |
| | LAVSLGGFESLAELPASMTHASVLKNDRDVLGISDTLIR |
| | LSVGLEDEEDLLEDLDQALKAAHPPSGSHSGGGSGGG |
| | YPYDVPDYA |
| mutant hCGL (with amino acid substitutions E59I, | MQEKDASSQGFLPHFQHFATQAIHVGQDPEQWTSR |
| S63L, L91M, R119D, K268R, T311G, E339V, and | AVVPPISLSTTFKQGAPGQHSGFIYSRLGNPTRNCLEK |
| I353S)-Iinker-HA | AVAALDGAKYCLAFASGMAATVTITHLLKAGDQIICM |
| (SEQ ID NO: 112) | DDVYGGTNDYFRQVASEFGLKISFVDCSKIKLLEAAITP |
| | ETKLVWIETPTNPTQKVIDIEGCAHIVHKHGDIILVVDN |
| | TFMSPYFQRPLALGADISMYSATKYMNGHSDVVMGL |
| | VSVNCESLHNRLRFLQNSLGAVPSPIDCYLCNRGLKTL |
| | HVRMERHFKNGMAVAQFLESNPWVEKVIYPGLPSHP |
| | QHELVKRQCTGCGGMVTFYIKGTLQHAEIFLKNLKLFT |
| | LAVSLGGFESLAELPASMTHASVLKNDRDVLGISDTLIR |
| | LSVGLEDEEDLLEDLDQALKAAHPPSGSHSGGGSGGG |
| | YPYDVPDYA |

### Amino Acid Transporters

In one aspect, the disclosure provides an engineered erythroid cell comprising a first exogenous polypeptide comprising a homocysteine or serine transporter, or a variant thereof.

In another aspect, the disclosure provides an erythroid cell engineered to degrade homocysteine and/or its metabolites, wherein the cell comprises a first exogenous polypeptide comprising a homocysteine degrading polypeptide, or a variant thereof, and further comprises one or more (e.g, two, three, four, five, or more) additional exogenous polypeptides, each comprising one or more amino acid transporters, or a variant thereof.

In another aspect, the disclosure provides an erythroid cell engineered to degrade homocysteine and/or its metabolites, wherein the cell comprises a first exogenous polypeptide comprising a homocysteine degrading polypeptide, or a variant thereof, and further comprises a second exogenous polypeptide comprising a homocysteine transporter, or a variant thereof.

In another aspect, the disclosure provides an erythroid cell engineered to degrade homocysteine and/or its metabolites, wherein the cell comprises a first exogenous polypeptide comprising a homocysteine degrading polypeptide, or a variant thereof, and further comprises a second exogenous polypeptide comprising a serine transporter, or a variant thereof.

In another aspect, the disclosure provides an erythroid cell engineered to degrade homocysteine and/or its metabolites, wherein the cell comprises a first exogenous polypeptide comprising a homocysteine degrading polypeptide, or a variant thereof, and further comprises a second exogenous polypeptide comprising a homocysteine transporter, or a variant thereof, and a third exogenous polypeptide comprising a serine transporter, or a variant thereof.

In another aspect, the disclosure provides an erythroid cell comprising at least one exogenous polypeptide comprising a cystathionine degrading polypeptide, and at least one exogenous polypeptide comprising an amino acid transporter. In some embodiments, the disclosure provides an erythroid cell (e.g., an enucleated erythroid cell) comprising: a first exogenous polypeptide, wherein the first exogenous polypeptide comprises a cystathionine degrading polypeptide (e.g., a cystathionine gamma-lyase or variant thereof described herein); and a second exogenous polypeptide, wherein the second exogenous polypeptide comprises an amino acid transporter (e.g., a homocysteine transporter or a serine transporter, e.g., LAT1). In some embodiments, the disclosure provides an erythroid cell (e.g., an enucleated erythroid cell) comprising: a first exogenous polypeptide, wherein the first exogenous polypeptide comprises a cystathionine degrading polypeptide (e.g., a cystathionine gamma-lyase or variant thereof described herein); a second exogenous polypeptide, wherein the second exogenous polypeptide comprises an amino acid transporter (e.g., a homocysteine transporter or a serine transporter, e.g., LAT1); and a third exogenous polypeptide, wherein the third exogenous polypeptide comprises an amino acid transporter (e.g., a homocysteine transporter or a serine transporter, e.g., CD98).

Amino acid transporters are membrane transport proteins that transport amino acids. They are mainly members of the solute carrier family. Amino acid transporters are found in fungi, plants, and animals (Wipf et al., 2002 TRENDS in Biochemical Science, 27(3); the contents of which are hereby incorporated herein by reference).

In some embodiments, an engineered erythroid cell of the disclosure comprises an exogenous polypeptide comprising an amino acid transporter, e.g., a homocysteine transporter and/or a serine transporter, selected from the group consisting of sodium-coupled neutral amino acid transporter 1 (SLC38A1) (SAT1), Sodium-coupled neutral amino acid transporter 2 (SLC38A2) (SAT2), sodium-coupled neutral amino acid transporter 4 (SLC38A4) (SAT4), neutral amino acid transporter A (SLC1A4) (ASCT1), neutral amino acid transporter B(0) (SLC1A5) (ASCT2), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1), excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), Proton-coupled amino acid transporter 1 (SLC36A1), proton-coupled amino acid transporter 2 (SLC36A2), sodium- and chloride-dependent neutral and basic amino acid transporter B(0+) (SLC6A14), Y+L amino acid transporter 1 (SLC7A7) Y+L amino acid transporter 2 (SLC7A6), organic anion transporter 1 (SLC22A6) (OAT1), T-type amino acid transporter (SLC16A10) (TAT1).

In one embodiment, an engineered erythroid cell of the disclosure comprises an exogenous polypeptide comprising large neutral amino acids transporter small subunit 1 (SLC7A5) (LAT1).

In one embodiment, an engineered erythroid cell provided herein comprises an exogenous polypeptide comprising a human homocysteine transporter or serine transporter.

In some preferred embodiments, the erythroid cell of the disclosure comprises at least one exogenous polypeptide comprising an amino acid transporter selected from those set forth in Table 6, below. In some embodiments, the amino acid transporter comprises the amino acid sequence of any one of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:48, SEQ ID NO:49, SEQ ID NO:59, SEQ ID NO:60, or SEQ ID NO:61, or a variant thereof.

TABLE 6
Exemplary Homocysteine, homocystine & serine transporter and/or transporter
adapter sequences

| SEQ. ID | | Amino acid sequence |
|---|---|---|
| NO | | |
| 13 | Homo sapiens Sodium- | MMHFKSGLEL TELQNMTVPE DDNISNDSND FTEVENGQIN |
| | coupled neutral amino | SKFISDRESR RSLTNSHLEK KKCDEYIPGT TSLGMSVFNL |
| | acid transporter 1 | SNAIMGSGIL GLAFALANTG ILLFLVLLTS VTLLSIYSIN |
| | (SLC38A1) (SAT1) | LLLICSKETG CMVYEKLGEQ VFGTTGKFVI |
| | | FGATSLQNTG AMLSYLFIVK NELPSAIKFL MGKEETFSAW |
| | | YVDGRVLVVI VTFGIILPLC LLKNLGYLGY TSGFSLSCMV |
| | | FFLIVVIYKK FQIPCIVPEL NSTISANSTN ADTCTPKYVT |
| | | FNSKTVYALP TIAFAFVCHP SVLPIYSELK DRSQKKMQMV |
| | | SNISFFAMFV MYFLTAIFGY LTFYDNVQSD LLHKYQSKDD |
| | | ILILTVRLAV IVAVILTVPV LFFTVRSSLF ELAKKTKFNL |
| | | CRHTVVTCIL LVVINLLVIF IPSMKDIFGV VGVTSANMLI |
| | | FILPSSLYLK ITDQDGDKGT QRIWAALFLG LGVLFSLVSI |
| | | PLVIYDWACS SSSDEGH |
| 14 | Homo sapiens Sodium- | MKKAEMGRFS ISPDEDSSSY SSNSDFNYSY PTKQAALKSH |
| | coupled neutral amino | YADVDPENQN FLLESNLGKK KYETEFHPGT TSFGMSVFNL |
| | acid transporter 2 | SNAIVGSGIL GLSYAMANTG IALFIILLTF VSIFSLYSVH |
| | (SLC38A2) (SAT2) | LLLKTANEGG SLLYEQLGYK AFGLVGKLAA SGSITMQNIG |
| | | AMSSYLFIVK YELPLVIQAL TNIEDKTGLW YLNGNYLVLL |
| | | VSLVVILPLS LFRNLGYLGY TSGLSLLCMV FFLIVVICKK |
| | | FQVPCPVEAA LIINETINTT LTQPTALVPA LSHNVTENDS |
| | | CRPHYFIFNS QTVYAVPILI FSFVCHPAVL PIYEELKDRS |
| | | RRRMMNVSKI SFFAMFLMYL LAALFGYLTF YEHVESELLH |
| | | TYSSILGTDI LLLIVRLAVL MAVTLTVPVV IFPIRSSVTH |
| | | LLCASKDFSW WRHSLITVSI LAFTNLLVIF VPTIRDIFGF |
| | | IGASAASMLI FILPSAFYIK LVKKEPMKSV QKIGALFFLL |
| | | SGVLVMTGSM ALIVLDWVHN APGGGH |
| 15 | Homo sapiens Sodium- | MDPMELRNVN IEPDDESSSG ESAPDSYIGI GNSEKAAMSS |
| | coupled neutral amino | QFANEDTESQ KFLTNGFLGK KKLADYADEH HPGTTSFGMS |
| | acid transporter 4 | SFNLSNAIMG SGILGLSYAM ANTGIILFII MLLAVAILSL |
| | (SLC38A4) (SAT3) | YSVHLLLKTA KEGGSLIYEK LGEKAFGWPG KIGAFVSITM |
| | | QNIGAMSSYL FIIKYELPEV IRAFMGLEEN TGEWYLNGNY |
| | | LIIFVSVGII LPLSLLKNLG YLGYTSGFSL TCMVFFVSVV |
| | | IYKKFQIPCP LPVLDHSVGN LSFNNTLPMH VVMLPNNSES |
| | | SDVNFMMDYT HRNPAGLDEN QAKGSLHDSG |
| | | VEYEAHSDDK CEPKYFVFNS RTAYAIPILV FAFVCHPEVL |
| | | PIYSELKDRS RRKMQTVSNI SITGMLVMYL LAALFGYLTF |
| | | YGEVEDELLH AYSKVYTLDI PLLMVRLAVL VAVTLTVPIV |
| | | LFPIRTSVIT LLFPKRPFSW IRHFLIAAVL IALNNVLVIL |
| | | VPTIKYIFGF IGASSATMLI FILPAVFYLK LVKKETFRSP |
| | | QKVGALIFLV VGIFFMIGSM ALIIIDWIYD PPNSKHH |
| 16 | Homo sapiens Neutral | MEKSNETNGY LDSAQAGPAA GPGAPGTAAG RARRCAGFLR |
| | amino acid transporter | RQALVLLTVS GVLAGAGLGA ALRGLSLSRT QVTYLAFPGE |
| | A (SLC1A4) (ASCT1) | MLLRMLRMII LPLVVCSLVS GAASLDASCL GRLGGIAVAY |
| | | FGLTTLSASA LAVALAFIIK PGSGAQTLQS |
| | | SDLGLEDSGP PPVPKETVDS FLDLARNLFP SNLVVAAFRT |
| | | YATDYKVVTQ NSSSGNVTHE KIPIGTEIEG MNILGLVLFA |
| | | LVLGVALKKL GSEGEDLIRF FNSLNEATMV LVSWIMWYVP |
| | | VGIMFLVGSK IVEMKDIIVL VTSLGKYIFA SILGHVIHGG |
| | | IVLPLIYFVF TRKNPFRFLL GLLAPFATAF ATCSSSATLP |
| | | SMMKCIEENN GVDKRISRFI LPIGATVNMD GAAIFQCVAA |
| | | VFIAQLNNVE LNAGQIFTIL VTATASSVGA AGVPAGGVLT |
| | | IAIILEAIGL PTHDLPLILA VDWIVDRTTT VVNVEGDALG |
| | | AGILHHLNQK ATKKGEQELA EVKVEAIPNC KSEEETSPLV |
| | | THQNPAGPVA SAPELESKES VL |
| 17 | Homo sapiens Neutral | MVADPPRDSK GLAAAEPTAN GGLALASIED QGAAAGGYCG |
| | amino acid transporter | SRDQVRRCLR ANLLVLLTVV AVVAGVALGL GVSGAGGALA |
| | B(0) (SLC1A5) (ASCT2) | LGPERLSAFV FPGELLLRLL |
| | | RMIILPLVVC SLIGGAASLD PGALGRLGAW ALLFFLVTTL |
| | | LASALGVGLA LALQPGAASA AINASVGAAG SAENAPSKEV |
| | | LDSFLDLARN IFPSNLVSAA FRSYSTTYEE RNITGTRVKV |
| | | PVGQEVEGMN ILGLVVFAIV FGVALRKLGP EGELLIRFFN |
| | | SFNEATMVLV SWIMWYAPVG IMFLVAGKIV EMEDVGLLFA |
| | | RLGKYILCCL LGHAIHGLLV LPLIYFLFTR KNPYRFLWGI |
| | | VTPLATAFGT SSSSATLPLM MKCVEENNGV AKHISRFILP |
| | | IGATVNMDGA ALFQCVAAVF IAQLSQQSLD FVKIITILVT |
| | | ATASSVGAAG IPAGGVLTLA IILEAVNLPV |
| | | DHISLILAVD WLVDRSCTVL NVEGDALGAG LLQNYVDRTE |
| | | SRSTEPELIQ VKSELPLDPL PVPTEEGNPL LKHYRGPAGD |
| | | ATVASEKESV M |
| 18 | Homo sapiens Large | MAGAGPKRRA LAAPAAEEKE EAREKMLAAK SADGSAPAGE |
| | neutral amino acids | GEGVTLQRNI TLLNGVAIIV GTIIGSGIFV TPTGVLKEAG |
| | transporter small | SPGLALVVWA ACGVFSIVGA LCYAELGTTI SKSGGDYAYM |
| | subunit 1 (SLC7A5) | LEVYGSLPAF LKLWIELLII RPSSQYIVAL |
| | (LAT1) | VFATYLLKPL FPTCPVPEEA AKLVACLCVL LLTAVNCYSV |
| | | KAATRVQDAF AAAKLLALAL IILLGFVQIG KGDVSNLDPN |
| | | FSFEGTKLDV GNIVLALYSG LFAYGGWNYL NFVTEEMINP |
| | | YRNLPLAIII SLPIVTLVYV LTNLAYFTTL STEQMLSSEA |
| | | VAVDFGNYHL GVMSWIIPVF VGLSCFGSVN GSLFTSSRLF |
| | | FVGSREGHLP SILSMIHPQL LTPVPSLVFT CVMTLLYAFS |
| | | KDIFSVINFF SFFNWLCVAL AIIGMIWLRH RKPELERPIK |
| | | VNLALPVFFI LACLFLIAVS FWKTPVECGI GFTIILSGLP |
| | | VYFFGVWWKN KPKWLLQGIF STTVLCQKLM QVVPQET |
| 19 | Homo sapiens Large | MEEGARHRNN TEKKHPGGGE SDASPEAGSG GGGVALKKEI |
| | neutral amino acids | GLVSACGIIV GNIIGSGIFV SPKGVLENAG SVGLALIVWI |
| | transporter small | VTGFITVVGA LCYAELGVTI PKSGGDYSYV KDIFGGLAGF |
| | subunit 2 (SLC7A8) | LRLWIAVLVI YPTNQAVIAL TFSNYVLQPL |
| | (LAT2) | FPTCFPPESG LRLLAAICLL LLTWVNCSSV RWATRVQDIF |
| | | TAGKLLALAL IIIMGIVQIC KGEYFWLEPK NAFENFQEPD |
| | | IGLVALAFLQ GSFAYGGWNF LNYVTEELVD PYKNLPRAIF |
| | | ISIPLVTFVY VFANVAYVTA MSPQELLASN AVAVTFGEKL |
| | | LGVMAWIMPI SVALSTFGGV NGSLFTSSRL FFAGAREGHL |
| | | PSVLAMIHVK RCTPIPALLF TCISTLLMLV TSDMYTLINY |
| | | VGFINYLFYG VTVAGQIVLR WKKPDIPRPI KINLLFPIIY |
| | | LLFWAFLLVF SLWSEPVVCG IGLAIMLTGV PVYFLGVYWQ |
| | | HKPKCFSDFI ELLTLVSQKM CVVVYPEVER GSGTEEANED |
| | | MEEQQQPMYQ PTPTKDKDVA GQPQP |
| 20 | Homo sapiens | MTKSNGEEPK MGGRMERFQQ GVRKRTLLAK KKVQNITKED |
| | Excitatory amino acid | VKSYLFRNAF VLLTVTAVIV GTILGFTLRP YRMSYREVKY |
| | transporter 1 (SLC1A3) | FSFPGELLMR MLQMLVLPLI ISSLVTGMAA LDSKASGKMG |
| | (EAAT1) | MRAVVYYMTT TIIAVVIGII IVIIIHPGKG |
| | | TKENMHREGK IVRVTAADAF LDLIRNMFPP NLVEACFKQF |
| | | KTNYEKRSFK VPIQANETLV GAVINNVSEA METLTRITEE |
| | | LVPVPGSVNG VNALGLVVFS MCFGFVIGNM KEQGQALREF |
| | | FDSLNEAIMR LVAVIMWYAP VGILFLIAGK |
| | | IVEMEDMGVI GGQLAMYTVT VIVGLLIHAV IVLPLLYFLV |
| | | TRKNPWVFIG GLLQALITAL GTSSSSATLP ITFKCLEENN |
| | | GVDKRVTRFV LPVGATINMD GTALYEALAA IFIAQVNNFE |
| | | LNFGQIITIS ITATAASIGA AGIPQAGLVT MVIVLTSVGL |
| | | PTDDITLIIA VDWFLDRLRT TTNVLGDSLG AGIVEHLSRH |
| | | ELKNRDVEMG NSVIEENEMK KPYQLIAQDN ETEKPIDSET |
| | | KM |
| 21 | Homo sapiens | MASTEGANNM PKQVEVRMHD SHLGSEEPKH |
| | Excitatory amino acid | RHLGLRLCDK LGKNLLLTLT VFGVILGAVC GGLLRLASPI |
| | transporter 2 (SLC1A2) | HPDVVMLIAF PGDILMRMLK MLILPLIISS LITGLSGLDA |
| | (EAAT2) | KASGRLGTRA MVYYMSTTII AAVLGVILVL AIHPGNPKLK |
| | | KQLGPGKKND EVSSLDAFLD LIRNLFPENL VQACFQQIQT |
| | | VTKKVLVAPP PDEEANATSA VVSLLNETVT EVPEETKMVI |
| | | KKGLEFKDGM NVLGLIGFFI AFGIAMGKMG DQAKLMVDFF |
| | | NILNEIVMKL VIMIMWYSPL GIACLICGKI |
| | | IAIKDLEVVA RQLGMYMVTV IIGLIIHGGI FLPLIYFVVT |
| | | RKNPFSFFAG IFQAWITALG TASSAGTLPV TFRCLEENLG |
| | | IDKRVTRFVL PVGATINMDG TALYEAVAAI FIAQMNGVVL |
| | | DGGQIVTVSL TATLASVGAA SIPSAGLVTM LLILTAVGLP |
| | | TEDISLLVAV DWLLDRMRTS VNVVGDSFGA GIVYHLSKSE |
| | | LDTIDSQHRV HEDIEMTKTQ SIYDDMKNHR ESNSNQCVYA |
| | | AHNSVIVDEC KVTLAANGKS ADCSVEEEPW KREK |
| 22 | Homo sapiens | MGKPARKGCE WKRFLKNNWV LLSTVAAVVL GITTGVLVRE |
| | Excitatory amino acid | HSNLSTLEKF YFAFPGEILM RMLKLIILPL IISSMITGVA |
| | transporter 3 (SLC1A1) | ALDSNVSGKI GLRAVVYYFC TTLIAVILGI VLVVSIKPGV |
| | (EAAT3) | TQKVGEIART GSTPEVSTVD AMLDLIRNMF |
| | | PENLVQACFQ QYKTKREEVK PPSDPEMNMT EESFTAVMTT |
| | | AISKNKTKEY KIVGMYSDGI NVLGLIVFCL VFGLVIGKMG |
| | | EKGQILVDFF NALSDATMKI VQIIMCYMPL GILFLIAGKI |
| | | IEVEDWEIFR KLGLYMATVL TGLAIHSIVI |
| | | LPLIYFIVVR KNPFRFAMGM AQALLTALMI SSSSATLPVT |
| | | FRCAEENNQV DKRITRFVLP VGATINMDGT ALYEAVAAVF |
| | | IAQLNDLDLG IGQIITISIT ATSASIGAAG VPQAGLVTMV |
| | | IVLSAVGLPA EDVTLIIAVD WLLDRFRTMV NVLGDAFGTG |
| | | IVEKLSKKEL EQMDVSSEVN IVNPFALEST ILDNEDSDTK |
| | | KSYVNGGFAV DKSDTISFTQ TSQF |
| 23 | Homo sapiens | MSSHGNSLFL RESGQRLGRV GWLQRLQESL QQRALRTRLR |
| | Excitatory amino acid | LQTMTLEHVL RFLRRNAFIL LTVSAVVIGV SLAFALRPYQ |
| | transporter 4 (SLC1A6) | LTYRQIKYFS FPGELLMRML QMLVLPLIVS SLVTGMASLD |
| | (EAAT4) | NKATGRMGMR AAVYYMVTTI IAVFIGILMV TIIHPGKGSK |
| | | EGLHREGRIE TIPTADAFMD LIRNMFPPNL VEACFKQFKT |
| | | QYSTRVVTRT MVRTENGSEP GASMPPPFSV ENGTSFLENV |
| | | TRALGTLQEM LSFEETVPVP GSANGINALG LVVFSVAFGL |
| | | VIGGMKHKGR VLRDFFDSLN EAIMRLVGII IWYAPVGILF |
| | | LIAGKILEME DMAVLGGQLG MYTLTVIVGL FLHAGIVLPL |
| | | IYFLVTHRNP FPFIGGMLQA LITAMGTSSS SATLPITFRC |
| | | LEEGLGVDRR ITRFVLPVGA TVNMDGTALY EALAAIFIAQ |
| | | VNNYELNLGQ ITTISITATA ASVGAAGIPQ AGLVTMVIVL |
| | | TSVGLPTEDI TLIIAVDWFL DRLRTMTNVL GDSIGAAVIE |
| | | HLSQRELELQ EAELTLPSLG KPYKSLMAQE KGASRGRGGN |
| | | ESAM |
| 24 | Homo sapiens | MVPHAILARG RDVCRRNGLL ILSVLSVIVG CLLGFFLRTR |
| | Excitatory amino acid | RLSPQEISYF QFPGELLMRM LKMMILPLVV SSLMSGLASL |
| | transporter 5 (SLC1A7) | DAKTSSRLGV LTVAYYLWTT FMAVIVGIFM VSIIHPGSAA |
| | (EAAT5) | QKETTEQSGK PIMSSADALL DLIRNMFPAN LVEATFKQYR |
| | | TKTTPVVKSP KVAPEEAPPR RILIYGVQEE NGSHVQNFAL |
| | | DLTPPPEVVY KSEPGTSDGM NVLGIVFFSA TMGIMLGRMG |
| | | DSGAPLVSFC QCLNESVMKI VAVAVWYFPF GIVFLIAGKI |
| | | LEMDDPRAVG KKLGFYSVTV |
| | | VCGLVLHGLF ILPLLYFFIT KKNPIVFIRG ILQALLIALA |
| | | TSSSSATLPI TFKCLLENNH IDRRIARFVL PVGATINMDG |
| | | TALYEAVAAI FIAQVNNYEL DFGQIITISI TATAASIGAA |
| | | GIPQAGLVTM VIVLTSVGLP TDDITLIIAV DWALDRFRTM |
| | | INVLGDALAA GIMAHICRKD FARDTGTEKL LPCETKPVSL |
| | | QEIVAAQQNG CVKSVAEASE LTLGPTCPHH VPVQVEQDEE |
| | | LPAASLNHCT IQISELETNV |
| 25 | Homo sapiens 4F2 cell- | MELQPPEASI AVVSIPRQLP GSHSEAGVQG LSAGDDSELG |
| | surface antigen heavy | SHCVAQTGLE LLASGDPLPS ASQNAEMIET GSDCVTQAGL |
| | chain (SLC3A2) CD98 | QLLASSDPPA LASKNAEVTG TMSQDTEVDM KEVELNELEP |
| | | EKQPMNAASG AAMSLAGAEK NGLVKIKVAE DEAEAAAAAK |
| | | FTGLSKEELL KVAGSPGWVR TRWALLLLFW LGWLGMLAGA |
| | | VVIIVRAPRC RELPAQKWWH TGALYRIGDL QAFQGHGAGN |
| | | LAGLKGRLDY LSSLKVKGLV LGPIHKNQKD DVAQTDLLQI |
| | | DPNFGSKEDF DSLLQSAKKK |
| | | SIRVILDLTP NYRGENSWFS TQVDTVATKV KDALEFWLQA |
| | | GVDGFQVRDI ENLKDASSFL AEWQNITKGF SEDRLLIAGT |
| | | NSSDLQQILS LLESNKDLLL TSSYLSDSGS TGEHTKSLVT |
| | | QYLNATGNRW CSWSLSQARL LTSFLPAQLL RLYQLMLFTL |
| | | PGTPVFSYGD EIGLDAAALP GQPMEAPVML WDESSFPDIP |
| | | GAVSANMTVK GQSEDPGSLL SLFRRLSDQR SKERSLLHGD |
| | | FHAFSAGPGL FSYIRHWDQN ERFLVVLNFG DVGLSAGLQA |
| | | SDLPASASLP AKADLLLSTQ PGREEGSPLE LERLKLEPHE |
| | | GLLLRFPYAA |
| 26 | Homo sapiens Sodium- | MEAPLQTEMV ELVPNGKHSE GLLPVITPMA GNQRVEDPAR |
| | coupled neutral amino | SCMEGKSFLQ KSPSKEPHFT DFEGKTSFGM SVFNLSNAIM |
| | acid transporter 3 | GSGILGLAYA MANTGIILFL FLLTAVALLS SYSIHLLLKS |
| | (SLC38A3) (SN1) | SGVVGIRAYE QLGYRAFGTP GKLAAALAIT |
| | | LQNIGAMSSY LYIIKSELPL VIQTFLNLEE KTSDWYMNGN |
| | | YLVILVSVTI ILPLALMRQL GYLGYSSGFS LSCMVFFLIA |
| | | VIYKKFHVPC PLPPNFNNTT GNFSHVEIVK EKVQLQVEPE |
| | | ASAFCTPSYF TLNSQTAYTI PIMAFAFVCH PEVLPIYTEL |
| | | KDPSKKKMQH ISNLSIAVMY IMYFLAALFG YLTFYNGVES |
| | | ELLHTYSKVD PFDVLILCVR VAVLTAVTLT VPIVLFPVRR |
| | | AIQQMLFPNQ EFSWLRHVLI AVGLLTCINL LVIFAPNILG |
| | | IFGVIGATSA PFLIFIFPAI FYFRIMPTEK EPARSTPKIL |
| | | ALCFAMLGFL LMTMSLSFII IDWASGTSRH GGNH |
| 27 | Homo sapiens Sodium- | MELQDPKMNG ALPSDAVGYR QEREGFLPSR GPAPGSKPVQ |
| | coupled neutral amino | FMDFEGKTSF GMSVFNLSNA IMGSGILGLA YAMAHTGVIF |
| | acid transporter 5 | FLALLLCIAL LSSYSIHLLL TCAGIAGIRA YEQLGQRAFG |
| | (SLC38A5) (5N2) | PAGKVVVATV ICLHNVGAMS SYLFIIKSEL |
| | | PLVIGTFLYM DPEGDWFLKG NLLIIIVSVL IILPLALMKH |
| | | LGYLGYTSGL SLTCMLFFLV SVIYKKFQLG CAIGHNETAM |
| | | ESEALVGLPS QGLNSSCEAQ MFTVDSQMSY TVPIMAFAFV |
| | | CHPEVLPIYT ELCRPSKRRM QAVANVSIGA MFCMYGLTAT |
| | | FGYLTFYSSV KAEMLHMYSQ KDPLILCVRL AVLLAVTLTV |
| | | PVVLFPIRRA LQQLLFPGKA FSWPRHVAIA LILLVLVNVL |
| | | VICVPTIRDI FGVIGSTSAP SLIFILPSIF YLRIVPSEVE |
| | | PFLSWPKIQA LCFGVLGVLF MAVSLGFMFA NWATGQSRMS |
| | | GH |
| 28 | Homo sapiens Asc-type | MAGHTQQPSG RGNPRPAPSP SPVPGTVPGA SERVALKKEI |
| | amino acid transporter | GLLSACTIII GNIIGSGIFI SPKGVLEHSG SVGLALFVWV |
| | 1 (SLC7A10) (Asci) | LGGGVTALGS LCYAELGVAI PKSGGDYAYV TEIFGGLAGF |
| | | LLLWSAVLIM YPTSLAVISM TFSNYVLQPV FPNCIPPTTA |
| | | SRVLSMACLM LLTWVNSSSV RWATRIQDMF TGGKLLALSL |
| | | IIGVGLLQIF QGHFEELRPS NAFAFWMTPS VGHLALAFLQ |
| | | GSFAFSGWNF LNYVTEEMVD ARKNLPRAIF ISIPLVTFVY |
| | | TFTNIAYFTA MSPQELLSSN AVAVTFGEKL LGYFSWVMPV |
| | | SVALSTFGGI NGYLFTYSRL CFSGAREGHL PSLLAMIHVR |
| | | HCTPIPALLV CCGATAVIML VGDTYTLINY VSFINYLCYG |
| | | VTILGLLLLR WRRPALHRPI KVNLLIPVAY LVFWAFLLVF |
| | | SFISEPMVCG VGVIIILTGV PIFFLGVFWR SKPKCVHRLT |
| | | ESMTHWGQEL CFVVYPQDAP EEEENGPCPP SLLPATDKPS |
| | | KPQ |
| 29 | Homo sapiens b(0,+)- | MGDTGLRKRR EDEKSIQSQE PKTTSLQKEL GLISGISIIV |
| | type amino acid | GTIIGSGIFV SPKSVLSNTE AVGPCLIIWA ACGVLATLGA |
| | transporter 1 (SLC7A9) | LCFAELGTMI TKSGGEYPYL MEAYGPIPAY LFSWASLIVI |
| | | KPTSFAIICL SFSEYVCAPF YVGCKPPQIV VKCLAAAAIL |
| | | FISTVNSLSV RLGSYVQNIF TAAKLVIVAI IIISGLVLLA |
| | | QGNTKNFDNS FEGAQLSVGA ISLAFYNGLW |
| | | AYDGWNQLNY ITEELRNPYR NLPLAIIIGI PLVTACYILM |
| | | NVSYFTVMTA TELLQSQAVA VTFGDRVLYP ASWIVPLFVA |
| | | FSTIGAANGT CFTAGRLIYV AGREGHMLKV LSYISVRRLT |
| | | PAPAIIFYGI IATIYIIPGD INSLVNYFSF AAWLFYGLTI |
| | | LGLIVMRFTR KELERPIKVP VVIPVLMTLI SVFLVLAPII |
| | | SKPTWEYLYC VLFILSGLLF YFLFVHYKFG WAQKISKPIT |
| | | MHLQMLMEVV PPEEDPE |
| 30 | Homo sapiens Neutral | MAEDKSKRDS IEMSMKGCQT NNGFVHNEDI LEQTPDPGSS |
| | and basic amino acid | TDNLKHSTRG ILGSQEPDFK GVQPYAGMPK EVLFQFSGQA |
| | transport protein rBAT | RYRIPREILF WLTVASVLVL IAATIAIIAL SPKCLDWWQE |
| | (SLC3A1) | GPMYQIYPRS FKDSNKDGNG DLKGIQDKLD YITALNIKTV |
| | | WITSFYKSSL KDFRYGVEDF REVDPIFGTM EDFENLVAAI |
| | | HDKGLKLIID FIPNHTSDKH IWFQLSRTRT GKYTDYYIWH |
| | | DCTHENGKTI |
| | | PPNNWLSVYG NSSWHFDEVR NQCYFHQFMK |
| | | EQPDLNFRNP DVQEEIKEIL RFWLTKGVDG FSLDAVKFLL |
| | | EAKHLRDEIQ VNKTQIPDTV TQYSELYHDF TTTQVGMHDI |
| | | VRSFRQTMDQ YSTEPGRYRF MGTEAYAESI DRTVMYYGLP |
| | | FIQEADFPFN NYLSMLDTVS GNSVYEVITS WMENMPEGKW |
| | | PNWMIGGPDS SRLTSRLGNQ YVNVMNMLLF TLPGTPITYY |
| | | GEEIGMGNIV AANLNESYDI |
| | | NTLRSKSPMQ WDNSSNAGFS EASNTWLPTN |
| | | SDYHTVNVDV QKTQPRSALK LYQDLSLLHA NELLLNRGWF |
| | | CHLRNDSHYV VYTRELDGID RIFIVVLNFG ESTLLNLHNM |
| | | ISGLPAKMRI RLSTNSADKG SKVDTSGIFL DKGEGLIFEH |
| | | NTKNLLHRQT AFRDRCFVSN RACYSSVLNI LYTSC |
| 31 | Homo sapiens Proton- | MSTQRLRNED YHDYSSTDVS PEESPSEGLN NLSSPGSYQR |
| | coupled amino acid | FGQSNSTTWF QTLIHLLKGN IGTGLLGLPL AVKNAGIVMG |
| | transporter 1 | PISLLIIGIV AVHCMGILVK CAHHFCRRLN KSFVDYGDTV |
| | (SLC36A1) | MYGLESSPCS WLRNHAHWGR RVVDFFLIVT QLGFCCVYFV |
| | | FLADNFKQVI EAANGTTNNC HNNETVILTP TMDSRLYMLS |
| | | FLPFLVLLVF IRNLRALSIF SLLANITMLV SLVMIYQFIV |
| | | QRIPDPSHLP LVAPWKTYPL FFGTAIFSFE GIGMVLPLEN |
| | | KMKDPRKFPL ILYLGMVIVT ILYISLGCLG YLQFGANIQG |
| | | SITLNLPNCW LYQSVKLLYS IGIFFTYALQ FYVPAEIIIP |
| | | FFVSRAPEHC ELVVDLFVRT VLVCLTCILA ILIPRLDLVI |
| | | SLVGSVSSSA LALIIPPLLE VTTFYSEGMS PLTIFKDALI |
| | | SILGFVGFVV GTYEALYELI QPSNAPIFIN STCAFI |
| 32 | Homo sapiens Proton- | MSVTKSTEGP QGAVAIKLDL MSPPESAKKL ENKDSTFLDE |
| | coupled amino acid | SPSESAGLKK TKGITVFQAL IHLVKGNMGT GILGLPLAVK |
| | transporter 2 | NAGILMGPLS LLVMGFIACH CMHILVKCAQ RFCKRLNKPF |
| | (SLC36A2) | MDYGDTVMHG LEANPNAWLQ NHAHWGRHIV |
| | | SFFLIITQLG FCCVYIVFLA DNLKQVVEAV NSTTNNCYSN |
| | | ETVILTPTMD SRLYMLSFLP FLVLLVLIRN LRILTIFSML |
| | | ANISMLVSLV IIIQYITQEI PDPSRLPLVA SWKTYPLFFG |
| | | TAIFSFESIG VVLPLENKMK NARHFPAILS LGMSIVTSLY |
| | | IGMAALGYLR FGDDIKASIS LNLPNCWLYQ SVKLLYIAGI |
| | | LCTYALQFYV PAEIIIPFAI SRVSTRWALP LDLSIRLVMV |
| | | CLTCLLAILI PRLDLVISLV GSVSGTALAL IIPPLLEVTT |
| | | FYSEGMSPLT IFKDALISIL GFVGFVVGTY QALDELLKSE |
| | | DSHPFSNSTT FVR |
| 33 | Homo sapiens Sodium- | MDKLKCPSFF KCREKEKVSA SSENFHVGEN DENQDRGNWS |
| | and chloride- | KKSDYLLSMI GYAVGLGNVW RFPYLTYSNG GGAFLIPYAI |
| | dependent neutral and | MLALAGLPLF FLECSLGQFA |
| | basic amino acid | SLGPVSVWRI LPLFQGVGIT MVLISIFVTI YYNVIIAYSL |
| | transporter B(0+) | YYMFASFQSE LPWKNCSSWS DKNCSRSPIV THCNVSTVNK |
| | (SLC6A14) | GIQEIIQMNK SWVDINNFTC INGSEIYQPG QLPSEQYWNK |
| | | VALQRSSGMN ETGVIVWYLA LCLLLAWLIV GAALFKGIKS |
| | | SGKVVYFTAL FPYVVLLILL VRGATLEGAS KGISYYIGAQ |
| | | SNFTKLKEAE VWKDAATQIF YSLSVAWGGL VALSSYNKFK |
| | | NNCFSDAIVV CLTNCLTSVF AGFAIFSILG HMAHISGKEV |
| | | SQVVKSGFDL AFIAYPEALA QLPGGPFWSI LFFFMLLTLG |
| | | LDSQFASIET ITTTIQDLFP KVMKKMRVPI TLGCCLVLFL |
| | | LGLVCVTQAG IYWVHLIDHF CAGWGILIAA ILELVGIIWI |
| | | YGGNRFIEDT EMMIGAKRWI FWLWWRACWF VITPILLIAI |
| | | FIWSLVQFHR PNYGAIPYPD WGVALGWCMI VFCIIWIPIM |
| | | AIIKIIQAKG NIFQRLISCC RPASNWGPYL EQHRGERYKD |
| | | MVDPKKEADH EIPTVSGSRK PE |
| 34 | Homo sapiens Y+L | MVDSTEYEVA SQPEVETSPL GDGASPGPEQ VKLKKEISLL |
| | amino acid transporter | NGVCLIVGNM IGSGIFVSPK GVLIYSASFG LSLVIWAVGG |
| | 1 (SLC7A7) | LFSVFGALCY AELGTTIKKS GASYAYILEA FGGFLAFIRL |
| | | WTSLLIIEPT SQAIIAITFA NYMVQPLFPS CFAPYAASRL |
| | | LAAACICLLT FINCAYVKWG TLVQDIFTYA KVLALIAVIV |
| | | AGIVRLGQGA STHFENSFEG SSFAVGDIAL ALYSALFSYS |
| | | GWDTLNYVTE EIKNPERNLP LSIGISMPIV TIIYILTNVA |
| | | YYTVLDMRDI LASDAVAVTF ADQIFGIFNW IIPLSVALSC |
| | | FGGLNASIVA ASRLFFVGSR EGHLPDAICM IHVERFTPVP |
| | | SLLFNGIMAL IYLCVEDIFQ LINYYSFSYW FFVGLSIVGQ |
| | | LYLRWKEPDR PRPLKLSVFF PIVFCLCTIF LVAVPLYSDT |
| | | INSLIGIAIA LSGLPFYFLI IRVPEHKRPL YLRRIVGSAT |
| | | RYLQVLCMSV AAEMDLEDGG EMPKQRDPKS N |
| 35 | Homo sapiens Y + L | MEAREPGRPT PTYHLVPNTS QSQVEEDVSS PPQRSSETMQ |
| | amino acid transporter | LKKEISLLNG VSLVVGNMIG SGIFVSPKGV LVHTASYGMS |
| | 2 (SLC7A6) | LIVWAIGGLF SVVGALCYAE LGTTITKSGA SYAYILEAFG |
| | | GFIAFIRLWV SLLVVEPTGQ AllAITFANY IIQPSFPSCD |
| | | PPYLACRLLA AACICLLTFV NCAYVKWGTR VQDTFTYAKV |
| | | VALIAIIVMG LVKLCQGHSE HFQDAFEGSS WDMGNLSLAL |
| | | YSALFSYSGW DTLNFVTEEI KNPERNLPLA IGISMPIVTL |
| | | IYILTNVAYY TVLNISDVLS SDAVAVTFAD QTFGMFSWTI |
| | | PIAVALSCFG GLNASIFASS RLFFVGSREG HLPDLLSMIH |
| | | IERFTPIPAL LFNCTMALIY LIVEDVFQLI NYFSFSYWFF |
| | | VGLSVVGQLY LRWKEPKRPR PLKLSVFFPI VFCICSVFLV |
| | | IVPLFTDTIN SLIGIGIALS GVPFYFMGVY LPESRRPLFI |
| | | RNVLAAITRG TQQLCFCVLT ELDVAEEKKD ERKTD |
| 48 | Homo sapiens Organic | MAFNDLLQQVGGVGRFQQIQVTLVVLPLLLMASHNTLQNFT |
| | anion transporter 1 | AAIPTHHCRPPADANLSKNGGLEVWLPRDRQGQPESCLRFTS |
| | (SLC22A6) | PQWGLPFLNGTEANGTGATEPCTDGWIYDNSTFPSTIVTEWD |
| | | LVCSHRALRQLAQSLYMVGVLLGAMVFGYLADRLGRRKVLIL |
| | | NYLQTAVSGTCAAFAPNFPIYCAFRLLSGMALAGISLNCMTLN |
| | | VEWMPIHTRACVGTLIGYVYSLGQFLLAGVAYAVPHWRHLQL |
| | | LVSAPFFAFFIYSWFFIESARWHSSSGRLDLTLRALQRVARING |
| | | KREEGAKLSMEVLRASLQKELTMGKGQASAMELLRCPTLRHL |
| | | FLCLSMLWFATSFAYYGLVMDLQGFGVSIYLIQVIFGAVDLPA |
| | | KLVGFLVINSLGRRPAQMAALLLAGICIL |
| | | LNGVIPQDQSIVRTSLAVLGKGCLAASFNCIFLYTGELYPTMIR |
| | | QTGMGMGSTMARVGSIVSPLVSMTAELYPSMPLFIYGAVPV |
| | | AASAVTVLLPETLGQPLPDTVQDLESRWAPTQKEAGIYPRKGK |
| | | QTRQQQEHQKYMVPLQASAQEKNGL |
| 49 | Homo sapiens | mvlsqeepdsargtseaqplgpaptgaapp pgpgpsdspe |
| | T-type amino acid | aavekvevelagpataepheppeppeggwg wlvmlaamwc |
| | transporter (SLC16A10) | ngsvfgiqnacgvlfvsmle tfgskdddkmvfktawvgslsmgmiffccp |
| | | ivsvftdlfg crktavvgaa vgfvglmsss fvssieplyl tygiifacgc |
| | | sfayqpslvi lghyfkkrlg lvngivtagssvftillpll lrvlidsvgl fytlrvlcif |
| | | mfvflagft yrplatstkd kesggsgsslfsrkkfsppk kifnfaifkv |
| | | tayavwavgiplalfgyfvp yvhlmkhynerfqdeknkevvlmcigvtsg |
| | | vgrllfgria dyvpgvkkvylqvlsfffig lmsmmiplcsifgaliavcl |
| | | imglfdgcfi simapiafel vgaqdvsqaigfllgfmsipmtvgppiagl |
| | | lrdklgsydvafylagyppl iggavlcfip wihskkqreiskttgkekme |
| | | kmlenqnsllssssgmfkkesdsii |
| 59 | Homo sapiens AGT1 | MDRGEKIQLK RVFGYWWGTS FLLINIIGAG |
| | (SLC7A13) | IFVSPKGVLA YSCMNVGVSL |
| | | CVWAGCAILA MTSTLCSAEI SISFPCSGAQ |
| | | YYFLKRYFGS TVAFLNLWTS |
| | | LFLGSGVVAG QALLLAEYSI QPFFPSCSVP |
| | | KLPKKCLALA MLWIVGILTS |
| | | RGVKEVTWLQ IASSVLKVSI LSFISLTGVV |
| | | FLIRGKKENV ERFQNAFDAE |
| | | LPDISHLIQA IFQGYFAYSG GACFTLIAGE |
| | | LKKPRTTIPK CIFTALPLVT |
| | | VVYLLVNISY LTVLTPREIL SSDAVAITWA |
| | | DRAFPSLAWI MPFAISTSLF |
| | | SNLLISIFKS SRPIYLASQE GQLPLLFNTL |
| | | NSHSSPFTAV LLLVTLGSLA |
| | | IILTSLIDLI NYIFFTGSLW SILLMIGILR |
| | | RRYQEPNLSI PYKVFLSFPL |
| | | ATIVIDVGLV VIPLVKSPNV HYVYVLLLVL |
| | | SGLLFYIPLI HFKIRLAWFE |
| | | KMTCYLQLLF NICLPDVSEE |
| 60 | Homo sapiens xCT | MVRKPVVSTI SKGGYLQGNV NGRLPSLGNK |
| | cystine/glutamate | EPPGQEKVQL KRKVTLLRGV |
| | transporter (SLC7A11) | SIIIGTIIGA GIFISPKGVL QNTGSVGMSL |
| | | TIWTVCGVLS LFGALSYAEL |
| | | GTTIKKSGGH YTYILEVFGP LPAFVRVWVE |
| | | LLIIRPAATA VISLAFGRYI |
| | | LEPFFIQCEI PELAIKLITA VGITVVMVLN |
| | | SMSVSWSARI QIFLTFCKLT |
| | | AILIIIVPGV MQLIKGQTQN FKDAFSGRDS |
| | | SITRLPLAFY YGMYAYAGWF |
| | | YLNFVTEEVE NPEKTIPLAI CISMAIVTIG |
| | | YVLTNVAYFT TINAEELLLS |
| | | NAVAVTFSER LLGNFSLAVP IFVALSCFGS |
| | | MNGGVFAVSR LFYVASREGH |
| | | LPEILSMIHV RKHTPLPAVI VLHPLTMIML |
| | | FSGDLDSLLN FLSFARWLFI |
| | | GLAVAGLIYL RYKCPDMHRP FKVPLFIPAL |
| | | FSFTCLFMVA LSLYSDPFST |
| | | GIGFVITLTG VPAYYLFIIW DKKPRWFRIM |
| | | SEKITRTLQI ILEVVPEEDK |
| | | L |
| 61 | Homo sapiens Solute | MAALAAAAKK VWSARRLLVL LFTPLALLPV |
| | carrier family 13 | VFALPPKEGR CLFVILLMAV |
| | member 3 (SLC13A3) | YWCTEALPLS VTALLPIVLF PFMGILPSNK |
| | | VCPQYFLDTN FLFLSGLIMA |
| | | SAIEEWNLHR RIALKILMLV GVQPARLILG |
| | | MMVTTSFLSM WLSNTASTAM |
| | | MLPIANAILK SLFGQKEVRK DPSQESEENT |
| | | AAVRRNGLHT VPTEMQFLAS |
| | | TEAKDHPGET EVPLDLPADS RKEDEYRRNI |
| | | WKGFLISIPY SASIGGTATL |
| | | TGTAPNLILL GQLKSFFPQC DVVNFGSWFI |
| | | FAFPLMLLFL LAGWLWISFL |
| | | YGGLSFRGWR KNKSEIRTNA EDRARAVIRE |
| | | EYQNLGPIKF AEQAVFILFC |
| | | MFAILLFTRD PKFIPGWASL FNPGFLSDAV |
| | | TGVAIVTILF FFPSQRPSLK |
| | | WWFDFKAPNT ETEPLLTWKK AQETVPWNII |
| | | LLLGGGFAMA KGCEESGLSV |
| | | WIGGQLHPLE NVPPALAVLL ITVVIAFFTE |
| | | FASNTATIII FLPVLAELAI |
| | | RLRVHPLYLM IPGTVGCSFA FMLPVSTPPN |
| | | SIAFASGHLL VKDMVRTGLL |
| | | MNLMGVLLLS LAMNTWAQTI FQLGTFPDWA |
| | | DMYSVNVTAL PPTLANDTFR |
| | | TL |

In one embodiment, the amino acid transporter comprises a sodium-coupled neutral amino acid transporter 1 (SLC38A1) (SAT1) comprising or consisting of the amino acid sequence of SEQ ID NO:13, or a variant thereof. In another embodiment, the amino acid transporter comprises a sodium-coupled neutral amino acid transporter 2 (SLC38A2) (SAT2) comprising or consisting of the amino acid sequence of SEQ ID NO:14, or a variant thereof. In another embodiment, the amino acid transporter comprises a sodium-coupled neutral amino acid transporter 4 (SLC38A4) (SATS) comprising or consisting of the amino acid sequence of SEQ ID NO:15, or a variant thereof. In another embodiment, the amino acid transporter comprises a neutral amino acid transporter A (SLC1A4) (ASCT1) comprising or consisting of the amino acid sequence of SEQ ID NO:16, or a variant thereof. In another embodiment, the amino acid transporter comprises a neutral amino acid transporter B(0) (SLC1A5) (ASCT2) comprising or consisting of the amino acid sequence of SEQ ID NO:17, or a variant thereof. In another embodiment, the amino acid transporter comprises a large neutral amino acid transporter small subunit 1 (SLC7A5) (LAT1) comprising or consisting of the amino acid sequence of SEQ ID NO:18, or a variant thereof. In another embodiment, the amino acid transporter comprises a large neutral amino acids transporter small subunit 2 (SLC7A8) (LAT2) comprising or consisting of the amino acid sequence of SEQ ID NO:19, or a variant thereof. In another embodiment, the amino acid transporter comprises an excitatory amino acid transporter 1 (SLC1A3) (EAAT1) comprising or consisting of the amino acid sequence of SEQ ID NO:20, or a variant thereof. In another embodiment, the amino acid transporter comprises an excitatory amino acid transporter 2 (SLC1A2) (EAAT2) comprising or consisting of the amino acid sequence of SEQ ID NO:21, or a variant thereof. In another embodiment, the amino acid transporter comprises and excitatory amino acid transporter 3 (SLC1A1) (EAAT3) comprising or consisting of the amino acid sequence of SEQ ID NO:22, or a variant thereof. In another embodiment, the amino acid transporter comprises an excitatory amino acid transporter 4 (SLC1A6) (EAAT4) comprising or consisting of the amino acid sequence of SEQ ID NO:23, or a variant thereof. In another embodiment, the amino acid transporter comprises an excitatory amino acid transporter 5 (SLC1A7) (EAAT5) comprising or consisting of the amino acid sequence of SEQ ID NO:24, or a variant thereof. In another embodiment, the amino acid transporter comprises a 4F2 cell-surface antigen heavy chain (SLC3A2) CD98 comprising or consisting of the amino acid sequence of SEQ ID NO:25, or a variant thereof. In another embodiment, the amino acid transporter comprises a sodium-coupled neutral amino acid transporter 3 (SLC38A3) (SN1) comprising or consisting of the amino acid sequence of SEQ ID NO:26, or a variant thereof. In another embodiment, the amino acid transporter comprises a sodium-coupled neutral amino acid transporter 5 (SLC38A5) (SN2) comprising or consisting of the amino acid sequence of SEQ ID NO:27, or a variant thereof. In another embodiment, the amino acid transporter comprises an Asc-type amino acid transporter 1 (SLC7A10) (Asc1) comprising or consisting of the amino acid sequence of SEQ ID NO:28, or a variant thereof. In another embodiment, the amino acid transporter comprises a b(0,+)-type amino acid transporter 1 (SLC7A9) comprising or consisting of the amino acid sequence of SEQ ID NO:29, or a variant thereof. In another embodiment, the amino acid transporter comprises a neutral and basic amino acid transport protein rBAT (SLC3A1) comprising or consisting of the amino acid sequence of SEQ ID NO:30, or a variant thereof. In another embodiment, the amino acid transporter comprises a proton-coupled amino acid transporter 1 (SLC36A1) comprising or consisting of the amino acid sequence of SEQ ID NO:31, or a variant thereof. In another embodiment, the amino acid transporter comprises a proton-coupled amino acid transporter 2 (SLC36A2) comprising or consisting of the amino acid sequence of SEQ ID NO:32, or a variant thereof. In another embodiment, the amino acid transporter comprises a sodium- and chloride-dependent neutral and basic amino acid transporter B(0+) (SLC6A14) comprising or consisting of the amino acid sequence of SEQ ID NO:33, or a variant thereof. In another embodiment, the amino acid transporter comprises a Y+L amino acid transporter 1 (SLC7A7) comprising or consisting of the amino acid sequence of SEQ ID NO:34, or a variant thereof. In another embodiment, the amino acid transporter comprises a Y+L amino acid transporter 2 (SLC7A6) comprising or consisting of the amino acid sequence of SEQ ID NO:35, or a variant thereof. In another embodiment, the amino acid transporter comprises an organic anion transporter 1 (SLC22A6) comprising or consisting of the amino acid sequence of SEQ ID NO: 48. In another embodiment, the amino acid transporter comprises a T-type amino acid transporter (SLC16A10) comprising or consisting of the amino acid sequence of SEQ ID NO: 49. In one embodiment, the amino acid transporter comprises a Homo sapiens AGT1 (SLC7A13) comprising or consisting of the amino acid sequence of SEQ ID NO:59. In another embodiment, the amino acid transporter comprises a Homo sapiens xCT cystine/glutamate transporter (SLC7A11) comprising or consisting of the amino acid sequence of SEQ ID NO:60. In another embodiment, the amino acid transporter comprises a Homo sapiens solute carrier family 13 member 3 (SLC13A3) comprising or consisting of the amino acid sequence of SEQ ID NO:61.

In some embodiments, the amino acid transporter is a variant of a wild-type amino acid transporter comprising an amino acid sequence having at least 40%, at least 50%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:48, SEQ ID NO:49, SEQ ID NO:59, SEQ ID NO:60, and SEQ ID NO:61. In some embodiments, the variant amino acid transporter is capable of transporting an amino acid (e.g., homocysteine or serine) across a membrane (e.g., a plasma membrane).

In some embodiments, the amino acid transporter comprises the full length or a fragment of the sodium-coupled neutral amino acid transporter 1 (SLC38A1) (SAT1), sodium-coupled neutral amino acid transporter 2 (SLC38A2) (SAT2), sodium-coupled neutral amino acid transporter 4 (SLC38A4) (SAT4), neutral amino acid transporter A (SLC1A4) (ASCT1), neutral amino acid transporter B(0) (SLC1A5) (ASCT2), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1), excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, Sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), proton-coupled amino acid transporter 1 (SLC36A1), proton-coupled amino acid transporter 2 (SLC36A2), sodium- and chloride-dependent neutral and basic amino acid transporter B(0+) (SLC6A14), Y+L amino acid transporter 1 (SLC7A7), Y+L amino acid transporter 2 (SLC7A6), organic anion transporter 1 (SLC22A6), T-type amino acid transporter (SLC16A10), Homo sapiens AGT1 (SLC7A13), Homo sapiens xCT cystine/glutamate transporter (SLC7A11), Homo sapiens solute carrier family 13 member 3 (SLC13A3) proteins. In some embodiments, the fragment of the amino acid transporter comprises at least 10, at least 20, at least 30, at least 40, at least 50, at least 60, at least 70, at least 80, at least 90, at least 100, at least 110, at least 120, at least 130, at least 140, at least 150 or at least 160 amino acids (e.g., contiguous amino acids) of a wild-type amino acid transporter. In some embodiments, the fragment of the amino acid transporter comprises fewer than 20, fewer than 30, fewer than 40, fewer than 50, fewer than 60, fewer than 70, fewer than 80, fewer than 90, fewer than 100, fewer than 110, fewer than 120, fewer than 130, fewer than 140, fewer than 150 or fewer than 160 amino acids of a wild-type amino acid transporter. In some embodiments, fragments or variants of the amino acid transporter retain at least 50%, at least 55%, at least 60%, at least 65%, at least 70%, at least 75%, at least 80%, at least 85%, at least 90%, at least 95%, at least 98% or at least 99% of the function (e.g., amino acid transport (e.g., import or export) capability) of the wild-type amino acid transporter.

In general, a variant amino acid transporter, may be produced, for example, to enhance production of the protein in an engineered cell, to improve turnover/half-life of the protein or mRNA encoding the protein, and/or to modulate (enhance or reduce) the activity of the amino acid transporter. The amino acid transporter may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding an amino acid transporter (e.g., a homocysteine or serine transporter) as described herein. In some embodiments, the invention provides an engineered erythroid cell prepared by using a nucleic acid sequence encoding an amino acid transporter (e.g., a homocysteine or serine transporter) as described herein. In some embodiments, the nucleic acid sequence encodes an amino acid transporter (e.g. sodium-coupled neutral amino acid transporter 1 (SLC38A1) (SAT1), Sodium-coupled neutral amino acid transporter 2 (SLC38A2) (SAT2), Sodium-coupled neutral amino acid transporter 4 (SLC38A4) (SAT3), Neutral amino acid transporter A (SLC1A4) (ASCT1), Large neutral amino acids transporter small subunit 1 (SLC7A5) (LAT1), Large neutral amino acids transporter small subunit 2 (SLC7A8) (LAT2), Excitatory amino acid transporter 1 (SLC1A3) (EAAT1), Excitatory amino acid transporter 2 (SLC1A2) (EAAT2), Excitatory amino acid transporter 3 (SLC1A1) (EAAT3), Excitatory amino acid transporter 4 (SLC1A6) (EAAT4), Excitatory amino acid transporter 5 (SLC1A7) (EAAT5), 4F2 cell-surface antigen heavy chain (SLC3A2) CD98, Sodium-coupled neutral amino acid transporter 3 (SLC38A3) (SN1), Sodium-coupled neutral amino acid transporter 5 (SLC38A5) (SN2), Asc-type amino acid transporter 1 (SLC7A10) (Asc1), b(0,+)-type amino acid transporter 1 (SLC7A9), Neutral and basic amino acid transport protein rBAT (SLC3A1), Proton-coupled amino acid transporter 1 (SLC36A1), Proton-coupled amino acid transporter 2 (SLC36A2), Sodium- and chloride-dependent neutral and basic amino acid transporter B(0+) (SLC6A14), Y+L amino acid transporter 1 (SLC7A7) Y+L amino acid transporter 2 (SLC7A6), Organic anion transporter 1 (SLC22A6) (OAT1), T-type amino acid transporter (SLC16A10) (TAT1), AGT1 (SLC7A13), xCT cystine/glutamate transporter (SLC7A11), Solute carrier family 13 member 3 (SLC13A3)) as described herein.

In some embodiments, the amino acid transporter is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%. at least 97%, at least 98%, at least 99%, or 100% identical to the corresponding amino acid transporter nucleic acid that encodes a protein that possesses a function of an amino acid transporter described herein, e.g., the regulation of amino acid transport , or amino acid transport (e.g., import or export) capability).

In some embodiments, an engineered erythroid cell or an enucleated cell comprises an exogenous polypeptide comprising an amino acid transporter (e.g., a homocysteine and/or serine transporter), or a variant thereof, that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP), a homocysteine degrading polypeptide, or a cystathionine degrading polypeptide). The polypeptide of interest may be disposed in any configuration of the exogenous polypeptide (e.g., the polypeptide of interest may be fused to the N-terminus or C-terminus of the cystathionine degrading polypeptide).

In some embodiments, the exogenous polypeptide may include a linker (e.g., a linker described herein) disposed between the amino acid transporter and the at least one polypeptide of interest. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 95), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGG (SEQ ID NO: 96), GGGGSGGGGS (SEQ ID NO: 97), GSGSGSGSGS (SEQ ID NO: 98), PSTSTST (SEQ ID NO: 99), and EIDKPSQ (SEQ ID NO: 100), and multimers thereof.

In some embodiments, the exogenous polypeptide comprises a transmembrane domain or a transmembrane polypeptide (e.g., SMIM1, GPA, or Kell) and an amino acid transporter. In some embodiments, the transmembrane domain is derived from GPA. For example, in some embodiments, the transmembrane domain is derived from GPA and comprises or consists of the amino acid sequence:

(SEQ ID NO: 102)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

In some embodiments, the transmembrane domain is derived from SMIM1. For example, in some embodiments, the transmembrane domain comprises or consists of the amino acid sequence:

(SEQ ID NO: 103)
MQPQESHVHYSRWEDGSRDGVSLGAVSSTEEASRCRRISQRLCTGKLGIA
MKVLGGVALFWIIFILGYLTGYYVHKCK

In some embodiments, the transmembrane domain or transmembrane polypeptide is disposed in the exogenous polypeptide such that the amino acid transporter present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the amino acid transporter present in the exogenous polypeptide locates in the outer surface of the cell (e.g., facing the extracellular milieu of the cell). In some embodiments, the exogenous polypeptide does not include a transmembrane domain or a transmembrane polypeptide (e.g., a transmembrane domain that is heterologous to the amino acid transporter). In some embodiments, the exogenous polypeptide does not include a polypeptide that is endogenous to the cell. In some embodiments, a linker (e.g., any linker provided herein) is disposed between the transmembrane domain or transmembrane polypeptide and the amino acid transporter.

In some embodiments, the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA. For example, in some embodiments, the leader or signal sequence is derived from GPA and comprises or consists of the amino acid sequence MYGKIIFVLLLSEIVSISA (SEQ ID NO: 101).

In some embodiments, the nucleic acid sequence encoding the exogenous polypeptides or a component thereof as provided herein is codon optimized (e.g., for expression in a mammalian cell (e.g., a nucleated erythroid cell). For example, in some embodiments, the nucleic acid sequence encoding the amino acid transporter is codon optimized. In other embodiments, the nucleic acid sequence encoding the exogenous polypeptide or a component thereof is not codon optimized. For example, in some embodiments, the nucleic acid sequence encoding the amino acid transporter is not codon optimized.

Various methods and software programs can be used to determine the homology between two or more peptides or nucleic acids, such as NCBI BLAST, Clustal W, MAFFT, Clustal Omega, AlignMe, Praline, or another suitable method or algorithm. In some embodiments, percent identity is calculated by FastDB based upon the following parameters: mismatch penalty of 1; gap penalty of 1; gap size penalty of 0.33; and joining penalty of 30.

An example of a useful algorithm is PILEUP. PILEUP creates a multiple sequence alignment from a group of related sequences using progressive, pairwise alignments. It can also plot a tree showing the clustering relationships used to create the alignment. Useful PILEUP parameters including a default gap weight of 3.00, a default gap length weight of 0.10, and weighted end gaps.

Another example of a useful algorithm is the BLAST algorithm. A useful example of a BLAST program is the WU-BLAST-2 program. WU-BLAST-2 uses several search parameters, most of which are set to the default values. The adjustable parameters are set with the following values: overlap span=1, overlap fraction=0.125, word threshold (T)=ll. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

An additional useful tool is Clustal, a series of commonly used computer programs for multiple sequence alignment. Recent versions of Clustal include ClustalW, ClustalX and Clustal Omega. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

### Polypeptides and Nucleic Acids

In one aspect, the disclosure provides isolated homocysteine reducing polypeptides, homocysteine degrading polypeptides, homocysteine transporters or serine transporters, described herein. In some embodiments, the homocysteine reducing polypeptides comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a homocysteine reducing polypeptide described herein. In some embodiments, the homocysteine degrading polypeptides comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a homocysteine degrading polypeptide described herein. In some embodiments, the homocysteine transporters comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a homocysteine transporter described herein. In some embodiments, the serine transporters comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a serine transporter described herein. In some embodiments, the homocysteine reducing polypeptides, homocysteine degrading polypeptides, homocysteine transporters or serine transporters are recombinantly produced. Methods for producing recombinant proteins are known in the art and described herein.

In another aspect, the disclosure provides nucleic acids (e.g., DNA or RNA (e.g., mRNA)) encoding a homocysteine reducing polypeptide described herein. In another aspect, the disclosure provides nucleic acids (e.g., DNA or RNA (e.g., mRNA)) encoding a homocysteine degrading polypeptide described herein. In another aspect, the disclosure provides nucleic acids (e.g., DNA or RNA (e.g., mRNA)) encoding a homocysteine transporter described herein. In another aspect, the disclosure provides nucleic acids (e.g., DNA or RNA (e.g., mRNA)) encoding a serine transporter described herein. In some embodiments, the nucleic acids are codon-optimized for expression in a desired cell type (e.g., a bacterial or mammalian cell).

### Specific Activity of Homocysteine Degrading Polypeptide

Specific activity can be defined as the number of enzyme units per milligram of protein, where one unit of activity is defined as degradation of 1 μmol of homocysteine per minute.

In one aspect, the disclosure provides an erythroid cell engineered to reduce homocysteine levels, comprising a first exogenous polypeptide comprising a homocysteine degrading polypeptide (e.g., a cystathionine beta-synthase (CBS) polypeptide, or a variant thereof, or a methionine gamma-lyase (MGL) polypeptide, or variant thereof). In one embodiment, the CBS polypeptide included in an engineered erythroid cell of the invention has a specific activity of about 1 μmol/min/mg when measured using 200 μM homocysteine and 100 μM serine. In one embodiment, the exogenous polypeptide comprising a CBS (e.g, a truncated CBS polypeptide) in an engineered erythroid cell of the invention has a specific activity of between 0.01 and 100 μmol/min/mg, or between about 0.1 and 10 μmol/min/mg, or between about 0.5 and 5 μmol/min/mg, or between about 0.5 and 2 μmol/min/mg, when measured using 200 μM homocysteine and 100 μM serine. In one embodiment, the exogenous polypeptide comprising a CBS (e.g., a truncated polypeptide) included in an engineered erythroid cell of the invention has a specific activity of at least about 0.01 μmol/min/mg, at least about 0.1 μmol/min/mg, at least about 0.5 μmol/min/mg, at least about 1 μmol/min/mg, or at least about 2 μmol/min/mg, or at least about 5 umol/min/mg, or at least about 10 μmol/min/mg, or at least about 50 μmol/min/mg, when measured using 200 μM homocysteine and 100 μM serine. In one embodiment, the specific activity is at neutral pH (e.g., at pH 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0).

### Transport Rate of Homocysteine or Serine

In one aspect, the disclosure provides an engineered erythroid cell comprising a first exogenous polypeptide comprising a homocysteine transporter, or a variant thereof. The rate that the homocysteine transporter transports homocysteine from outside the erythroid cell to inside the erythroid cell can be reported in mole transported per minute per cell (μmole/min/cell).

In one aspect, the disclosure provides an engineered erythroid cell comprising a first exogenous polypeptide comprising a serine transporter, or a variant thereof. The rate that the serine transporter transports serine from outside the erythroid cell to inside the erythroid cell can be reported in mole transported per minute per cell (μmole/min/cell).

In one embodiment, the homocysteine transporter transports homocysteine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the homocysteine transporter transports homocysteine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the homocysteine transporter transports homocysteine from outside the erythroid cell to the inside of the erythroid cell at a rate of about 1.2×10e-11 μmole/min/cell. In one embodiment, the homocysteine transporter transports homocysteine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the homocysteine transporter transports homocysteine from outside the erythroid cell to the inside of the erythroid cell at a rate of at least 1.2×10e-11 μmole/min/cell.

In one embodiment, the serine transporter transports serine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the serine transporter transports serine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the serine transporter transports serine from outside the erythroid cell to the inside of the erythroid cell at a rate of about 1.2×10e-11 μmole/min/cell. In one embodiment, the serine transporter transports serine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the serine transporter transports serine from outside the erythroid cell to the inside of the erythroid cell at a rate of at least 1.2×10e-11 μmole/min/cell.

In one aspect, the disclosure provides an erythroid cell engineered to reduce homocysteine levels, comprising a first exogenous polypeptide comprising a homocysteine reducing polypeptide, or a variant thereof, further comprising a second exogenous polypeptide, wherein the second exogenous polypeptides is an amino acid transporter. In one embodiment, the second exogenous polypeptide is a homocysteine transporter. In one embodiment, the homocysteine transporter transports homocysteine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the homocysteine transporter transports homocysteine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the homocysteine transporter transports homocysteine from outside the erythroid cell to the inside of the erythroid cell at a rate of about 1.2×10e-11 μmole/min/cell. In one embodiment, the homocysteine transporter transports homocysteine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the homocysteine transporter transports homocysteine from outside the erythroid cell to the inside of the erythroid cell at a rate of at least 1.2×10e-11 μmole/min/cell.

In another embodiment, the second exogenous polypeptide is a serine transporter. In one embodiment, the serine transporter transports serine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the serine transporter transports serine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the serine transporter transports serine from outside the erythroid cell to the inside of the erythroid cell at a rate of about 1.2×10e-11 μmole/min/cell. In one embodiment, the serine transporter transports serine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the serine transporter transports serine from outside the erythroid cell to the inside of the erythroid cell at a rate of at least 1.2×10e-11 μmole/min/cell.

In one aspect, the disclosure provides an erythroid cell engineered to reduce homocysteine levels, comprising a first exogenous polypeptide comprising a homocysteine degrading polypeptide, or variant thereof, wherein the homocysteine degrading polypeptide, or variant thereof, is not a cystathionine beta-synthase, further comprising a second exogenous polypeptide, wherein the second exogenous polypeptides is an amino acid transporter. In one embodiment, the second exogenous polypeptide is a homocysteine transporter. In one embodiment, the homocysteine transporter transports homocysteine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the homocysteine transporter transports homocysteine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the homocysteine transporter transports homocysteine from outside the erythroid cell to the inside of the erythroid cell at a rate of about 1.2×10e-11 μmole/min/cell. In one embodiment, the homocysteine transporter transports homocysteine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the homocysteine transporter transports homocysteine from outside the erythroid cell to the inside of the erythroid cell at a rate of at least 1.2×10e-11 μmole/min/cell. In another embodiment, the second exogenous polypeptide is a serine transporter. In one embodiment, the serine transporter transports serine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the serine transporter transports serine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the serine transporter transports serine from outside the erythroid cell to the inside of the erythroid cell at a rate of about 1.2×10e-11 μmole/min/cell. In one embodiment, the serine transporter transports serine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the serine transporter transports serine from outside the erythroid cell to the inside of the erythroid cell at a rate of at least 1.2×10e-11 μmole/min/cell.

In certain embodiments, homocysteine is effectively transported into the engineered erythroid cell without the inclusion of an exogenous polypeptide comprising a homocysteine transporter or serine transporter. Without being bound by theory, in some embodiments, the measured homocysteine transport into the erythroid cells that are not engineered to include a homocysteine or a serine transporter is sufficiently close to a target rate (e.g. a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell; preferably, a rate of between about 1×10e-11 to about 3×10e-11) that inclusion of an exogenous polypeptide comprising a homocysteine transporter or serine transporter is not needed. In certain embodiments, the measured homocysteine transport into the erythroid cells that are not engineered to include a homocysteine or a serine transporter is at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell, or between about 1×e-11 to about 3×e-11. In one embodiment, the homocysteine is transported from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the homocysteine is transported from outside the erythroid cell to the inside of the erythroid cell at a rate of about 1×10e-11, 1.1×10e-11, 1.2×10e-11, 1.3×10e-11, 1.4×10e-11, 1.5×10e-11, 1.6×10e-11, 1.7×10e-11, 1.8×10e-11, 1.9×10e-11, 2×10e-11, 2.1×10e-11, 2.2×10e-11, 2.3×10e-11, 2.4×10e-11, 2.5×10e-11, 2.6×10e-11, 2.7×10e-11, 2.8×10e-11, 2.9×10e-11, or 3×10e-11.

In one aspect, the disclosure provides an erythroid cell engineered to reduce homocysteine levels, comprising a first exogenous polypeptide comprising a cystathionine beta-synthase (CBS) polypeptide, or variant thereof, further comprising a second exogenous polypeptide, wherein the second exogenous polypeptides is an amino acid transporter. In one aspect, the disclosure provides an erythroid cell engineered to reduce homocysteine levels, comprising a first exogenous polypeptide comprising a methionine gamma-lyase (MGL) polypeptide, or variant thereof, further comprising a second exogenous polypeptide, wherein the second exogenous polypeptides is an amino acid transporter. In one aspect, the disclosure provides an erythroid cell engineered to reduce homocysteine levels, comprising a first exogenous polypeptide comprising a cystathionine beta-synthase (CBS) polypeptide, or variant thereof, a second exogenous polypeptide, wherein the second exogenous polypeptides is an amino acid transporter, and a third exogenous polypeptide, wherein the third exogenous polypeptide is a cystathionine degrading polypeptide (e.g. cystathionine gamma-lyase). In one embodiment, the second exogenous polypeptide is a homocysteine transporter. In one embodiment, the homocysteine transporter transports homocysteine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the homocysteine transporter transports homocysteine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the homocysteine transporter transports homocysteine from outside the erythroid cell to the inside of the erythroid cell at a rate of about 1.2×10e-11 μmole/min/cell. In one embodiment, the homocysteine transporter transports homocysteine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the homocysteine transporter transports homocysteine from outside the erythroid cell to the inside of the erythroid cell at a rate of at least 1.2×10e-11 μmole/min/cell. In another embodiment, the second exogenous polypeptide is a serine transporter. In one embodiment, the serine transporter transports serine from outside the erythroid cell to the inside of the erythroid cell at a rate of between about 1×10e-10 to about 1×10e-12 μmole/min/cell, between about 1×10e-10 to about 1×10e-11 μmole/min/cell, between about 1×10e-11 to about 1×10e-12 μmole/min/cell. In one embodiment, the serine transporter transports serine from outside the erythroid cell to the inside of the erythroid cell at a rate of about 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, 9.5×10e-11, or 1×10e-12. In one embodiment, the serine transporter transports serine from outside the erythroid cell to the inside of the erythroid cell at a rate of about 1.2×10e-11 μmole/min/cell. In one embodiment, the serine transporter transports serine from outside the erythroid cell to the inside of the erythroid cell at a rate of at least 1×10e-10, 1.5×10e-10, 2×10e-10, 2.5×10e-10, 3×10e-10, 3.5×10e-10, 4×10e-10, 4.5×10e-10, 5×10e-10, 5.5×10e-10, 6×10e-10, 6.5×10e-10, 7×10e-10, 7.5×10e-10, 8×10e-10, 8.5×10e-10, 9×10e-10, 9.5×10e-10, 1×10e-11, 1.5×10e-11, 2×10e-11, 2.5×10e-11, 3×10e-11, 3.5×10e-11, 4×10e-11, 4.5×10e-11, 5×10e-11, 5.5×10e-11, 6×10e-11, 6.5×10e-11, 7×10e-11, 7.5×10e-11, 8×10e-11, 8.5×10e-11, 9×10e-11, or 9.5×10e-11. In one embodiment, the serine transporter transports serine from outside the erythroid cell to the inside of the erythroid cell at a rate of at least 1.2×10e-11 μmole/min/cell.

### Polypeptide Copy Number

It will be appreciated by one skilled in the art that use of recombinant DNA technologies can improve control of expression of transfected nucleic acid molecules by manipulating, for example, the number of copies of the nucleic acid molecules within the host cell.

In one embodiment, an erythroid cell engineered to reduce homocysteine levels, comprising a first exogenous polypeptide comprising a homocysteine reducing polypeptide, or a variant thereof, comprises between about 100,000 to about 600,000 copies of the first exogenous polypeptide, for example about 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In one embodiment, the engineered erythroid cell comprises between about 100,000-600,000, between about 100,000-500,000, between about 100,000-400,000, between about 150,000-300,000, or between 150,000-200,000 copies of the first exogenous polypeptide. In one embodiment, the engineered erythroid cell comprises at least about 150,000 copies of the first exogenous polypeptide. In one embodiment, the engineered erythroid cell comprises at least about 150,000 copies of the first exogenous polypeptide. In one embodiment, the engineered erythroid cell comprises at least about 200,000 copies of the first exogenous polypeptide. In one embodiment, the engineered erythroid cell comprises at least about 250,000 copies of the first exogenous polypeptide. In one embodiment, the engineered erythroid cell comprises at least about 300,000 copies of the first exogenous polypeptide. In one embodiment, the engineered erythroid cell comprises at least about 400,000 copies of the first exogenous polypeptide. In one embodiment, the engineered erythroid cell comprises at least about 500,000 copies of the first exogenous polypeptide. In one embodiment, the engineered erythroid cell is an enucleated cell. In one embodiment, the engineered erythroid cell is a nucleated cell.

In one embodiment, an erythroid cell engineered to reduce homocysteine levels, comprising a first exogenous polypeptide comprising a homocysteine degrading polypeptide, or variant thereof, wherein the homocysteine degrading polypeptide, or variant thereof, is not a cystathionine beta-synthase, comprises between about 100,000 to about 600,000 copies of the first exogenous polypeptide, for example about 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In one embodiment, the engineered erythroid cell comprises between about 100,000-600,000, between about 100,000-500,000, between about 100,000-400,000, between about 150,000-300,000, or between 150,000-200,000 copies of the first exogenous polypeptide. In one embodiment, the engineered erythroid cell comprises at least about 150,000 copies of the first exogenous polypeptide. In one embodiment, the engineered erythroid cell comprises at least about 150,000 copies of the first exogenous polypeptide. In one embodiment, the engineered erythroid cell comprises at least about 200,000 copies of the first exogenous polypeptide. In one embodiment, the engineered erythroid cell comprises at least about 250,000 copies of the first exogenous polypeptide. In one embodiment, the engineered erythroid cell comprises at least about 300,000 copies of the first exogenous polypeptide. In one embodiment, the engineered erythroid cell comprises at least about 400,000 copies of the first exogenous polypeptide. In one embodiment, the engineered erythroid cell comprises at least about 500,000 copies of the first exogenous polypeptide.

In one embodiment, an erythroid cell engineered to reduce homocysteine levels, comprising a first exogenous polypeptide comprising a cystathionine beta-synthase (CBS) polypeptide, or variant thereof, comprises between about 110,00 to about 600,000, or between about 150,000 to about 600,000 copies of the first exogenous polypeptide, for example about 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In one embodiment, the engineered erythroid cell comprises between about 150,000-600,000, between about 150,000-500,000, between about 150,000-400,000, between about 150,000-300,000, or between 150,000-200,000 copies of the first exogenous polypeptide. In one embodiment, the engineered erythroid cell comprises at least about 150,000 copies of the first exogenous polypeptide. In one embodiment, the engineered erythroid cell comprises at least about 150,000 copies of the first exogenous polypeptide. In one embodiment, the engineered erythroid cell comprises at least about 200,000 copies of the first exogenous polypeptide. In one embodiment, the engineered erythroid cell comprises at least about 250,000 copies of the first exogenous polypeptide. In one embodiment, the engineered erythroid cell comprises at least about 300,000 copies of the first exogenous polypeptide. In one embodiment, the engineered erythroid cell comprises at least about 400,000 copies of the first exogenous polypeptide. In one embodiment, the engineered erythroid cell comprises at least about 500,000 copies of the first exogenous polypeptide.

In one embodiment, an erythroid cell engineered to reduce homocysteine levels, comprising a first exogenous polypeptide comprising a methionine gamma-lyase (MGL) polypeptide, or variant thereof, comprises between about 110,00 to about 600,000, or between about 150,000 to about 600,000 copies of the first exogenous polypeptide, for example about 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In one embodiment, the engineered erythroid cell comprises between about 150,000-600,000, between about 150,000-500,000, between about 150,000-400,000, between about 150,000-300,000, or between 150,000-200,000 copies of the first exogenous polypeptide. In one embodiment, the engineered erythroid cell comprises at least about 150,000 copies of the first exogenous polypeptide. In one embodiment, the engineered erythroid cell comprises at least about 150,000 copies of the first exogenous polypeptide. In one embodiment, the engineered erythroid cell comprises at least about 200,000 copies of the first exogenous polypeptide. In one embodiment, the engineered erythroid cell comprises at least about 250,000 copies of the first exogenous polypeptide. In one embodiment, the engineered erythroid cell comprises at least about 300,000 copies of the first exogenous polypeptide. In one embodiment, the engineered erythroid cell comprises at least about 400,000 copies of the first exogenous polypeptide. In one embodiment, the engineered erythroid cell comprises at least about 500,000 copies of the first exogenous polypeptide.

In one embodiment, an engineered erythroid cell comprising a first exogenous polypeptide comprising a homocysteine or serine transporter, or a variant thereof, comprises at least about 10,000 copies of the first exogenous polypeptide. In one embodiment, the erythroid cell comprises at least about 20,000 copies of the first exogenous polypeptide. In one embodiment, the erythroid cell comprises at least about 30,000 copies of the first exogenous polypeptide. In one embodiment, the erythroid cell comprises about 10,000-100,000 copies of the first exogenous polypeptide, for example about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 copies of the first polypeptide

In one embodiment, the first exogenous polypeptide is present at a copy number of no more than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% greater, or no more than 2, 3, 4, 5, 6, 7, 8, 9, 10, 20, 25, 30, 35, 40, 45, 50, 60, 70, 80, 90, 100, 150, 200, 300, 400, 500, or 1000 times greater than the copy number of the second exogenous polypeptide or the copy number of the third exogenous polypeptide. In one embodiment, the second exogenous polypeptide is present at a copy number of no more than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% greater, or no more than 2, 3, 4, 5, 6, 7, 8, 9, 10, 20, 25, 30, 35, 40, 45, 50, 60, 70, 80, 90, 100, 150, 200, 300, 400, 500, or 1000 times greater than the copy number of the first exogenous polypeptide.

In some embodiments, the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, or from about 100:1to 1:100 by weight or by copy number.

In some embodiments, the engineered erythroid cell comprises a first exogenous polypeptide, wherein the first exogenous polypeptide is present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In some embodiments, the engineered erythroid cell comprises a first exogenous polypeptide, and further comprises a second exogenous polypeptide, wherein the first and second exogenous polypeptides are present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In some embodiments, the engineered erythroid cell comprises a first exogenous polypeptide, a second exogenous polypeptide and a third exogenous polypeptide, wherein the first, second and third exogenous polypeptides are present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In one embodiment of the various aspects and embodiments above, the engineered erythroid cell is an enucleated cell. In one embodiment of the various aspects and embodiments above, the engineered erythroid cell is a nucleated cell.

### In Vivo Half-Life

In one embodiment, the invention provides an engineered erythroid cell, comprising at a first exogenous polypeptide comprising a homocysteine degrading polypeptide, or variant thereof, a second exogenous polypeptide comprising a homocysteine transporter, or a variant thereof, and/or a third exogenous transporter comprising a serine transporter, or a variant thereof. In one embodiment, the first exogenous polypeptide, the second exogenous polypeptide, and/or the third exogenous polypeptide, included in an engineered erythroid cell have a prolonged in vivo half-life as compared to a corresponding exogenous polypeptide (e.g., the homocysteine degrading polypeptide, the homocysteine transporter, and/or the serine transporter) that is administered by itself (i.e., not on or in a cell described herein). In one embodiment, the first exogenous polypeptide, the second exogenous polypeptide, and/or the third exogenous polypeptide have an in vivo half-life that is longer than the half-life of the first exogenous polypeptide, the second exogenous polypeptide, and/or the third exogenous polypeptide, or a pegylated version of the first exogenous polypeptide, the second exogenous polypeptide, and/or the third exogenous polypeptide, which are not included in an engineered erythroid cell.

In one embodiment, the first exogenous polypeptide, the second exogenous polypeptide, and/or the third exogenous polypeptide have an in vivo half-life of between about 24 hours and 60 days. In another embodiment, the first exogenous polypeptide, the second exogenous polypeptide, and/or the third exogenous polypeptide have an in vivo half-life of at least 24 hours. In another embodiment, the first exogenous polypeptide, the second exogenous polypeptide, and/or the third exogenous polypeptide have an in vivo half-life of greater than 36 hours. In another embodiment, the first exogenous polypeptide, the second exogenous polypeptide, and/or the third exogenous polypeptide have an in vivo half-life of greater than 48 hours. In another embodiment, the first exogenous polypeptide, the second exogenous polypeptide, and/or the third exogenous polypeptide have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide, the second exogenous polypeptide, and/or the third exogenous polypeptide have an in vivo half-life of 1 week, 2 weeks, 3 weeks, 1 month, 2 months, 3 months, 4 months or longer.

In some embodiments, the engineered erythroid cell comprises a first exogenous polypeptide, wherein the first exogenous polypeptide has an in vivo half-life of at least 24 hours. In another embodiment, the first exogenous polypeptide has an in vivo half-life of greater than 36 hours. In another embodiment, the first exogenous polypeptide has an in vivo half-life of greater than 48 hours. In another embodiment, the first exogenous polypeptide has an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide has an in vivo half-life of 1 week, 2 weeks, 3 weeks, 1 month, 2 months, 3 months, 4 months or longer. In one embodiment, the first exogenous polypeptide comprises a homocysteine reducing polypeptide, or a variant thereof. In another embodiment, the first exogenous polypeptide comprises a homocysteine degrading polypeptide, or a variant thereof. In another embodiment, the first exogenous polypeptide comprises a cystathionine beta-synthase (CBS) polypeptide, or variant thereof. In another embodiment, the first exogenous polypeptide comprises a methionine gamma-lyase (MGL) polypeptide, or variant thereof. In another embodiment, the first exogenous polypeptide comprises a homocysteine or serine transporter, or variant thereof.

In some embodiments, the engineered erythroid cell comprises a first exogenous polypeptide and further comprises a second exogenous polypeptide, wherein the first and second exogenous polypeptides have an in vivo half-life of at least 24 hours. In another embodiment, the first and second exogenous polypeptides have an in vivo half-life of greater than 36 hours. In another embodiment, the first and second exogenous polypeptides have an in vivo half-life of greater than 48 hours. In another embodiment, the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first and second exogenous polypeptides have an in vivo half-life of 1 week, 2 weeks, 3 weeks, 1 month, 2 months, 3 months, 4 months or longer. In one embodiment, the first exogenous polypeptide comprises a homocysteine reducing polypeptide, or a variant thereof. In another embodiment, the first exogenous polypeptide comprises a homocysteine degrading polypeptide, or a variant thereof. In another embodiment, the first exogenous polypeptide comprises a cystathionine beta-synthase (CBS) polypeptide, or variant thereof. In another embodiment, the first exogenous polypeptide comprises a methionine gamma-lyase (MGL) polypeptide, or variant thereof. In one embodiment, the second exogenous polypeptide comprises an amino acid transporter, or a variant thereof.

In some embodiments, the engineered erythroid cell comprises a first exogenous polypeptide, a second exogenous polypeptide and a third exogenous polypeptide, wherein the first exogenous polypeptide, second exogenous polypeptide and third exogenous polypeptide have an in vivo half-life of at least 24 hours. In another embodiment, the first exogenous polypeptide, second exogenous polypeptide and third exogenous polypeptide have an in vivo half-life of greater than 36 hours. In another embodiment, the first exogenous polypeptide, second exogenous polypeptide and third exogenous polypeptide have an in vivo half-life of greater than 48 hours. In another embodiment, the first exogenous polypeptide, second exogenous polypeptide and third exogenous polypeptide have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide, second exogenous polypeptide and third exogenous polypeptide have an in vivo half-life of 1 week, 2 weeks, 3 weeks, 1 month, 2 months, 3 months, 4 months or longer. In one embodiment, the first exogenous polypeptide comprises a homocysteine degrading polypeptide, or variant thereof. In one embodiment, the second exogenous polypeptide comprises a homocysteine transporter, or a variant thereof. In another embodiment, the third exogenous transporter comprises a serine transporter, or a variant thereof. In one embodiment, the first exogenous polypeptide comprises a homocysteine degrading polypeptide, or variant thereof. In one embodiment, the second exogenous polypeptide comprises a homocysteine transporter, or a variant thereof. In another embodiment, the third exogenous transporter comprises a cystathionine degrading polypeptide, or variant thereof.

In some embodiments, the engineered erythroid cell comprises a first exogenous polypeptide, a second exogenous polypeptide, a third exogenous polypeptide, and a fourth exogenous polypeptide, wherein the first exogenous polypeptide, second exogenous polypeptide, third exogenous polypeptide and fourth exogenous polypeptide have an in vivo half-life of at least 24 hours. In another embodiment, the first exogenous polypeptide, second exogenous polypeptide, third exogenous polypeptide and fourth exogenous polypeptides have an in vivo half-life of greater than 36 hours. In another embodiment, the first exogenous polypeptide, second exogenous polypeptide, third exogenous polypeptide and fourth exogenous polypeptide have an in vivo half-life of greater than 48 hours. In another embodiment, the first exogenous polypeptide, second exogenous polypeptide, third exogenous polypeptide and fourth exogenous polypeptide have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In another embodiment, the first exogenous polypeptide, second exogenous polypeptide, third exogenous polypeptide and fourth exogenous polypeptide have an in vivo half-life of 1 week, 2 weeks, 3 weeks, 1 month, 2 months, 3 months, 4 months or longer. In one embodiment, the first exogenous polypeptide comprises a homocysteine degrading polypeptide, or variant thereof. In one embodiment, the second exogenous polypeptide comprises a homocysteine transporter, or a variant thereof. In another embodiment, the third exogenous transporter comprises a serine transporter, or a variant thereof. In one embodiment, the first exogenous polypeptide comprises a homocysteine degrading polypeptide, or variant thereof. In one embodiment, the second exogenous polypeptide comprises a homocysteine transporter, or a variant thereof. In one embodiment, the third exogenous transporter comprises a serine transporter, or a variant thereof. In one embodiment, the fourth exogenous transporter comprises a cystathionine degrading polypeptide, or a variant thereof.

### Modifications

One or more of the exogenous proteins may have post-translational modifications characteristic of eukaryotic cells, e.g., mammalian cells, e.g., human cells. In some embodiments, one or more (e.g., 2, 3, 4, 5, 6, 7, 8, 9, 10, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments of the above aspects and embodiments, the engineered erythroid cell is an enucleated cell, e.g., reticulocyte or erythrocyte. In some embodiments of the above aspects and embodiments, the engineered erythroid cell is a nucleated cell.

### Populations of Engineered Erythroid Cells

In one aspect, the invention features cell populations comprising the engineered erythroid cells of the invention, e.g., a plurality or population of the engineered erythroid cells. In various embodiments, the engineered erythroid cell population comprises predominantly enucleated cells, predominantly nucleated cells, or a mixture of enucleated and nucleated cells. In such cell populations, the enucleated cells can comprise reticulocytes, erythrocytes, or a mixture of reticulocytes and erythrocytes. In one embodiment, the enucleated cells are reticulocytes. In one embodiment, the enucleated cells are erythrocytes.

In one embodiment, the engineered erythroid cell population consists essentially of enucleated cells. In one embodiment, the engineered erythroid cell population comprises predominantly or substantially enucleated cells. For example, in one embodiment, the population of engineered erythroid cells comprises at least about 80% or more enucleated cells. In some embodiments, the population provided herein comprises at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 80% enucleated cells. In some embodiments, the population of engineered erythroid cells comprises greater than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In one embodiment, the population of engineered erythroid cells comprises less than about 20% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, or less than about 20% nucleated cells. In one embodiment, the population of engineered erythroid cells comprises less than about 1% nucleated cells. In one embodiment, the population of engineered erythroid cells comprises less than about 2% nucleated cells. In one embodiment, the population of engineered erythroid cells comprises less than about 3% nucleated cells. In one embodiment, the population of engineered erythroid cells comprises less than about 4% nucleated cells. In one embodiment, the population of engineered erythroid cells comprises less than about 5% nucleated cells. In one embodiment, the population of engineered erythroid cells comprises less than about 10% nucleated cells. In one embodiment, the population of engineered erythroid cells comprises less than about 15% nucleated cells. In some embodiments, the population of engineered erythroid cells comprises between 0% and 20% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells of the invention, wherein the population of engineered erythroid cells comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells of the invention, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In another embodiment, the engineered erythroid cell population comprises predominantly or substantially nucleated cells. In one embodiment, the engineered erythroid cell population consists essentially of nucleated cells. In various embodiments, the nucleated cells in the engineered erythroid cell population are erythrocyte (or fully mature red blood cell) precursor cells. In embodiments, the erythroid precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

It will be understood that during the preparation of the engineered erythroid cells of the invention, some fraction of cells may not become conjugated with an exogenous polypeptide or transduced to include an exogenous polypeptide. Accordingly, in some embodiments, a population of engineered erythroid cells provided herein comprises a mixture of engineered erythroid cells and unmodified erythroid cells, i.e., some fraction of cells in the population will not comprise, present, or express an exogenous polypeptide. For example, a population of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the population are not engineered. In embodiments, a single unit dose of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the dose are not engineered.

### II. Methods of Making Engineered Erythroid Cells

Various methods of making engineered erythroid cells, e.g., enucleated erythroid cells, or enucleated cells, are contemplated by the present disclosure.

Methods of manufacturing enucleated erythroid cells comprising an exogenous agent (e.g., a polypeptide) are described, e.g., in International Application Publication Nos. WO2015/073587 and WO2015/153102, each of which is incorporated by reference in its entirety.

In some embodiments, hematopoietic progenitor cells, e.g., CD34⁺ hematopoietic progenitor cells (e.g., human (e.g., adult human) or mouse cells), are contacted with a nucleic acid or nucleic acids encoding one or more exogenous polypeptides, and the cells are allowed to expand and differentiate in culture. In some embodiments, the CD34⁺ cells are immortalized, e.g., comprise a human papilloma virus (HPV; e.g., HPV type 16) E6 and/or E7 genes. In some embodiments, the immortalized CD34⁺ hematopoietic progenitor cell is a BEL-A cell line cell (see Trakarnasanga et al. (2017) Nat. Commun. 8: 14750). Additional immortalized CD34⁺ hematopoietic progenitor cells are described in U.S. Pat. Nos. 9,951,350, and 8,975,072. In some embodiments, an immortalized CD34⁺ hematopoietic progenitor cell is contacted with a nucleic acid or nucleic acids encoding one or more exogenous polypeptides, and the cells are allowed to expand and differentiate in culture.

In one aspect, the disclosure provides an engineered enucleated cell, comprising a first exogenous polypeptide comprising a homocysteine reducing polypeptide, or a variant thereof, produced by a process comprising introducing an exogenous nucleic acid encoding the first exogenous polypeptide into a nucleated erythroid cell; and culturing the nucleated erythroid cell under conditions suitable for enucleation of the nucleated erythroid cell and for production of the first exogenous polypeptide.

In another aspect, the disclosure provides an engineered enucleated cell (e.g., engineered enucleated erythroid cell), comprising a first exogenous polypeptide comprising a homocysteine degrading polypeptide, or variant thereof, wherein the homocysteine degrading polypeptide, or variant thereof, is not a cystathionine beta-synthase, produced by a process comprising introducing an exogenous nucleic acid encoding the first exogenous polypeptide into a nucleated erythroid cell; and culturing the nucleated erythroid cell under conditions suitable for enucleation of the nucleated erythroid cell and for production of the first exogenous polypeptide.

In another aspect, the invention provides an engineered enucleated cell, comprising a first exogenous polypeptide comprising a cystathionine beta-synthase (CBS) polypeptide, or variant thereof, produced by a process comprising introducing an exogenous nucleic acid encoding the first exogenous polypeptide into a nucleated erythroid cell; and culturing the nucleated erythroid cell under conditions suitable for enucleation of the nucleated erythroid cell and for production of the first exogenous polypeptide.

In another aspect, the invention provides an engineered enucleated cell (e.g., engineered enucleated erythroid cell), comprising a first exogenous polypeptide comprising a methionine gamma-lyase polypeptide, or variant thereof, produced by a process comprising introducing an exogenous nucleic acid encoding the first exogenous polypeptide into a nucleated erythroid cell; and culturing the nucleated erythroid cell under conditions suitable for enucleation of the nucleated erythroid cell and for production of the first exogenous polypeptide.

In another aspect, the invention provides an engineered enucleated cell (e.g., engineered enucleated erythroid cell), comprising a first exogenous polypeptide comprising a homocysteine or serine transporter, or a variant thereof, produced by a process comprising introducing an exogenous nucleic acid encoding the first exogenous polypeptide into a nucleated erythroid cell; and culturing the nucleated erythroid cell under conditions suitable for enucleation of the nucleated erythroid cell and for production of the first exogenous polypeptide.

In another aspect, the invention provides an engineered enucleated cell (e.g., engineered enucleated erythroid cell), comprising a first exogenous polypeptide comprising a homocysteine reducing polypeptide, or a variant thereof, and a second exogenous polypeptide comprising an amino acid transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; and culturing the nucleated erythroid cell under conditions suitable for enucleation of the nucleated erythroid cell and for production of the first exogenous polypeptide and the second exogenous polypeptide.

In another aspect, the invention provides an engineered enucleated cell (e.g., engineered enucleated erythroid cell), comprising a first exogenous polypeptide comprising a homocysteine degrading polypeptide, or variant thereof, and a second exogenous polypeptide comprising an amino acid transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; and culturing the nucleated erythroid cell under conditions suitable for enucleation of the nucleated erythroid cell and for production of the first exogenous polypeptide and the second exogenous polypeptide.

In another aspect, the invention provides an engineered enucleated cell (e.g., engineered enucleated erythroid cell), comprising a first exogenous polypeptide comprising a cystathionine beta-synthase (CBS) polypeptide, or variant thereof, and a second exogenous polypeptide comprising an amino acid transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; and culturing the nucleated erythroid cell under conditions suitable for enucleation of the nucleated erythroid cell and for production of the first exogenous polypeptide and the second exogenous polypeptide. In one embodiment, the second exogenous polypeptide is a homocysteine transporter or a serine transporter.

In another aspect, the invention provides an engineered enucleated cell (e.g., engineered enucleated erythroid cell), comprising a first exogenous polypeptide comprising a methionine gamma-lyase (MGL) polypeptide, or variant thereof, and a second exogenous polypeptide comprising an amino acid transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; and culturing the nucleated erythroid cell under conditions suitable for enucleation of the nucleated erythroid cell and for production of the first exogenous polypeptide and the second exogenous polypeptide. In one embodiment, the second exogenous polypeptide is a homocysteine transporter or a serine transporter.

In another aspect, the invention provides an engineered enucleated cell (e.g., engineered enucleated erythroid cell), comprising a first exogenous polypeptide comprising a cystathionine beta-synthase (CBS) polypeptide, or a variant thereof, and a second exogenous polypeptide comprising a homocysteine transporter, or a variant thereof, and a third exogenous polyeptide comprising a cystathionine gamma lyase, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; introducing an exogenous nucleic acid encoding the third exogenous polypeptide into a nucleated erythroid cell; culturing the nucleated erythroid cell under conditions suitable for enucleation of the nucleated erythroid cell and for production of the first exogenous polypeptide, the second exogenous polypeptide and the third exogenous polypeptide.

In another aspect, the invention provides an engineered enucleated cell (e.g., engineered enucleated erythroid cell), comprising at a first exogenous polypeptide comprising a cystathionine beta-synthase (CBS) polypeptide, or a variant thereof, and a second exogenous polypeptide comprising a homocysteine transporter, or a variant thereof, a third exogenous polypeptide comprising a serine transporter, and a fourth exogenous polyeptide comprising a cystathionine gamma lyase, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; introducing an exogenous nucleic acid encoding the third exogenous polypeptide into a nucleated erythroid cell; introducing an exogenous nucleic acid encoding the fourth exogenous polypeptide into a nucleated erythroid cell; culturing the nucleated erythroid cell under conditions suitable for enucleation of the nucleated erythroid cell and for production of the first exogenous polypeptide, the second exogenous polypeptide, the third exogenous polypeptide and the fourth exogenous polypeptide.

In another aspect, the invention provides an engineered enucleated cell (e.g., engineered enucleated erythroid cell), comprising at a first exogenous polypeptide comprising a homocysteine reducing polypeptide, or a variant thereof, and a second exogenous polypeptide comprising a homocysteine transporter, or a variant thereof, and a third exogenous polypeptide comprising a serine transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; introducing an exogenous nucleic acid encoding the third exogenous polypeptide into a nucleated erythroid cell; culturing the nucleated erythroid cell under conditions suitable for enucleation of the nucleated erythroid cell and for production of the first exogenous polypeptide, the second exogenous polypeptide and the third exogenous polypeptide.

In another aspect, the invention provides an engineered enucleated cell (e.g., engineered enucleated erythroid cell), comprising at a first exogenous polypeptide comprising a homocysteine degrading polypeptide, or variant thereof, and a second exogenous polypeptide comprising a homocysteine transporter, or a variant thereof, and a third exogenous transporter comprising a serine transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; introducing an exogenous nucleic acid encoding the third exogenous polypeptide into a nucleated erythroid cell; culturing the nucleated erythroid cell under conditions suitable for enucleation of the nucleated erythroid cell and for production of the first exogenous polypeptide, the second exogenous polypeptide and the third exogenous polypeptide.

In another aspect, the invention provides an engineered enucleated cell (e.g., engineered enucleated erythroid cell), comprising at a first exogenous polypeptide comprising a cystathionine beta-synthase (CBS) polypeptide, or variant thereof, and a second exogenous polypeptide comprising a homocysteine transporter, or a variant thereof, and a third exogenous transporter comprising a serine transporter, or a variant thereof, produced by a process comprising introducing an exogenous nucleic acid encoding the first exogenous polypeptide into a nucleated erythroid cell; introducing an exogenous nucleic acid encoding the second exogenous polypeptide into a nucleated erythroid cell; introducing an exogenous nucleic acid encoding the third exogenous polypeptide into a nucleated erythroid cell; culturing the nucleated erythroid cell under conditions suitable for enucleation of the nucleated erythroid cell and for production of the first exogenous polypeptide, the second exogenous polypeptide and the third exogenous polypeptide.

The processes of making the engineered enucleated cells are described in more detail below.

### Physical Characteristics of Engineered Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an engineered erythroid cell e.g., an enucleated erythroid cell, that includes an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the engineered erythroid cell, e.g., enucleated erythroid cell, comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the engineered erythroid cell, e.g., enucleated erythroid cell, exhibits substantially the same osmotic membrane fragility as an isolated, uncultured erythroid cell that does not comprise an exogenous polypeptide. In some embodiments, the population of engineered erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the engineered erythroid cell, e.g., enucleated erythroid cell, has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the engineered erythroid cell, e.g., enucleated cell, has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the engineered erythroid cell, e.g., enucleated cell, has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, an engineered erythroid cell (e.g., engineered enucleated erythroid cell) or an engineered enucleated cell, or a population of engineered erythroid cells or engineered enucleated cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA (i.e., CD235a⁺) cells. In some embodiments, the population of engineered erythroid cells (engineered enucleated erythroid cells) or engineered enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD34⁻/CD36⁻ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻ cells.

In some embodiments, a population of engineered erythroid cells (engineered enucleated erythroid cells) or engineered enucleated cells comprising erythroid cells comprises less than about 10%, 9%, 8%, 7%, 6%, 5%, 4%, 3%, 2%, or 1% echinocytes.

In some embodiments, a population of engineered erythroid cells (e.g. artificial antigen presenting cells as described herein) comprising erythroid cells comprises less than about 10%, 9%, 8%, 7%, 6%, 5%, 4%, 3%, 2%, or 1% echinocytes.

In some embodiments, a population of engineered erythroid cells (engineered enucleated erythroid cells) or engineered enucleated cells comprises less than about 10%, 9%, 8%, 7%, 6%, 5%, 4%, 3%, 2%, or 1% pyrenocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated. In some embodiments, the engineered erythroid cell is an enucleated cell. In some embodiments, the engineered erythroid cell is a nucleated cell.

### Isolating Erythrocytes

Mature erythrocytes may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van der Berg et al., Clin. Chem. 33:1081-1082 (1987); Bar-Zvi et al., J. Biol. Chem. 262:17719-17723 (1987); Goodman et al., Exp. Biol. Med. 232:1470-1476 (2007)).

Erythrocytes may be isolated from whole blood by simple centrifugation (See, e.g., van der Berg et al., Clin. Chem. 33:1081-1082 (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, erythrocytes may be isolated using density gradient centrifugation with various separation mediums such as, for example, Ficoll, Hypaque, Histopaque, Percoll, Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, erythrocytes may be isolated by centrifugation using a Percoll step gradient (See, e.g., Bar-Zvi et al., J. Biol. Chem. 262:17719-17723 (1987)). For example, fresh blood is mixed with an anticoagulant solution containing 75 mM sodium citrate and 38 mM citric acid and the cells washed briefly in Hepes-buffered saline. Leukocytes and platelets are removed by adsorption with a mixture of α-cellulose and Sigmacell (1:1). The erythrocytes are further isolated from reticulocytes and residual white blood cells by centrifugation through a 45/75% Percoll step gradient for 10 min at 2500 rpm in a Sorvall SS34 rotor. The erythrocytes are recovered in the pellet while reticulocytes band at the 45/75% interface and the remaining white blood cells band at the 0/45% interface. The Percoll is removed from the erythrocytes by several washes in Hepes-buffered saline. Other materials that may be used to generate density gradients for isolation of erythrocytes include OPTIPREP, a 60% solution of iodixanol in water (from Axis-Shield, Dundee, Scotland).

Erythrocytes may be separated from reticulocytes, for example, using flow cytometry

(See, e.g., Goodman el al., Exp. Biol. Med. 232:1470-1476 (2007)). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the erythrocytes from the white blood cells. The resulting cell pellet is resuspended in RPMI supplemented with 10% fetal bovine serum and sorted on a FACS instrument such as, for example, a Becton Dickinson FACSCalibur (BD Biosciences, Franklin Lakes, N.J., USA) based on size and granularity.

Erythrocytes may be isolated by immunomagnetic depletion (See, e.g., Goodman, el al., (2007) Exp. Biol. Med. 232:1470-1476). In this instance, magnetic beads with cell-type specific antibodies are used to eliminate non-erythrocytes. For example, erythrocytes are isolated from the majority of other blood components using a density gradient as described herein followed by immunomagnetic depletion of any residual reticulocytes. The cells are pre-treated with human antibody serum for 20 min at 25° C. and then treated with antibodies against reticulocyte specific antigens such as, for example, CD71 and CD36. The antibodies may be directly attached to magnetic beads or conjugated to PE, for example, to which magnetic beads with anti-PE antibody will react. The antibody-magnetic bead complex is able to selectively extract residual reticulocytes, for example, from the erythrocyte population.

Erythrocytes may also be isolated using apheresis. The process of apheresis involves removal of whole blood from a patient or donor, separation of blood components using centrifugation or cell sorting, withdrawal of one or more of the separated portions, and transfusion of remaining components back into the patient or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods may be necessary to achieve the appropriate degree of cell purity.

Reticulocytes are immature red blood cells and compose approximately 1% of the red blood cells in the human body. Reticulocytes develop and mature in the bone marrow. Once released into circulation, reticulocytes rapidly undergo terminal differentiation to mature erythrocytes. Like mature erythrocytes, reticulocytes do not have a cell nucleus.

Reticulocytes of varying age may be isolated from peripheral blood based on the differences in cell density as the reticulocytes mature. Reticulocytes may be isolated from peripheral blood using differential centrifugation through various density gradients. For example, Percoll gradients may be used to isolate reticulocytes (See, e.g., Noble el al., Blood 74:475-481 (1989)). Sterile isotonic Percoll solutions of density 1.096 and 1.058 g/m1 are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250×g for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al., Blood 76:2397-2403 (1990)). This approach takes advantage of the large number of transferrin receptors that are expressed on the surface of reticulocytes relative to erythrocytes prior to maturation. Magnetic beads coated with an antibody to the transferrin receptor may be used to selectively isolate reticulocytes from a mixed blood cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell fraction. The beads and red blood cells are incubated at 22° C. with gentle mixing for 60-90 min followed by isolation of the beads with attached reticulocytes using a magnetic field. The isolated reticulocytes may be removed from the magnetic beads using, for example, DETACHaBEAD solution (from Invitrogen, Carlsbad, Calif., USA). Alternatively, reticulocytes may be isolated from in vitro growth and maturation of CD34+ hematopoietic stem cells using the methods described herein.

Terminally-differentiated, enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable methods.

### Vehicles for Polypeptides Described Herein

While in many embodiments herein, the one or more (e.g., two or more) exogenous polypeptides (e.g. a homocysteine reducing polypeptide, a homocysteine degrading polypeptide, a homocysteine transporter or a serine transporter) are situated on or in an enucleated erythroid cell, it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more exogenous polypeptides described herein. In some embodiments, the vehicle comprises two or more exogenous polypeptides described herein, e.g., any pair of exogenous polypeptides described herein.

In one aspect, one or more polypeptides described herein are loaded onto, attached (e.g., immobilized or conjugated) to the surface of, and/or enclosed in a non-cellular delivery vehicle. The non-cellular delivery vehicle can be, for example, a nanolipidgel, a polymeric particle, an agarose particle, a latex particle, a silica particle, a liposome, or a multilamellar vesicles. In some embodiments, the non-cellular delivery vehicle comprises or consists of a nanoparticle of from about 1 nm to about 900 nm in diameter. In some embodiments, the non-cellular delivery vehicle comprises an average diameter of from about 0.1 to about 20 microns (such as from about 0.5 microns to about 10 microns, e.g., about 5 microns or less (e.g., about 2.5 to about 5 microns)). In some embodiments, the non-cellular delivery vehicle comprises an average diameter of from about 1μm to about 10 μm. In some embodiments, the non-cellular delivery vehicle comprises a biodegradable polymer. In some embodiments, the non-cellular delivery vehicle comprises a natural polymer. In some embodiments, the non-cellular delivery vehicle comprises a synthetic polymer. Representative polymers include, but are not limited to, a poly(hydroxy acid), a polyhydroxyalkanoate, a polycaprolactone, a polycarbonate, a polyamide, a polyesteramide, poly(acrylamide), poly(ester), poly(alkylcyanoacrylates), poly(lactic acid) (PLA), poly(glycolic acids) (PGA), and poly(D,L-lactic-co-glycolic acid) (PLGA), and combinations thereof. In some embodiments, the non-cellular delivery vehicle comprises agarose, latex, or polystyrene. One or more of the polypeptides described herein can be conjugated to a non-cellular delivery vehicle using standard methods known in the art (see, e.g., Ulbrich et al. (2016) Chem Rev. 116(9): 5338-431). Conjugation can be either covalent or non-covalent. For example, in embodiments in which the non-cellular delivery vehicle is a liposome, a polypeptide described herein may be attached to the liposome via a polyethylene glycol (PEG) chain. Conjugation of a polypeptide to a liposome can also involve thioester bonds, for example by reaction of thiols and maleimide groups. Cross-linking agents can be used to create sulfhydryl groups for attachment of polypeptides to non-cellular delivery vehicles (see, e.g., Paszko and Senge (2012) Curr. Med. Chem. 19(31): 5239-77). In some embodiments, the non-cellular delivery vehicles comprising one or more of the polypeptides described herein may be used in any of therapeutic methods provided herein.

### Heterogeneous Populations of Cells

While in many embodiments herein, the one or more (e.g., two or more) exogenous polypeptides (e.g. a homocysteine reducing polypeptide, a homocysteine degrading polypeptide, a homocysteine transporter or a serine transporter), are situated on or in a single cell, it is understood that any polypeptide or combination of polypeptides described herein can also be situated on a plurality of cells. For instance, in some aspects, the disclosure provides a plurality of erythroid cells, wherein a first cell of the plurality comprises a first exogenous polypeptide and a second cell of the plurality comprises a second exogenous polypeptide. In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Erythroid Precursor Cells

Provided herein are engineered erythroid precursor cells, and methods of making the engineered erythroid precursor cells, reticulocytes and erythrocytes.

Pluripotent stem cells give rise to erythrocytes by the process of erythropoiesis. The stem cell looks like a small lymphocyte and lacks the functional capabilities of the erythrocyte. The stem cells have the capacity of infinite division, something the mature cells lack. Some of the daughter cells arising from the stem cell acquire erythroid characters over generations and time. Most of the erythroid cells in the bone marrow have a distinct morphology but commitment to erythroid maturation is seen even in cells that have not acquired morphological features distinctive of the erythroid lineage. These cells are recognized by the type of colonies they form in vitro. Two such cells are recognized. Burst-forming unit erythroid (BFU-E) arise from the stem cell and gives rise to colony-forming unit erythroid (CFU-E). CFU-E gives rise to pronormoblast, the most immature of erythroid cells with a distinct morphology. BFU-E and CFU-E form a very small fraction of bone marrow cells. Morphologically five erythroid precursors are identifiable in the bone marrow stained with Romanovsky stains. The five stages from the most immature to the most mature are the proerythroblast, the basophilic normoblast (early erythroblast), polychromatophilic normoblast (intermediate erythroblast), orthochromatophilic normoblast (late erythroblast) and reticulocyte. BFU-E (burst forming unit-erythroid), CFU-E (erythroid colony-forming unit), pronormoblast (proerythroblast), basophilic normoblast, polychromatophilic normoblast and orthochromatophilic normoblast are lineage restricted.

Table 7 below summarizes the morphological features of erythroid precursor cells and erythrocytes.

TABLE 7
Morphological features of erythroid precursor and erythroid cells

| Cell | Nucleus |
|---|---|
| Hematopoietic stem cell (HSC) | Yes |
| CMP (Common myeloid progenitor) | Yes |
| CFU-S (spleen colony forming cell; | Yes; Can differentiate into |
| myeloid precursor cell) | erythrocytes, platelets, |
| | macrophages. |
| BFU-E (burst forming unit-erythroid) | Yes |
| CFU-E (erythroid colony-forming | Yes |
| unit) | |
| Pronormoblast (proerythroblast) | Yes; fine chromatin, many |
| | nucleoli |
| Basophilic Normoblast | Yes; granular chromatin, no |
| | nucleoli |
| Polychromatophilic Normoblast | Yes; chromatin is visibly |
| | clumped with dark staining |
| | areas |
| Orthochromatophilic normoblast | Yes; featureless nucleus with |
| | dense chromatin |
| Reticulocyte | No Nucleus |
| Erythrocyte (fully matured RBC) | No Nucleus |

Normal human erythrocytes express CD36, an adhesion molecule of monocytes, platelets, and endothelial cells (van Schravendijk MR et al., Blood. 1992 Oct. 15; 80(8):2105-14). Accordingly, in some embodiments, an anti-CD36 antibody can be used to identify human erythrocytes.

Any type of cell known in the art that is capable of differentiating into an erythrocyte, i.e., any erythroid precursor cell, can be modified in accordance with the methods described herein to produce engineered erythroid precursor cells. In certain embodiments, the erythroid precursor cells modified in accordance with the methods described herein are cells that are in the process of differentiating into an erythrocyte, i.e., the cells are of a type known to exist during mammalian erythropoiesis. For example, the cells may be pluripotent hematopoietic stem cells (HSCs) or CD34+ cells, multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The modified erythroid precursor cells provided herein can be differentiated into engineered reticulocytes or erythrocytes in vitro using methods known in the art, i.e., using molecules known to promote erythropoiesis, e.g., SCF, Erythropoietin, IL-3, and/or GM-CSF, described herein below. Alternatively, the modified erythroid precursor cells are provided in a composition of the invention, and are capable of differentiating into erythrocytes upon administration to a subject in vivo.

In some embodiments, the erythroid precursor cells, e.g., hematopoietic stem cells, are from an O-negative donor. In some embodiments, the erythroid precursor cells lack (e.g., do not express or encode) A and/or B antigen.

### Culturing

Sources for generating engineered erythroid cells described herein include circulating erythroid cells. A suitable cell source may be isolated from a subject as described herein from patient-derived hematopoietic or erythroid progenitor cells, derived from immortalized erythroid cell lines, or derived from induced pluripotent stem cells, optionally cultured and differentiated. Methods for generating erythrocytes using cell culture techniques are well known in the art, e.g., Giarratana et al., Blood 2011, 118:5071, Huang et al., Mol Ther 2013, epub ahead of print September 3, or Kurita et al., PLOS One 2013, 8:e59890. Protocols vary according to growth factors, starting cell lines, culture period, and morphological traits by which the resulting cells are characterized. Culture systems have also been established for blood production that may substitute for donor transfusions (Fibach et al. 1989 Blood 73:100). Recently, CD34+ cells were differentiated to the reticulocyte stage, followed by successful transfusion into a human subject (Giarratana et al., Blood 2011, 118:5071).

Provided herein are culturing methods for erythroid cells and engineered erythroid cells. Erythroid cells can be cultured from hematopoietic progenitor cells, including, for example, CD34+ hematopoietic progenitor cells (Giarratana et al., Blood 2011, 118:5071), induced pluripotent stem cells (Kurita et al., PLOS One 2013, 8:e59890), and embryonic stem cells (Hirose et al. 2013 Stem Cell Reports 1:499). Cocktails of growth and differentiation factors that are suitable to expand and differentiate progenitor cells are known in the art. Examples of suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from hematopoietic progenitors, such as CD34+ cells, by contacting the progenitor cells with defined factors in a multi-step culture process. For example, erythroid cells can be cultured from hematopoietic progenitors in a three-step process.

The first step may comprise contacting the cells in culture with stem cell factor (SCF) at 1-1000 ng/mL, erythropoietin (EPO) at 1-100 U/mL, and interleukin-3 (IL-3) at 0.1-100 ng/mL. The first step optionally comprises contacting the cells in culture with a ligand that binds and activates a nuclear hormone receptor, such as e.g., the glucocorticoid receptor, the estrogen receptor, the progesterone receptor, the androgen receptor, or the pregnane x receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane x receptor ligand, such as, e.g., rifampicin at 10 nM-100 μM, hyperforin at 10 nM-100 St. John's Wort (hypericin) at 10 nM-100 μM, or vitamin E-like molecules, such as, e.g., tocopherol at 10 nM-100 The first step may also optionally comprise contacting the cells in culture with an insulin-like molecule, such as, e.g., insulin at 1-50 μ.g/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step further may optionally comprise contacting the cells in culture with transferrin at 0.1-5 mg/mL.

The first step may optionally comprise contacting the cells in culture with one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may typically be supplied at a concentration of 0.1-100 ng/mL. The first step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The second step may comprise contacting the cells in culture with stem cell factor (SCF) at 1-1000 ng/mL and erythropoietin (EPO) at 1-100 U/mL. The second step may also optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The second step may further optionally comprise contacting the cells in culture with transferrin at 0.1-5 mg/mL. The second may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The third step may comprise contacting the cells in culture with erythropoietin (EPO) at 1-100 U/mL. The third step may optionally comprise contacting the cells in culture with stem cell factor (SCF) at 1-1000 ng/mL. The third step may further optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The third step may also optionally comprise contacting the cells in culture with transferrin at 0.1-5 mg/mL. The third step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

In some embodiments, methods of expansion and differentiation of the engineered erythroid cells comprising an erythroid cell presenting one or more exogenous polypeptides, do not include culturing the engineered erythroid cells in a medium comprising a myeloproliferative receptor (mpl) ligand.

The culture process may optionally comprise contacting cells by a method known in the art with a molecule, e.g., a DNA molecule, an RNA molecule, a mRNA, an siRNA, a microRNA, a lncRNA, a shRNA, a hormone, or a small molecule, that activates or knocks down one or more genes. Target genes can include, for example, genes that encode a transcription factor, a growth factor, or a growth factor receptor, including but not limited to, e.g., GATA1, GATA2, CMyc, hTERT, p53, EPO, SCF, insulin, EPO-R, SCF-R, transferrin-R, insulin-R.

In some embodiments, CD34+ cells are placed in a culture containing varying amounts of IMDM, FBS, glutamine, BSA, holotransferrin, insulin, dexamethasone, .beta.-estradiol, IL-3, SCF, and erythropoietin, in three separate differentiation stages for a total of 22 days.

In some embodiments, CD34+ cells are placed in a culture containing varying amounts of IMDM, FBS, glutamine, BSA, holotransferrin, insulin, dexamethasone, .beta.-estradiol, IL-3, SCF, and thrombopoietin, in three separate differentiation stages for a total of 14 days.

In some embodiments, CD34+ cells are placed in a culture containing varying amounts of IMDM, FBS, glutamine, BSA, holotransferrin, insulin, dexamethasone, .beta.-estradiol, IL-3, SCF, and GCSF, in three separate differentiation stages for a total of 15 days.

In some embodiments, the erythroid cells are expanded at least 100, 1000, 2000, 5000, 10,000, 20,000, 50,000, or 100,000 fold (and optionally up to 100,000, 200,000, or 500,000 fold). The number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, it may be desirable during culturing to only partially differentiate the erythroid progenitor cells, e.g., hematopoietic stem cells, in vitro, allowing further differentiation, e.g., differentiation into reticulocytes or fully mature erythrocytes, to occur upon introduction to a subject in vivo (See, e.g., Neildez-Nguyen et al., Nature Biotech. 20:467-472 (2002)). It will be understood that, in various embodiments of the invention, maturation and/or differentiation in vitro may be arrested at any stage desired. For example, isolated CD34+ hematopoietic stem cells may be expanded in vitro as described elsewhere herein, e.g., in medium containing various factors, including, for example, interleukin 3, Flt3 ligand, stem cell factor, thrombopoietin, erythropoietin, transferrin, and insulin growth factor, to reach a desired stage of differentiation. The resulting engineered erythroid cells may be characterized by the surface expression of CD36 and GPA, and other characteristics specific to the particular desired cell type, and may be transfused into a subject where terminal differentiation to mature erythrocytes is allowed to occur.

In some embodiments, engineered erythroid cells are partially expanded from erythroid progenitor cells to any stage of maturation prior to but not including enucleation, and thus remain nucleated cells, e.g., erythroid precursor cells. In certain embodiments, the resulting cells are nucleated and erythroid lineage restricted. In certain embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The final differentiation steps, including enucleation, occur only after administration of the engineered erythroid cell to a subject, that is, in such embodiments, the enucleation step occurs in vivo. In another embodiment, engineered erythroid cells are expanded and differentiated in vitro through the stage of enucleation to become, e.g., reticulocytes. In such embodiments where the engineered erythroid cells are differentiated to the stage of reticuloyctes, the final differentiation step to become erythrocytes occurs only after administration of the engineered erythroid cell to a subject, that is, the terminal differentiation step occurs in vivo. In another embodiment, engineered erythroid cells are expanded and differentiated in vitro through the terminal differentiation stage to become erythrocytes.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. In some embodiments, such engineered platelets including exogenous polypeptides as described herein are considered to be encompassed by the present invention.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. In some embodiments, such engineered platelets including exogenous polypeptides as described herein are considered to be encompassed by the present invention.

In some embodiments of the above aspects and embodiments, the engineered erythroid cell is an enucleated cell. In some embodiments of the above aspects and embodiments, the engineered erythroid cell is a nucleated cell.

In some embodiments, an enucleated cell provided herein is a platelet. Methods of manufacturing platelets in vitro are known in the art (see, e.g., Wang and Zheng (2016) Springerplus 5(1): 787, and U.S. Pat. No. 9,574,178). Methods of manufacturing platelets including an exogenous polypeptide are described, e.g., in International Patent Application Publication Nos. WO2015/073587 and WO2015/153102, each of which is incorporated by reference in its entirety. Platelet production is in part regulated by signaling mechanisms induced by interaction between thrombopoietin (TPO) and its cellular receptor TPOR/MPUc-MPL. In addition, multiple cytokines (e.g., stem cell factor (SCF), IL-1, IL-3, IL-6, IL-11, leukemia inhibiting factor (LIF), G-CSF, GM-CSF, M-CSF, erythropoietin (EPO), kit ligand, and interferon) have been shown to possess thrombocytopoietic activity.

In some embodiments, platelets are generated from hematopoietic progenitor cells, such as CD34⁺ hematopoietic stem cells, induced pluripotent stem cells or embryonic stem cells. In some embodiments, platelets are produced by contacting the progenitor cells with defined factors in a multi-step culture process. In some embodiments, the multi-step culture process comprises: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate progenitor cells and produce platelets are known in the art. Examples of suitable expansion and differentiation factors include, but are not limited to, stem cell factor (SCF), Flt-3/Flk-2 ligand (FL), TPO, IL-11, IL-3, IL-6, and IL-9. For instance, in some embodiments, platelets may be produced by seeding CD34⁺ HSCs in a serum-free medium at 2-4×10⁴ cells/mL, and refreshing the medium on culture day 4 by adding an equal volume of media. On culture day 6, cells are counted and analyzed: 1.5×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

### Expression of Exogenous Polypeptides

In some embodiments, the engineered erythroid cells described herein are generated by contacting a suitable isolated cell, e.g., an erythroid cell, a reticulocyte, an erythroid precursor cell, a platelet, or a platelet precursor, with an exogenous nucleic acid encoding a polypeptide of the disclosure (e.g. a homocysteine reducing polypeptide, a homocysteine degrading polypeptide, e.g., a cystathionine beta-synthase (CBS) polypeptide or a methionine gamma-lyase (MGL), a cystathionine degrading polypeptide, and/or an amino acid transporter).

In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleate erythroid cell, a nucleated platelet precursor cell, or a reticulocyte. In some embodiments, the exogenous polypeptide is contacted with a primary platelet, a nucleated erythroid cell, a nucleated platelet precursor cell, a reticulocyte, or an erythrocyte.

An exogenous polypeptide may be expressed from a transgene introduced into an erythroid cell by electroporation, chemical or polymeric transfection, viral transduction, mechanical membrane disruption, or other method; an exogenous polypeptide that is expressed from mRNA that is introduced into a cell by electroporation, chemical or polymeric transfection, viral transduction, mechanical membrane disruption, or other method; an exogenous polypeptide that is over-expressed from the native locus by the introduction of an external factor, e.g., a transcriptional activator, transcriptional repressor, or secretory pathway enhancer; and/or a polypeptide that is synthesized, extracted, or produced from a production cell or other external system and incorporated into the erythroid cell.

In certain embodiments, the introducing step comprises viral transduction. In another embodiment, the introducing step comprises electroporation. In another embodiment, the introducing step comprises utilizing one or more of: liposome mediated transfer, adenovirus, adeno-associated virus, herpes virus, a retroviral based vector, lipofection, and a lentiviral vector.

In some embodiments, the introducing step comprises introducing the first exogenous nucleic acid encoding the first exogenous polypeptide by transfection of a lentiviral vector.

Exogenous polypeptides (e.g. a homocysteine degrading polypeptide, a homocysteine tranporter or a serine transporter) can be introduced by transfection of single or multiple copies of genes, transduction with a virus, or electroporation in the presence of DNA or RNA. Methods for expression of exogenous proteins in mammalian cells are well known in the art. For example, expression of exogenous factor IX in hematopoietic cells is induced by viral transduction of CD34+ progenitor cells, see Chang et al., Nat Biotechnol 2006, 24:1017.

In some embodiments, when there are more than one polypeptides (e.g. two or more) the polypeptides may be encoded in a single nucleic acid, e.g. a single vector. When a homocysteine degrading polypeptide and a homocysteine tranporter or a serine transporter are encoded in the same vector, there are multiple possible sub-strategies useful for this method of co-expression. In some embodiments, the single vector has a separate promoter for each gene, has two proteins that are initially transcribed into a single polypeptide having a protease cleavage site in the middle (e.g. a T2A site), so that subsequent proteolytic processing yields two proteins, or any other suitable configuration. In some embodiments, the recombinant nucleic acid comprises a gene encoding a first exogenous polypeptide,wherein the first exogenous polypeptide is a homocysteine degrading polypeptide, and a gene encoding a second exogenous polypeptide, wherein the second exogenous polypeptide is a homocysteine transporter or a serine transporter, wherein the second gene is separated from the gene encoding the exogenous polypeptide by a viral-derived T2A sequence (gagggcagaggaagtcttctaacatgcggtgacgtggaggsgsstcccggccct (SEQ ID NO: 62)) that is post-translationally cleaved into two mature proteins.

For dual expression via 2 promoters, the MSCV promoter may be used as promoter #1 and the EF1 promoter as promoter #2, although the disclosure is not to be limited by these two exemplary promoters. Another strategy is to express both a homocysteine degrading polypeptide a homocysteine transporter or a serine transporter proteins by inserting an internal ribosome entry site (IRES) between the two genes. Still another strategy is to express a homocysteine degrading polypeptide a homocysteine transporter or a serine transporter as direct peptide fusions separated by a linker.

In some embodiments, the two or more polypeptides are encoded in two or more nucleic acids, e.g., each vector encodes one of the polypeptides.

In some embodiments, the three or more polypeptides are encoded in three or more nucleic acids, e.g. each vector encodes one of the polypeptides.

In certain embodiments, the lentiviral vector comprises a promoter selected from the group consisting of beta-globin promoter, murine stem cell virus (MSCV) promoter, Gibbon ape leukemia virus (GALV) promoter, human elongation factor lalpha (EFlalpha) promoter, CAG CMV immediate early enhancer and the chicken beta-actin (CAG), and human phosphoglycerate kinase 1 (PGK) promoter.

Nucleic acids such as DNA expression vectors or mRNA for producing the exogenous polypeptides may be introduced into progenitor cells (e.g., an erythroid cell progenitor or a platelet progenitor and the like) that are suitable to produce the exogenous polypeptides described herein. The progenitor cells can be isolated from an original source or obtained from expanded progenitor cell population via routine recombinant technology as provided herein. In some instances, the expression vectors can be designed such that they can incorporate into the genome of cells by homologous or non-homologous recombination by methods known in the art.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture.

According to some embodiments, one or more exogenous polypeptides may be cloned into plasmid constructs for transfection. Methods for transferring expression vectors into cells that are suitable to produce the engineered erythroid cells described herein include, but are not limited to, viral mediated gene transfer, liposome mediated transfer, transformation, gene guns, transfection and transduction, e.g., viral mediated gene transfer such as the use of vectors based on DNA viruses such as adenovirus, adenoassociated virus and herpes virus, as well as retroviral based vectors. Examples of modes of gene transfer include e.g., naked DNA, CaPO₄ precipitation, DEAE dextran, electroporation, protoplast fusion, lipofection, and cell microinjection.

According to some embodiments, recombinant DNA encoding each exogenous polypeptide may be cloned into a lentiviral vector plasmid for integration into erythroid cells. In some embodiments, the lentiviral vector comprises DNA encoding a single exogenous polypeptide for integration into erythroid cells. For example, in some embodiments, the lentiviral vector comprises DNA encoding a homocysteine reducing polypeptide or a homocysteine degrading polypeptide, e.g., a cystathionine beta-synthase (CBS) polypeptide or a methionine gamma-lyase (MGL) polypeptide, for integration into erythroid cells. In some embodiments, the lentiviral vector comprises DNA encoding an amino acid transporter for integration into erythroid cells. In some embodiments, the lentiviral vector comprises DNA encoding a cystathionine degrading polypeptide for integration into erythroid cells. In other embodiments, the lentiviral vector comprises two, three, four or more exogenous polypeptides as described herein for integration into erythroid cells. For example, in some embodiments, the lentiviral vector comprises DNA encoding a homocysteine degrading polypeptide and a homocysteine transporter or a serine transporter polypeptide for integration into erythroid cells. In some embodiments, the lentiviral vector comprises DNA encoding a homocysteine degrading polypeptide, DNA encoding a homocysteine transporter or a serine transporter polypeptide, and DNA encoding a cystathionine degrading polypeptide for integration into erythroid cells. According to some embodiments, recombinant DNA encoding the one or more exogenous polypeptides may be cloned into a plasmid DNA construct encoding a selectable trait, such as an antibiotic resistance gene. According to some embodiments, recombinant DNA encoding the exogenous polypeptides may be cloned into a plasmid construct that is adapted to stably express each recombinant protein in the erythroid cells.

According to some embodiments, the lentiviral system may be employed where the transfer vector with exogenous polypeptides sequences (e.g., one, two, three, four or more exogenous polypeptide sequences), an envelope vector, and a packaging vector are each transfected into host cells for virus production. According to some embodiments, the lentiviral vectors may be transfected into host cells by any of calcium phosphate precipitation transfection, lipid based transfection, or electroporation, and incubated overnight. For embodiments where the exogenous polypeptide sequence may be accompanied by a fluorescence reporter, inspection of the host cells for florescence may be checked after overnight incubation. The culture medium of the host cells comprising virus particles may be harvested 2 or 3 times every 8-12 hours and centrifuged to sediment detached cells and debris. The culture medium may then be used directly, frozen or concentrated as needed.

A progenitor cell subject to transfer of an exogenous nucleic acid that encodes an exogenous polypeptide can be cultured under suitable conditions allowing for differentiation into mature red blood cells, e.g., the in vitro culturing process described herein. The resulting red blood cells display proteins associated with mature erythrocytes, e.g., hemoglobin, glycophorin A, and exogenous polypeptides which can be validated and quantified by standard methods (e.g., Western blotting or FACS analysis). Isolated mature red blood cells comprising a first exogenous polypeptide, a second exogenous polypeptide, or both a first and a second exogenous polypeptide are non-limiting examples of engineered erythroid cells of the disclosure.

In some embodiments, the engineered erythroid cell is generated by contacting an erythroid precursor cell with an exogenous nucleic acid encoding an exogenous polypeptide. In some embodiments, the exogenous polypeptide is encoded by an RNA which is contacted with an erythroid precursor cell.

Isolated erythroid precursor cells may be transfected with mRNA encoding one or more exogenous polypeptides to generate an engineered erythroid cell. Messenger RNA may be derived from in vitro transcription of a cDNA plasmid construct containing the coding sequence corresponding to the one or more exogenous polypeptides. For example, the cDNA sequence corresponding to the exogenous polypeptide may be inserted into a cloning vector containing a promoter sequence compatible with specific RNA polymerases. For example, the cloning vector ZAP EXPRESS pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the exogenous polypeptide. The mRNA is transcribed from the linear DNA template using a commercially available kit such as, for example, the RNAMAXX High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase I to eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding the one or more exogenous polypeptides may be introduced into reticulocytes using a variety of approaches including, for example, lipofection and electroporation (van Tandeloo et al., Blood 98:49-56 (2001)). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C(Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891 (2001)). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the useful conditions for transfection of a particular mRNA into a reticulocyte. In general, the electroporation parameters required to efficiently transfect cells with mRNA appear to be less detrimental to cells than those required for electroporation of DNA (van Tandeloo et al., Blood 98:49-56 (2001)).

Alternatively, mRNA may be transfected into an erythroid precursor cell using a peptide-mediated RNA delivery strategy (see, e.g., Bettinger et al., Nucleic Acids Res. 29:3882-3891 (2001)). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

In some embodiments, the engineered erythroid cell is generated by contacting a suitable isolated erythroid precursor cell or a platelet precursor cell with an exogenous nucleic acid encoding one or more exogenous polypeptides. In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleated erythroid cell, or a nucleated platelet precursor cell.

The one or more exogenous polypeptides may be genetically introduced into an erythroid precursor cell, a platelet precursor cell, or a nucleated erythroid cell prior to terminal differentiation using a variety of DNA techniques, including transient or stable transfections and gene therapy approaches. The exogenous polypeptides may be expressed on the surface and/or in the cytoplasm of mature red blood cell or platelet.

Viral gene transfer may be used to transfect the cells with DNA encoding one or more exogenous polypeptides. A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example (See, e.g., Osten et al., HEP 178:177-202 (2007)). Retroviruses, for example, efficiently transduce mammalian cells including human cells and integrate into chromosomes, conferring stable gene transfer.

One or more exogenous polypeptides may be transfected into an erythroid precursor cell, a platelet precursor cell, or a nucleated erythroid cell, expressed and subsequently retained and exhibited in a mature red blood cell or platelet. A suitable vector is the Moloney murine leukemia virus (MMLV) vector backbone (Malik et al., Blood 91:2664-2671 (1998)). Vectors based on MMLV, an oncogenic retrovirus, are currently used in gene therapy clinical trials (Hossle et al., News Physiol. Sci. 17:87-92 (2002)). For example, a DNA construct containing the cDNA encoding an exogenous polypeptide can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid precursor cell, a platelet precursor cell, or a nucleated erythroid cell that has been isolated and cultured or has been freshly isolated as described herein. The expression of the exogenous polypeptide may be monitored using FACS analysis (fluorescence-activated cell sorting), for example, with a fluorescently labeled antibody directed against the exogenous polypeptide, if it is located on the surface of the engineered erythroid cell Similar methods may be used to express an exogenous polypeptide that is located in the inside of the engineered erythroid cell.

Optionally, a fluorescent tracking molecule such as, for example, green fluorescent protein (GFP) may be transfected using a viral-based approach (Tao et al., Stem Cells 25:670-678 (2007)). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce e.g., an erythroid precursor cell, a platelet precursor cell, or a nucleated erythroid cell. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al., Leukemia 13:605-613 (1999)).

Nonviral vectors may be used to introduce genetic material into suitable erythroid cells, platelets or precursors thereof to generate engineered erythroid cells described herein. Nonviral-mediated gene transfer differs from viral-mediated gene transfer in that the plasmid vectors contain no proteins, are less toxic and easier to scale up, and have no host cell preferences. The “naked DNA” of plasmid vectors is by itself inefficient in delivering genetic material encoding a polypeptide to a cell and therefore is combined with a gene delivery method that enables entry into cells. A number of delivery methods may be used to transfer nonviral vectors into suitable erythroid cells, platelets or precursors thereof including chemical and physical methods.

A nonviral vector encoding one or more exogenous polypeptides may be introduced into suitable erythroid cells, platelets or precursors thereof using synthetic macromolecules such as cationic lipids and polymers (Papapetrou et al., Gene Therapy 12:S118-S130 (2005)). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al., Gene Therapy 6:931-938 (1999)). For erythroid cells, platelets or precursors thereof the plasmid DNA (approximately 0.5 μg in 25-100 μL of a serum free medium, such as, for example, OptiMEM (Invitrogen, Carlsbad, Calif.)) is mixed with a cationic liposome (approximately 4 μg in 25 μL of serum free medium) such as the commercially available transfection reagent Lipofectamine™ (Invitrogen, Carlsbad, Calif.) and allowed to incubate for at least 20 min to form complexes. The DNA/liposome complex is added to suitable erythroid cells, platelets or precursors thereof and allowed to incubate for 5-24 hours, after which time transgene expression of the polypeptide may be assayed. Alternatively, other commercially available liposome transfection agents may be used (e.g., In vivo GeneSHUTTLE, Qbiogene, Carlsbad, Calif.).

Optionally, a cationic polymer such as, for example, polyethylenimine (PEI) may be used to efficiently transfect erythroid cell progenitor cells, for example hematopoietic and umbilical cord blood-derived CD34+ cells (See, e.g., Shin et al., Biochim. Biophys. Acta 1725:377-384 (2005)). Human CD34+ cells are isolated from human umbilical cord blood and cultured in Iscove's modified Dulbecco's medium supplemented with 200 ng/ml stem cell factor and 20% heat-inactivated fetal bovine serum. Plasmid DNA encoding the exogenous polypeptide is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μl of PEI stock solution contains 10 nmol amine nitrogen. The isolated CD34+ cells are seeded with the DNA/cationic complex, centrifuged at 280×g for 5 min and incubated in culture medium for 4 or more h until gene expression of the polypeptide is assessed.

A plasmid vector may be introduced into suitable erythroid cells, platelets or precursors thereof using a physical method such as particle-mediated transfection, “gene gun”, biolistics, or particle bombardment technology (Papapetrou, et al., (2005) Gene Therapy 12:S118-S130). In this instance, DNA encoding the polypeptide is absorbed onto gold particles and administered to cells by a particle gun. This approach may be used, for example, to transfect erythroid progenitor cells, e.g., hematopoietic stem cells derived from umbilical cord blood (See, e.g., Verma et al., Gene Therapy 5:692-699 (1998)). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+ enriched cells may be cultured as described herein. For transfection, plasmid DNA encoding the polypeptide is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into suitable erythroid cells, platelets or precursors thereof. Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser XCELL, BioRad, Hercules, Calif.; Cellject Duo, Thermo Science, Milford, Mass.). Alternatively, efficient electroporation of isolated CD34+ cells may be performed using the following parameters: 4 mm cuvette, 1600 μF, 550 V/cm, and 10 μg of DNA per 500 μl of cells at 1×10⁵ cells/ml (Oldak et al., Acta Biochimica Polonica 49:625-632 (2002)).

Nucleofection, a form of electroporation, may also be used to transfect suitable erythroid cells, platelets or precursors thereof. In this instance, transfection is performed using electrical parameters in cell-type specific solutions that enable DNA (or other reagents) to be directly transported to the nucleus thus reducing the risk of possible degradation in the cytoplasm. For example, a Human CD34 CELL NUCLEOFECTOR Kit (from Amaxa Inc.) may be used to transfect suitable erythroid cells, platelets or precursors thereof. In this instance, 1-5×10⁶ cells in Human CD34 Cell NUCLEOFECTOR Solution are mixed with 1-5 μg of DNA and transfected in the NUCLEOFECTOR instrument using preprogrammed settings as determined by the manufacturer.

Erythroid cells, platelets or precursors thereof may be non-virally transfected with a conventional expression vector which is unable to self-replicate in mammalian cells unless it is integrated in the genome. Alternatively, erythroid cells, platelets or precursors thereof may be transfected with an episomal vector which may persist in the host nucleus as autonomously replicating genetic units without integration into chromosomes (Papapetrou et al., Gene Therapy 12:S118-S130 (2005)). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al., Exp. Hematol. 33:1470-1476 (2005)).

Exogenous nucleic acids encoding one or more exogenous polypeptides may be assembled into expression vectors by standard molecular biology methods known in the art, e.g., restriction digestion, overlap-extension PCR, and Gibson assembly.

Exogenous nucleic acids may comprise a gene encoding one or more exogenous polypeptides that are not normally expressed on the cell surface, e.g., of an erythroid cell, fused to a gene that encodes an endogenous or native membrane protein, such that the exogenous polypeptide is expressed on the cell surface. For example, an exogenous gene encoding an exogenous polypeptide can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly4Ser]3 (SEQ ID NO: 63) commonly used in generating single-chain antibody fragments from full-length antibodies (Antibody Engineering: Methods & Protocols, Lo 2004), or ala-gly-ser-thr polypeptides such as those used to generate single-chain Arc repressors (Robinson & Sauer, PNAS 1998). In some embodiments, the flexible linker provides the polypeptide with more flexibility and steric freedom than the equivalent construct without the flexible linker.

An epitope tag may be placed between two fused genes, such as, e.g., a nucleic acid sequence encoding an HA epitope tag—amino acids YPYDVPDYA (SEQ ID NO: 64), a CMyc tag—amino acids EQKLISEEDL (SEQ ID NO: 65), or a Flag tag—amino acids DYKDDDDK (SEQ ID NO: 66). The epitope tag may be used for the facile detection and quantification of expression using antibodies against the epitope tag by flow cytometry, western blot, or immunoprecipitation.

In some embodiments, the engineered erythroid cell comprises one or more exogenous polypeptides and at least one other heterologous polypeptide. The at least one other heterologous polypeptide can be a fluorescent protein. The fluorescent protein can be used as a reporter to assess transduction efficiency. In some embodiments, the fluorescent protein is used as a reporter to assess expression levels of the exogenous polypeptide if both are made from the same transcript. In some embodiments, the at least one other polypeptide is heterologous and provides a function, such as, e.g., multiple antigens, multiple capture targets, enzyme cascade.

In certain embodiments, the engineered erythroid cell is a cell that presents a first exogenous polypeptide that is conjugated with a second exogenous polypeptide. Conjugation may be achieved chemically or enzymatically. Chemical conjugation may be accomplished by covalent bonding of the exogenous antigen-presenting polypeptide to one or more exogenous polypeptides, with or without the use of a linker. Chemical conjugation may be accomplished by the covalent bonding of an exogenous polypeptide and a binding pair member, with or without the use of a linker. Chemical conjugation may be accomplished by the covalent bonding of a coinhibitory polypeptide and a binding pair member, with or without the use of a linker. The formation of such conjugates is within the skill of artisans and various techniques are known for accomplishing the conjugation, with the choice of the particular technique being guided by the materials to be conjugated. The addition of amino acids to the polypeptide (C- or N-terminal) which contain ionizable side chains, e.g., aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, e.g., homo- or hetero-bi-functional PEG (e.g., Lutolf and Hubbell, Biomacromolecules 2003; 4:713-22, Hermanson, Bioconjugate Techniques, London. Academic Press Ltd; 1996).

Other molecular fusions may be formed between exogenous polypeptides and include direct or indirect conjugation. The exogenous polypeptides may be directly conjugated to each other or indirectly through a linker. The linker may be a peptide, a polymer, an aptamer, or a nucleic acid. The polymer may be, e.g., natural, synthetic, linear, or branched. Exogenous polypeptides can comprise a heterologous fusion protein that comprises a first polypeptide and a second polypeptide with the fusion protein comprising the polypeptides directly joined to each other or with intervening linker sequences and/or further sequences at one or both ends. The conjugation to the linker may be through covalent bonds or ionic bonds.

Erythroid cells described herein can also be produced using coupling reagents to link an exogenous polypeptide to a cell. For instance, click chemistry can be used. Thus, in some embodiments, any one or combination of exogenous polypeptides described herein may be conjugated onto the surface of an erythroid cell (e.g., an enucleated erythroid cell) using click chemistry. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the erythroid cells. Click chemistry and other conjugation methods for functionalizing erythroid cells is described in International Application No. PCT/US2018/000042, which claims priority U.S. Provisional Application No. 62/460589, filed Feb. 17, 2017 and U.S. Provisional Application No. 62/542142, filed Jul. 8, 2017, incorporated by reference in their entireties herein.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an exogenous polypeptide coupled to a second coupling reagent e.g., under conditions suitable for reaction of the first coupling reagent with the second coupling reagent. In embodiments, two or more exogenous polypeptides are coupled to the cell (e.g., using click chemistry). In embodiments, a first exogenous polypeptide is coupled to the cell (e.g., using click chemistry) and a second exogenous polypeptide comprises a polypeptide expressed from an exogenous nucleic acid.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (https://clickchemistrytools.com/) or Lahann, J (ed) (2009) Click Chemistry for Biotechnology and Materials Science, each of which is incorporated herein by reference in its entirety.

In another embodiment, the exogenous polypeptide is attached to an erythroid cell via a covalent attachment to generate an engineered erythroid cell comprising an erythroid cell presenting, e.g. comprising on the cell surface, one or more exogenous polypeptides (e.g. a first exogenous polypeptide and a second exogenous polypeptide). For example, the exogenous polypeptide may be derivatized and bound to the erythroid cell or platelet using a coupling compound containing an electrophilic group that will react with nucleophiles on the erythroid cell or platelet to form the interbonded relationship. Representative of these electrophilic groups are αβ unsaturated carbonyls, alkyl halides and thiol reagents such as substituted maleimides. In addition, the coupling compound can be coupled to an exogenous polypeptide via one or more of the functional groups in the polypeptide such as amino, carboxyl and tyrosine groups. For this purpose, coupling compounds should contain free carboxyl groups, free amino groups, aromatic amino groups, and other groups capable of reaction with enzyme functional groups. Highly charged exogenous polypeptides can also be prepared for immobilization on, e.g., erythroid cells or platelets through electrostatic bonding to generate an engineered erythroid cell. Examples of these derivatives would include polylysyl and polyglutamyl enzymes.

The choice of the reactive group embodied in the derivative depends on the reactive conditions employed to couple the electrophile with the nucleophilic groups on the erythroid cell or platelet for immobilization. A controlling factor is the desire not to inactivate the coupling agent prior to coupling of the exogenous polypeptide immobilized by the attachment to the erythroid cell or platelet. Such coupling immobilization reactions can proceed in a number of ways. Typically, a coupling agent can be used to form a bridge between the exogenous polypeptide and the erythroid cell or platelet. In this case, the coupling agent should possess a functional group such as a carboxyl group which can be caused to react with the exogenous polypeptide. One way of preparing the exogenous polypeptide for conjugation includes the utilization of carboxyl groups in the coupling agent to form mixed anhydrides which react with the exogenous polypeptide, in which use is made of an activator which is capable of forming the mixed anhydride. Representative of such activators are isobutylchloroformate or other chloroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the exogenous polypeptide to yield the reactive derivative which in turn can react with nucleophilic groups on the erythroid cell or platelet to immobilize the exogenous polypeptide.

Functional groups on an exogenous polypeptide, such as carboxyl groups can be activated with carbodiimides and the like activators. Subsequently, functional groups on the bridging reagent, such as amino groups, will react with the activated group on the polypeptide to form the reactive derivative. In addition, the coupling agent should possess a second reactive group which will react with appropriate nucleophilic groups on the erythroid cell or platelet to form the bridge. Typical of such reactive groups are alkylating agents such as iodoacetic acid, αβ unsaturated carbonyl compounds, such as acrylic acid and the like, thiol reagents, such as mercurials, substituted maleimides and the like.

Alternatively, functional groups on the exogenous polypeptide can be activated so as to react directly with nucleophiles on, e.g., erythroid cells or platelets to obviate the need for a bridge-forming compound. For this purpose, use is made of an activator such as Woodward's Reagent K or the like reagent which brings about the formation of carboxyl groups in the exogenous polypeptide into enol esters, as distinguished from mixed anhydrides. The enol ester derivatives of exogenous polypeptides subsequently react with nucleophilic groups on, e.g., an erythroid cell or platelet to effect immobilization of the exogenous polypeptide, thereby creating an engineered erythroid cell.

In some embodiments, the engineered erythroid cell comprising an exogenous polypeptide (e.g. a first and/or a second exogenous polypeptide) is generated by contacting an erythroid cell with an exogenous polypeptide and optionally a payload, wherein contacting does not include conjugating the exogenous polypeptide to the erythroid cell using an attachment site comprising Band 3 (CD233), aquaporin-1, Glut-1, Kidd antigen, RhAg/R1i50 (CD241), R1i (CD240), Rh30CE (CD240CE), Rh30D (CD240D), Kx, glycophorin B (CD235b), glycophorin C (CD235c), glycophorin D (CD235d), Kell (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scianna, MER2, stomatin, BA-1 (CD24), GPIV (CD36), CD108, CD139, or H antigen (CD173).

In some embodiments, the engineered erythroid cell comprises an erythroid cell presenting, e.g. comprising on the cell surface, one or more exogenous polypeptides, wherein the one or more exogenous polypeptides are enzymatically conjugated onto the cell.

In specific embodiments, the exogenous polypeptide can be conjugated to the surface of, e.g., an erythroid cell or platelet by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme to connect one polypeptide containing the acceptor sequence LPXTG (SEQ ID NO: 67) or LPXTA (SEQ ID NO: 68) with a polypeptide containing the N-terminal donor sequence GGG, see e.g., Swee et al., PNAS 2013. The methods also include combination methods, such as e.g., sortase-mediated conjugation of Click Chemistry handles (an azide and an alkyne) on the antigen and the cell, respectively, followed by a cyclo-addition reaction to chemically bond the antigen to the cell, see e.g., Neves et al., Bioconjugate Chemistry, 2013. Sortase-mediated modification of proteins is described in International Application No. PCT/US2014/037545 and International Application No. PCT/US2014/037554, both of which are incorporated by reference in their entireties herein.

In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on or in e.g., an erythroid cell or platelet, either intracellularly or extracellularly. Many catalytic bond-forming polypeptides exist, including transpeptidases, sortases, and isopeptidases, including those derived from Spy0128, a protein isolated from Streptococcus pyogenes.

In some embodiments, any of the polypeptides described herein are not conjugated to the cell using a sortase.

It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Asp117 on SpyTag and Lys31 on SpyCatcher (Zakeri and Howarth, JACS 2010, 132:4526). The reaction is compatible with the cellular environment and highly specific for protein/peptide conjugation (Zakeri, B.; Fierer, J. O.; Celik, E.; Chittock, E. C.; Schwarz-Linek, U.; Moy, V. T.; Howarth, M. Proc. Natl. Acad. Sci. U.S.A. 2012, 109, E690-E697). SpyTag and SpyCatcher has been shown to direct post-translational topological modification in elastin-like protein. For example, placement of SpyTag at the N-terminus and SpyCatcher at the C-terminus directs formation of circular elastin-like proteins (Zhang et al, Journal of the American Chemical Society, 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this document, when SpyTag and SpyCatcher are used, it is to be understood that the complementary molecule could be substituted in its place.

A catalytic bond-forming polypeptide, such as a SpyTag/SpyCatcher system, can be used to attach the exogenous polypeptide to the surface of a cell, e.g., an erythroid cell, to generate an engineered erythroid cell. The SpyTag polypeptide sequence can be expressed on the extracellular surface of the erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. The nucleic acid sequence encoding the SpyTag fusion can be expressed within an engineered erythroid cell. An exogenous polypeptide can be fused to SpyCatcher. The nucleic acid sequence encoding the SpyCatcher fusion can be expressed and secreted from the same erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous polypeptide to the surface of the erythroid cell to form an engineered erythroid cell.

In some embodiments, the SpyTag polypeptide may be expressed as a fusion to the N terminus of glycophorin A under the control of the Gata1 promoter in an erythroid cell. An exogenous polypeptide, fused to the SpyCatcher polypeptide sequence can be expressed under the control of the Gata1 promoter in the same erythroid cell. Upon expression of both fusion polypeptides, an isopeptide bond will be formed between the SpyTag and SpyCatcher polypeptides, forming a covalent bond between the erythroid cell surface and the exogenous polypeptide.

In another embodiment, the SpyTag polypeptide may be expressed as a fusion to the N terminus of glycophorin A under the control of the Gata1 promoter in an erythroid cell. An exogenous polypeptide fused to the SpyCatcher polypeptide sequence can be expressed in a suitable mammalian cell expression system, for example HEK293 cells. Upon expression of the SpyTag fusion polypeptide on the erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid cell surface and the exogenous polypeptide.

Exogenous polypeptides can be detected on the engineered erythroid cells. The presence of the exogenous polypeptide can be validated and quantified using standard molecular biology methods, e.g., Western blotting or FACS analysis. Exogenous polypeptides present in the intracellular environment may be quantified upon cell lysis or using fluorescent detection.

In some embodiments of the above aspects and embodiments, the engineered erythroid cell is an enucleated cell, e.g., erythrocyte or reticulocyte. In some embodiments of the above aspects and embodiments, the engineered erythroid cell is a nucleated cell.

### III. Methods of Use

The present disclosure provides methods of treating or preventing homocystinuria in a subject, comprising administering to the subject the engineered erythroid cell as described herein, in an amount effective to treat or prevent homocystiuria in the subject.

The present disclosure also provides methods of reducing homocysteine concentrations in a subject (e.g., in the blood, plasma or serum of the subject), comprising administering to the subject the engineered erythroid cell as described herein, in an amount effective to reduce homocysteine concentrations in the subject. In some embodiments, the subject has or is at risk of developing homocystinuria. In some embodiments, the subject has or is at risk of developing B6 (also known as pyroxidine)-responsive homocystinuria. In some embodiments, the subject has or is at risk of developing B6-non-responsive homocystinuria.

In some embodiments, the disclosure provides methods of treating or preventing a symptom or condition associated with homocystinuria in a subject, comprising administering to the subject an engineered erythroid cell as described herein. In some embodiments, the symptom or condition is selected from failure to thrive, an eye disorder (e.g. myopia (nearsightedness), dislocation of the lens of the eye (ectopia lentis), glaucoma, optic atrophy, retinal detachment, or cataracts), neurodegeneration, a psychiatric disorder, developmental delay, or intellectual disability, a central nervous system disorder (e.g. mental retardation or seizures), aberrant musculoskeletal development (e.g. Marfanoid features (e.g., marganoid habitus) or excessive height), a skeletal disorder (e.g. pectus excavatum, carinatum, genu valgum, scoliosis, kyphoscoliosis, or osteoporosis), cardiovascular disease (e.g. hypertension, myocardial infarction, or thromboembolism), stroke, vitamin B12 deficiency, folate deficiency, and renal failure.

In some embodiments, the engineered erythroid cells described herein may be used to reduce concentrations of homocysteine in a subject, e.g., in the plasma or serum of the subject. Homocysteine concentration may be reduced in a subject (e.g., in the blood, plasma, or serum of the subject) by administering the engineered erythroid cells described herein below about 5%, 10%, 15%, 20%, 25%, 30%, 33%, 35%, 40%, 45%, 50%, 52% 55%, 60%, 65%, 67%, 69%, 70%, 74%, 75%, 76%, 77%, 80%, 85%, 90%, 95% or more than 95%, as compared to the homocysteine concentration in the subject (e.g., in the blood, plasma, or serum of the subject) prior to administration of the engineered erythroid cells.

In one embodiment, the engineered erythroid cells described herein may be used to cause a change such as an increase or a decrease in any one or more of the metabolites of homocysteine in the subject, e.g., in the plasma or serum of the subject. As a non-limiting example, homocysteine metabolites include, e.g., disulfide homocystine (Hcy-S—S-Hcy), mixed disulfide of Hcy and Cys (Hcy-S—S-Cys), mixed disulfide of Hcy with plasma protein (S-Hcy-protein), Hcy-thiolactone, N-Hcy-protein, Nε-Hcy-Lys, AdoHcy, cystathionine, homocysteine sulfinic acid, homocysteic acid, and methionine, any of which may be decreased by 5%, 10%, 15%, 20%, 25%, 30%, 33%, 35%, 40%, 45%, 50%, 52% 55%, 60%, 65%, 67%, 69%, 70%, 74%, 75%, 76%, 77%, 80%, 85%, 90%, 95%, 99% or 100% compared to an untreated subject or to the same subject prior to administration of an engineered cell (or population of engineered cells described herein).

The present disclosure also provides methods of reducing the level of methionine in a subject, e.g., in the plasma or serum of the subject, comprising administering to the subject the engineered erythroid cell as described herein, in an amount effective to reduce the level of methionine in the subject. Methionine may be reduced below about 5%, 10%, 15%, 20%, 25%, 30%, 33%, 35%, 40%, 45%, 50%, 52% 55%, 60%, 65%, 67%, 69%, 70%, 74%, 75%, 76%, 77%, 80%, 85%, 90%, 95% or more than 95% in a subject using the engineered erythroid cells described herein.

The present disclosure also relates to methods of regulating biological processes, including cystathionine production, by regulating the expression and/or activity of a homocysteine degrading polypeptide. This embodiment can generally include the use (e.g., administration) of engineered erythroid cells described herein, e.g., cells comprising a first exogenous polypeptide comprising a homocysteine reducing polypeptide, or a variant thereof. This embodiment can generally include the use (e.g., administration) of engineered erythroid cells described herein, comprising a first exogenous polypeptide comprising a homocysteine degrading polypeptide, or variant thereof, optionally a second exogenous polypeptide comprising a homocysteine transporter, or variants thereof, and/or a third polypeptide comprising a serine transporter, or variants thereof. The therapeutic compositions comprising the engineered erythroid cells described herein are useful in a method of regulating the production of cystathionine, mediated by or associated with, the expression and biological activity of a homocysteine degrading polypeptide.

The presence and level of homocysteine in a biological sample (e.g., plasma) can be detected and measured using a wide variety of means, including techniques to quantitate total homocysteine (Hcy) and methods for distinguishing between the free (reduced and disulfide) and protein-bound (primarily albumin) forms. Such methods are described, for example, in Ueland, et al., Clin. Chem. 39(9):1764-1779 (1993), which is incorporated by reference in its entirety herein. An enzymatic assay for homocysteine is described in U.S. Pat. No. 6,063,581, where homocysteine is assayed indirectly by measuring the product concentration following the enzyme catalyzed conversion of homocysteine to S-adenosyl homocysteine. High performance liquid chromatographic (“HPLC”) methods for Hcy and Cys are also known in the art. This analytical method discriminates between Hcy and Cys by differential adsorption and elution of the compounds on a chromatographic support. Andersson, et al., (1993) Clin. Chem. 39(8):1590-1597 describes the determination of total, free and reduced Hcy and Cys. Hcy and Cys analysis by means of a gas chromatograph-mass spectrometer is described in U.S. Pat. No. 4,940,658. PCT/US92/05727 describes a chromatographic assay for cystathionine, the intermediary amino acid between Hcy and Cys produced in the metabolism of methionine. Fiskerstrand, et al., Clin. Chem. 39(2):263-271 (1993) describes a fully automated analysis of total Hcy involving fluorescent labeling of serum thiols, followed by chromatographic separation of the Hcy derivative from the other sulfur-containing compounds.

Identification of homocysteine by HPLC methods often involves derivatization with fluorescent reagents, or a radioenzymatic technique (Refsum, et al., (1985) Clin. Chem. 31(4) 624-628). In addition, identification of Cys by protein sequence analysis involves derivatization with alkylating reagents (Jue, et al., (1993) Analytical Biochemistry 210:39-44).

Techniques for handling undesirable cross-reactants are known and described in the art, for example, in U.S. Pat. No. 4,952,336, which describes a method of pre-treating a sample with an aqueous periodate solution to eliminate cross-reactants in an amphetamine-methamphetamine immunoassay. PCT/GB90/01649 discloses to an immunoassay wherein the level of interference from rheumatoid factor is reduced by pretreating the sample with a reducing agent. U.S. Pat. No. 4,978,632 discloses an immunoassay wherein the level of interference from blood and blood products is eliminated by pretreating the sample with an oxidizing agent. These pretreatment methods only affect the cross-reactants; none of the methods affect the analyte.

Treatment of Conditions that Would Benefit From Degradation of Homocysteine (Hcy) and its Metabolites

Methods of administering engineered erythroid cells comprising (e.g. presenting) an exogenous agent (e.g. polypeptides) are described, e.g., in WO2015/073587 and WO2015/153102, each of which is incorporated by reference in its entirety.

In some embodiments, the engineered erythroid cells (e.g., engineered enucleated cells) described herein are administered to a subject, e.g., a mammal, e.g., a human. Exemplary mammals that can be treated include without limitation, humans, domestic animals (e.g. dogs, cats and the like), farm animals (e.g. cows, sheep, pigs, horses and the like) and laboratory animals (e.g. monkey, rats, mice, rabbits, guinea pigs and the like). The methods described herein are applicable to both human therapy and veterinary applications.

In one aspect, the present disclosure provides a method of treating or preventing homocystinuria in a subject, comprising administering to the subject an engineered erythroid cell as described herein (e.g. an engineered erythroid cell comprising a homocysteine degrading enzyme, e.g. CBS, optionally in combination with a homocysteine and/or serine transporter) in an amount effective to treat or prevent homocystinuria in the subject.

In one embodiment, the present disclosure provides a method of treating or preventing homocystinuria in a subject, comprising administering to the subject an engineered erythroid cell as described herein (e.g. an engineered erythroid cell comprising a homocysteine degrading enzyme, e.g. CBS, optionally in combination with a homocysteine and/or serine transporter) in an amount effective to treat or prevent symptomatic homocystinuria in the subject.

In another embodiment, the present disclosure provides a method of treating or preventing homocystinuria in a subject who is non-responsive to B6 therapy, comprising administering to the subject an engineered erythroid cell as described herein (e.g. an engineered erythroid cell comprising a homocysteine degrading enzyme, e.g. CBS, optionally in combination with a homocysteine and/or serine transporter) in an amount effective to treat or prevent homocystinuria in the subject.

### Homocystinuria

Homocystinuria refers to a group of disorders that result in high levels of circulating homocysteine (Hcy) and its metabolites (e.g. homocysteine-cysteine complex etc.). The majority of homocystinuria patients have one or more mutations in a gene that regulates the production of cystathionine beta-synthase (CBS). However, homocystinuria may also be caused by factors other than CBS deficiency, as described below. A buildup of homocysteine results in a wide range of deforming and debilitating symptoms. By age three, failure to thrive is generally apparent, and partial dislocation of the lens of the eyes and severe myopia are common. As with most of the inborn errors of metabolism (e.g., phenylketonuria (PKU) that results in decreased metabolism of the amino acid phenylalanine) without treatment children may be affected by progressive and severe neurodegeneration. Many will develop psychiatric disturbances and seizures. A failure to effectively treat patients over time can also result in aberrant musculoskeletal development including Marfanoid features (characterized by abnormally long limbs and digits) and scoliosis (spinal curvature). Perhaps most concerning however is that affected individuals suffer from extreme hypertension and are at an elevated risk for the development of thromboembolisms. If untreated, approximately 50% of patients will have a thromboembolic event and the overall mortality rate is approximately 20% by age 30 (death is predominantly due to cerebrovascular or cardiovascular causes). It is not unusual for a previously undiagnosed individual to present in adult years with only a thromboembolic event.

A. CBS-Deficient Homocystinuria (CBSDH)

Deficiency of CBS is the most common cause of inherited homocystinuria, a serious life threatening disease that results in severely elevated homocysteine levels in plasma, tissues and urine. The primary health problems associated with CBS-deficient homocystinuria (CBSDH) include: cardiovascular disease with a predisposition to thrombosis, resulting in a high rate of mortality in untreated and partially treated patients; connective tissue problems affecting the ocular system with progressive myopia and lens dislocation; connective tissue problems affecting the skeleton characterized by marfanoid habitus, osteoporosis, and scoliosis; and central nervous system problems, including mental retardation and seizures. See U.S. Pat. No. 9,447,406, which is incorporated herein by reference in its entirety.

The therapeutic resolution of CBS-associated homocystinuria is dependent upon the type of mutations present in the CBS gene. Approximately 160 pathogenic mutations of the CBS gene have been identified to date in humans. There are three groups of pathogenic mutations associated with CBSDH. One group of mutations is classified as “pyridoxine-responsive,” where CBS enzyme function can be restored by high dose Vitamin B6 therapy. This treatment can be effective, but does not always mitigate the pathological events in these patients, and some of the events occur even in these patients over time. The second group of functional mutations is represented by the “C-terminal CBS mutants” that are defective in their ability to respond to post-translational up-regulation by S-adenosylmethionine. Patients with this class of mutations usually lack the mental retardation and connective tissue aspects of the phenotype. This class is detected after measurement of plasma Hcy levels following an idiopathic thrombotic event before the age of 40 years (Maclean et al., 2002, Hum Mutat. 19: 641-55). The final group of CBSDH mutations is “classical homocystinuria,” which represents the most severe form of the disease. For these latter two groups of patients, Vitamin B6 therapy in isolation does not effectively lower serum Hcy levels.

The pathophysiology of homozygous CBS deficiency is complex, but there is a consensus that the fundamental instigator of end-organ injury is an extreme elevation of serum Hcy. The toxicity of profound elevations in blood and tissue concentrations of Hcy may ensue from the molecular reactivity and biological effects of Hcy per se or from its metabolites (e.g., Hcy-thiolactone) that affect a number of biological processes (Jakubowski et al., 2008, FASEB J 22: 4071-6). Abnormalities in chronic platelet aggregation, changes in vascular parameters, and endothelial dysfunction have all been described in patients with homocystinuria.

Confirmation of CBS deficiency cannot be based on a single method as each technique gives normal results in some patients with CBS deficiency. The gold standard for confirming CBS deficiency is generally considered to be the determination of cystathionine production from Hcy and serine in cultured fibroblasts using radioactive or deuterium labelled substrates (Kraus, 1987, Methods Enzymol. 143:388-394). The fibroblast CBS activity may, however, be normal in mild forms of the disease, despite biochemical and clinical abnormalities and mutations in the CBS gene. In one study, three of 13 CBS deficient patients had normal CBS activity in fibroblasts (Mendes et al., 2013, J Inherit Metab Dis 37:245-54). Two rapid stable isotope assays have been developed for measuring activity of CBS released from liver and other organs into plasma (Krijt et al., 2011, J Inherit Metab Dis. 2011; 34:49-55; and Alcaide et al., 2015, Clin Chim Acta. 438:261-265). Studies on patients with 27 different genotypes showed that sensitivity of the plasma assay was 100% for detecting pyridoxine non-responsive patients but only 86% for the pyridoxine responders. Sequencing of the CBS gene is considered the gold standard in molecular diagnostics; however, pathogenic variants may not be detected in one of the parental alleles in up to 7-10% of CBS deficient patients (Magner et al., 2011, J Inherit Metab Dis. 34:33-37). In summary, if one of these techniques (enzyme or DNA analysis) does not confirm a diagnosis of CBS deficiency, the other test should be done in a patient with metabolite abnormalities suggestive of this disease.

B. Other Causes of Homocystinuria

Increased plasma tHcy concentrations are not specific for CBS deficiency, as there are many other genetic, nutritional and pharmacological factors as well as several diseases associated with tHcy elevation (Refsum et al., 2004, J Pediatr. 144:830-832). Confirmation of CBS deficiency should be accompanied by excluding other causes of hyperhomocysteinemia; the balance of these two approaches depends on the degree of clinical suspicion of CBS deficiency.

Nutritional causes of hyperhomocysteinemia are common, notably vitamin B12 deficiency and, less often, folate deficiency. These deficiencies may be identified by measuring serum vitamin B12 and/or transcobalamin II, plasma or urine methylmalonic acid and serum folates (Refsum et al., 2004, cited above). Patients with vitamin B12 deficiency can have tHcy up to 450 μmol/L (Stabler, 2013, N Engl J Med. 368:2041-2042). Folate deficiency is particularly likely to cause elevated tHcy concentrations in subjects who are homozygous for the common c.677C>T variant in the MTHFR gene. Renal failure is another frequent cause of hyperhomocysteinemia and can be identified by measuring the serum creatinine concentration. The patient's history is also important as it may reveal other diseases associated with hyperhomocysteinemia or the administration of drugs such as nitrous oxide, methotrexate and other folate antagonists (Rasmussen et al., 2000, Ann Clin Biochem. 37:627-648; Refsum et al., 2004, cited above).

Analysis of additional metabolites can usually distinguish CBS deficiency from genetic and nutritional disorders in the Hcy remethylation pathway. Low normal or decreased plasma Met concentrations and elevated plasma cystathionine (determined by LC-MS/MS or GC-MS) indicate a disturbance in the remethylation pathway; simultaneous elevation of methylmalonic acid in plasma and/or urine suggests more specifically disorders of vitamin B12 supply, transport or intracellular metabolism with impaired synthesis of both methylcobalamin and adenosylcobalamin (Stabler et al., 2013, JIMD Rep. 11:149-163).

### Diagnosis and Selection of Subjects for Treatment

In some embodiments, subjects that have been diagnosed with homocystinuria are treated using the methods described herein. Methods for diagnosing homocystinuria are known in the art and are described, for example, in Moris et al. (2017) J. Inherit. Metab. Dis. 40: 49-74 and Huemer et al. (2015) J. Inherit. Metab. Dis. 38: 1007-19, each incorporated herein by reference. For example, homocystinuria caused by a CBS deficiency results in increased homocysteine and increased methionine in a subject.

Homocystinuria in neonatal subjects can be diagnosed by detecting increased methionine concentration in dried blood spots from the subjects using tandem mass spectrometry (MS/MS). For example, methionine concentration in the blood above between 39 and 67 μmol/L (e.g., 39 μmol/L, 40 μmol/L, 45 μmol/L, 50 μmol/L, 55 μmol/L, 60 μmol/L, 65 μmol/L, or 67 μmol/L) is considered abnormal and may be used to diagnose homocystinuria. Thus, in some embodiments, a subject (e.g., a neonatal subject) may be selected for treatment using the engineered erythroid cells provided herein by detecting a blood methionine concentration in the subject (e.g., using tandem mass spectrometry) above between about 39 and about 67 μmol/L (e.g., about 39 μmol/L, about 40 μmol/L, about 45 μmol/L, about 50 μmol/L, about 55 μmol/L, about 60 μmol/L, about 65 μmol/L, or about 67 μmol/L). In some embodiments of the methods described herein, a subject having a blood methionine concentration above about 39 μmol/L, about 40 μmol/L, about 45 μmol/L, about 50 μmol/L, about 55 μmol/L, about 60 μmol/L, about 65 μmol/L, or about 67 μmol/L, is selected for administration of an engineered erythroid cell provided herein.

Alternatively, homocystinuria in neonatal subjects can be diagnosed by detecting the total homocysteine concentration in a dried blood spot from the subjects using liquid chromatography with tandem mass spectrometry (LC-MS-MS) using a modified stable isotope dilution technique, as described, e.g., in Gempel et al. (2000) Clin. Chem. 46: 122-123, and Gan-Schreier et al. (2010) J. Pediatr. 156(3): 427-32, each incorporated herein by reference. Total homocysteine concentration in the blood above 10 μmol/L is considered abnormal and may be used to diagnose homocystinuria. Thus, in some embodiments, a subject (e.g., a neonatal subject) may be selected for treatment using the engineered erythroid cells provided herein by detecting a blood total homocysteine concentration in the subject (e.g., LC-MS-MS) above about 10 μmol/L. In some embodiments of the methods described herein, a subject having a blood total homocysteine concentration above about 10 μmol/L is selected for administration of an engineered erythroid cell provided herein.

Homocystinuria (e.g., classic homocystinuria) can also be diagnosed by measuring total homocysteine concentration in the plasma from a subject. Preferably, the total homocysteine concentration is determined using a biological sample (e.g., a blood, plasma or serum sample) from a subject that has not received pyroxidine supplementation for the two-week (i.e., 14 days) preceding the collection of the biological sample from the subject. For example, in neonatal subjects, a plasma total homocysteine concentration from about 50 μmol/L to greater than 100 μmol/L is considered abnormal and can be used to diagnose homocystinuria (see, e.g., Sacharow et al. 2004). In non-neonatal subjects (e.g., adult subjects), a plasma total homocysteine concentration above 100 μmol/L is considered abnormal and can be used to diagnose homocystinuria. In contrast, normal subjects (e.g., neonatal and non-neonatal subjects) have a plasma total homocysteine concentration below 15 μmol/L. Thus, in some embodiments, a subject (e.g., a neonatal subject) may be selected for treatment using the engineered erythroid cells provided herein by detecting a plasma total homocysteine concentration in the subject above about 50 μmol/L. In some embodiments of the methods described herein, a subject (e.g., a neonatal subject) having a plasma total homocysteine concentration above about 50 μmol/L is selected forIadministration of an engineered erythroid cell provided herein. In some embodiments, a subject (e.g., a non-neonatal subject (e.g., an adult subject)) may be selected for treatment using the engineered erythroid cells provided herein by detecting a plasma total homocysteine concentration in the subject above about 100 μmol/L. In some embodiments of the methods described herein, a subject (e.g., a non-neonatal subject (e.g., an adult subject)) having a plasma total homocysteine concentration above about 100 μmol/L is selected for administration of an engineered erythroid cell provided herein.

In addition, homocystinuria (e.g., classic homocystinuria) can be diagnosed by measuring methionine concentration in the plasma from a subject. Preferably, the total homocysteine concentration is determined using a biological sample (e.g., a blood, plasma or serum sample) from a subject that has not received pyroxidine supplementation for the two-week (i.e., 14 days) preceding the collection of the biological sample from the subject. In neonatal subjects, a plasma methionine concentration from about 200 to about 1500 μmol/L (from about 3 to about 23 mg/dL) is considered abnormal and can be used to diagnose homocystinuria (see, e.g., Sacharow et al. 2004). In non-neonatal subjects (e.g., adult subjects), a plasma methionine concentration above 50 μmol/L (above 0.7 mg/dL) is considered abnormal and can be used to diagnose homocystinuria. In contrast, normal subjects (e.g., neonatal and non-neonatal subjects) have a plasma methionine concentration between about 10 to about 40 μmol/L (0.2-0.6 mg/dL). Thus, in some embodiments, a subject (e.g., a neonatal subject) may be selected for treatment using the engineered erythroid cells provided herein by detecting a plasma methionine concentration in the subject between about 200 to about 1500 μmol/L (between about 3 to about 23 mg/dL). In some embodiments of the methods described herein, a subject (e.g., a neonatal subject) having a plasma methionine concentration between about 200 to about 1500 μmol/L (between about 3 to about 23 mg/dL) is selected forIadministration of an engineered erythroid cell provided herein. In some embodiments, a subject (e.g., a non-neonatal subject (e.g., an adult subject)) may be selected for treatment using the engineered erythroid cells provided herein by detecting a plasma methionine concentration in the subject above about 50 μmol/L (above about 0.7 mg/dL). In some embodiments of the methods described herein, a subject (e.g., a non-neonatal subject (e.g., an adult subject)) having a plasma methionine concentration above about 50 μmol/L (above about 0.7 mg/dL) is selected for administration of an engineered erythroid cell provided herein.

Homocystinuria caused by a CBS deficiency is inherited in an autosomal recessive manner. The human CBS gene has been mapped to 21q22.3. Over 160 mutations in the CBS gene have been identified (see, e.g., the CBS mutation database available on the world wide web at cbs.lf1.cuni.cz). The most common pathogenic variants of the CBS gene result in the following amino acid residue substitutions: I1e278Thr (e.g., caused by the nucleotide change c.833T>C), Gly307Ser (e.g., caused by the nucleotide change c919G>A), and Arg336Cys (caused by the nucleotide change c.1006C>T). Methods for analyzing the genotype of a subject are known in the art and include, for example, next-generation (NGS)/massively parallel sequencing (MPS), and bi-directional Sanger sequence analysis. In some embodiments of the methods described herein, the subject comprises at least one allele comprising a pathogenic variant of a CBS gene. In some embodiments of the methods described herein, the subject is homozygote for a pathogenic variant of a CBS gene. In some embodiments of the methods described herein, the subject is homozygote for an allele comprising a mutation causing the CBS amino acid residue substitution I1e278Thr. In some embodiments of the methods described herein, the subject is homozygote for an allele comprising a mutation causing the CBS amino acid residue substitution Gly307Ser. In some embodiments of the methods described herein, the subject is homozygote for an allele comprising a mutation causing the CBS amino acid residue substitution Arg336Cys.

In some embodiments, administration of the engineered erythroid cells as described herein to a subject reduces the level of plasma tHcy in the subject. Normal levels of plasma total homocysteine (tHcy) in healthy subjects (e.g., non-neonatal subjects) are usually below 5-15 μM. In some embodiments, the threshold of tHcy above which a metabolic disorder of homocysteine metabolism should be suspected (e.g., in a neonatal subject) and a specific therapy initiated is approximately 50 μM (e.g., in a neonatal subject) or 100 μM (e.g., in non-neonatal subjects). However, subjects may present with levels of 500 μM homocysteine or higher. Accordingly, in some embodiments, the subject has a plasma tHcy level less than about 500, 450, 400, 350, 300, 250, 200, 150, 100, 90, 80, 70, 60, 50, 45, 40, 35, 30, 25, 20, 25, 20, 19, 18, 17, 16, 15, 14, 13, 12, 11, 10, 9, 8, 7, 6, 5, 4, 3, 2 or 1 μM, after administering the engineered erythroid cells to the subject. Any of these values may be used to define a range for the plasma tHcy level in the subject after administering the engineered erythroid cells to the subject. For example, in some embodiments, the subject has a plasma tHcy level from 5 to 15 μM, from 5 to 50 μM, from 10 to 50 μM, or from 15 to 50 μM after administering the engineered erythroid cells to the subject. In a particular embodiment, the subject has a plasma tHcy level less than about 50 μM after administering the engineered erythroid cells to the subject.

In some embodiments, the engineered erythroid cells described herein may be used to reduce tHcy levels in a subject with homocystinuria, for example, a subject with CBS-deficient homocystinuria (CBSDH). The tHcy levels may be reduced to 1, 2, 3, 4, 5, 6, 7, 8, 9, 10, 11, 12, 13, 14, 15, 16, 17, 18, 19, 20 21, 22, 23, 24, 15, 26, 27, 28, 29, 30, 31, 32, 33, 34, 35, 36, 37, 38, 39, 40, 41, 42, 43, 44, 45, 46, 47, 48, 49, 50, 51, 52, 53, 54, 1-5, 1-10, 1-20, 1-30, 1-40, 1-50, 2-5, 2-10, 2-20, 2-30, 2-40, 2-50, 3-5, 3-10, 3-20, 3-30, 3-40, 3-50, 4-6, 4-10, 4-20, 4-30, 4-40, 4-50, 5-7, 5-10, 5-20, 5-30, 5-40, 5-50, 6-8, 6-10, 6-20, 6-30, 6-40, 6-50, 7-10, 7-20, 7-30, 7-40, 7-50, 8-10, 8-20, 8-30, 8-40, 8-50, 9-10, 9-20, 9-30, 9-40, 9-50, 10-20, 10-30, 10-40, 10-50, 20-30, 20-40, 20-50, 30-40, 30-50 or 40-50 times the level of homocysteine in a healthy subject or in the same subject prior to treatment (e.g., by administering an engineered erythroid cell provided herein).

In some embodiments, administering the engineered erythroid cells to a subject decrease homocysteine levels by 5%, 6%, 7%, 8%, 9%, 10%, 11%, 12%, 13%, 14%, 15%, 16%, 17%, 18%, 19%, 20%, 21%, 22%, 23%, 54%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% , 81%, 82%, 83%, 84%, 85%, 86%, 87%, 88%, 89%, 90%, 91%, 92%, 93%, 94%, 95%, 96%, 97%, 98%, 99%, 100%, 150%, 200%, 300%, 400%, 500%, 1,000% or more.

### Dosing

The present disclosure is based, in part, on experimentation to determine the target homocysteine degrading activity of an engineered erythroid cell needed to achieve clinical efficacy, and to establish clinical dosing feasibility.

In accordance with the present invention, a suitable single dose size is a dose that results in regulation of CBS activity or formation of cystathionine or cysteine in a patient, or in the amelioration of at least one symptom of a condition in the patient, when administered one or more times over a suitable time period. Doses can vary depending upon the disease being treated. One of skill in the art can readily determine appropriate single dose sizes for a given patient based on the size of a patient and the route of administration.

In one aspect of the invention, a suitable single dose of a therapeutic composition of the present invention is an amount that, when administered by any route of administration, regulates at least one parameter of CBS expression or biological activity in the cells of the patient as described above, as compared to a patient which has not been administered with the therapeutic composition of the present invention (i.e., a pre-determined control patient or measurement), as compared to the patient prior to administration of the composition, or as compared to a standard established for the particular disease, patient type and composition.

Each dose of engineered erythroid cells can be administered at intervals such as once daily, once weekly, twice weekly, or twice monthly. In a particular embodiment, the engineered erythroid cells are administered to a subject once per month. In one embodiment, the engineered erythroid cells are administered to the subject about once every four weeks.

Methods of administering engineered erythroid cells (e.g. reticulocytes) comprising (e.g. expressing) exogenous agent (e.g. polypeptides) are described, for example, in WO2015/073587 and WO2015/153102.

In one embodiment, a dose of engineered erythroid cells as described herein comprises about 1×10¹⁰-1×10¹² engineered erythroid cells per dose, for example about 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In some embodiments, a dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises an exogenous polypeptide comprising a homocysteine degrading polypeptide (e.g. CBS or MGL), or a variant thereof, where the dose of engineered erythroid cells as described herein comprises about 1×10¹⁰-1×10¹² engineered erythroid cells per dose, for example about 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises an exogenous polypeptide comprising a homocysteine degrading polypeptide (e.g. CBS or MGL), or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises an exogenous polypeptide comprising a homocysteine reducing polypeptide, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises an exogenous polypeptide comprising a homocysteine reducing polypeptide, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises an exogenous polypeptide comprising a homocysteine transporter, or a variant thereof, where the dose of engineered erythroid cells as described herein comprises about 1×10¹⁰ 1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises an exogenous polypeptide comprising a homocysteine transporter, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises an exogenous polypeptide comprising a serine transporter, or a variant thereof, where the dose of engineered erythroid cells as described herein comprises about 1×10¹⁰ 1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises an exogenous polypeptide comprising a serine transporter, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises an exogenous polypeptide comprising a cystathionine degrading polypeptide (e.g. CGL), or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises an exogenous polypeptide comprising a cystathionine degrading polypeptide (e.g. CGL), or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose.

The engineered erythroid cell may also comprise combinations of a homocysteine degrading polypeptide (e.g. CBS), a homocysteine reducing polypeptide, a homocysteine transporter, and a serine transporter. For example, in one embodiment, the engineered erythroid cell comprises a first exogenous polypeptide comprising a homocysteine degrading polypeptide (e.g. CBS or MGL), or a variant thereof, and a second exogenous polypeptide comprising a homocysteine transporter, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises a first exogenous polypeptide comprising a homocysteine degrading polypeptide (e.g. CBS or MGL), or a variant thereof, and a second exogenous polypeptide comprising a homocysteine transporter, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises a first exogenous polypeptide comprising a homocysteine degrading polypeptide (e.g. CBS or MGL), or a variant thereof, and a second exogenous polypeptide comprising a serine transporter, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises a first exogenous polypeptide comprising a first exogenous polypeptide comprising a homocysteine degrading polypeptide (e.g. CBS or MGL), or a variant thereof, and a second exogenous polypeptide comprising a serine transporter, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises a first exogenous polypeptide comprising a homocysteine reducing polypeptide, or a variant thereof, and a second exogenous polypeptide comprising a homocysteine transporter, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises a first exogenous polypeptide comprising a first exogenous polypeptide comprising a homocysteine reducing polypeptide, or a variant thereof, and a second exogenous polypeptide comprising a homocysteine transporter, or a variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose. In one embodiment, the engineered erythroid cell comprises a first exogenous polypeptide comprising a homocysteine reducing polypeptide, or a variant thereof, and a second exogenous polyeptide comprising a serine transporter, or variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In one embodiment, the engineered erythroid cell comprises a first exogenous polypeptide comprising a first exogenous polypeptide comprising a homocysteine reducing polypeptide, or a variant thereof, and a second exogenous polyeptide comprising a serine transporter, or variant thereof, where the dose of engineered erythroid cells as described herein comprises about 0.3×10¹¹ to about 1×10¹¹ engineered erythroid cells per dose, for example about 0.3×10¹¹ cells/dose, about 0.4×10¹¹ cells/dose, about 0.5×10¹¹ cells/dose, about 0.6×10¹¹ cells/dose, about 0.7×10¹¹ cells/dose, about 0.8×10¹¹ cells/dose, about 0.9×10¹¹ cells/dose, about 1.0×10¹¹ cells/dose.

Exemplary doses of engineered erythroid cells as described herein can be 1×10¹⁰ cells/dose, 2×10¹⁰ cells/dose, 3×10¹⁰ cells/dose, 4×10¹⁰ cells/dose, 5×10¹⁰ cells/dose, 6×10¹⁰ cells/dose, 7×10¹⁰ cells/dose, 8×10¹⁰ cells/dose, 9×10¹⁰ cells/dose, 1×10¹¹ cells/dose, 2×10¹¹ cells/dose, 3×10¹¹ cells/dose, 4×10¹¹ cells/dose, 5×10¹¹ cells/dose, 6×10¹¹ cells/dose, 7×10¹¹ cells/dose, 8×10¹¹ cells/dose, 9×10¹¹ cells/dose, 1×10¹² cells/dose or more. In certain embodiments, exemplary doses of engineered erythroid cells as described herein can be 0.3×10¹¹ cells/dose, 0.4×10¹¹ cells/dose, 0.5×10¹¹ cells/dose, 0.6×10¹¹ cells/dose, 0.7×10¹¹ cells/dose, 0.8×10¹¹ cells/dose, 0.9×10¹¹ cells/dose, 1×10¹¹ cells/dose.

In some embodiments, the dose of engineered erythroid cells is determined based on the target homocysteine degrading activity per engineered erythroid cell, where homocysteine degrading activity is based on the break down of homocysteine by a homocysteine degrading polypeptide (e.g., CBS or MGL) inside the engineered erythroid cells. The target homocysteine activity may be determined at the homocysteine degrading polypeptide's Vmax. In some embodiments, the target homocysteine degrading activity is determined at a homocysteine concentration of 10, 20, 30, 40, 50, 60, 70, 80, 90, 100, 150, 200, 250, 300, 350, 400, 450 or 500 μM. In a particular embodiment, the target homocysteine degrading activity is determined at a homocysteine concentration of 200 μM. In a particular embodiment, the target homocysteine degrading activity is determined at a homocysteine concentration of 100 μM. In a particular embodiment, the target homocysteine degrading activity is determined at a homocysteine concentration of 50 μM. In a particular embodiment, the target homocysteine degrading activity is determined at a homocysteine concentration of 25 μM. In a particular embodiment, the target homocysteine degrading activity is determined at a homocysteine concentration of 10 μM. In certain embodiments, the homocysteine degrading polypeptide, or variant thereof, is selected from the group consisting of sulfide:quinone reductase, or a variant thereof, methionine synthase, or a variant thereof, 5-methyltetrahydropteroyltriglutamate-homocysteine S-methyltransferase, or a variant thereof, adenosylhomocysteinase, or a variant thereof, cystathionine gamma-lyase, or a variant thereof, methionine gamma-lyase, or a variant thereof, L-amino-acid oxidase, or a variant thereof, thetin-homocysteine S-methyltransferase, or a variant thereof, betaine-homocysteine S-methyltransferase, or a variant thereof, homocysteine S-methyltransferase, or a variant thereof, 5-methyltetrahydropteroyltriglutamate-homocysteine S-methyltransferase, or a variant thereof, selenocysteine Se-methyltransferase, or a variant thereof, cystathionine gamma-synthase, or a variant thereof, O-acetylhomoserine aminocarboxypropyltransferase, or a variant thereof, asparagine-oxo-acid transaminase, or a variant thereof, glutamine-phenylpyruvate transaminase, or a variant thereof, 3-mercaptopyruvate sulfurtransferase, or a variant thereof, homocysteine desulfhydrase, cystathionine beta-lyase, or a variant thereof, amino-acid racemase, or a variant thereof, methionine-tRNA ligase, or a variant thereof, glutamate-cysteine ligase, or a variant thereof, N-(5-amino-5-carboxypentanoyl)-L-cysteinyl-D-valine synthase, or a variant thereof, L-isoleucine 4-hydroxylase, or a variant thereof, L-lysine N6-monooxygenase (NADPH), or a variant thereof, methionine decarboxylase, or a variant thereof, 2,2-dialkylglycine decarboxylase (pyruvate), or a variant thereof, and cysteine synthase (CysO), or a variant thereof.

In other certain embodiments, the homocysteine degrading enzyme is cystathionine beta-synthase. In other certain embodiments, the homocysteine degrading enzyme is methionine gamma-lyase.

In one embodiment, the engineered erythroid cell has a homocysteine degrading activity of between about 1e-12 units/cell and about 1e-9 units/cell. In one embodiment, the engineered erythroid cell has a homocysteine degrading activity of between about 1e-12 and 1e-10 units/cell.

In one embodiment, the engineered erythroid cell has a homocysteine degrading activity of between about 5e-12 and 5e-11 units/cell. In one embodiment, the engineered erythroid cell has about 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units of homocysteine degrading activity per cell. In one embodiment, the engineered erythroid cell has a homocysteine degrading activity of between about 1e-11 to 3e-11 units/cell. In one embodiment, the engineered erythroid cell has about 1e-11, 1.1 e-11, 1.2e-11, 1.3e-11, 1.4e-11, 1.5e-11, 1.6e-11, 1.7e-11, 1.8e-11, 1.9e-11, 2.0e-11, 2.1 e-11, 2.2e-11, 2.3e-11, 2.4e-11, 2.5e-11, 2.6e-11, 2.7e-11, 2.8e-11, 2.9e-11 or 3.0e-11 units of homocysteine degrading activity per cell. In one embodiment, the engineered erythroid cell has at least 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units of homocysteine degrading activity per cell.

In one embodiment, the engineered erythroid cell comprises an exogenous polypeptide comprising a homocysteine degrading polypeptide (e.g. CBS or MGL), or a variant thereof, where the engineered erythroid cell has about 1-2 e-11 units of homocysteine degrading activity per cell, for example, about 1.0e-11, 1.1 e-11, 1.2e-11, 1.3e-11, 1.4e-11, 1.5e-11, 1.6e-11, 1.7e-11, 1.8e-11, 1.9e-11, or 2.0e-11 units of homocysteine degrading activity per cell. In one embodiment, the engineered erythroid cell has a homocysteine degrading activity of between about 1e-11 to 3e-11 units/cell. In one embodiment, the engineered erythroid cell has about 1e-11, 1.1 e-11, 1.2e-11, 1.3e-11, 1.4e-11, 1.5e-11, 1.6e-11, 1.7e-11, 1.8e-11, 1.9e-11, 2.0e-11, 2.1 e-11, 2.2e-11, 2.3e-11, 2.4e-11, 2.5e-11, 2.6e-11, 2.7e-11, 2.8e-11, 2.9e-11 or 3.0e-11 units of homocysteine degrading activity per cell. In one embodiment, the engineered erythroid cell has a homocysteine degrading activity of between about 1e-12 and 1e-10 units/cell. In one embodiment, the engineered erythroid cell has a homocysteine degrading activity of between about 5e-12 and 5e-11 units/cell. In one embodiment, the engineered erythroid cell has at least 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units of homocysteine degrading activity per cell.

In one embodiment, the engineered erythroid cell comprises a first exogenous polypeptide comprising a homocysteine degrading polypeptide (e.g. CBS or MGL), or a variant thereof, and a second exogenous polypeptide comprising a homocysteine and serine transporter, or variant thereof, where the engineered erythroid cell has about 1-2×10⁻¹¹ units of homocysteine degrading activity per cell, for example about 1.0e-11, 1.1 e-11, 1.2e-11, 1.3e-11, 1.4e-11, 1.5e-11, 1.6e-11, 1.7e-11, 1.8e-11, 1.9e-11, or 2.0e-11 units of homocysteine degrading activity per cell. In one embodiment, the engineered erythroid cell has a homocysteine degrading activity of between about 1e-12 and 1e-10 units/cell. In one embodiment, the engineered erythroid cell has a homocysteine degrading activity of between about 1e-11 to 3e-11 units/cell. In one embodiment, the engineered erythroid cell has about 1e-11, 1.1 e-11, 1.2e-11, 1.3e-11, 1.4e-11, 1.5e-11, 1.6e-11, 1.7e-11, 1.8e-11, 1.9e-11, 2.0e-11, 2.1 e-11, 2.2e-11, 2.3e-11, 2.4e-11, 2.5e-11, 2.6e-11, 2.7e-11, 2.8e-11, 2.9e-11 or 3.0e-11 units of homocysteine degrading activity per cell. In one embodiment, the engineered erythroid cell has a homocysteine degrading activity of between about 5e-12 and 5e-11 units/cell. In one embodiment, the engineered erythroid cell has at least 1e-12, 2e-12, 3e-12, 4e-12, 5e-12, 6e-12, 7e-12, 8e-12, 9e-12, 1.0e-11, 1.1e-11, 1.2e-11, 1.3e-11, 1.4e-11, or 1.5e-11 units of homocysteine degrading activity per cell.

In some embodiments, the effective amount of engineered erythroid cells is based on units of homocysteine degrading activity per dose. In some embodiments, the effective amount of engineered erythroid cells comprises 0.1-100 units, or 1-10 units of homocysteine degrading activity per dose, for example, at least 0.1, 0.5, 1, 2, 3, 4, 5, 6, 7, 8, 9, 10, 15, 20, 25, 30, 35, 40, 45, 46, 47, 48, 49, 50, 51, 52, 53, 54, 55, 60, 65, 70, 75, 80, 85, 90, 95 or 100 units of homocysteine degrading activity per dose. In one embodiment, the engineered erythroid cell comprises an exogenous polypeptide comprising a homocysteine degrading polypeptide, or a variant thereof, where the effective amount of engineered erythroid cells comprises 0.1-100 units, or 1-10 units of homocysteine degrading activity per dose, for example, at least 0.1, 0.5, 1, 2, 3, 4, 5, 6, 7, 8, 9, 10, 15, 20, 25, 30, 35, 40, 45, 46, 47, 48, 49, 50, 51, 52, 53, 54, 55, 60, 65, 70, 75, 80, 85, 90, 95 or 100 units of homocysteine degrading activity per dose. In some embodiments, the engineered erythroid cell comprises a first exogenous polypeptide comprising a homocysteine degrading polypeptide (e.g. CBS or MGL), or a variant thereof, and a second exogenous polypeptide comprising a homocysteine transporter, or variant thereof, where the effective amount of engineered erythroid cells comprises 0.1-100 units, or 1-10 units of homocysteine degrading activity per dose, for example, at least 0.1, 0.5, 1, 2, 3, 4, 5, 6, 7, 8, 9, 10, 15, 20, 25, 30, 35, 40, 45, 46, 47, 48, 49, 50, 51, 52, 53, 54, 55, 60, 65, 70, 75, 80, 85, 90, 95 or 100 units of homocysteine degrading activity per dose. In some embodiments, the engineered erythroid cell comprises a first exogenous polypeptide comprising a homocysteine degrading polypeptide (e.g. CBS or MGL), or a variant thereof, and a second exogenous polypeptide comprising a serine transporter, or variant thereof, where the effective amount of engineered erythroid cells comprises 0.1-100 units, or 1-10 units of homocysteine degrading activity per dose, for example, at least 0.1, 0.5, 1, 2, 3, 4, 5, 6, 7, 8, 9, 10, 15, 20, 25, 30, 35, 40, 45, 46, 47, 48, 49, 50, 51, 52, 53, 54, 55, 60, 65, 70, 75, 80, 85, 90, 95 or 100 units of homocysteine degrading activity per dose.

In one example, administration of the engineered erythroid cell is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear.

Any one of the doses provided herein for an engineered erythroid cell as described herein can be used in any one of the methods or kits provided herein. Generally, when referring to a dose to be administered to a subject the dose is a label dose. Thus, in any one of the methods provided herein the dose(s) are label dose(s).

Also provided herein are a number of possible dosing schedules. Accordingly, any one of the subjects provided herein may be treated according to any one of the dosing schedules provided herein. As an example, any one of the subject provided herein may be treated with an engineered erythroid cell as described herein. In certain embodiments, the engineered erythroid cell comprises an exogenous polypeptide comprising a homocysteine degrading polypeptide (e.g. CBS or MGL). In certain embodiments, the engineered erythroid cell comprises an exogenous polypeptide comprising a cystathioinine degrading polypeptide (e.g. CGL). In certain embodiments, the engineered erythroid cell comprises an exogenous polypeptide comprising a homocysteine reducing polypeptide. In certain embodiments, the engineered erythroid cell comprises an exogenous polypeptide comprising a homocysteine transporter. In certain embodiments, the engineered erythroid cell comprises an exogenous polypeptide comprising a serine transporter. In certain embodiments, the engineered erythroid cell comprises a first exogenous polypeptide comprising a homocysteine degrading polypeptide (e.g. CBS or MGL), or a variant thereof, and a second exogenous polypeptide comprising a homocysteine transporter, or a variant thereof. In certain embodiments, the engineered erythroid cell comprises a first exogenous polypeptide comprising a cystathionine degrading polypeptide (e.g. CGL), or a variant thereof, and a second exogenous polypeptide comprising a homocysteine transporter, or a variant thereof. In certain embodiments, the engineered erythroid cell comprises a first exogenous polypeptide comprising a homocysteine degrading polypeptide (e.g. CBS or MGL), or a variant thereof, and a second exogenous polypeptide comprising a serine transporter, or a variant thereof. In certain embodiments, the engineered erythroid cell comprises a first exogenous polypeptide comprising a homocysteine reducing polypeptide, or a variant thereof, and a second exogenous polypeptide comprising a homocysteine transporter, or a variant thereof. In certain embodiments, the engineered erythroid cell comprises a first exogenous polypeptide comprising a homocysteine reducing polypeptide, or a variant thereof, and a second exogenous polypeptide comprising a serine transporter, or a variant thereof.

The mode of administration for the composition(s) of any one of the treatment methods provided may be by intravenous administration, such as an intravenous infusion that, for example, may take place over about 1 hour. Additionally, any one of the methods of treatment provided herein may also include administration of an additional therapeutic, as described in more detail below. The administration of the additional therapeutic may be according to any one of the applicable treatment regimens provided herein.

In some embodiments of any one of the methods provided herein, the level of homocysteine is measured in the subject at one or more time points before, during and/or after the treatment period.

### Subjects

The methods described herein are intended for use with any subject that may experience the benefits of these methods. Thus, “subjects,” “patients,” and “individuals” (used interchangeably) include humans as well as non-human subjects, particularly domesticated animals. Subjects provided herein can be in need of treatment according to any one of the methods or compositions or kits provided herein. Such subjects include those with elevated homocysteine levels relative to a healthy subject, for example elevated homocysteine levels in blood, serum, plasma, tissue and/or urine relative to a healthy subject. Such subjects include those with homocystinuria. It is within the skill of a clinician to be able to determine subjects in need of a treatment as provided herein.

In some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. In some embodiments, the subject and/or animal is a human. In some embodiments, the human is a pediatric human. In some embodiments, the pediatric human is an infant. In other embodiments, the pediatric human is a toddler. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal. In certain embodiments, the subject is a human subject having gout or another disease or condition associated with hyperuricemia. In other certain embodiments, the subject is a human subject having chronic refractory gout.

In some embodiments, any one of the subjects for treatment as provided in any one of the methods provided has been diagnosed with a disease or disorder selected from the group consisting of failure to thrive, an eye disorder (e.g. myopia (nearsightedness), dislocation of the lens of the eye (ectopia lentis), glaucoma, optic atrophy, retinal detachment, or cataracts), neurodegeneration, a psychiatric disorder, a central nervous system disorder (e.g. mental retardation or seizures), aberrant musculoskeletal development (e.g. Marfanoid features), a skeletal disorder (e.g. pectus excavatum, carinatum, genu valgum, scoliosis, kyphoscoliosis, or osteoporosis), cardiovascular disease (e.g. hypertension, myocardial infarction, or thromboembolism), stroke, vitamin B12 deficiency, folate deficiency, and renal failure.

In some embodiments, the subject has or is at risk of having an elevated homocysteine level relative to a healthy subject, e.g., an elevated level of homocysteine in blood, plasma, serum, tissue or urine. In some embodiments, homocysteine levels in plasma greater than 50 μM are indicative that a subject may be a candidate for treatment with any one of the methods or compositions or kits described herein. In some embodiments, the subject has a plasma tHcy level greater than about 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450 or 500 μM prior to administering the engineered erythroid cells described herein to the subject. In some embodiments, the subject has a plasma tHcy level less than about 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450 or 500 μM prior to administering the engineered erythroid cells to the subject. Any of these values may be used to define a range for the plasma tHcy level in the subject prior to administering the engineered erythroid cells to the subject. For example, in some embodiments, the subject has a plasma tHcy level from 50 to 100 μM, from 50 to 200 μM, or from 50 to 500 μM prior to administering the engineered erythroid cells to the subject. In a particular embodiment, the subject has a plasma tHcy level greater than about 50 μM prior to administering the engineered erythroid cells to the subject.

In some embodiments, the subject has, or is at risk of having, homocystinuria. In some embodiments, the subject has, or is at risk of having, a condition associated with homocystinuria.

For example, in some embodiments, the subject has, or is at risk of having, failure to thrive. In some embodiments, the subject has, or is at risk of having, an eye disorder (e.g. myopia (nearsightedness), dislocation of the lens of the eye (ectopia lentis), glaucoma, optic atrophy, retinal detachment, or cataracts). In some embodiments, the subject has, or is at risk of having, neurodegeneration. In some embodiments, the subject has, or is at risk of having, a psychiatric disorder. In some embodiments, the subject has, or is at risk of having a central nervous system disorder (e.g. mental retardation or seizures). In some embodiments, the subject has, or is at risk of having, aberrant musculoskeletal development (e.g. Marfanoid features). In some embodiments, the subject has, or is at risk of having, a skeletal disorder (e.g. pectus excavatum, carinatum, genu valgum, scoliosis, kyphoscoliosis, or osteoporosis). In some embodiments, the subject has, or is at risk of having, cardiovascular disease (e.g. hypertension, myocardial infarction or thromboembolism). In some embodiments, the subject has had, or is at risk of having, a stroke. In some embodiments, the subject has, or is at risk of having, vitamin B12 deficiency. In some embodiments, the subject has, or is at risk of having, folate deficiency. In some embodiments, the subject has, or is at risk of having, renal failure.

In some embodiments, the subject has been treated with a therapeutic that increases homocysteine levels. Therapeutics that increase homocysteine levels include, but are not limited to, nitrous oxide, methotrexate, and folate antagonists.

In some embodiments, the subject is selected for treatment with an erythroid cell engineered to degrade homocysteine as described herein. In some embodiments, the subject is selected for treatment with an erythroid cell engineered to reduce homocysteine levels in the subject as described herein. In some embodiments, the subject is selected for treatment with an erythroid cell engineered to reduce homocysteine metabolite levels in the subject as described herein. In one embodiment, the subject is selected for treatment of homocystinuria with an engineered erythroid cell as described herein. In one embodiment, the subject is selected for treatment of a disorder associated with homocystinuria, for example, failure to thrive, an eye disorder (e.g. myopia (nearsightedness), dislocation of the lens of the eye (ectopia lentis), glaucoma, optic atrophy, retinal detachment, or cataracts), neurodegeneration, a psychiatric disorder, a central nervous system disorder (e.g. mental retardation or seizures), aberrant musculoskeletal development (e.g. Marfanoid features), a skeletal disorder (e.g. pectus excavatum, carinatum, genu valgum, scoliosis, kyphoscoliosis, or osteoporosis), cardiovascular disease (e.g. hypertension, myocardial infarction, or thromboembolism), stroke, vitamin B12 deficiency, folate deficiency, or renal failure, with an engineered erythroid cell of the present disclosure.

In certain embodiments, the methods of the present disclosure provide treatment of homocystinuria and diseases or disorders associated with homocystinuria to human patients suffering therefrom. Accordingly, in some embodiments, the treatment population is human subjects diagnosed with homocystinuria or a disease or disorder associated with homocystinuria for example, failure to thrive, an eye disorder (e.g. myopia (nearsightedness), dislocation of the lens of the eye (ectopia lentis), glaucoma, optic atrophy, retinal detachment, or cataracts), neurodegeneration, a psychiatric disorder, a central nervous system disorder (e.g. mental retardation or seizures), aberrant musculoskeletal development (e.g. Marfanoid features), a skeletal disorder (e.g. pectus excavatum, carinatum, genu valgum, scoliosis, kyphoscoliosis, or osteoporosis), cardiovascular disease (e.g. hypertension, myocardial infarction, thromboembolism), stroke, vitamin B12 deficiency, folate deficiency, or renal failure.

### IV. Pharmaceutical Compositions

The present disclosure encompasses the preparation and use of pharmaceutical compositions comprising an engineered erythroid cell (e.g., engineered enucleated cells) of the disclosure as an active ingredient. Such a pharmaceutical composition may consist of the active ingredient alone, as a combination of at least one active ingredient (e.g., an effective dose of an engineered erythroid cell) in a form suitable for administration to a subject, or the pharmaceutical composition may comprise the active ingredient and one or more pharmaceutically acceptable carriers, one or more additional (active and/or inactive) ingredients, or some combination of these.

In one aspect, a pharmaceutical composition comprises a plurality of the engineered erythroid cells described herein, and a pharmaceutically acceptable carrier. In one embodiment, the pharmaceutical composition comprises a therapeutically effective dose of the engineered erythroid cells. In one embodiment, the pharmaceutical composition comprises between 1e10 and 1e12 engineered erythroid cells. In one embodiment, the pharmaceutical composition comprises at least 1e10, 2e10, 3e10, 4e10, 5e10, 6 e10, 7e10, 8e10, 9e10, or 1e11 cells. In one embodiment, the pharmaceutical composition comprises 1×10¹⁰ cells, 2×10¹⁰ cells, 3×10¹⁰ cells, 4×10¹⁰ cells, 5×10¹⁰ cells, 6×10¹⁰ cells, 7×10¹⁰ cells, 8×10¹⁰ cells, 9×10¹⁰ cells, 1×10¹¹ cells, 2×10¹¹ cells, 3×10¹¹ cells, 4×10¹¹ cells, 5×10¹¹ cells, 6×10¹¹ cells, 7×10¹¹ cells, 8×10¹¹ cells/dose, 9×10¹¹ cells, 1×10¹² cells, or more. In certain embodiments, the pharmaceutical composition comprises 0.3×10¹¹ cells, 0.4×10¹¹ cells, 0.5×10¹¹ cells, 0.6×10¹¹ cells, 0.7×10¹¹ cells, 0.8×10¹¹ cells/dose, 0.9×10¹¹ cells, 1×10¹¹ cells.

In one embodiment, the plurality of engineered erythroid cells have an average homocysteine degrading activity of between 1e-10 and 1e-12 units per cell, for example an average homocysteine degrading activity of about 1e-10, 2e-10, 3e-10, 4e-10, 5e-10, 6e-10, 7e-10, 8e-10, 9e-10, 1e-11, 2e-11, 3e-11, 4e-11, 5e-11, 6e-11, 7e-11, 8e-11, 9e-11, or 1e-12 units/cell. In one embodiment, the plurality of engineered erythroid cells have an average homocysteine degrading activity of between 5e-10 and 5e-11 units per cell. In on embodiment, the plurality of engineered erythroid cells have an average homocysteine degrading activity of at least 1e-10, 2e-10, 3e-10, 4e-10, 5e-10, 6e-10, 7e-10, 8e-10, 9e-10, 1e-11, 2e-11, 3e-11, 4e-11, 5e-11, 6e-11, 7e-11, 8e-11, 9e-11, or 1e-12 units/cell.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a patient subcutaneously, intradermally, intramuscularly, by intravenous (i.v.) injection, or intraperitoneally. The pharmaceutical compositions may be injected directly into a tumor or lymph node.

In one embodiment, the pharmaceutical composition comprising a plurality of the engineered erythroid cells described herein, is administered intravenously.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the disclosure may be prepared, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered.

In addition to the active ingredient, a pharmaceutical composition of the disclosure may further comprise a second pharmaceutically active agents.

In one embodiment, the second agent is precursor vitamin B6 (pyridoxine). In one embodiment, the second agent is betaine.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The engineered erythroid cell of the disclosure can be administered to an animal, preferably a human.

The engineered erythroid cell may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

In certain embodiments, the engineered erythroid cell is administered to the subject about once every four weeks.

An engineered erythroid cell may be co-administered with the various other compounds (e.g. other therapeutic agents). Alternatively, the compound(s) may be administered an hour, a day, a week, a month, or even more, in advance of the engineered erythroid cell, or any permutation thereof. Further, the compound(s) may be administered an hour, a day, a week, or even more, after administration of the engineered erythroid cell, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the engineered erythroid cells, and the like.

Further, it would be appreciated by one skilled in the art, based upon the disclosure provided herein, that where the engineered erythroid cell is to be administered to a mammal, the cells are treated so that they are in a “state of no growth”; that is, the cells are incapable of dividing when administered to a mammal. As disclosed elsewhere herein, the cells can be irradiated to render them incapable of growth or division once administered into a mammal. Other methods, including haptenization (e.g., using dinitrophenyl and other compounds), are known in the art for rendering cells to be administered, especially to a human, incapable of growth, and these methods are not discussed further herein. Moreover, the safety of administration of engineered erythroid cells that have been rendered incapable of dividing in vivo has been established in Phase I clinical trials using engineered erythroid cell transfected with plasmid vectors encoding some of the molecules discussed herein.

In some embodiments, the disclosure features a pharmaceutical composition comprising a plurality of the engineered erythroid cells described herein, and a pharmaceutical carrier. In other embodiments, the disclosure features a pharmaceutical composition comprising a population of engineered erythroid cells as described herein, and a pharmaceutical carrier. It will be understood that any single engineered erythroid cell, plurality of engineered erythroid cells, or population of engineered erythroid cells as described elsewhere herein may be present in a pharmaceutical composition of the invention.

In some embodiments, the pharmaceutical compositions provided herein comprise engineered (i.e. modified) erythroid cells and unmodified erythroid cells. For example, a single unit dose of erythroid cells (e.g., modified and unmodified erythroid cells) can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered enucleated erythroid cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

In some embodiments of the above aspects and embodiments, the engineered erythroid cell is an enucleated cell. In some embodiments of the above aspects and embodiments, the engineered erythroid cell is a nucleated cell.

### Combination Therapies

According to some embodiments, the disclosure provides methods that further comprise administering one or more additional therapies (e.g. one or more additional therapeutics) to a subject. In some embodiments, the disclosure pertains to co-administration and/or co-formulation.

Additional therapies or therapeutics for homocystinuria or conditions associated with homocystinuria, may be administered to any one of the subjects provided herein, such as for the reduction of homocysteine levels in the subject. Any one of the methods provided herein may include the administration of one or more of these additional therapies or therapeutics. In some embodiments, any one of the methods provided herein do not comprise the concomitant administration of an additional therapy or therapeutic. Examples of additional therapies or therapeutics include, but are not limited to, the therapies and therapeutics described below. Other examples will be known to those of skill in the art.

Co-administration refers to the administration of two or more components simultaneously or with a time lapse between administration such as 1 second, 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, or more than 3 days.

Additional therapeutics for homocystinuria include administration of vitamin B6 (e.g., pyridoxine) to the subject. Precursor vitamin B6 (pyridoxine) can help to relieve some of the clinical symptoms of disease for approximately half of homocystinuria patients by increasing residual CBS activity. (Barber and Spaeth, 1969. J. Pediatr. 75:463-478; Mudd et al., 1985, Am. J. Hum. Genet. 37:1-31). Accordingly, in some embodiments, the subject is responsive to vitamin B6. In other embodiments, the subject is not responsive to vitamin B6. In some embodiments, the methods provided herein comprise co-administering vitamin B6 (e.g., pyridoxine) to a subject (e.g., concurrently or consecutively). In some embodiments, the methods provided herein comprise selecting a subject that is being treated with pyridoxine. In some embodiments, the subject is administered or is being treated with between about 100 to about 200 mg daily of pyridoxine. In some embodiments, the subject (e.g., an adult subject) is administered or is being treated with between about 500 to about 1,000 mg daily of pyridoxine.

Additional therapies for homocystinuria include a stringent protein restricted diet along with a methionine-free amino acid formulation supplement. Intake of meat, dairy products and other food high in natural protein is prohibited. Daily consumption of a poorly palatable, synthetic metabolic formula containing amino acids and micronutrients is required to prevent secondary malnutrition. This diet may be further supplemented with folic acid and/or cysteine. This diet has been shown to slow the progression of disease as well as to reverse some of the symptoms. Indeed, even in subjects that are responsive to vitamin B6, a complementary moderately protein-restricted diet is often necessary to achieve full metabolic control. (Picker, J. D., and Levy, H. L., 2004, Homocystinuria Caused by Cystathionine Beta-Synthase Deficiency, In GeneReviews. R. A. Pagon, M. P. Adam, T. D. Bird, C. R. Dolan, C. T. Fong, and K. Stephens, eds. (Seattle (Wash.): University of Washington, Seattle). Accordingly, in some embodiments, vitamin B6 administration is further combined with a stringent protein restricted diet along with a methionine-free amino acid formulation supplement. In some embodiments, the methods provided herein comprise instructing a subject to consume a methionine-restricted diet. In some embodiments, the methods provided herein comprise selecting a subject that is consuming a methionine-restricted diet

Additional therapeutics for homocystinuria also include cysteine, folic acid (folate) and vitamin B12 (e.g., hydroxocobalamin (also known as vitamin B12a and hydroxycobalamin). Adding cysteine to the diet can be helpful to reduce oxidative stress, since glutathione is synthesized from cysteine. Folate and vitamin B12 optimize the conversion of homocysteine to methionine by methionine synthase, thus helping to decrease the plasma homocysteine concentration. See, Sacharow et al. “Homocystinuria Caused by Cystathionine Beta-Synthase Deficiency,” (2004 Jan. 15 [Updated 2017 May 18]). In: Adam M P, Ardinger H H, Pagon R A, et al., editors. GeneReviews® [Internet]. Seattle (WA): University of Washington, Seattle; 1993-2019 (available on the world wide web at ncbi.nlm.nih.gov/books/NBK1524/), the entire contents of which are incorporated herein by reference. In some embodiments, the methods provided herein comprise administering folate and/or vitamin B12 (e.g., pyridoxine) to the subject. In some embodiments, the methods provided herein comprise selecting a subject that is being treated with folate and/or vitamin B12 (e.g., pyridoxine). In some embodiments, the subject is administered or is being treated with between about 5 mg daily of folic acid. In some embodiments, the subject is administered or is being treated with from about 100 μg to about 1 mg per month of hydroxocobalamin.

Additional therapeutics for homocystinuria further include betaine (N,N,N-trimethylglycine). Betaine anhydrous (for oral solution) was approved by the FDA in 1996 and marketed as CYSTADANE™ by Recordati Rare Diseases Inc. Betaine provides an alternate metabolic pathway through which the body can convert homocysteine into methionine. Specifically, betaine serves as a methyl donor for the remethylation of Hcy to Met catalyzed by betaine-homocysteinemethyltransferase in the liver (Wilcken et al., 1983, N Engl J Med 309, (8), 448-53). The product registration of CYSTADANE™ was based on a series of uncontrolled observational studies which suggested significant reductions in homocysteine from pre-treatment levels. For children the initial betaine dose is 50 mg/kg twice daily, adjusted according to response (increased weekly by 50 mg/kg increments). For adults the initial dose is 3 g twice daily. The dose and frequency are adjusted according to biochemical response. There is unlikely to be any benefit in exceeding a dose of 150-200 mg/kg/day (See Morris et al., 2017, J Inherit Metab Dis. 40:49-74). Despite the reported efficacy of betaine treatment, there is preclinical data to suggest that patients tolerize to betaine supplementation and that efficacy wanes over time. Accordingly, in some embodiments, a subject is not responsive to betaine, or has lost responsiveness to betaine over time. Side effects of betaine supplementation are generally limited, but some patients may develop body odor and cerebral edema, which occurs as a result of hypermethioninemia (See Morris et al., cited above). In some embodiments, the methods provided herein comprise administering betaine to the subject. In some embodiments, the methods provided herein comprise selecting a subject that is being treated with betaine. In some embodiments, the subject (e.g., a non-adult subject) is administered or is being treated with an initial dose of between about 50 mg/kg of betaine twice daily, followed by a weekly dose of 50 mg/kg of betaine. In some embodiments, the subject (e.g., an adult subject) is administered or is being treated with a dose of betaine about 3 g twice daily. In some embodiments, the subject (e.g., an adult subject) is administered or is being treated with a dose of between about 150 to about 200 mg/kg/day of betaine.

Different types of products have been developed for use in diets of infants and of older persons with inborn errors of metabolism. In infants or toddlers, several products are available commercially for the nutritional support of homocystinuria. Since soy protein is low in methionine, soy protein isolate has been used to prepare a low-methionine infant formula, Low Methionine Diet Powder (Mead Johnson Corp., Evansville, Id., U.S.A.).

Monitoring of a subject, such as the measurement of homocysteine levels in blood, plasma, serum, tissue or urine, may be an additional step further comprised in any one of the methods provided herein. Methods of measuring homocysteine in biological fluids and tissues are known in the art and are described, for example, in Sawula, et al., 2008, Acta Biochimica Polonica 55(1): 119-125; and Morris et al., cited above.

Blood samples for measuring plasma tHcy may be prepared as follows. Venous blood is drawn into an anticoagulant such as EDTA, heparin or citrate. The sample is centrifuged within 1 hour if stored at room temperature since red blood cells generate Hcy at a rate of about 1-2 μmol/L/hr in unseparated whole blood or within 8 hours if blood with anticoagulants is stored at 4° C.; alternatively, serum may be used (Refsum et al., 2004, J Pediatr. 144:830-832). After centrifugation the tHcy in plasma or serum is stable for at least 4 days at room temperature, for several weeks at 4° C. and several years at −20° C. (Refsum et al., cited above). While strict observation of pre-analytical conditions may be important for research studies, differences in plasma tHcy concentrations due to suboptimal pre-analytical procedures or diurnal variation, fed state, pregnancy or posture are relatively minor and unlikely to compromise the diagnosing of CBS deficiency in typical cases (Refsum et al., cited above).

An alternative screening approach for determining tHcy is the analysis of dried blood spots (DBS) obtained by sampling capillary or venous blood on cards used in neonatal screening (Turgeon et al., 2010, Clin Chem. 2010; 56:1686-1695). This is especially useful in clinical situations when pre-analytical and sample transport conditions cannot be met. A local reference range for tHcy concentrations in DBS is established, since tHcy concentrations are 30-40% lower compared to plasma, due to lower concentration of tHcy in erythrocytes.

### V. Kits

The disclosure includes various kits which comprise an engineered erythroid cell of the disclosure, and optionally further include nucleic acids encoding the exogenous polypeptides. Although exemplary kits are described below, the contents of other useful kits will be apparent to the skilled artisan in light of the present disclosure. Each of these kits is included within the disclosure.

In one embodiment, the kit optionally includes a device suitable for administration of the composition (or one or more agents of a combination therapy), e.g., a syringe or other suitable delivery device. Moreover, in embodiments the kit further comprises an instructional material which describe the use of the kit to perform the methods described herein. These instructions simply embody the disclosure provided herein.

In one embodiment, the kit includes a pharmaceutically-acceptable carrier. The composition is provided in an appropriate amount as set forth elsewhere herein. Further, the route of administration and the frequency of administration are as previously set forth elsewhere herein.

The kit encompasses an engineered erythroid cell comprising a wide plethora of molecules, such as, but not limited to, the exogenous polypeptides set forth herein. However, the skilled artisan armed with the teachings provided herein, would readily appreciate that the disclosure is in no way limited to these, or any other, combination of molecules. Rather, the combinations set forth herein are for illustrative purposes and they in no way limit the combinations encompassed by the present disclosure.

Further, the kit comprises a kit where each molecule to be transduced into the engineered erythroid cell is provided as an isolated nucleic acid encoding a molecule, a vector comprising a nucleic acid encoding a molecule, and any combination thereof, including where at least two molecules are encoded by a contiguous nucleic acid and/or are encoded by the same vector.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1

### Generation of Erythroid Cells Genetically Engineered to Comprise Cystathionine Beta-Synthase

### Production of Lentiviral Vector

A lentiviral vector is constructed with a gene encoding cystathionine beta-synthase under the control of the MSCV promoter. Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C.

### Production of Cystathionine Beta-Synthase

The production of cystathionine beta-synthase is assessed via intracellular staining using antibody specific for the cystathionine beta-synthase produced and analysis via flow cytometry or western blot analysis with cystathionine beta-synthase antibody following SDS-PAGE separation.

### Example 2

### Generation of Erythroid Cells Genetically Engineered to Comprise Methionine Gamma-Lyase

### Production of Lentiviral Vector

A lentiviral vector is constructed with a gene encoding methionine gamma-lyase under the control of the MSCV promoter. Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C.

### Expression of Methionine Gamma-Lyase

The expression of methionine gamma-lyase is assessed via intracellular staining using antibody specific for the methionine gamma-lyase expressed and analysis via flow cytometry or western blot analysis with methionine gamma-lyase antibody following SDS-PAGE separation.

### Example 3

### Generation of Erythroid Cells Genetically Engineered to Comprise Homocysteine and/or Serine Transporter

### Production of Lentiviral Vector

A lentiviral vector is constructed with a gene or multiple genes encoding homocysteine and/or serine transporter under the control of the MSCV promoter. Lentivirus is produced in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C.

Expression of Homocysteine and/or Serine Transporter

The expression of homocysteine and/or serine transporter is assessed either by intracellular or extracellular staining using antibody specific for the serine or homocysteine transporter expressed and analysis via flow cytometry.

### Example 4

### Generation of Erythroid Cells Genetically Engineered to Comprise Cystathionine Beta-Synthase and Homocysteine and/or Serine Transporter by Expression in Separate Lentiviral Vectors

Co-expression of cystathionine beta-synthase and homocysteine and/or serine transporter in engineered erythroid cells can be accomplished in two alternate ways. In this example, the cystathionine beta-synthase and homocysteine and/or serine transporter are present on two separate lentivirus vectors, and the two different lentiviruses are mixed at transduction.

### Production of Lentiviral Vector

A lentiviral vector is constructed with a gene encoding cystathionine beta-synthase under the control of the MSCV promoter, as described above in Example 1. Further, a lentiviral vector is constructed with gene encoding homocysteine and/or serine transporter under the control of the MSCV promoter, as described in Example 3. Each lentivirus is produced separately in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the cystathionine beta-synthase and homocysteine/serine transporter are expressed from two separate vectors, the two lentiviruses produced from the corresponding vectors are combined together for the transduction step.

Expression of Cystathionine Beta-Synthase and Homocysteine and/or Serine Transporter:

The expression of cystathionine beta-synthase is assessed via intracellular staining using antibody specific for the cystathionine beta-synthase expressed and analysis via flow cytometry or western blot analysis with cystathionine beta-synthase antibody following SDS-PAGE separation. The expression of homocysteine and/or serine transporter is assessed either by intracellular or extracellular staining using antibody specific for the homocysteine and/or serine transporter expressed and analysis via flow cytometry.

### Example 5

### Generation of Erythroid Cells Genetically Engineered to Comprise Cystathionine Beta-Synthase and Homocysteine and/or Serine Transporter by Expression in the Same Lentiviral Vector

In this example, co-expression of cystathionine beta-synthase and homocysteine and/or serine transporter in erythroid cells is accomplished by including both cystathionine beta-synthase and homocysteine and/or serine transporter in the same lentiviral vector.

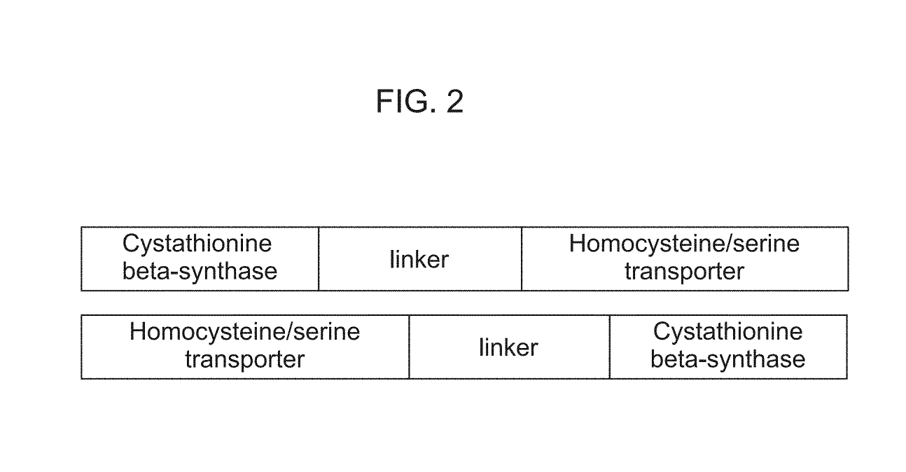

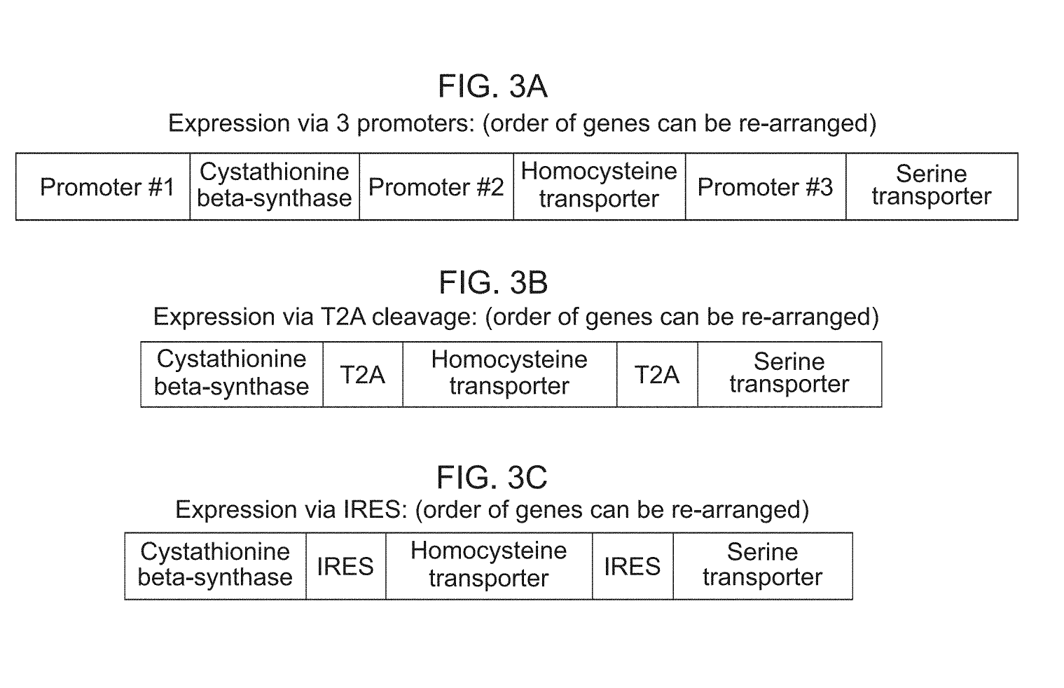

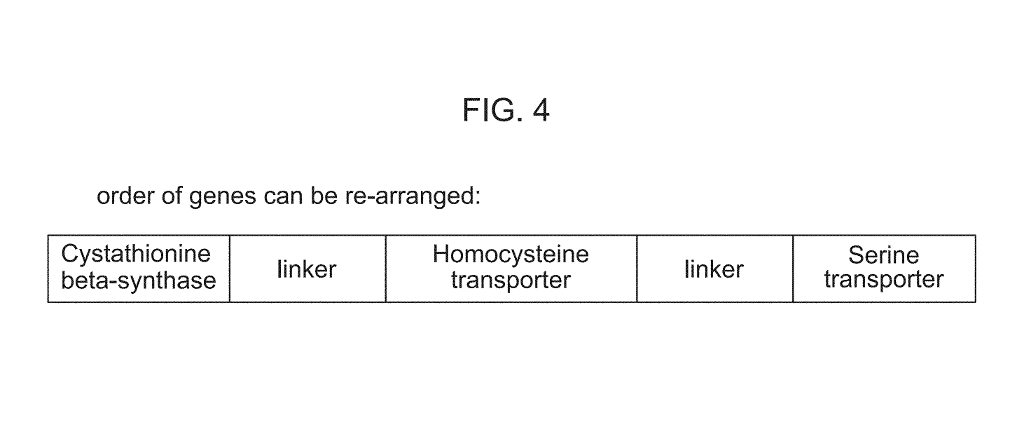

Three strategies for co-expression of cystathionine beta-synthase and homocysteine and/or serine transporter from a single vector are outlined in FIG. 2A, FIG. 2B and FIG. 2C, all of which lead to the translation of two separate polypeptide chains, corresponding to the cystathionine beta-synthase and homocysteine and/or serine transporter proteins. The first strategy involves expression of cystathionine beta-synthase and homocysteine and/or serine transporter from two different promoters (FIG. 2A). The second approach involves dual expression of cystathionine beta-synthase and homocysteine and/or serine transporter via a T2A cleavage (FIG. 2B). The third strategy involves dual expression of cystathionine beta-synthase and homocysteine and/or serine transporter via an IRES (FIG. 2C). Alternatively, cystathionine beta-synthase and homocysteine and/or serine transporter can be expressed as direct polypeptide fusions separated by a linker, as shown in FIG. 3.

### Production of Lentiviral Vector:

A lentiviral vector is constructed with a gene encoding cystathionine beta-synthase and a gene encoding homocysteine and/or serine transporter directly fused via a linker (FIG. 3), or a gene encoding cystathionine beta-synthase and a gene encoding homocysteine and/or serine transporter separated by the T2A sequence (FIG. 2B) or IRES (FIG. 2C) under the control of the MSCV promoter. Alternatively, a lentiviral vector is constructed containing a cystathionine beta-synthase and a homocysteine and/or serine transporter under the control of two different promoters (FIG. 2A). Lentivirus is produced in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this example, since the cystathionine beta-synthase and homocysteine and/or serine transporter are both expressed from the same lentiviral vector, only one lentivirus produced from that vector is used for the transduction step.

Expression of Cystathionine Beta-Synthase and Homocysteine and/or Serine Transporter:

The expression of cystathionine beta-synthase is assessed via intracellular staining using antibody specific for the cystathionine beta-synthase expressed and analysis via flow cytometry or western blot analysis with cystathionine beta-synthase antibody following SDS-PAGE separation. The expression of homocysteine and/or serine transporter is assessed either by intracellular or extracellular staining using antibody specific for the uric acid transporter expressed and analysis via flow cytometry.

### Example 6

### Generation of Erythroid Cells Genetically Engineered to Comprise Methionine Gamma-Lyase and Homocysteine Transporter by Expression in Separate Lentiviral Vectors

In this example, the methionine gamma-lyase and homocysteine transporter are present on two separate lentivirus vectors, and the two different lentiviruses are mixed at transduction.

### Production of Lentiviral Vector

A lentiviral vector is constructed with a gene encoding methionine gamma-lyase under the control of the MSCV promoter, as described above in Example 2. Further, a lentiviral vector is constructed with gene encoding homocysteine transporter under the control of the MSCV promoter, as described in Example 3. Each lentivirus is produced separately in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the methionine gamma-lyase and homocysteine transporter are expressed from two separate vectors, the two lentiviruses produced from the corresponding vectors are combined together for the transduction step.

### Expression of Methionine Gamma-Lyase and Homocysteine Transporter:

The expression of methionine gamma-lyase is assessed via intracellular staining using antibody specific for the methionine gamma-lyase expressed and analysis via flow cytometry or western blot analysis with methionine gamma-lyase antibody following SDS-PAGE separation. The expression of homocysteine transporter is assessed either by intracellular or extracellular staining using antibody specific for the homocysteine and/or serine transporter expressed and analysis via flow cytometry.

### Example 8

### Generation of Erythroid Cells Genetically Engineered to Comprise Cystathionine Beta-Synthase, Homocysteine and/or Serine Transporter and Cystathionine Gamma-Lyase

In this example, a cystathionine beta-synthase and cystathionine gamma-lyase fusion are present in one lentiviral vector, and a homocysteine and/or serine transporter is present in another lentiviral vector, and two different lentiviruses are mixed at transduction.

### Production of Lentiviral Vector

A lentiviral vector is constructed with a fusion of gene encoding CBS and gene encoding CGL, preferably in the orientation (N terminus)-CBS-linker-CGL-(C terminus). Further, a lentiviral vector is constructed with gene encoding homocysteine and/or serine transporter under the control of the MSCV promoter, as described in Example 3. Each lentivirus is produced separately in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 μm filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the cystathionine beta-synthase and cystathionine gamma-lyase fusion are present in one lentiviral vector, and a homocysteine and/or serine transporter is present in another lentiviral vector, the two lentiviruses produced from the corresponding vectors are combined together for the transduction step.

### Expression of Methionine Gamma-Lyase and Homocysteine Transporter:

The expression of cystathionine beta-synthase and cystathionine gamma-lyase is assessed via intracellular staining using antibody specific for the cystathionine beta-synthase and cystathionine gamma-lyase expressed and analysis via flow cytometry or western blot analysis with cystathionine beta-synthase and cystathionine gamma-lyase antibody following SDS-PAGE separation. The expression of homocysteine and/or serine transporter is assessed either by intracellular or extracellular staining using antibody specific for the homocysteine and/or serine transporter expressed and analysis via flow cytometry.

### Example 9

### Generation of Erythroid Cells Genetically Engineered to Comprise Human Cystathionine Beta-Synthase.

### Production of Lentiviral Vector

A lentiviral vector was constructed containing genes for the expression of truncated human cystathionine beta-synthase (1-413) C-terminally fused to eGFP via a (Gly₂-Ser)₂-Gly₂ (SEQ ID NO: 69) linker (CBS-eGFP).

Lentivirus was produced in HEK-293T cells by transfecting the cells with pPACKH1 (System Biosciences) or in-house made packaging vector mix and the constructed lentivirus vector using TransIT-LT1 transfection reagent (Mirus). After 12-14 hour incubation, cells were placed in fresh culturing medium. The virus supernatant was collected 48 hours post-medium change by centrifugation at 1,500 rpm for 5 minutes. The supernatant was collected, filtered through 0.45 μm filter, and frozen in aliquots in −80° C.

### Expansion and Differentiation of Erythroid Cells:

Human CD34+ cells derived from mobilized peripheral blood cells from normal human donors were purchased frozen from Fred Hutchinson Cancer Research Center. The expansion/differentiation procedure comprises 3 stages. In the first stage, thawed CD34+ erythroid precursors were cultured in Iscove's MDM medium supplemented with recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 5 days. In the second stage, erythroid cells were cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 9 days. In the third stage, erythroid cells were cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 5E5 cells/mL for 10-12 days. Fresh differentiation medium was added to the cultures on various days. The cultures were maintained at 37° C. in 5% CO2 incubator.

### Transduction of Erythroid Precursor Cells:

Erythroid precursor cells were transduced on day 7 of the culture process described above. Erythroid cells in culturing medium were combined with the lentivirus for cystathionine beta-synthase with 1 mg/mL poloxamer 338 and incubated overnight at 37° C. The following day, erythroid cells were gently spun down at 2000 rpm for 5 minutes, supernatant removed, cells were re-suspended in fresh erythroid differentiation medium and cultured further at 37° C.

### Expression of Cystathionine Beta-Synthase on Erythroid Cells:

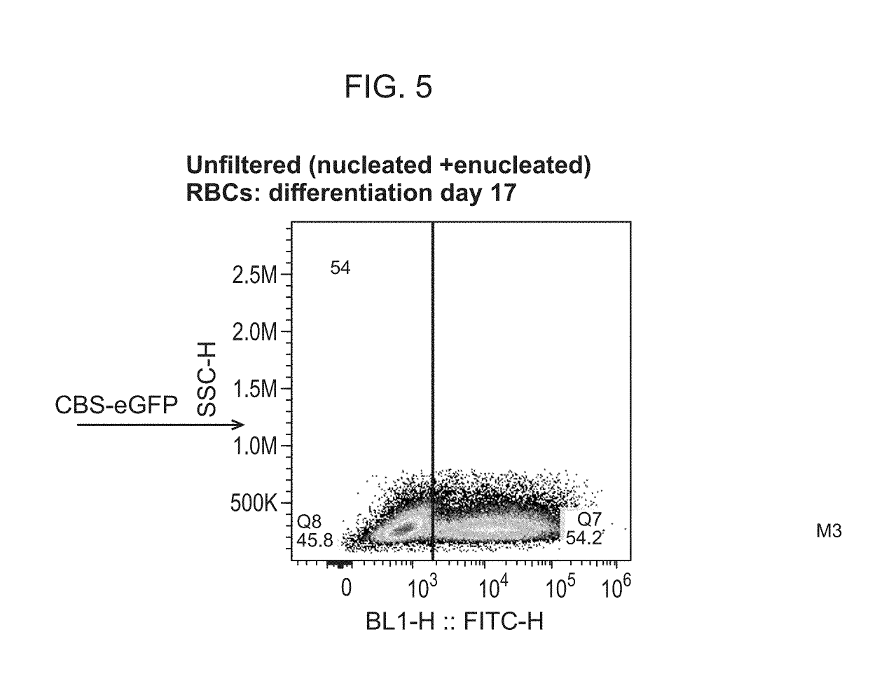

FIG. 5 shows the presence of truncated human CBS-eGFP in a mixture of nucleated and engineered erythroid cells. Using eGFP-conjugated bead standards, the cells that comprise CBS-eGFP, were determined to include the protein at ˜300,000 copies per cell.

### Example 10

### Activity of Engineered Erythroid Cells Comprising Human Cystathionine Beta-Synthase

Engineered erythroid cells comprising human cystathionine beta-synthase, e.g., truncated CBS, are prepared as described in any of Examples 1, 3, 4 and 5.

The activity of the cystathionine beta-synthase is assessed by performing a cystathionine beta-synthase activity assay with intact and/or lysed engineered erythroid cells expressing cystathionine beta-synthase. In this assay, the engineered erythroid cells comprising cystathionine beta-synthase are lysed and/or kept intact and incubated with 200 μM homocysteine and 200 μM serine at 37° C. in a neutral buffer or human serum. At various time points the enzymatic reaction is stopped via incubation at high temperature, protein precipitation via the addition of acid, ethanol, organic solvent or a similar agent, or another method that quenches cystathionine beta-synthase enzymatic activity. Cystathionine beta-synthase activity is assessed either by degradation of homocysteine and/or serine over time or by the appearance of cystathionine over time. One unit of activity is defined as degradation of 1 umole of homocysteine per minute.

### Example 11

### Rate of Homocystine Transport into Erythroid Cells that do Not Comprise Homocysteine and/or Serine Transporter

The endogenous rate of transport of homocystine into erythroid cells that have not been engineered to comprise homocystine or serine transporter was determined by performing a homocysteine transport assay. In this assay, cells were incubated with heavy-isotope homocysteine at 37° C. At various time points, cells were quickly washed with ice-cold buffer, lysed, and the amount of heavy-isotope homocysteine in the cells was measured by mass spectrometry.

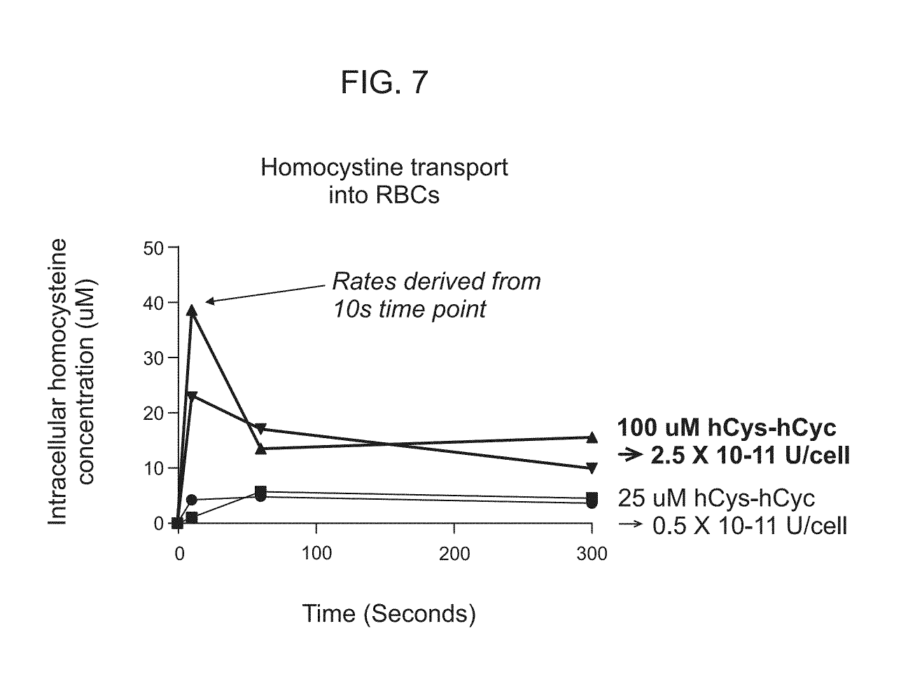

Specifically, erythroid cells were cultured with homocystine at a concentration of 100 μM or 25 μM. Intracellular homocysteine concentration (μM) was measured between 0 and 300 seconds. At 100 μM, the rate of homocystine transport over 300 seconds was 2.5×10⁻¹¹ U/cell. At 25 μM, the rate of homocystine transport over 300 seconds was 0.5×10⁻¹¹ U/cell. Thus, the measured rates of homocystine transport were within or close to the target rate of 1-3×10-11 U/cell. The results of this experiment are shown in FIG. 7.

### Example 12

### Activity of Engineered Erythroid Cells Comprising Homocysteine and/or Serine Transporter

Engineered erythroid cells comprising homocysteine and/or serine transporter are prepared as described in Example 3.

The activity of the homocysteine and/or serine transporter (expressed by itself/themselves) is assessed by performing a homocysteine and/or serine transport assay. In this assay, cells are incubated with heavy-isotope homocysteine or heavy-isotope serine at 37° C. At various time points, cells are quickly washed with ice-cold buffer, lysed, and the amount of heavy-isotope homocysteine or serine in the cells is measured by mass spectrometry.

### Example 13

### Activity of Engineered Erythroid Cells Comprising Cystathionine Beta-Synthase and homocysteine and/or Serine Transporter

Engineered erythroid cells comprising cystathionine beta-synthase and homocysteine and/or serine transporter are prepared as described in Example 3 or 4.

The combined activity of cystathionine beta-synthase and homocysteine and/or serine transporter is assessed by performing a cystathionin beta-synthase activity assay using intact erythroid cells engineered to comprise cystathionine beta-synthase and homocysteine and/or serine transporter. In this assay, intact engineered erythroid cells are incubated with 200 μM homocysteine and 200 μM serine at 37° C. in a biological neutral-pH buffer or human serum. At various time points the cells are lysed and the enzymatic reaction is stopped via protein-precipitating addition of acid, ethanol, organic solvent, or a similar agent. Cystathionine beta-synthase activity is assessed either by degradation of homocysteine and/or serine over time, or by the appearance of cystathionine over time. One unit of activity is defined as degradation of 1 umole of homocysteine per minute.

### Example 14

### Dose Estimation for Engineered Erythroid Cells Comprising Cystathionine Beta-Synthase

Engineered erythroid cells comprising cystathionine beta-synthase and possibly homocysteine and/or serine transporter(s) are prepared as described in Example 3 and 4.

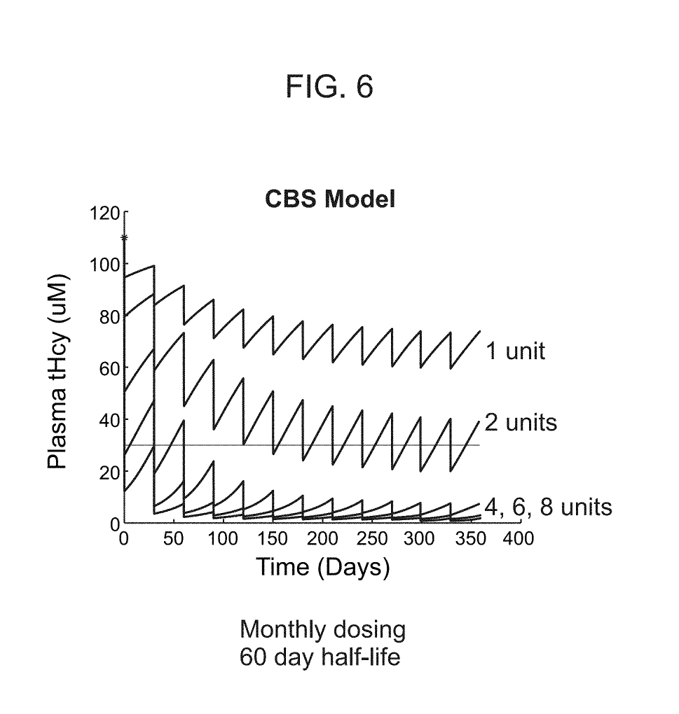

A dose estimation graph, depicted in FIG. 6, shows that the target activity per dose of engineered erythroid cells comprising cystathionine beta-synthase is at least approximately 1-2 units, where projections were made at 200 μM homocysteine and 100 μM serine. This dose modeling calculation assumes (i) a 60 day half-life of the engineered erythroid cells, and (ii) the dose being administered monthly. A concentration of 200 μM homocysteine includes the free and bound levels of homocysteine.

More specifically, the target activity of engineered erythroid cells comprising cystathionine beta-synthase was calculated based upon a homocysteine concentration of 200 μM, since that concentration is the physiological homocysteine concentration of people with homocystinuria. At 200 uM homocysteine and 100 μM serine, the activity of the enzyme is expected to be 3.3% of the value measured at Vmax. This value was determined by carrying out an enzyme kinetics calculation using the Formula: v=Vmax*([S]*[tHcy])/(Kmser*[tHcy]*[S]+[S]*[H]). Thus, the target activity calculated based upon a homocysteine concentration of 200 uM is ˜1.5 units per dose. If a dose is taken to be approximately 1e11 engineered erythroid cells, then the target activity of an engineered erythroid cells is approximately 1.5e-11 units/cell.

Next, experiments were carried out to determine whether sufficient enzyme levels in the erythroid cell could be achieved in order to obtain the target activity. The same 200 uM homocysteine concentration was used to experimentally determine the specific activity of the enzyme present in cells. The specific activity of truncated human cystathionine beta synthase comprised in mammalian cells using 200 μM homocysteine and 100 μM serine was measured and determined to be ˜1 unit/mg. Given this specific activity, it was calculated that erythroid cells would need to comprise ˜190,000 copies/cell in order for one dose of engineered erythroid cells (1e11 cells) to have an overall target activity of 1.5 units.

### Example 15

### Activity of Engineered Erythroid Cells in a Hyperhomocysteinemia Mouse Model

Engineered enucleated erythroid cells comprising an exogenous polypeptide comprising a cystathionine beta-synthase (CBS), and optionally an exogenous polypeptide comprising a cystathionine gamma-lyase and/or an exogenous polypeptide comprising an amino acid transporter (e.g., a homocysteine(ine) transporter) are generated as described in the previous examples.

Hyperhomocysteinemia is induced in mice by administering a high methionine diet (as described, e.g., in Dayal and Lentz (2008) Arterioscler. Thromb. Vasc. Biol. 28(9): 1596-1605, incorporated herein by reference. Total, as well as free (non-protein bound), homocysteine levels in plasma is monitored using tandem mass spectrometry analysis. A group of 10 animals for each condition are tested (group 1: unmodified enucleated erythroid cells; group 2: positive control—recombinant CBS; groups 3-5: different doses of engineered enucleated erythroid cells comprising an exogenous polypeptide comprising a cystathionine beta-synthase, and optionally an exogenous polypeptide comprising a cystathionine gamma-lyase and/or an exogenous polypeptide comprising an amino acid transporter (e.g., a homocysteine(ine) transporter). Mice are also treated with clodronate liposomes as well as cobra venom factor prior to and around the time of erythroid cell injection. Cells or recombinant CBS are administered via intravenous injection. Following treatment, whole blood from mice is periodically collected and the concentration of total and free homocysteine in plasma is determined via tandem mass spectrometry. Administration of engineered erythroid cells comprising an exogenous polypeptide comprising a cystathionine beta-synthase, and optionally an exogenous polypeptide comprising a cystathionine gamma-lyase and/or an exogenous polypeptide comprising an amino acid transporter (e.g., a homocysteine(ine) transporter) is expected to decrease plasma homocysteine levels.

### Example 16

### Conjugation of Cystathionine-Gamma Lyase (Or Mutants Thereof) or Cystathionine Beta Synthase (Or Mutants Thereof) to Erythroid Cells Using Click Chemistry

Cystathionine-gamma lyase (CGL) (or mutants thereof) or cystathionine beta synthase (CBS) (or mutants thereof) are expressed in E. coli as 6× His tagged fusions, where the tag is cleavable by the TEV protease. The proteins are further purified using nickel or cobalt-immobilized metal affinity chromatography followed by size exclusion chromatography. Lastly, the His-tag is cleaved off of the recombinant CGL or CBS proteins using TEV protease. The CGL or CBS protein is placed in a buffer without primary amines, and is incubated with 0.5-15 fold excess of DBCO-NHS for 30 minutes, for DBCO-labeling of the protein. The protein is then processed using a desalting column to remove unreacted NHS reagent.

The engineered enucleated erythroid cells comprising an exogenous polypeptide comprising CGL or CBS are generated using click chemistry. Whole blood is passed through a deleukocyting filter to isolate red blood cells. PBS washed red blood cells are labeled with 6-azidohexanoic acid sulfo-NHS ester in the presence of sodium bicarbonate at room temperature for 30 minutes. The cells are subsequently washed with PBS. Labeled cells are incubated with DBCO-labeled CGL or CBS protein at room temperature for 1 hour, for conjugation of the protein to the cells. Finally, the cells are washed again with PBS.

The engineered enucleated erythroid cells comprising an exogenous polypeptide comprising CGL or CBS are incubated with anti-CGL or anti-CBS fluorescent antibodies, and are analyzed via flow-cytometry to assess the CGL or CBS conjugation to the cells. An activity assay is also performed on the protein conjugated cells to assess the activity of the conjugated enzyme. In this assay, the cells are incubated at 37° C. with homocysteine in human serum, and the decrease in homocysteine concentration over time is monitored via mass spectrometry.

1. An enucleated cell engineered to reduce homocysteine levels, comprising a first exogenous polypeptide comprising a homocysteine reducing polypeptide, or a variant thereof, wherein the homocysteine reducing polypeptide is selected from the group consisting of: methionine adenosyltransferase, alanine transaminase, L-alanine-L-anticapsin ligase, L-cysteine desulfidase, methylenetetrahydrofolate reductase, and 5-methyltetrahydrofolate-homocysteine methyltransferase reductase, or variants thereof.

2.-11. (canceled)

12. An enucleated cell engineered to reduce homocysteine levels, comprising a first exogenous polypeptide comprising a homocysteine degrading polypeptide, or variant thereof, wherein the homocysteine degrading polypeptide, or variant thereof, is not cystathionine beta-synthase.

13. The engineered enucleated cell of claim 12, wherein the homocysteine degrading polypeptide, or variant thereof, is selected from the group consisting of: sulfide:quinone reductase, or a variant thereof, methionine synthase, or a variant thereof, 5-methyltetrahydropteroyltriglutamate-homocysteine S-methyltransferase, or a variant thereof, adenosylhomocysteinase, or a variant thereof, cystathionine gamma-lyase, or a variant thereof, methionine gamma-lyase, or a variant thereof, L-amino-acid oxidase, or a variant thereof, thetin-homocysteine S-methyltransferase, or a variant thereof, betaine-homocysteine S-methyltransferase, or a variant thereof, homocysteine S-methyltransferase, or a variant thereof, 5-methyltetrahydropteroyltriglutamate-homocysteine S-methyltransferase, or a variant thereof, selenocysteine Se-methyltransferase, or a variant thereof, cystathionine gamma-synthase, or a variant thereof, O-acetylhomoserine aminocarboxypropyltransferase, or a variant thereof, asparagine-oxo-acid transaminase, or a variant thereof, glutamine-phenylpyruvate transaminase, or a variant thereof, 3-mercaptopyruvate sulfurtransferase, or a variant thereof, homocysteine desulfhydrase, cystathionine beta-lyase, or a variant thereof, amino-acid racemase, or a variant thereof, methionine-tRNA ligase, or a variant thereof, glutamate-cysteine ligase, or a variant thereof, N-(5-amino-5-carboxypentanoyl)-L-cysteinyl-D-valine synthase, or a variant thereof, L-isoleucine 4-hydroxylase, or a variant thereof, L-lysine N6-monooxygenase (NADPH), or a variant thereof, methionine decarboxylase, or a variant thereof, 2,2-dialkylglycine decarboxylase (pyruvate), or a variant thereof, and cysteine synthase (CysO), or a variant thereof.

14.-19. (canceled)

20. The engineered enucleated cell of claim 13, wherein the homocysteine degrading polypeptide, or variant thereof, is a cysteine synthase (CysO), or a variant thereof, and the CysO is an Aeropyrum pernix CysO, or a variant thereof.

21. The engineered enucleated cell of claim 20, wherein the Aeropyrum pernix CysO comprises the amino acid sequence set forth in SEQ ID NO:12.

22.-29. (canceled)

30. An enucleated cell engineered to reduce homocysteine levels, comprising a first exogenous polypeptide comprising a cystathionine beta-synthase (CBS) polypeptide, or variant thereof.

31.-45. (canceled)

46. The engineered enucleated cell of claim 30, wherein the homocysteine reducing polypeptide is a CBS variant, and wherein the CBS variant is a truncated cystathionine beta-synthase.

47. The engineered enucleated cell of claim 46, wherein the truncated cystathionine beta-synthase lacks a C-terminal regulatory domain or wherein the truncated cystathionine beta-synthase lacks an N-terminal heme-binding region.

48. (canceled)

49. The engineered enucleated cell of claim 46, wherein the truncated cystathionine beta-synthase comprises at least the proteolytically resistant core.

50. The engineered enucleated cell of claim 30, wherein the homocysteine reducing polypeptide is a CBS variant, and wherein the CBS variant comprises at least one mutated amino acid residue, wherein the at least one mutated amino acid residue comprises one or more cysteine residues.

51. (canceled)

52. The engineered enucleated cell of claim 30, wherein the cystathionine beta-synthase polypeptide is selected from the group consisting of: a Homo sapiens cystathionine beta-synthase, a Saccharomyces cerevisiae cystathionine beta synthase, a Mus musculus cystathionine beta-synthase, a Oryctolagus cuniculus cystathionine beta-synthase, a Mycobacterium tuberculosis cystathionine beta-synthase, a Rattus norvegicus cystathionine beta-synthase, a Dictyostellium discoideum cystathionine beta-synthase, a Drosophila melanogaster cystathionine beta-synthase, a Emericella nidulan cystathionine beta-synthase, a Monodelphis domestica cystathionine beta-synthase, and a Ornithorhynchus anatinus cystathionine beta-synthase.

53. The engineered enucleated cell of claim 52, wherein the Homo sapiens cystathionine beta-synthase comprises an amino acid sequence that is at least 95% identical to the amino acid sequence set forth in SEQ ID NO:1; wherein the Saccharomyces cerevisiae cystathionine beta-synthase comprises an amino acid sequence that is at least 95% identical to the amino acid sequence set forth in SEQ ID NO:2; wherein the Mus musculus cystathionine beta-synthase comprises an amino acid sequence that is at least 95% identical to the amino acid sequence set forth in SEQ ID NO:3; wherein the Oryctolagus cuniculus cystathionine beta-synthase comprises an amino acid sequence that is at least 95% identical to the amino acid sequence set forth in SEQ ID NO:4; wherein the Mycobacterium tuberculosis cystathionine beta-synthase comprises an amino acid sequence that is at least 95% identical to the amino acid sequence set forth in SEQ ID NO:5; wherein the Rattus norvegicus cystathionine beta-synthase comprises an amino acid sequence that is at least 95% identical to the amino acid sequence set forth in SEQ ID NO:6; wherein the Dictyostellium discoideum cystathionine beta-synthase comprises an amino acid sequence that is at least 95% identical to the amino acid sequence set forth in SEQ ID NO:7; wherein the Drosophila melanogaster cystathionine beta-synthase comprises an amino acid sequence that is at least 95% identical to the amino acid sequence set forth in SEQ ID NO:8; wherein the Emericella nidulan cystathionine beta-synthase comprises an amino acid sequence that is at least 95% identical to the amino acid sequence set forth in SEQ ID NO:9; wherein the Monodelphis domestica cystathionine beta-synthase comprises an amino acid sequence that is at least 95% identical to the amino acid sequence set forth in SEQ ID NO:10; or wherein the Ornithorhynchus anatinus cystathionine beta-synthase comprises an amino acid sequence that is at least 95% identical to the amino acid sequence set forth in SEQ ID NO:11.

54. (canceled)

55. (canceled)

56. The engineered enucleated cell of claim 30, wherein the homocysteine reducing polypeptide is a CBS variant comprising the amino acid sequence set forth in SEQ ID NO:1 with a C15S amino acid substitution.

57. The engineered enucleated cell of claim 30, wherein the homocysteine reducing polypeptide is a CBS variant, and wherein the CBS variant is a truncated Homo sapiens cystathionine beta-synthase.

58. The engineered enucleated cell of claim 57, wherein the truncated Homo sapiens cystathionine beta-synthase comprises amino acids 1-413 of SEQ ID NO:1.

59. The engineered enucleated cell of claim 58, wherein the truncated Homo sapiens cystathionine beta-synthase comprises a C15S amino acid substitution in SEQ ID NO:1.

60. The engineered enucleated cell of claim 57, wherein the truncated cystathionine beta-synthase comprises amino acid residues 40-413 of SEQ ID NO:1.

61. The engineered enucleated cell of claim 57, wherein the truncated cystathionine beta-synthase comprises or consists of amino acid residues 1-550, 1-543, 1-533, 1-523, 1-496, 1-488, 1-441, 40-551, 71-413, 71-551, 70-413, or 70-551 of SEQ ID NO:1.

62. (canceled)

63. An enucleated cell engineered to reduce homocysteine levels, comprising a first exogenous polypeptide comprising a methionine gamma-lyase, or variant thereof.

64.-71. (canceled)

72. The engineered enucleated cell of claim 63, wherein the first exogenous polypeptide comprises a methionine gamma-lyase variant comprising an amino acid substitution from a C to H at an amino acid residue corresponding to the amino acid residue at position 116 in SEQ ID NO: 37.

73. The engineered enucleated cell of claim 63, wherein the methionine gamma-lyase polypeptide is selected from the group consisting of: Pseudomonas putida methionine gamma-lyase, Saccharomyces cerevisiae methionine gamma-lyase, Fusobacterium nucleatum methionine gamma-lyase, Streptomyces ambofaciens methionine gamma-lyase, Clostridium saccharobutylicum methionine gamma-lyase, Bacillus mycoides methionine gamma-lyase, Bordetella trematum methionine gamma-lyase, Citrobacter freundii methionine gamma-lyase, Entamoeba histolytica methionine gamma-lyase, Yersinia frederiksenii methionine gamma-lyase, and Bacillus subtilis methionine gamma-lyase.

74. The engineered enucleated cell of claim 63, wherein the Pseudomonas putida methionine gamma-lyase comprises an amino acid sequence that is at least 95% identical to the amino acid sequences set forth in SEQ ID NO: 37; wherein the Fusobacterium nucleatum methionine gamma-lyase comprises an amino acid sequence that is at least 95% identical to the amino acid sequences set forth in SEQ ID NO: 38; wherein the Streptomyces ambofaciens methionine gamma-lyase comprises an amino acid sequence that is at least 95% identical to the amino acid sequences set forth in SEQ ID NO: 39; wherein the Clostridium saccharobutylicum methionine gamma-lyase comprises an amino acid sequence that is at least 95% identical to the amino acid sequences set forth in SEQ ID NO: 40; wherein the Bacillus mycoides methionine gamma-lyase comprises an amino acid sequence that is at least 95% identical to the amino acid sequences set forth in SEQ ID NO: 41; wherein the Bordetella trematum methionine gamma-lyase comprises an amino acid sequence that is at least 95% identical to the amino acid sequences set forth in SEQ ID NO: 42; wherein the Citrobacter freundii methionine gamma-lyase comprises an amino acid sequence that is at least 95% identical to the amino acid sequences set forth in SEQ ID NO: 43; wherein the Entamoeba histolytica methionine gamma-lyase comprises an amino acid sequence that is at least 95% identical to the amino acid sequences set forth in SEQ ID NO: 44; wherein the Yersinia frederiksenii methionine gamma-lyase comprises an amino acid sequence that is at least 95% identical to the amino acid sequences set forth in SEQ ID NO: 45; or wherein the Bacillus subtilis methionine gamma-lyase comprises an amino acid sequence that is at least 95% identical to the amino acid sequences set forth in SEQ ID NO: 46.

75. The engineered enucleated cell of claim 63, wherein the methionine gamma-lyase comprises an amino acid sequence that is at least 95% identical to the amino acid sequence set forth in SEQ ID NO: 47.

76. (canceled)

77. The engineered enucleated cell of claim 75, wherein the methionine gamma-lyase is a mutated methionine gamma-lyase, and wherein the mutation in methionine gamma-lyase is a C to H substitution at an amino acid corresponding to the amino acid at position 116 in SEQ ID NO: 37.

78. The engineered enucleated cell of claim 11, further comprising a second exogenous polypeptide and/or a third exogenous polypeptide, wherein the second exogenous polypeptide comprises a homocysteine transporter or a serine transporter, and wherein the third exogenous polypeptide comprises a homocysteine transporter or a serine transporter.

79.-92. (canceled)

93. The engineered enucleated cell of claim 1, further comprising an exogenous polypeptide comprising a cystathionine degrading polypeptide, or a variant thereof.

94. The engineered enucleated cell of claim 93, wherein the cystathionine degrading polypeptide is a cystathionine gamma-lyase, or a variant thereof.

95. The engineered enucleated cell of claim 94, wherein the cystathionine gamma-lyase polypeptide is selected from the group consisting of: Homo sapiens cystathionine gamma-lyase, Mus musculus cystathionine gamma-lyase, Rattus norvegicus cystathionine gamma-lyase, Saccharomyces cerevisiae cystathionine gamma-lyase, Neurospora crassa cystathionine gamma-lyase, Leishmania major cystathionine gamma-lyase, Corynebacterium ammoniagenes cystathionine gamma-lyase, Emericella nidulans cystathionine gamma-lyase, and Arabidopsis thaliana cystathionine gamma-lyase.

96. (canceled)

97. The engineered enucleated cell of claim 1, which is an erythroid cell or a platelet.

98. (canceled)

99. An engineered enucleated cell comprising a first exogenous polypeptide comprising a homocysteine or serine transporter, or a variant thereof, wherein the homocysteine or serine transporter is selected from the group consisting of: sodium-coupled neutral amino acid transporter 1 (SLC38A1) (SAT1), sodium-coupled neutral amino acid transporter 2 (SLC38A2) (SAT2), sodium-coupled neutral amino acid transporter 4 (SLC38A4) (SATS), neutral amino acid transporter A (SLC1A4) (ASCT1), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1),excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), proton-coupled amino acid transporter 1 (SLC36A1), proton-coupled amino acid transporter 2 (SLC36A2), sodium- and chloride-dependent neutral and basic amino acid transporter B(0+) (SLC6A14), Y+L amino acid transporter 1 (SLC7A7), Y+L amino acid transporter 2 (SLC7A6), organic anion transporter 1 (SLC22A6), and T-type amino acid transporter (SLC16A10).

100.-110. (canceled)

111. The engineered enucleated cell of claim 99, which is an erythroid cell or a platelet.

112.-114. (canceled)

115. A pharmaceutical composition comprising a plurality of engineered enucleated cells, and a pharmaceutically acceptable carrier, wherein the engineered enucleated cells comprise an exogenous polypeptide comprising a homocysteine reducing polypeptide, or a variant thereof, a homocysteine degrading polypeptide, or a variant thereof, a cystathionine beta-synthase, or a variant thereof, a methionine gamma-lyase, or a variant thereof, and/or a homocysteine or serine transporter, or a variant thereof, wherein the homocysteine or serine transporter is selected from the group consisting of: sodium-coupled neutral amino acid transporter 1 (SLC38A1) (SAT1), sodium-coupled neutral amino acid transporter 2 (SLC38A2) (SAT2), sodium-coupled neutral amino acid transporter 4 (SLC38A4) (SATS), neutral amino acid transporter A (SLC1A4) (ASCT1), large neutral amino acids transporter small subunit 1 (SLC7A5) (LAT1), large neutral amino acids transporter small subunit 2 (SLC7A8) (LAT2), excitatory amino acid transporter 1 (SLC1A3) (EAAT1),excitatory amino acid transporter 2 (SLC1A2) (EAAT2), excitatory amino acid transporter 3 (SLC1A1) (EAAT3), excitatory amino acid transporter 4 (SLC1A6) (EAAT4), excitatory amino acid transporter 5 (SLC1A7) (EAAT5), 4F2 cell-surface antigen heavy chain (SLC3A2) CD98, sodium-coupled neutral amino acid transporter 3 (SLC38A3) (SN1), sodium-coupled neutral amino acid transporter 5 (SLC38A5) (SN2), Asc-type amino acid transporter 1 (SLC7A10) (Asc1), b(0,+)-type amino acid transporter 1 (SLC7A9), neutral and basic amino acid transport protein rBAT (SLC3A1), proton-coupled amino acid transporter 1 (SLC36A1), proton-coupled amino acid transporter 2 (SLC36A2), sodium- and chloride-dependent neutral and basic amino acid transporter B(0+) (SLC6A14), Y+L amino acid transporter 1 (SLC7A7), Y+L amino acid transporter 2 (SLC7A6), organic anion transporter 1 (SLC22A6), and T-type amino acid transporter (SLC16A10).

116.-120. (canceled)

121. A method of treating or preventing homocystinuria in a subject, comprising administering to the subject a plurality of engineered enucleated cells, wherein the engineered enucleated cells comprise an exogenous polypeptide comprising a homocysteine reducing polypeptide, or a variant thereof, an exogenous polypeptide comprising a homocysteine degrading polypeptide, or a variant thereof, an exogenous polypeptide comprising a cystathionine beta-synthase, or a variant thereof, an exogenous polypeptide comprising a methionine gamma-lyase, or a variant thereof, and/or an exogenous polypeptide comprising a homocysteine or serine transporter, or a variant thereof, and wherein the plurality is in an amount effective to treat or prevent homocystinuria in the subject.

122. (canceled)

123. (canceled)

124. A method of reducing the level of homocysteine in a subject, comprising administering to the subject a plurality of engineered enucleated cells, wherein the engineered enucleated cells comprise an exogenous polypeptide comprising a homocysteine reducing polypeptide, or a variant thereof, an exogenous polypeptide comprising a homocysteine degrading polypeptide, or a variant thereof, an exogenous polypeptide comprising a cystathionine beta-synthase, or a variant thereof, an exogenous polypeptide comprising a methionine gamma-lyase, or a variant thereof, and/or an exogenous polypeptide comprising a homocysteine or serine transporter, or a variant thereof, and wherein the plurality is in an amount effective to reduce the level of homocysteine in the subject.

125. (canceled)

126. A method of reducing the level of methionine in a subject, comprising administering to the subject a plurality of engineered enucleated cells, wherein the engineered enucleated cells comprise an exogenous polypeptide comprising a homocysteine reducing polypeptide, or a variant thereof, an exogenous polypeptide comprising a homocysteine degrading polypeptide, or a variant thereof, an exogenous polypeptide comprising a cystathionine beta-synthase, or a variant thereof, an exogenous polypeptide comprising a methionine gamma-lyase, or a variant thereof, and/or an exogenous polypeptide comprising a homocysteine or serine transporter, or a variant thereof, and wherein the plurality is in an amount effective to reduce the level of methionine in the subject.

127.-148. (canceled)

149. A method of making an engineered enucleated cell, the method comprising:
introducing an exogenous nucleic acid encoding the first exogenous polypeptide into a nucleated erythroid cell, wherein the first exogenous polypeptide comprises a homocysteine reducing polypeptide, or a variant thereof, a homocysteine degrading polypeptide, or a variant thereof, a cystathionine beta-synthase, or a variant thereof, a methionine gamma-lyase, or a variant thereof, and/or a homocysteine or serine transporter, or a variant thereof, and
culturing the nucleated erythroid cell under conditions suitable for enucleation of the nucleated erythroid cell and for production of the first exogenous polypeptide, thereby making the enucleated cell.

150.-185. (canceled)

186. The engineered enucleated cell of claim 12, which is an erythroid cell or a platelet.

187. The engineered enucleated cell of claim 30, which is an erythroid cell or a platelet.

188. The engineered enucleated cell of claim 63, which is an erythroid cell or a platelet.

189. The engineered enucleated cell of claim 12, further comprising a second exogenous polypeptide and/or a third exogenous polypeptide, wherein the second exogenous polypeptide comprises a homocysteine transporter or a serine transporter, and wherein the third exogenous polypeptide comprises a homocysteine transporter or a serine transporter.

190. The engineered enucleated cell of claim 30, further comprising a second exogenous polypeptide and/or a third exogenous polypeptide, wherein the second exogenous polypeptide comprises a homocysteine transporter or a serine transporter, and wherein the third exogenous polypeptide comprises a homocysteine transporter or a serine transporter.

191. The engineered enucleated cell of claim 63, further comprising a second exogenous polypeptide and/or a third exogenous polypeptide, wherein the second exogenous polypeptide comprises a homocysteine transporter or a serine transporter, and wherein the third exogenous polypeptide comprises a homocysteine transporter or a serine transporter.

192. The engineered enucleated cell of claim 12, further comprising an exogenous polypeptide comprising a cystathionine degrading polypeptide, or a variant thereof.

193. The engineered enucleated cell of claim 30, further comprising an exogenous polypeptide comprising a cystathionine degrading polypeptide, or a variant thereof.


SEQUENCE LISTING (112 sequences)

SEQ ID NO: 1 - protein, 551 aa, Homo sapiens
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 2 - protein, 507 aa, Saccharomyces cerevisiae
MTKSEQQADSRHNVIDLVGNTPLIALKKLPKALGIKPQIYAKLELYNPGGSIKDRIAKSMVEEAEASGRIHPSRSTLIEPTSGNTGIGLALIGAIKGYRTIITLPEKMSNEKVSVLKALGAEIIRTPTAAAWDSPESHIGVAKKLEKEIPGAVILDQYNNMMNPEAHYFGTGREIQRQLEDLNLFDNLRAVVAGAGTGGTISGISKYLKEQNDKIQIVGADPFGSILAQPENLNKTDITDYKVEGIGYDFVPQVLDRKLIDVWYKTDDKPSFKYARQLISNEGVLVGGSSGSAFTAVVKYCEDHPELTEDDVIVAIFPDSIRSYLTKFVDDEWLKKNNLWDDDVLARFDSSKLEASTTKYADVFGNATVKDLHLKPVVSVKETAKVTDVIKILKDNGFDQLPVLTEDGKLSGLVTLSELLRKLSINNSNNDNTIKGKYLDFKKLNNFNDVSSYNENKSGKKKFIKFDENSKLSDLNRFFEKNSSAVITDGLKPIHIVTKMDLLSYLA

SEQ ID NO: 3 - protein, 561 aa, Mus musculus
MPSGTSQCEDGSAGGFQHLDMHSEKRQLEKGPSGDKDRVWIRPDTPSRCTWQLGRAMADSPHYHTVLTKSPKILPDILRKIGNTPMVRINKISKNAGLKCELLAKCEFFNAGGSVKDRISLRMIEDAERAGNLKPGDTIIEPTSGNTGIGLALAAAVKGYRCIIVMPEKMSMEKVDVLRALGAEIVRTPTNARFDSPESHVGVAWRLKNEIPNSHILDQYRNASNPLAHYDDTAEEILQQCDGKLDMLVASAGTGGTITGIARKLKEKCPGCKIIGVDPEGSILAEPEELNQTEQTAYEVEGIGYDFIPTVLDRAVVDKWFKSNDEDSFAFARMLIAQEGLLCGGSSGSAMAVAVKAARELQEGQRCVVILPDSVRNYMSKFLSDKWMLQKGFMKEELSVKRPWWWRLRVQELSLSAPLTVLPTVTCEDTIAILREKGFDQAPVVNESGAILGMVTLGNMLSSLLAGKVRPSDEVCKVLYKQFKPIHLTDTLGTLSHILEMDHFALVVHEQIQSRDQAWSGVVGGPTDCSNGMSSKQQMVFGVVTAIDLLNFVAAREQTQT

SEQ ID NO: 4 - protein, 551 aa, Oryctolagus cuniculus
MPSETAQAGEGPAGCPHLSGAQGSDRSLDQRPPGNKDAPERVWIRPDVPSRCTWELGRPVADSPHQHAALAKSPKILPDILQKIGDTPMVRINKIGKNFGLKCELLAKCEFFNAGGSVKDRISLRMIEDAERAGTLRPGDTIIEPTSGNTGIGLALAAAVKGYRCIIVMPEKMSLEKVDVLRALGAEIVRTPTNARFDSPESHVGVAWRLKQEIPNSHILDQYRNASNPLAHYDTTAEEILQQCDGKLDMLVASAGTGGTITGIARKLKEKCPGCQIIGVDPEGSILAEPEELNQTEVTAYEVEGIGYDFIPTVLDRTVVDRWFKSTDKEAFAFARMLIAQEGLLCGGSAGSAVAVAVKAAQELQEGQRCVVILPDSVRNYMSKFLSDRWMLQKGFLEEEELSVKRPWWWHLRVQELSLSVPLTVLPGVTCSDTIDILRGKGFDQAPVVDETGEILGMVTLGNMLSSLLAGKVQPSDQVCKVLYKQFKQIRLTDTLGALSHILEMDHFALVVHEQIQYGGDEQPSKRQTVFGVVTAMDLLHFVASRGQDQQ

SEQ ID NO: 5 - protein, 464 aa, Mycobacterium tuberculosis
MRIAQHISELIGGTPLVRLNSVVPDGAGTVAAKVEYLNPGGSSKDRIAVKMIEAAEASGQLKPGGTIVEPTSGNTGVGLALVAQRRGYKCVFVCPDKVSEDKRNVLIAYGAEVVVCPTAVPPHDPASYYSVSDRLVRDIDGAWKPDQYANPEGPASHYVTTGPEIWADTEGKVTHFVAGIGTGGTITGAGRYLKEVSGGRVRIVGADPEGSVYSGGAGRPYLVEGVGEDFWPAAYDPSVPDEIIAVSDSDSFDMTRRLAREEAMLVGGSCGMAVVAALKVAEEAGPDALIVVLLPDGGRGYMSKIFNDAWMSSYGFLRSRLDGSTEQSTVGDVLRRKSGALPALVHTHPSETVRDAIGILREYGVSQMPVVGAEPPVMAGEVAGSVSERELLSAVFEGRAKLADAVSAHMSPPLRMIGAGELVSAAGKALRDWDALMVVEEGKPVGVITRYDLLGFLSEGAGRR

SEQ ID NO: 6 - protein, 561 aa, Rattus norvegicus
MPSGTSQCEDGSAGCPQDLEVQPEKGQLEKGASGDKERVWISPDTPSRCTWQLGRPMADSPHYHTVPTKSPKILPDILRKIGNTPMVRINRISKNAGLKCELLAKCEFFNAGGSVKDRISLRMIEDAERAGTLKPGDTIIEPTSGNTGIGLALAAAVKGYRCIIVMPEKMSMEKVDVLRALGAEIVRTPTNARFDSPESHVGVAWRLKNEIPNSHILDQYRNASNPLAHYDDTAEEILQQCDGKVDMLVASAGTGGTITGIARKLKEKCPGCKIIGVDPEGSILAEPEELNQTEQTAYEVEGIGYDFIPTVLDRAVVDRWFKSNDDDSFAFARMLISQEGLLCGGSSGSAMAVAVKAAQELKEGQRCVVILPDSVRNYMSKFLSDKWMLQKGFMKEELSVKRPWWWHLRVQELSLSAPLTVLPTVTCEHTIAILREKGFDQAPVVNESGAILGMVTLGNMLSSLLAGKVRPSDEVCKVLYKQFKPIHLTDTLGMLSHILEMDHFALVVHEQIQSRDQAWSGVVGGPTDRNNGVSSKQLMVFGVVTAIDLLNFVAAREQTRK

SEQ ID NO: 7 - protein, 498 aa, Dictyostellium discoideum
MSAPEGPSKCTWTPNTTENTPHTTRRTPKKLIMDNILDNIGGTPLVRVNKVSSDLECELVAKCEFFNAGGSVKDRIGHRMIVDAEESGRIKKGDTLIEPTSGNTGIGLALTAAIKGYKMIITLPEKMSQEKVDVLKALGAEIIRTPTEAAFDAPESHIGVAKKLNSEIPNSHILDQYGNPSNPLAHYDGTAEELLEQCEGKIDMIVCTAGTGGTITGIARKIKERLPNCIVVGVDPHGSILAQPESLNNTNKSYKIEGIGYDFIPNVLERKLVDQWIKTDDKESFIMARRLIKEEGLLCGGSSGSAMVGALLAAKQLKKGQRCVVLLADSIRNYMTKHLNDDWLVDNGFVDPEYKTKDQQEEEKYHGATVKDLTLPKPITISATTTCAAAVQLLQQYGFDQLPVVSESKKVLGQLTLGNLLSHIASKKAVPTDAVSKVMFRFTKNEKYIPITQSTSLATLSKFFENHSSAIVTENDEIISIVTKIDLLTYLMKSQQKN

SEQ ID NO: 8 - protein, 522 aa, Drosophila melanogaster
MPQPKPYERPADFIDPGKPSKCKWHLGTAEKSPHIHRGIAHRQQITPNILEVIGCTPLVKLNNIPASDGIECEMYAKCEFLNPGGSVKDRIGYRMVQDAEEQGLLKPGYTIIEPTSGNTGIGLAMACAVKGYKCIIVMPEKMSNEKVSALRTLGAKIIRTPTEAAYDSPEGLIYVAQQLQRETPNSIVLDQYRNAGNPLAHYDGTAAEILWQLDNKVDMIVVSAGTAGTISGIGRKIKEQVPSCQIVGVDPYGSILARPAELNKTDVQFYEVEGIGYDFPPTVFDDTVVDVWTKIGDSDCFPMSRRLNAEEGLLCGGSSGGAMHAALEHARKLKKGQRCVVILPDGIRNYMTKFVSDNWMEARNFKEPVNEHGHWWWSLAIAELELPAPPVILKSDATVGEAIALMKKHRVDQLPVVDQDDGSVLGVVGQETLITQIVSMNRQQSDPAIKALNKRVIRLNESEILGKLARVLEVDPSVLILGKNPAGKVELKALATKLDVTTFIAAGKQKPKANGTTNGGSH

SEQ ID NO: 9 - protein, 533 aa, Emericella nidulan
MSSKTTPTVPPVALDSITQHIGNTPLVRLNKLPQNLGINATVYAKLEYFNAGGSVKDRIALRMIEEAERSGRIKPGDTLIEPTSGNTGIGLALVAAVKGYKTIITLPEKMSAEKVSVLRALNATIIRTPNEAAYDSPESHIGVAKRLEKELPNAHILDQYGNENNPLAHELGTAQEIWSQTKGQIKAIVAGAGTGGTITGLSRGLKKHNSNVQVIAADPQGSILALPAALNEEHANEPYKVEGIGYDFIPQVLDQHAVDKWYKTDDKESFQYARRLIAEEGLLVGGSSGSAIAALVKAARDNMFKEGDVVVVILPDSIRSYLTKFADDDWLAANDLLPSLPTEVILQSSNKQTQGQDDAFAGSKVSSLRLKPVTTVHSNVPCETAIEVMRDKGFDQLPVLAPSGRKLVGLVTLGNVLSRLTHNRATGRSPVADVMFDFRTISEVVTDPRDMGLASLEPNQNGRDAPKPQTRSRKFVEITMDTSLSVLNRFFEWNSAAIVTERDEGGTLRPVAVVTKVDLLTWMLHHKKNGASE

SEQ ID NO: 10 - protein, 566 aa, Monodelphis domestica
MPHEFPQIERENGVCPHLSSNSMENVAKETSSNCGNKVEKERKWIRPDNPSKCTWQLGKSLSESPHHHVSWTKTPKIIPDILKKIGDTPMVRINKIGKNFGLKCELLAKCEFFNAGGSVKDRISLRMVEDAERAGILKPGDTIIEPTSGNTGIGLALAAAVKGYRCIIVMPEKMSMEKVDVLRALGAEIVRTPTNARFDSPESHVGVAWRLKNEIPNSHILDQYRNASNPLTHYDSTAEEILQQCDGKLDMLVASAGTGGTITGIARKLKEKCPGCKIIGVDPEGSILAEPEELNQTDKTAYEVEGIGYDFIPTVLDRKVVDKWYKSNDEESFAFARMLIAQEGLLCGGSAGSSMSVAVKAAKELKEGQRCVVILPDSVRNYMSKFLSDKWMLQKGFVKEEDITITKPWWWHLKIQELSLSAPLTVLPTVTCERTIEILREKGFDQVPVVDESGVILGMVTLGNMLSSLLAGKVQPSDQVRNVIYKQFRQIHLTDTLGMLSHILETDHFALVVHEQIQSQDQSLSEMIGGISDHSNGNSILRQMVFGVVTAIDLLNFVAAREREQK

SEQ ID NO: 11 - protein, 579 aa, Ornithorhynchus anatinus
MPSELSKAEEKPGVCPHRPGNPAENGKSDTARCTGLSEKERKWIRPDMPSKCTWQLGKPASESPHYHVSLAQTPQIMPDILKKIGNTPLVRINKIGKAYGLKCELLAKCEFFNAGGSVKDRIGLRMLEDAERAGILKPGDTIIEPTSGNTGIGLALAAAVKGYRCIIVMPEKMSMEKVDVLRALGAEIVRTPTTASFDSPESHVGVAWRLKHEIPNSHILDQYRNASNPLAHYDTTAEEILQQCDGKLDMLVASAGTGGTITGIGRKLKEKCPGCKIIGVDPEGSLLAEPEDLNKTDKTIYEVEGIGYDFIPTVLDRAMVDKWYKCNDEESFAFARMLISQEGLLCGWGSSGSTMSLAVKAAQELKDGQRCVFILPDFVQKYMSKFLSDKWMLQKGFLKEEDIMINKPWWWHVKVQELSLSAPLTVLPAVTCERTIEILREKGFDQVPVVDESGVILGMVTLANMLSSLLAGKVRPSDQVRKVIYKQFQKIRLTDTLGKLSHILEIDHFALVVHEQIQSQDQALPAMVGGTTAGIQHGLENSSSSIDKPEHRGIDLKSSYRFIRHLSPESKHKFNAISA

SEQ ID NO: 12 - protein, 387 aa, Aeropyrum pernix
MALADISGYLDVLDSVRGFSYLENAREVLRSGEARCLGNPRSEPEYVKALYVIGASRIPVDGCSHTLEEGVFDISVPGEMVFPSPLDFFERGKPTPLVRSRLQLPNGVRVWLKLEWYNPFSLSVKDRPAVEIISRLSRRVEKGSLVADATSSNFGVALSAVARLYGYRARVYLPGAAEEFGKLLPRLLGAQVIVDPEAPSTVHLLPRVMKDSKNEGFVHVNQFYNDANFEAHMRGTAREIFVQSRRGGLALRGVAGSLGTSGHMSAAAFYLQSVDPSIRAVLVQPAQGDSIPGIRRVETGMLWINMLDISYTLAEVTLEEAMEAVVEVARSDGLVIGPSGGAAVKALAKKAAEGDLEPGDYVVVVPDTGFKYLSLVQNALEGAGDSV

SEQ ID NO: 13 - protein, 487 aa, Homo sapiens
MMHFKSGLELTELQNMTVPEDDNISNDSNDFTEVENGQINSKFISDRESRRSLTNSHLEKKKCDEYIPGTTSLGMSVFNLSNAIMGSGILGLAFALANTGILLFLVLLTSVTLLSIYSINLLLICSKETGCMVYEKLGEQVFGTTGKFVIFGATSLQNTGAMLSYLFIVKNELPSAIKFLMGKEETFSAWYVDGRVLVVIVTFGIILPLCLLKNLGYLGYTSGFSLSCMVFFLIVVIYKKFQIPCIVPELNSTISANSTNADTCTPKYVTFNSKTVYALPTIAFAFVCHPSVLPIYSELKDRSQKKMQMVSNISFFAMFVMYFLTAIFGYLTFYDNVQSDLLHKYQSKDDILILTVRLAVIVAVILTVPVLFFTVRSSLFELAKKTKFNLCRHTVVTCILLVVINLLVIFIPSMKDIFGVVGVTSANMLIFILPSSLYLKITDQDGDKGTQRIWAALFLGLGVLFSLVSIPLVIYDWACSSSSDEGH

SEQ ID NO: 14 - protein, 506 aa, Homo sapiens
MKKAEMGRFSISPDEDSSSYSSNSDFNYSYPTKQAALKSHYADVDPENQNFLLESNLGKKKYETEFHPGTTSFGMSVFNLSNAIVGSGILGLSYAMANTGIALFIILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSHNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNAPGGGH

SEQ ID NO: 15 - protein, 547 aa, Homo sapiens
MDPMELRNVNIEPDDESSSGESAPDSYIGIGNSEKAAMSSQFANEDTESQKFLTNGFLGKKKLADYADEHHPGTTSFGMSSFNLSNAIMGSGILGLSYAMANTGIILFIIMLLAVAILSLYSVHLLLKTAKEGGSLIYEKLGEKAFGWPGKIGAFVSITMQNIGAMSSYLFIIKYELPEVIRAFMGLEENTGEWYLNGNYLIIFVSVGIILPLSLLKNLGYLGYTSGFSLTCMVFFVSVVIYKKFQIPCPLPVLDHSVGNLSFNNTLPMHVVMLPNNSESSDVNFMMDYTHRNPAGLDENQAKGSLHDSGVEYEAHSDDKCEPKYFVFNSRTAYAIPILVFAFVCHPEVLPIYSELKDRSRRKMQTVSNISITGMLVMYLLAALFGYLTFYGEVEDELLHAYSKVYTLDIPLLMVRLAVLVAVTLTVPIVLFPIRTSVITLLFPKRPFSWIRHFLIAAVLIALNNVLVILVPTIKYIFGFIGASSATMLIFILPAVFYLKLVKKETFRSPQKVGALIFLVVGIFFMIGSMALIIIDWIYDPPNSKHH

SEQ ID NO: 16 - protein, 532 aa, Homo sapiens
MEKSNETNGYLDSAQAGPAAGPGAPGTAAGRARRCAGFLRRQALVLLTVSGVLAGAGLGAALRGLSLSRTQVTYLAFPGEMLLRMLRMIILPLVVCSLVSGAASLDASCLGRLGGIAVAYFGLTTLSASALAVALAFIIKPGSGAQTLQSSDLGLEDSGPPPVPKETVDSFLDLARNLFPSNLVVAAFRTYATDYKVVTQNSSSGNVTHEKIPIGTEIEGMNILGLVLFALVLGVALKKLGSEGEDLIRFFNSLNEATMVLVSWIMWYVPVGIMFLVGSKIVEMKDIIVLVTSLGKYIFASILGHVIHGGIVLPLIYFVFTRKNPFRFLLGLLAPFATAFATCSSSATLPSMMKCIEENNGVDKRISRFILPIGATVNMDGAAIFQCVAAVFIAQLNNVELNAGQIFTILVTATASSVGAAGVPAGGVLTIAIILEAIGLPTHDLPLILAVDWIVDRTTTVVNVEGDALGAGILHHLNQKATKKGEQELAEVKVEAIPNCKSEEETSPLVTHQNPAGPVASAPELESKESVL

SEQ ID NO: 17 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 18 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 19 - protein, 535 aa, Homo sapiens
MEEGARHRNNTEKKHPGGGESDASPEAGSGGGGVALKKEIGLVSACGIIVGNIIGSGIFVSPKGVLENAGSVGLALIVWIVTGFITVVGALCYAELGVTIPKSGGDYSYVKDIFGGLAGFLRLWIAVLVIYPTNQAVIALTFSNYVLQPLFPTCFPPESGLRLLAAICLLLLTWVNCSSVRWATRVQDIFTAGKLLALALIIIMGIVQICKGEYFWLEPKNAFENFQEPDIGLVALAFLQGSFAYGGWNFLNYVTEELVDPYKNLPRAIFISIPLVTFVYVFANVAYVTAMSPQELLASNAVAVTFGEKLLGVMAWIMPISVALSTFGGVNGSLFTSSRLFFAGAREGHLPSVLAMIHVKRCTPIPALLFTCISTLLMLVTSDMYTLINYVGFINYLFYGVTVAGQIVLRWKKPDIPRPIKINLLFPIIYLLFWAFLLVFSLWSEPVVCGIGLAIMLTGVPVYFLGVYWQHKPKCFSDFIELLTLVSQKMCVVVYPEVERGSGTEEANEDMEEQQQPMYQPTPTKDKDVAGQPQP

SEQ ID NO: 20 - protein, 542 aa, Homo sapiens
MTKSNGEEPKMGGRMERFQQGVRKRTLLAKKKVQNITKEDVKSYLFRNAFVLLTVTAVIVGTILGFTLRPYRMSYREVKYFSFPGELLMRMLQMLVLPLIISSLVTGMAALDSKASGKMGMRAVVYYMTTTIIAVVIGIIIVIIIHPGKGTKENMHREGKIVRVTAADAFLDLIRNMFPPNLVEACFKQFKTNYEKRSFKVPIQANETLVGAVINNVSEAMETLTRITEELVPVPGSVNGVNALGLVVFSMCFGFVIGNMKEQGQALREFFDSLNEAIMRLVAVIMWYAPVGILFLIAGKIVEMEDMGVIGGQLAMYTVTVIVGLLIHAVIVLPLLYFLVTRKNPWVFIGGLLQALITALGTSSSSATLPITFKCLEENNGVDKRVTRFVLPVGATINMDGTALYEALAAIFIAQVNNFELNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNETEKPIDSETKM

SEQ ID NO: 21 - protein, 574 aa, Homo sapiens
MASTEGANNMPKQVEVRMHDSHLGSEEPKHRHLGLRLCDKLGKNLLLTLTVFGVILGAVCGGLLRLASPIHPDVVMLIAFPGDILMRMLKMLILPLIISSLITGLSGLDAKASGRLGTRAMVYYMSTTIIAAVLGVILVLAIHPGNPKLKKQLGPGKKNDEVSSLDAFLDLIRNLFPENLVQACFQQIQTVTKKVLVAPPPDEEANATSAVVSLLNETVTEVPEETKMVIKKGLEFKDGMNVLGLIGFFIAFGIAMGKMGDQAKLMVDFFNILNEIVMKLVIMIMWYSPLGIACLICGKIIAIKDLEVVARQLGMYMVTVIIGLIIHGGIFLPLIYFVVTRKNPFSFFAGIFQAWITALGTASSAGTLPVTFRCLEENLGIDKRVTRFVLPVGATINMDGTALYEAVAAIFIAQMNGVVLDGGQIVTVSLTATLASVGAASIPSAGLVTMLLILTAVGLPTEDISLLVAVDWLLDRMRTSVNVVGDSFGAGIVYHLSKSELDTIDSQHRVHEDIEMTKTQSIYDDMKNHRESNSNQCVYAAHNSVIVDECKVTLAANGKSADCSVEEEPWKREK

SEQ ID NO: 22 - protein, 524 aa, Homo sapiens
MGKPARKGCEWKRFLKNNWVLLSTVAAVVLGITTGVLVREHSNLSTLEKFYFAFPGEILMRMLKLIILPLIISSMITGVAALDSNVSGKIGLRAVVYYFCTTLIAVILGIVLVVSIKPGVTQKVGEIARTGSTPEVSTVDAMLDLIRNMFPENLVQACFQQYKTKREEVKPPSDPEMNMTEESFTAVMTTAISKNKTKEYKIVGMYSDGINVLGLIVFCLVFGLVIGKMGEKGQILVDFFNALSDATMKIVQIIMCYMPLGILFLIAGKIIEVEDWEIFRKLGLYMATVLTGLAIHSIVILPLIYFIVVRKNPFRFAMGMAQALLTALMISSSSATLPVTFRCAEENNQVDKRITRFVLPVGATINMDGTALYEAVAAVFIAQLNDLDLGIGQIITISITATSASIGAAGVPQAGLVTMVIVLSAVGLPAEDVTLIIAVDWLLDRFRTMVNVLGDAFGTGIVEKLSKKELEQMDVSSEVNIVNPFALESTILDNEDSDTKKSYVNGGFAVDKSDTISFTQTSQF

SEQ ID NO: 23 - protein, 564 aa, Homo sapiens
MSSHGNSLFLRESGQRLGRVGWLQRLQESLQQRALRTRLRLQTMTLEHVLRFLRRNAFILLTVSAVVIGVSLAFALRPYQLTYRQIKYFSFPGELLMRMLQMLVLPLIVSSLVTGMASLDNKATGRMGMRAAVYYMVTTIIAVFIGILMVTIIHPGKGSKEGLHREGRIETIPTADAFMDLIRNMFPPNLVEACFKQFKTQYSTRVVTRTMVRTENGSEPGASMPPPFSVENGTSFLENVTRALGTLQEMLSFEETVPVPGSANGINALGLVVFSVAFGLVIGGMKHKGRVLRDFFDSLNEAIMRLVGIIIWYAPVGILFLIAGKILEMEDMAVLGGQLGMYTLTVIVGLFLHAGIVLPLIYFLVTHRNPFPFIGGMLQALITAMGTSSSSATLPITFRCLEEGLGVDRRITRFVLPVGATVNMDGTALYEALAAIFIAQVNNYELNLGQITTISITATAASVGAAGIPQAGLVTMVIVLTSVGLPTEDITLIIAVDWFLDRLRTMTNVLGDSIGAAVIEHLSQRELELQEAELTLPSLGKPYKSLMAQEKGASRGRGGNESAM

SEQ ID NO: 24 - protein, 560 aa, Homo sapiens
MVPHAILARGRDVCRRNGLLILSVLSVIVGCLLGFFLRTRRLSPQEISYFQFPGELLMRMLKMMILPLVVSSLMSGLASLDAKTSSRLGVLTVAYYLWTTFMAVIVGIFMVSIIHPGSAAQKETTEQSGKPIMSSADALLDLIRNMFPANLVEATFKQYRTKTTPVVKSPKVAPEEAPPRRILIYGVQEENGSHVQNFALDLTPPPEVVYKSEPGTSDGMNVLGIVFFSATMGIMLGRMGDSGAPLVSFCQCLNESVMKIVAVAVWYFPFGIVFLIAGKILEMDDPRAVGKKLGFYSVTVVCGLVLHGLFILPLLYFFITKKNPIVFIRGILQALLIALATSSSSATLPITFKCLLENNHIDRRIARFVLPVGATINMDGTALYEAVAAIFIAQVNNYELDFGQIITISITATAASIGAAGIPQAGLVTMVIVLTSVGLPTDDITLIIAVDWALDRFRTMINVLGDALAAGIMAHICRKDFARDTGTEKLLPCETKPVSLQEIVAAQQNGCVKSVAEASELTLGPTCPHHVPVQVEQDEELPAASLNHCTIQISELETNV

SEQ ID NO: 25 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 26 - protein, 504 aa, Homo sapiens
MEAPLQTEMVELVPNGKHSEGLLPVITPMAGNQRVEDPARSCMEGKSFLQKSPSKEPHFTDFEGKTSFGMSVFNLSNAIMGSGILGLAYAMANTGIILFLFLLTAVALLSSYSIHLLLKSSGVVGIRAYEQLGYRAFGTPGKLAAALAITLQNIGAMSSYLYIIKSELPLVIQTFLNLEEKTSDWYMNGNYLVILVSVTIILPLALMRQLGYLGYSSGFSLSCMVFFLIAVIYKKFHVPCPLPPNFNNTTGNFSHVEIVKEKVQLQVEPEASAFCTPSYFTLNSQTAYTIPIMAFAFVCHPEVLPIYTELKDPSKKKMQHISNLSIAVMYIMYFLAALFGYLTFYNGVESELLHTYSKVDPFDVLILCVRVAVLTAVTLTVPIVLFPVRRAIQQMLFPNQEFSWLRHVLIAVGLLTCINLLVIFAPNILGIFGVIGATSAPFLIFIFPAIFYFRIMPTEKEPARSTPKILALCFAMLGFLLMTMSLSFIIIDWASGTSRHGGNH

SEQ ID NO: 27 - protein, 472 aa, Homo sapiens
MELQDPKMNGALPSDAVGYRQEREGFLPSRGPAPGSKPVQFMDFEGKTSFGMSVFNLSNAIMGSGILGLAYAMAHTGVIFFLALLLCIALLSSYSIHLLLTCAGIAGIRAYEQLGQRAFGPAGKVVVATVICLHNVGAMSSYLFIIKSELPLVIGTFLYMDPEGDWFLKGNLLIIIVSVLIILPLALMKHLGYLGYTSGLSLTCMLFFLVSVIYKKFQLGCAIGHNETAMESEALVGLPSQGLNSSCEAQMFTVDSQMSYTVPIMAFAFVCHPEVLPIYTELCRPSKRRMQAVANVSIGAMFCMYGLTATFGYLTFYSSVKAEMLHMYSQKDPLILCVRLAVLLAVTLTVPVVLFPIRRALQQLLFPGKAFSWPRHVAIALILLVLVNVLVICVPTIRDIFGVIGSTSAPSLIFILPSIFYLRIVPSEVEPFLSWPKIQALCFGVLGVLFMAVSLGFMFANWATGQSRMSGH

SEQ ID NO: 28 - protein, 523 aa, Homo sapiens
MAGHTQQPSGRGNPRPAPSPSPVPGTVPGASERVALKKEIGLLSACTIIIGNIIGSGIFISPKGVLEHSGSVGLALFVWVLGGGVTALGSLCYAELGVAIPKSGGDYAYVTEIFGGLAGFLLLWSAVLIMYPTSLAVISMTFSNYVLQPVFPNCIPPTTASRVLSMACLMLLTWVNSSSVRWATRIQDMFTGGKLLALSLIIGVGLLQIFQGHFEELRPSNAFAFWMTPSVGHLALAFLQGSFAFSGWNFLNYVTEEMVDARKNLPRAIFISIPLVTFVYTFTNIAYFTAMSPQELLSSNAVAVTFGEKLLGYFSWVMPVSVALSTFGGINGYLFTYSRLCFSGAREGHLPSLLAMIHVRHCTPIPALLVCCGATAVIMLVGDTYTLINYVSFINYLCYGVTILGLLLLRWRRPALHRPIKVNLLIPVAYLVFWAFLLVFSFISEPMVCGVGVIIILTGVPIFFLGVFWRSKPKCVHRLTESMTHWGQELCFVVYPQDAPEEEENGPCPPSLLPATDKPSKPQ

SEQ ID NO: 29 - protein, 487 aa, Homo sapiens
MGDTGLRKRREDEKSIQSQEPKTTSLQKELGLISGISIIVGTIIGSGIFVSPKSVLSNTEAVGPCLIIWAACGVLATLGALCFAELGTMITKSGGEYPYLMEAYGPIPAYLFSWASLIVIKPTSFAIICLSFSEYVCAPFYVGCKPPQIVVKCLAAAAILFISTVNSLSVRLGSYVQNIFTAAKLVIVAIIIISGLVLLAQGNTKNFDNSFEGAQLSVGAISLAFYNGLWAYDGWNQLNYITEELRNPYRNLPLAIIIGIPLVTACYILMNVSYFTVMTATELLQSQAVAVTFGDRVLYPASWIVPLFVAFSTIGAANGTCFTAGRLIYVAGREGHMLKVLSYISVRRLTPAPAIIFYGIIATIYIIPGDINSLVNYFSFAAWLFYGLTILGLIVMRFTRKELERPIKVPVVIPVLMTLISVFLVLAPIISKPTWEYLYCVLFILSGLLFYFLFVHYKFGWAQKISKPITMHLQMLMEVVPPEEDPE

SEQ ID NO: 30 - protein, 685 aa, Homo sapiens
MAEDKSKRDSIEMSMKGCQTNNGFVHNEDILEQTPDPGSSTDNLKHSTRGILGSQEPDFKGVQPYAGMPKEVLFQFSGQARYRIPREILFWLTVASVLVLIAATIAIIALSPKCLDWWQEGPMYQIYPRSFKDSNKDGNGDLKGIQDKLDYITALNIKTVWITSFYKSSLKDFRYGVEDFREVDPIFGTMEDFENLVAAIHDKGLKLIIDFIPNHTSDKHIWFQLSRTRTGKYTDYYIWHDCTHENGKTIPPNNWLSVYGNSSWHFDEVRNQCYFHQFMKEQPDLNFRNPDVQEEIKEILRFWLTKGVDGFSLDAVKFLLEAKHLRDEIQVNKTQIPDTVTQYSELYHDFTTTQVGMHDIVRSFRQTMDQYSTEPGRYRFMGTEAYAESIDRTVMYYGLPFIQEADFPFNNYLSMLDTVSGNSVYEVITSWMENMPEGKWPNWMIGGPDSSRLTSRLGNQYVNVMNMLLFTLPGTPITYYGEEIGMGNIVAANLNESYDINTLRSKSPMQWDNSSNAGFSEASNTWLPTNSDYHTVNVDVQKTQPRSALKLYQDLSLLHANELLLNRGWFCHLRNDSHYVVYTRELDGIDRIFIVVLNFGESTLLNLHNMISGLPAKMRIRLSTNSADKGSKVDTSGIFLDKGEGLIFEHNTKNLLHRQTAFRDRCFVSNRACYSSVLNILYTSC

SEQ ID NO: 31 - protein, 476 aa, Homo sapiens
MSTQRLRNEDYHDYSSTDVSPEESPSEGLNNLSSPGSYQRFGQSNSTTWFQTLIHLLKGNIGTGLLGLPLAVKNAGIVMGPISLLIIGIVAVHCMGILVKCAHHFCRRLNKSFVDYGDTVMYGLESSPCSWLRNHAHWGRRVVDFFLIVTQLGFCCVYFVFLADNFKQVIEAANGTTNNCHNNETVILTPTMDSRLYMLSFLPFLVLLVFIRNLRALSIFSLLANITMLVSLVMIYQFIVQRIPDPSHLPLVAPWKTYPLFFGTAIFSFEGIGMVLPLENKMKDPRKFPLILYLGMVIVTILYISLGCLGYLQFGANIQGSITLNLPNCWLYQSVKLLYSIGIFFTYALQFYVPAEIIIPFFVSRAPEHCELVVDLFVRTVLVCLTCILAILIPRLDLVISLVGSVSSSALALIIPPLLEVTTFYSEGMSPLTIFKDALISILGFVGFVVGTYEALYELIQPSNAPIFINSTCAFI

SEQ ID NO: 32 - protein, 483 aa, Homo sapiens
MSVTKSTEGPQGAVAIKLDLMSPPESAKKLENKDSTFLDESPSESAGLKKTKGITVFQALIHLVKGNMGTGILGLPLAVKNAGILMGPLSLLVMGFIACHCMHILVKCAQRFCKRLNKPFMDYGDTVMHGLEANPNAWLQNHAHWGRHIVSFFLIITQLGFCCVYIVFLADNLKQVVEAVNSTTNNCYSNETVILTPTMDSRLYMLSFLPFLVLLVLIRNLRILTIFSMLANISMLVSLVIIIQYITQEIPDPSRLPLVASWKTYPLFFGTAIFSFESIGVVLPLENKMKNARHFPAILSLGMSIVTSLYIGMAALGYLRFGDDIKASISLNLPNCWLYQSVKLLYIAGILCTYALQFYVPAEIIIPFAISRVSTRWALPLDLSIRLVMVCLTCLLAILIPRLDLVISLVGSVSGTALALIIPPLLEVTTFYSEGMSPLTIFKDALISILGFVGFVVGTYQALDELLKSEDSHPFSNSTTFVR

SEQ ID NO: 33 - protein, 642 aa, Homo sapiens
MDKLKCPSFFKCREKEKVSASSENFHVGENDENQDRGNWSKKSDYLLSMIGYAVGLGNVWRFPYLTYSNGGGAFLIPYAIMLALAGLPLFFLECSLGQFASLGPVSVWRILPLFQGVGITMVLISIFVTIYYNVIIAYSLYYMFASFQSELPWKNCSSWSDKNCSRSPIVTHCNVSTVNKGIQEIIQMNKSWVDINNFTCINGSEIYQPGQLPSEQYWNKVALQRSSGMNETGVIVWYLALCLLLAWLIVGAALFKGIKSSGKVVYFTALFPYVVLLILLVRGATLEGASKGISYYIGAQSNFTKLKEAEVWKDAATQIFYSLSVAWGGLVALSSYNKFKNNCFSDAIVVCLTNCLTSVFAGFAIFSILGHMAHISGKEVSQVVKSGFDLAFIAYPEALAQLPGGPFWSILFFFMLLTLGLDSQFASIETITTTIQDLFPKVMKKMRVPITLGCCLVLFLLGLVCVTQAGIYWVHLIDHFCAGWGILIAAILELVGIIWIYGGNRFIEDTEMMIGAKRWIFWLWWRACWFVITPILLIAIFIWSLVQFHRPNYGAIPYPDWGVALGWCMIVFCIIWIPIMAIIKIIQAKGNIFQRLISCCRPASNWGPYLEQHRGERYKDMVDPKKEADHEIPTVSGSRKPE

SEQ ID NO: 34 - protein, 511 aa, Homo sapiens
MVDSTEYEVASQPEVETSPLGDGASPGPEQVKLKKEISLLNGVCLIVGNMIGSGIFVSPKGVLIYSASFGLSLVIWAVGGLFSVFGALCYAELGTTIKKSGASYAYILEAFGGFLAFIRLWTSLLIIEPTSQAIIAITFANYMVQPLFPSCFAPYAASRLLAAACICLLTFINCAYVKWGTLVQDIFTYAKVLALIAVIVAGIVRLGQGASTHFENSFEGSSFAVGDIALALYSALFSYSGWDTLNYVTEEIKNPERNLPLSIGISMPIVTIIYILTNVAYYTVLDMRDILASDAVAVTFADQIFGIFNWIIPLSVALSCFGGLNASIVAASRLFFVGSREGHLPDAICMIHVERFTPVPSLLFNGIMALIYLCVEDIFQLINYYSFSYWFFVGLSIVGQLYLRWKEPDRPRPLKLSVFFPIVFCLCTIFLVAVPLYSDTINSLIGIAIALSGLPFYFLIIRVPEHKRPLYLRRIVGSATRYLQVLCMSVAAEMDLEDGGEMPKQRDPKSN

SEQ ID NO: 35 - protein, 515 aa, Homo sapiens
MEAREPGRPTPTYHLVPNTSQSQVEEDVSSPPQRSSETMQLKKEISLLNGVSLVVGNMIGSGIFVSPKGVLVHTASYGMSLIVWAIGGLFSVVGALCYAELGTTITKSGASYAYILEAFGGFIAFIRLWVSLLVVEPTGQAIIAITFANYIIQPSFPSCDPPYLACRLLAAACICLLTFVNCAYVKWGTRVQDTFTYAKVVALIAIIVMGLVKLCQGHSEHFQDAFEGSSWDMGNLSLALYSALFSYSGWDTLNFVTEEIKNPERNLPLAIGISMPIVTLIYILTNVAYYTVLNISDVLSSDAVAVTFADQTFGMFSWTIPIAVALSCFGGLNASIFASSRLFFVGSREGHLPDLLSMIHIERFTPIPALLFNCTMALIYLIVEDVFQLINYFSFSYWFFVGLSVVGQLYLRWKEPKRPRPLKLSVFFPIVFCICSVFLVIVPLFTDTINSLIGIGIALSGVPFYFMGVYLPESRRPLFIRNVLAAITRGTQQLCFCVLTELDVAEEKKDERKTD

SEQ ID NO: 36 - skipped

SEQ ID NO: 37 - protein, 397 aa, Pseudomonas putida
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKAS

SEQ ID NO: 38 - protein, 394 aa, Fusobacterium nucleatum
MEMKKSGLGTTAIHAGTLKNLYGTLAMPIYQTSTFIFDSAEQGGRRFALEEAGYIYTRLGNPTTTVLENKIAALEEGEAGIAMSSGMGAISSTLWTVLKAGDHVVTDKTLYGCTFALMNHGLTRFGVEVTFVDTSNLEEVKNAMKKNTRVVYLETPANPNLKIVDLEALSKIAHTNPNTLVIVDNTFATPYMQKPLKLGVDIVVHSATKYLNGHGDVIAGLVVTRQELADQIRFVGLKDMTGAVLGPQEAYYIIRGLKTFEIRMERHCKNARTIVDFLNKHPKVEKVYYPGLETHPGYEIAKKQMKDFGAMISFELKGGFEAGKTLLNNLKLCSLAVSLGDTETLIQHPASMTHSPYTKEEREVAGITDGLVRLSVGLENVEDIIADLEQGLEK

SEQ ID NO: 39 - protein, 452 aa, Streptomyces ambofaciens
MSQPIDSVTAGHTPEGAPAEPDAPGWSFETRQLHAGAAPDTATGARATPIYQTTSFVFQDTQHAADLFSLAEPGNIYTRIHNPTQDVFEQRVASLEGGVAAVALASGQAAETLAVLTLAGAGDHIVSSASLYGGTYNLFRHTLPRFGIEVSFVDDPDDIEAWRAAIRPGTKALFAETLGNPRGNVLDVRAVADVAHEAGVPLIVDNTVPTPYLLRPIEHGADIVVHSATKFLGGHGTTIGGVVVDGGTFDFGAHAERFPGFTEPDPSYHGLRYWPALGPGAFAVKLRVQLLRDLGPALSPHSAFLLLQGVETLSLRIERHSANAQALAEWLEQRDEVAAVHYPGLPSNRWYDAGQRYLPRGGGAVLAFELRDGVKAGKRFVDAVELFSHLANIGDVRSLIIHPASTTHSQLDEEQLAATGASPGLVRLSVGIENLADLKADLEAGFRAAKGA

SEQ ID NO: 40 - protein, 420 aa, Clostridium saccharobutylicum
MKIETKCLHEGYKPQNGEPVALPIYQSITYRYDNADEIGKLFDLTSSGHMYSRISNPTVAAVEEKIAALEGGVAALCTTSGQAAALISILNILSAGDHIVSVSTIYGGTINLFAVTLKKFGIQCTFVDAEASEEEIQKAFKANTKVVFGETIANPAIAIFDIEKFANIAHKNNVPLIIDNTFATPILCRPIEFGADIVIHSTSKYMDGHAVQIGGVIVDSGKFDWTNGKFPEFTEPDASYHGIVYTEKFGSAAYITKARVQLMRDLGAYPSANAAFLLNLGLESLPVRIEKHCRNAEAVAEFLNSNDKVEFVNYPTIKCNKYNDLAKKYLPNGCSGVISFSIKGDRDDAIRFMNSLKLIENVVHVADIRTSVLHPASSTHRQLTDEQLVAAGITPGLIRISVGLENIDDIIEDIKQALEQ

SEQ ID NO: 41 - protein, 390 aa, Bacillus mycoides
MKKKHMETALIHHGYTSEEHKGSLTPPLFQTSTFTFETAQQGEASFAGVDPSYIYSRLGNPTVKLFEERMAVLEGGEEALAFGSGMAAISATLIGFLKAGDHIICSNGLYGCTYGFLEVLEEKFMITHSFCDMETETDIENKIRPNTKLIFVETPINPTMKLIDLEKVIGVAKRNDLLVIVDNTFCSPYLQRPLELGCDAVVHSATKYIGGHGDVVAGVTICKTKALAEKIRPMRKDIGGIMAPFDAWLLLRGLKTLAVRMDRHCDNAEKIVSFLKNHDAVEGVWYPEGELASRQMKRGGGVISFSIKGGKEETQAFINDLYFITIAVSLGDTETLIQHPATMTHAAIPADLRKEMGIYDNLIRLSVGLESWEDIVSDLEQALKKISTVN

SEQ ID NO: 42 - protein, 429 aa, Bordetella trematum
MSDSNKNWRLETIAVHGGYRPDPTTRAVAVPIYQTVAYAFDDTQHGADLFDLKVPGNIYTRIMNPTNDVLEQRVAALEGGIAALALASGQAAVTYSILTLAEAGDNIVSSSTLYGGTYNLLAHTLPQYGISTRFADPSDLASFERQIDARTKAIFAESVGNPLGNVTDIQALADLAHRHGVPLIVDNTVPSPYLLRPIEHGADIVVQSLTKYLGGHGTSLGGAIIDSGKFPWAQHKERFKRLNEPDVSYHGVVYTEAFGAAAYIGRARVVPLRNTGAALSPFNAFQILQGIETLALRVDRIVENAVRIAQYLKQHPKVEWVNYAGLPEHPDHALVQKYLGGKAPGLFTFGVKGGREAGARFQDALNLFTRLVNIGDAKSLATHPASTTHRQLNPEELAKAGVREETVRLSIGIEHIDDLIADLDQALAQ

SEQ ID NO: 43 - protein, 398 aa, Citrobacter freundii
MSDCRTYGFNTQIVHAGQQPDPSTGALSTPIFQTSTFVFDSAEQGAARFALEESGYIYTRLGNPTTDALEKKLAVLERGEAGLATASGISAITTTLLTLCQQGDHIVSASAIYGCTHAFLSHSMPKFGINVRLVDAGKPEEIRAAMRPETKVVYIETPANPTLSLVDIETVAGIAHQQGALLVVDNTFMSPYCQQPLQLGADIVVHSVTKYINGHGDVIGGIIVGKQEFIDQARFVGLKDITGGCMSPFNAWLTLRGVKTLGIRMERHCENALKIARFLEGHPSITRVYYPGLSSHPQYELGQRQMSLPGGIISFEIAGGLEAGRRMINSVELCLLAVSLGDTETLIQHPASMTHSPVAPEERLKAGITDGLIRLSVGLEDPEDIINDLEHAIRKATF

SEQ ID NO: 44 - protein, 389 aa, Entamoeba histolytica [sequence omitted]

SEQ ID NO: 45 - protein, 424 aa, Yersinia frederiksenii
MKLETLSIHAGYSPDPTTKAVAVPIYQTSSFAFDDTQHGADLFDLKVAGNIYSRIMNPTNDVLEKRVAALEGGIAALAVASGMAAITYAIQTIAEVGDNIVSVAKLYGGTYNLLAHTLPRYGIETRFADHDDIAALEALIDDRTKAVFCESIGNPAGNIVDLKKLADAAHRHGVPLIVDNTVATPILCRPFEHGADIVVHSLTKYIGGHGSSIGGIVVDSGKFPWAQYPERFAQLNTPDPSYHGVTYTEQFGAAAYIGRCRVVPLRNTGAALSPFNAFLILQGLETLALRMERHTENALKVAHYLQNHPQVSWVKYAGLPDHPEHELAQRYFGGKPASILSFGIHGGQEAGGRFIDALNLIVRLVNIGDAKSLACHPASTTHRQLNDEELIKAGVPRDMVRLSIGIEHIDDILNDLAQALDAAK

SEQ ID NO: 46 - protein, 431 aa, Bacillus subtilis
MGESWGKGTICVQGGYTPKNGEPRVLPLYQSTTYKYDTSDDLAALFNLEAEGYIYTRIGNPTLAAFEQKLAELEGGVGAVATASGQAAIMLAVLNICSSGDHLLCSSTVYGGTFNLFGVSLRKLGIDVTFFNPNLTADEIVALANDKTKLVYAESLGNPAMNVLNFKEFSDAAKELEVPFIVDNTLATPYLCQAFEHGANIIVHSTTKYIDGHASSLGGIVIDGGNFDWTNGKYPELVEPDPSYHGVSYVQNFGAAAYIVKARVQLLRDYGNCMSPFNAYISNIGLETLHLRMERHSENALAVAKWLADHERIEWVNYPGLDSNENYSLAQKYLKKGASGVLTFGIKGGLEAAKEFIANVKLATLVTHVADARTCVIHPASTTHRQLSAEDQRLAGVTSDLIRLSVGIEDVSDIIADLEAALVGGKQHADH

SEQ ID NO: 47 - protein, 397 aa, Pseudomonas putida
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGHTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKAS

SEQ ID NO: 48 - protein, 563 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRWAPTQKEAGIYPRKGKQTRQQQEHQKYMVPLQASAQEKNGL

SEQ ID NO: 49 - protein, 515 aa, Homo sapiens
MVLSQEEPDSARGTSEAQPLGPAPTGAAPPPGPGPSDSPEAAVEKVEVELAGPATAEPHEPPEPPEGGWGWLVMLAAMWCNGSVFGIQNACGVLFVSMLETFGSKDDDKMVFKTAWVGSLSMGMIFFCCPIVSVFTDLFGCRKTAVVGAAVGFVGLMSSSFVSSIEPLYLTYGIIFACGCSFAYQPSLVILGHYFKKRLGLVNGIVTAGSSVFTILLPLLLRVLIDSVGLFYTLRVLCIFMFVLFLAGFTYRPLATSTKDKESGGSGSSLFSRKKFSPPKKIFNFAIFKVTAYAVWAVGIPLALFGYFVPYVHLMKHVNERFQDEKNKEVVLMCIGVTSGVGRLLFGRIADYVPGVKKVYLQVLSFFFIGLMSMMIPLCSIFGALIAVCLIMGLFDGCFISIMAPIAFELVGAQDVSQAIGFLLGFMSIPMTVGPPIAGLLRDKLGSYDVAFYLAGVPPLIGGAVLCFIPWIHSKKQREISKTTGKEKMEKMLENQNSLLSSSSGMFKKESDSII

SEQ ID NO: 50 - protein, 405 aa, Homo sapiens
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 51 - protein, 398 aa, Mus musculus
MQKDASLSGFLPSFQHFATQAIHVGQEPEQWNSRAVVLPISLATTFKQDFPGQSSGFEYSRSGNPTRNCLEKAVAALDGAKHSLAFASGLAATITITHLLKAGDEIICMDEVYGGTNRYFRRVASEFGLKISFVDCSKTKLLEAAITPQTKLVWIETPTNPTLKLADIGACAQIVHKRGDIILVVDNTFMSAYFQRPLALGADICMCSATKYMNGHSDVVMGLVSVNSDDLNSRLRFLQNSLGAVPSPFDCYLCCRGLKTLQVRMEKHFKNGMAVARFLETNPRVEKVVYPGLPSHPQHELAKRQCSGCPGMVSFYIKGALQHAKAFLKNLKLFTLAESLGGYESLAELPAIMTHASVPEKDRATLGINDTLIRLSVGLEDEQDLLEDLDRALKAAHP

SEQ ID NO: 52 - protein, 398 aa, Rattus norvegicus
MQKDASSSGFLPSFQHFATQAIHVGQEPEQWSSRAVVLPISLATTFKQDSPGQSSGFVYSRSGNPTRNCLEKAVAALDGAKHCLTFASGLAATTTITHLLKAGDEVICMDEVYGGTNRYFRRVASEFGLKISFVDCSKTKLLEAAITPQTKLVWIETPTNPTLKLADIKACAQIVHKHKDIILVVDNTFMSAYFQRPLALGADICMCSATKYMNGHSDVVMGLVSVNSDDLNERLRFLQNSLGAVPSPFDCYLCCRGLKTLQIRMEKHFRNGMAVARFLESNPRVEKVIYPGLPSHPQHELAKRQCTGCPGMVSFYIKGTLQHAQVFLKNIKLFALAESLGGYESLAELPAIMTHASVPEKDRATLGISDTLIRLSVGLEDEKDLLEDLGQALKAAHP

SEQ ID NO: 53 - protein, 392 aa, Saccharomyces cerevisiae
MTLQESDKFATKAIHAGEHVDVHGSVIEPISLSTTFKQSSPANPIGTYEYSRSQNPNRENLERAVAALENAQYGLAFSSGSATTATILQSLPQGSHAVSIGDVYGGTHRYFTKVANAHGVETSFTNDLLNDLPQLIKENTKLVWIETPTNPTLKVTDIQKVADLIKKHAAGQDVILVVDNTFLSPYISNPLNFGADIVVHSATKYINGHSDVVLGVLATNNKPLYERLQFLQNAIGAIPPFDAWLTHRGLKTLHLRVRQAALSANKIAEFLAADKENVVAVNYPGLKTHPNYDVVLKQHRDALGGGMISFRIKGGAEAASKFASSTRLFTLAESLGGIESLLEVPAVMTHGGIPKEARASGVFDDLVRISVGIEDTDDLLEDIKQALKQATN

SEQ ID NO: 54 - protein, 417 aa, Neurospora crassa
MTASSDHPITTPPRAPSPVHGFGTLAVHAGSPHDPATGAVIEAISLSTTFAQTAVGKPVGEFEYSRSSNPNRANFEKMVAALEHAKYALAYSSGSATTANILQSLAAGSHVISVSDVYGGTHRYFTQVAKAHGVKVTFTPEIEVDIRDHITDATKLVWIETPSNPTLRLVDIRAVATAAHERGILVVVDNTFLSPYVQNPLDHGADIVVHSVTKYINGHSDVVMGVAAFNSDELYARLSFLQNAIGAVPSAFDSWLAHRGAKTLHLRAREATTNATAIAHALEASPLVISVNYPGLESHPHRAIALKQHRNGMGGGMLSFRIHGGHAAAEKFCQYTKIFTLAESLGGVESLCEIPSSMTHAGIPKAQREAVGIFDDLVRISCGVEDAEDLKADVLQALERAVADAANGVSNGVNGTH

SEQ ID NO: 55 - protein, 389 aa, Leishmania major
MSSQQHLVSDFTAGSGSWLPQSQGFDTLQVHAGVRPDPVTGAILTPIYQSTTFVQESINSYQAKGYSYTRSANPTVAVLEQKLCALENGSYCTVYNTGMAATTTAISSFMNAGDHAILTNCCYGGTNRACRVFFSRLGMEFTFVDMRDPQNVIDSIKPNTKLVISETPANPTLILIDVAAVSKICKERGIVHMCDNTFATAYIMRPLDHGADVTLISTTKYVDGHDMTVGGALVTNSKELDAKVRLTQNILGNVMSPQVAFLQLQTVKTMSLRVTKQSHNAQKIAEFLETHRAVDRVVYPGGMLWFEVKGGTAAGRRLMDTVPRPWSLCENLGASESIITCPSVMTHANMTSEDRMKVGITDGFVRVSCGIEDVDDLIAALKVAMDALV

SEQ ID NO: 56 - protein, 393 aa, Corynebacterium ammoniagenes
MKFPTQDELRARFTRKWTQFEPDVLPLFIAESDFPTAPVIKEVLLDYTERECFGYSPAPGTVDLGGAVSEFHAKRYGWRPDPKKVFWIGDVVRGLLLGIQYFTRPDSPVVVPLPAYPPFLELPETAGRKRIDVGVSPANGDLKEGEKPRLDLESIERAFADGAGSILLANPYNPLGYVFDEEHLHELVELADKYDARILSDEIHAPLVLDGQHISVAGLSDAAARVTMTITATSKAWNFAGLKCAQIIFSNDEDVKTWNSLPHVAQDGVGTLGIIAAEAAYRDGIPHLEEEIEYLRSTRDWLVEELPKRIPGLVTSKPDSTYLLWLDFRNTAIGDKEHPAAWLIQHARVAFNEGVTFGEGGAHQARMNFATSPEILEEAIDRIATAIAKVSDN

SEQ ID NO: 57 - protein, 419 aa, Emericella nidulans
MTMMAGHPDLPSNGQNGDSNTHQQRQFATLAVHAGAPHDPTTGAVIAPISLSTTFAQESVGKPVGLYEYTRSSNPNRDNFEEAVASLEHAKYALAFSSGSATTATILHSLAPGSHVVSVSDVYGGTHRYFTKVAAAHGVNVSFSSCLELDVEKLIRPNETKLVWIETPSNPTLALVDIRKVAAVAHRHGVLVVVDNTFMSPYVQNPLDHGADVVIHSVTKYINGHSDVLMGVAAFNSDELKERFTFLQNAIGAVPSPFDCWLAHRGLKTLHLRAREATANATAVALALESSPHVISVNYPGLNSHPNREIAVKQHRKGMGGGMLSFRIKGGHKAAHLFCEYTKIFTLAESLGGVESLCEVPSSMTHAGIPKEEREAAGVYDDLVRMSCGIEDVEDLTADTMQALERAVAASQALENGSA

SEQ ID NO: 58 - protein, 454 aa, Arabidopsis thaliana
MEAGERRNGDSMSHNHRAPKKPRLAGLLTESDIDSEFAHHQTGVARINNGSFGCCPGSVLEAQREWQLRYLRQPDEFYFNGLRRGLLASRTVISDLINADDVDEVSLVDNATTAAAIVLQKVGRCFSEGKYKKEDTVVMFHCAFQSVKKSIQAYVSRVGGSTVEVRLPFPVNSNEEIISKFREGLEKGRANGRTVRLAIIDHITSMPCVLMPVRELVKICREEGVEQVFVDAAHAIGSVKVDVKEIGADYYVSNLHKWFFCPPSIAFFYCKKRGSESDVHHPVVSHEFGNGLPIESAWIGTRDYSSQLVVPSVMEFVNRFEGGMEGIMMKNHDEAVRMGLMLADAWGTNLGSPPEMCVGMVMIGLPSKLCVGSDEDAIKLRSYLRVHYSVEVPVFYLGLRDGEEGVKDKDSGLITAYVRISHQVYNKTEDYERLRDAITELVKDQMTCQNLPAL

SEQ ID NO: 59 - protein, 470 aa, Homo sapiens
MDRGEKIQLKRVFGYWWGTSFLLINIIGAGIFVSPKGVLAYSCMNVGVSLCVWAGCAILAMTSTLCSAEISISFPCSGAQYYFLKRYFGSTVAFLNLWTSLFLGSGVVAGQALLLAEYSIQPFFPSCSVPKLPKKCLALAMLWIVGILTSRGVKEVTWLQIASSVLKVSILSFISLTGVVFLIRGKKENVERFQNAFDAELPDISHLIQAIFQGYFAYSGGACFTLIAGELKKPRTTIPKCIFTALPLVTVVYLLVNISYLTVLTPREILSSDAVAITWADRAFPSLAWIMPFAISTSLFSNLLISIFKSSRPIYLASQEGQLPLLFNTLNSHSSPFTAVLLLVTLGSLAIILTSLIDLINYIFFTGSLWSILLMIGILRRRYQEPNLSIPYKVFLSFPLATIVIDVGLVVIPLVKSPNVHYVYVLLLVLSGLLFYIPLIHFKIRLAWFEKMTCYLQLLFNICLPDVSEE

SEQ ID NO: 60 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 61 - protein, 602 aa, Homo sapiens
MAALAAAAKKVWSARRLLVLLFTPLALLPVVFALPPKEGRCLFVILLMAVYWCTEALPLSVTALLPIVLFPFMGILPSNKVCPQYFLDTNFLFLSGLIMASAIEEWNLHRRIALKILMLVGVQPARLILGMMVTTSFLSMWLSNTASTAMMLPIANAILKSLFGQKEVRKDPSQESEENTAAVRRNGLHTVPTEMQFLASTEAKDHPGETEVPLDLPADSRKEDEYRRNIWKGFLISIPYSASIGGTATLTGTAPNLILLGQLKSFFPQCDVVNFGSWFIFAFPLMLLFLLAGWLWISFLYGGLSFRGWRKNKSEIRTNAEDRARAVIREEYQNLGPIKFAEQAVFILFCMFAILLFTRDPKFIPGWASLFNPGFLSDAVTGVAIVTILFFFPSQRPSLKWWFDFKAPNTETEPLLTWKKAQETVPWNIILLLGGGFAMAKGCEESGLSVWIGGQLHPLENVPPALAVLLITVVIAFFTEFASNTATIIIFLPVLAELAIRLRVHPLYLMIPGTVGCSFAFMLPVSTPPNSIAFASGHLLVKDMVRTGLLMNLMGVLLLSLAMNTWAQTIFQLGTFPDWADMYSVNVTALPPTLANDTFRTL

SEQ ID NO: 62 - DNA, 54 bp, Thosea asigna virus
gagggcagaggaagtcttctaacatgcggtgacgtggaggsgsstcccggccct

SEQ ID NO: 63 - protein, 15 aa, Artificial Sequence
  Description of Artificial Sequence: Synthetic peptide
GGGGSGGGGSGGGGS

SEQ ID NO: 64 - protein, 9 aa, Artificial Sequence
  Description of Artificial Sequence: Synthetic peptide
YPYDVPDYA

SEQ ID NO: 65 - protein, 10 aa, Artificial Sequence
  Description of Artificial Sequence: Synthetic peptide
EQKLISEEDL

SEQ ID NO: 66 - protein, 8 aa, Artificial Sequence
  Description of Artificial Sequence: Synthetic peptide
DYKDDDDK

SEQ ID NO: 67 - protein, 5 aa, Artificial Sequence
  Description of Artificial Sequence: Synthetic peptide
  MOD_RES (3)..(3): Any amino acid
LPXTG

SEQ ID NO: 68 - protein, 5 aa, Artificial Sequence
  Description of Artificial Sequence: Synthetic peptide
  MOD_RES (3)..(3): Any amino acid
LPXTA

SEQ ID NO: 69 - protein, 8 aa, Artificial Sequence
  Description of Artificial Sequence: Synthetic peptide
GGSGGSGG

SEQ ID NO: 70 - protein, 405 aa, Pongo abelii
MQEKEASSQGFLPHFQHFATQAIHVGQEPEQWTSRAVVPPISPSVTFKQGAPGQHSGFVYSRSGNPTRNCLEKAVAALDGAKYCLAFASGLAATVTITHLLKAGDQIICMDDVYAGTNRYFRQVASEFGLKISFVDCSKIKLLEAAITPETKLVWIETPTNPTQKMTDIEACAHIVHKHGDIILVVDNTFMSPYFQRPLALGADICMCSATKYMNGHSDVVMGLVSVNCESLYNRLRFLQNSLGAVPSPIDCYLCNRGLKTLQVRMEKHFKNGMAVAQFLESNPWVEKVIYPGLPSHPQHELVKRQCTGCTGMVTFYIKGTLQHAEIFLKNLKLFTLAESLGGFESLVELPAVMTHASVLKKDRDVLGISDTLIRLSVGLEDEEDLLEDLDQALKAAHPPSGSHS

SEQ ID NO: 71 - protein, 405 aa, Macaca fascicularis
MQEKDASSQGFLPHFQHFATQAIHVGQEPEQWTSRAVVPLISLSTTFKQAAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPVLKMIDIEACAHIVHKRGDIILVVDNTFMSPYFQRPLALGADICMCSATKYMNGHSDVVMGLVSVNCERLHNRLRFLQNSLGAVPSPLDCYLCNRGLKTLHVRMEKHFKNGMAVAQFLESNPGVEKVIYPGLPSHPQHELAKRQCTGCTGMITFYIKGTLQHAEIFLKNLKLFTLAESLGGFESLVELPAIMTHASVPKNDRDVLGISDTLIRLSVGLEDEKDLLEDLDQALKAAHPPSGSHN

SEQ ID NO: 72 - protein, 405 aa, Pan troglodytes
MQEKDASSQGFLPHFQHFATQAIHVGQDPEQWTSRALVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 73 - protein, 405 aa, Pan paniscus
MQEKDASSQGFLPHFQHFATQAIHVGQDPEQWTSKALVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVIGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 74 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRSGNPTRNCLEKAVAALDGAKYCLAFASGLAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

SEQ ID NO: 75 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 76 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 77 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 78 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 79 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRSGNPTRNCLEKAVAALDGAKYCLAFASGLAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFDLAVSLGGFESLAELPAIMTHASVLKNDRDVLGISDTLIRLSVGLEDEEDLLEDLDQALKAAHPPSGSHS

SEQ ID NO: 80 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFDLAVSLGGFESLAELPASMTHASVLKNDRDVLGISDTLIRLSVGLEDEEDLLEDLDQALKAAHPPSGSHS

SEQ ID NO: 81 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFDLAVSLGGFESLAELPASMTHASVLKNDRDVLGISDTLIRLSVGLEDEEDLLEDLDQALKAAHPPSGSHS

SEQ ID NO: 82 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFDLAVSLGGFESLAELPASMTHASVLKNDRDVLGISDTLIRLSVGLEDEEDLLEDLDQALKAAHPPSGSHS

SEQ ID NO: 83 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFDLAVSLGGFESLAELPASMTHASVLKNDRDVLGISDTLIRLSVGLEDEEDLLEDLDQALKAAHPPSGSHS

SEQ ID NO: 84 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRSGNPTRNCLEKAVAALDGAKYCLAFASGLAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFELAVSLGGFESLAELPAIMTHASVLKNDRDVLGISDTLIRLSVGLEDEEDLLEDLDQALKAAHPPSGSHS

SEQ ID NO: 85 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFELAVSLGGFESLAELPASMTHASVLKNDRDVLGISDTLIRLSVGLEDEEDLLEDLDQALKAAHPPSGSHS

SEQ ID NO: 86 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFELAVSLGGFESLAELPASMTHASVLKNDRDVLGISDTLIRLSVGLEDEEDLLEDLDQALKAAHPPSGSHS

SEQ ID NO: 87 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFNYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFELAVSLGGFESLAELPASMTHASVLKNDRDVLGISDTLIRLSVGLEDEEDLLEDLDQALKAAHPPSGSHS

SEQ ID NO: 88 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFELAVSLGGFESLAELPASMTHASVLKNDRDVLGISDTLIRLSVGLEDEEDLLEDLDQALKAAHPPSGSHS

SEQ ID NO: 89 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 90 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNHYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 91 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNGYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 92 - protein, 405 aa, Artificial Sequence [sequence omitted]

SEQ ID NO: 93 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLENSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 94 - protein, 405 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFEYSRSGNPIRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLENSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 95 - protein, 10 aa, Artificial Sequence
  Description of Artificial Sequence: Synthetic peptide
  MOD_RES (4)..(5): Any amino acid
GGGXXGGGGS

SEQ ID NO: 96 - protein, 9 aa, Artificial Sequence
  Description of Artificial Sequence: Synthetic peptide
GGGGSGGGG

SEQ ID NO: 97 - protein, 10 aa, Artificial Sequence
  Description of Artificial Sequence: Synthetic peptide
GGGGSGGGGS

SEQ ID NO: 98 - protein, 10 aa, Artificial Sequence
  Description of Artificial Sequence: Synthetic peptide
GSGSGSGSGS

SEQ ID NO: 99 - protein, 7 aa, Artificial Sequence
  Description of Artificial Sequence: Synthetic peptide
PSTSTST

SEQ ID NO: 100 - protein, 7 aa, Artificial Sequence
  Description of Artificial Sequence: Synthetic peptide
EIDKPSQ

SEQ ID NO: 101 - protein, 19 aa, Artificial Sequence
  Description of Artificial Sequence: Synthetic peptide
MYGKIIFVLLLSEIVSISA

SEQ ID NO: 102 - protein, 131 aa, Artificial Sequence
  Description of Artificial Sequence: Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 103 - protein, 78 aa, Artificial Sequence
  Description of Artificial Sequence: Synthetic polypeptide
MQPQESHVHYSRWEDGSRDGVSLGAVSSTEEASRCRRISQRLCTGKLGIAMKVLGGVALFWIIFILGYLTGYYVHKCK

SEQ ID NO: 104 - protein, 582 aa, Artificial Sequence
  Description of Artificial Sequence: Synthetic polypeptide
MYGKIIFVLLLSEIVSISAMQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGGSGGGGYPYDVPDYAGGGGSGGGGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 105 - protein, 563 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGGSGGGGYPYDVPDYAGGGGSGGGGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 106 - protein, 582 aa, Artificial Sequence
  Description of Artificial Sequence: Synthetic polypeptide
MYGKIIFVLLLSEIVSISAMQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGGSGGGGYPYDVPDYAGGGGSGGGGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 107 - protein, 563 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGGSGGGGYPYDVPDYAGGGGSGGGGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 108 - protein, 510 aa, Artificial Sequence
  Description of Artificial Sequence: Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGYPYDVPDYAGGGGSGGGGMQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 109 - protein, 510 aa, Artificial Sequence
  Description of Artificial Sequence: Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGYPYDVPDYAGGGGSGGGGMQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHS

SEQ ID NO: 110 - protein, 421 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHSGGGSGGGYPYDVPDYA

SEQ ID NO: 111 - protein, 421 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNAYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGSGGGYPYDVPDYA

SEQ ID NO: 112 - protein, 421 aa, Artificial Sequence
  Description of Artificial Sequence: Synthetic polypeptide
MQEKDASSQGFLPHFQHFATQAIHVGQDPEQWTSRAVVPPISLSTTFKQGAPGQHSGFIYSRLGNPTRNCLEKAVAALDGAKYCLAFASGMAATVTITHLLKAGDQIICMDDVYGGTNDYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMERHFKNGMAVAQFLESNPWVEKVIYPGLPSHPQHELVKRQCTGCGGMVTFYIKGTLQHAEIFLKNLKLFTLAVSLGGFESLAELPASMTHASVLKNDRDVLGISDTLIRLSVGLEDEEDLLEDLDQALKAAHPPSGSHSGGGSGGGYPYDVPDYA